(19) United States
(12) Patent Application Publication
(10) Pub. No.: US 2015/0266939 A1
(43) Pub. Date: Sep. 24, 2015

(54) CELL PENETRATING COMPOSITIONS FOR DELIVERY OF INTRACELLULAR ANTIBODIES AND ANTIBODY-LIKE MOIETIES AND METHODS OF USE

(71) Applicant: Permeon Biologics, Inc., Cambridge, MA (US)

(72) Inventors: Erik M. Vogan, Medford, MA (US); Alex Franzusoff, Nahant, MA (US); John Edwards, Singer Island, FL (US); Ann DeWitt, Cambridge, MA (US)

(21) Appl. No.: 14/385,072

(22) PCT Filed: Mar. 15, 2013
(86) PCT No.: PCT/US13/32686

§ 371 Date: Sep. 12, 2014

Related U.S. Application Data
(60) Provisional application No. 61/611,493, filed on Mar. 15, 2012.

(51) Int. Cl. C07K 14/50; C07K 14/435; C07K 16/18

(52) U.S. Cl. CPC C07K 14/50; C07K 16/18; C07K 14/43595; C07K 2317/622; C07K 2319/10

(57) ABSTRACT

The disclosure relates to a complex comprising a Surf+ Penetrating Polypeptide and an AAM moiety for intracellular delivery, and methods of use.

### CROSS-REFERENCE TO RELATED APPLICATION

This application claims the benefit of U.S. Provisional Application 61/611,493, filed Mar. 15, 2012, the entire contents of which are incorporated herein by reference

### BACKGROUND OF THE DISCLOSURE

The effectiveness of an agent intended for use as a therapeutic, diagnostic, or in other applications is often highly dependent on its ability to penetrate cellular membranes or tissues to access a target and/or induce a desired change in biological activity. Although many therapeutic drugs, diagnostic or other product candidates, whether protein, nucleic acid, small organic molecule, or small inorganic molecule, show promising biological activity in vitro, many fail to reach or penetrate target cells to achieve the desired effect, often due to physiochemical properties that result in inadequate biodistribution in vivo. Adequate delivery into a cell or cellular compartment of interest is a particularly acute problem for larger molecules, such as antibodies and antibody-like moieties.

In general, absent a specific receptor-mediated mechanism, proteins, such as antibodies, do not penetrate cells well. It is of great interest for protein-based therapeutics, diagnostics and biological assays to identify methods and compositions that facilitate delivery of polypeptides into a cell.

### SUMMARY OF THE DISCLOSURE

The present disclosure provides compositions and methods for delivering antibodies and antibody-mimic moieties (referred to herein as “AAM moieties” or “an AAM moiety”) into a cell. Without being bound by theory, the present disclosure is based, at least in part, on the discovery that an AAM moiety can be delivered into a cell by complexing the AAM moiety with a cell penetrating polypeptide having surface positive charge (referred to herein as a “Surf+ Penetrating Polypeptide”). The present disclosure is exemplary of the important applications of Intraphilin technology. Also provided are complexes, as well as methods for making and using such complexes comprising a Surf+ Penetrating Polypeptide portion and an AAM moiety portion.

In one aspect, the disclosure provides a complex comprising a Surf+ Penetrating Polypeptide and an AAM moiety that binds an intracellular target. In certain embodiments, the AAM moiety binds to an intracellular target distinct from the Surf+ Penetrating Polypeptide. In other words, the the target of the AAM moiety is not the Surf+ Penetrating Polypeptide to which that AAM moiety is complexed to.

In a related aspect, the disclosure provides a complex comprising (or consisting of) a first portion comprising a Surf+ Penetrating Polypeptide and a second portion comprising an AAM moiety that binds an intracellular target. In certain embodiments, the AAM moiety binds to an intracellular target distinct from the Surf+ Penetrating Polypeptide. In other words, the the target of the AAM moiety is not the the Surf+ Penetrating Polypeptide to which that AAM moiety is complexed to.

In another aspect, the disclosure provides a fusion protein comprising a Surf+ Penetrating Polypeptide and an AAM moiety that binds an intracellular target. In a related aspect, the disclosure provides a fusion protein comprising a first polypeptide portion comprising a Surf+ Penetrating Polypeptide and a second polypeptide portion comprising an AAM moiety that binds to an intracellular target. In some embodiments, the fusion protein is a single polypeptide chain.

In another aspect, the disclosure provides a complex comprising (a) a polypeptide selected from the group consisting of: agouti-signaling protein precursor, band 3 anion transport protein, B-cell lymphoma 6 protein isoform 1, BCL2/adenovirus E1B 19 kDa protein-interacting protein 3, beta-defensin 1 preproprotein, cathepsin E isoform a preproprotein, charged multivesicular body protein 6, cpG-binding protein isoform 2, C-X-C motif chemokine 10 precursor, epidermal growth factor receptor isoform a precursor, histone acetyltransferase MYST3, histone acetyltransferase p300, homeobox protein Nkx-3.1, lethal(3)malignant brain tumor-like protein 2, male-specific lethal 3 homolog isoform a, Na(+)/H(+) exchange regulatory cofactor NHE-RF1, peptidyl-prolyl cis-trans isomerase NIMA-interacting 1, peptidyl-prolyl cis-trans isomerase NIMA-interacting 1, POU domain class 2-associating factor 1, prostatic acid phosphatase isoform PAP precursor, receptor tyrosine-protein kinase erbB-2 isoform b, receptor tyrosine-protein kinase erbB-3 isoform 1 precursor, receptor tyrosine-protein kinase erbB-4 isoform JM-a/CVT-2 precursor, RING1 and YY1-binding protein, sterol regulatory element-binding protein 2, stromal cell-derived factor 1 isoform gamma, talin-1, T-cell surface glycoprotein CD4 isoform 1 precursor, transcription factor AP-1, transcription factor NF-E2 45 kDa subunit isoform 2, transcription factor Sp1 isoform b, voltage-dependent L-type calcium channel subunit alpha-1C isoform 23, zinc finger protein 224, zinc finger protein 268 isoform c, zinc finger protein 28 homolog, zinc finger protein 32, zinc finger protein 347 isoform a, zinc finger protein 347 isoform b, and zinc finger protein 40 and (b) an AAM moiety. In certain embodiments, the AAM moiety binds to an intracellular target distinct from the polypeptide associated with the AAM moiety in said complex and/or the complex is a fusion protein. In other words, the the target of the AAM moiety is not the the Surf+ Penetrating Polypeptide to which that AAM moiety is complexed to. Complexes and fusion proteins include, in certain embodiments, a single polypeptide chain.

In another aspect, the disclosure provides a complex comprising (a) a polypeptide selected from the group consisting of: agouti-signaling protein precursor, band 3 anion transport protein, B-cell lymphoma 6 protein isoform 1, BCL2/adenovirus E1B 19 kDa protein-interacting protein 3, beta-defensin 1 preproprotein, cathepsin E isoform a preproprotein, charged multivesicular body protein 6, cpG-binding protein isoform 2, C-X-C motif chemokine 10 precursor, epidermal growth factor receptor isoform a precursor, histone acetyltransferase MYST3, histone acetyltransferase p300, homeobox protein Nkx-3.1, lethal(3)malignant brain tumor-like protein 2, male-specific lethal 3 homolog isoform a, Na(+)/H(+) exchange regulatory cofactor NHE-RF1, peptidyl-prolyl cis-trans isomerase NIMA-interacting 1, peptidyl-prolyl cis-trans isomerase NIMA-interacting 1, POU domain class 2-associating factor 1, prostatic acid phosphatase isoform PAP precursor, receptor tyrosine-protein kinase erbB-2 isoform b, receptor tyrosine-protein kinase erbB-3 isoform 1 precursor, receptor tyrosine-protein kinase erbB-4 isoform JM-a/CVT-2 precursor, RING1 and YY1-binding protein, sterol regulatory element-binding protein 2, stromal cell-derived factor 1 isoform gamma, talin-1, T-cell surface glycoprotein CD4 isoform 1 precursor, transcription factor AP-1, transcription factor NF-E2 45 kDa subunit isoform 2, transcription factor Sp1 isoform b, voltage-dependent L-type calcium channel subunit alpha-1C isoform 23, zinc finger protein 224, zinc finger protein 268 isoform c, zinc finger protein 28 homolog, zinc finger protein 32, zinc finger protein 347 isoform a, zinc finger protein 347 isoform b, or zinc finger protein 40, or a domain of any of the foregoing having surface positive charge, a mass of at least 4 kDa and a charge/molecular weight ratio of at least 0.75 and (b) an AAM moiety. In certain embodiments, the AAM moiety binds to an intracellular target distinct from the polypeptide associated with the AAM moiety in said complex and/or the complex is a fusion protein. In other words, the target of the AAM moiety is not the the Surf+ Penetrating Polypeptide to which that AAM moiety is complexed to. Complexes and fusion proteins include, in certain embodiments, a single polypeptide chain.

In another aspect, the disclosure provides a complex comprising (a) a polypeptide comprising an amino acid sequence at least 85%, 90%, 95%, 96%, 97%, 98%, 99%, or 100& identical to any of the amino acid sequences set forth in Section 2 of the sequence listing and identified in such sequence listing by PDB identifier, or a domain thereof having surface positive charge, a mass of at least 4 kDa, and a charge/molecular weight ratio of at least 0.75 and (b) an AAM moiety. In certain embodiments, the AAM moiety binds to an intracellular target distinct from the polypeptide associated with the AAM moiety in said complex and/or the complex is a fusion protein. In other words, the target of the AAM moiety is not the the Surf+ Penetrating Polypeptide to which that AAM moiety is complexed to. In certain embodiments, the polypeptide of (a) comprises 1, 2, 3, 4, 5, 6, 7, 8, 9, or 10 amino acid substitutions relative to the sequence of any of the amino acid sequences set forth in Section 2 of the sequence listing and identified in such sequence listing by PDB identifier, or a domain thereof having surface positive charge, a mass of at least 4 kDa, and a charge/molecular weight ratio of at least 0.75. In certain embodiments, the amino acid substitutions are conservative substitutions. In other embodiments, at least half of the substitutions are conservative substitutions. In certain embodiments, the substitutions do not alter the net charge and/or charge/molecular weight of the polypeptide. In certain embodiments, the substitutions are intended to supercharge the polypeptide. Complexes and fusion proteins include, in certain embodiments, a single polypeptide chain.

In another aspect, the disclosure provides a complex comprising (a) a polypeptide comprising an amino acid sequence at least 85%, 90%, 92%, 95%, 96%, 97%, 98%, 99%, or 100% identical to any of the amino acid sequences set forth in Section 1 of the sequence listing and identified in such sequence listing by GenBank accession number, or a domain thereof having surface positive charge, a mass of at least 4 kDa, and a charge/molecular weight ratio of at least 0.75 and (b) an AAM moiety. In certain embodiments, the AAM moiety binds to an intracellular target distinct from the polypeptide associated with the AAM moiety in said complex and/or the complex is a fusion protein. In other words, the target of the AAM moiety is not the the Surf+ Penetrating Polypeptide to which that AAM moiety is complexed to. In certain embodiments, the polypeptide of (a) comprises 1, 2, 3, 4, 5, 6, 7, 8, 9, or 10 amino acid substitutions relative to the sequence any of the amino acid sequences set forth in Section 1 of the sequence listing and identified in such sequence listing by GenBank accession number, or a domain thereof having surface positive charge, a mass of at least 4 kDa, and a charge/molecular weight ratio of at least 0.75. In certain embodiments, the amino acid substitutions are conservative substitutions. In other embodiments, at least half of the substitutions are conservative substitutions. In certain embodiments, the substitutions do not alter the net charge and/or charge/molecular weight of the polypeptide. In certain embodiments, the substitutions are intended to supercharge the polypeptide.

In certain embodiments of any of the foregoing or following aspects or embodiments described herein, the complex comprises a linker (e.g., 1, 2, 3, 4, more than 4 linkers). For example, a linker may interconnect the first and second portions of the complex. Additionally or alternatively a linker may interconnect portions of the AAM moiety, such as a VH and VL domains of an scFv.

In certain embodiments of any of the foregoing or following aspects or embodiments described herein, the Surf+ Penetrating Polypeptide is a human polypeptide. In other embodiments, the Surf+ Penetrating Polypeptide is a non-human polypeptide (e.g., mouse, rat, non-human primate) or is a non-naturally occurring protein or is a prokaryotic protein. In certain embodiments, the Surf+Penetrating Polypeptide is a full-length, naturally occurring human polypeptide. In other embodiments, the Surf+ Penetrating Polypeptide is a domain of a full length, naturally occurring human polypeptide. In certain embodiments, the domain of a full length, naturally occurring human polypeptide has a charge/molecular weight ratio greater than that of the full length, naturally occurring human polypeptide. In other embodiments, the domain has a charge/molecular weight ratio of at least 0.75 but the full length, naturally occurring human polypeptide has a charge/molecular weight ratio of less than 0.75. In still other embodiments, the domain has a charge/molecular weight of at least 0.75 but the full length, naturally occurring polypeptide has a net negative charge. In addition to comparisons based on charge/molecular weight, domains (e.g., fragments have some level of structure) of full length polypeptide may be compared to their full length polypeptide based on differences in net charge (e.g., the domain has a greater or lesser net charge; the domain has a net positive charge where the full length polypeptide has a net negative charge).

In certain embodiments of any of the foregoing or following aspects or embodiments described herein, the Surf+ Penetrating Polypeptide is a domain of a full length, naturally occurring human protein, and the complex does not include the full length, naturally occurring human protein. In other embodiments, the Surf+ Penetrating Polypeptide is a domain of a full length, naturally occurring human protein, and wherein the complex does not include sufficient additional amino acid sequence from said full length, naturally occurring human protein contiguous with said domain such that the charge/molecular weight of the first portion would be less than 0.75.

In certain embodiments of any of the foregoing or following aspects or embodiments described herein, the Surf+ Penetrating Polypeptide is a domain of a full length polypeptide, and the domain is less than or about 300, 250, 200, 175, 150, 140, 130, 125, 120, 110, or less than 100 amino acid residues. In other embodiments, the Surf+ Penetrating Polypeptide is a domain of a full length polypeptide, and the domain is less than or about 90, 80, 75, 70, 65, 60, 55, 50, or 45 amino acid residues. Of course, Surf+ Penetrating Polypeptides have a minimal mass of 4 kDa, and thus a suitable domain for use as a Surf+ Penetrating Polypeptide has a mass of at least 4 kDa. Moreover, Surf Penetrating Polypeptides have surface positive charge and charge/molecular weight ratio of at least 0.75. Thus, suitable domains for use as a Surf+ Penetrating Polypeptide also meet this criteria. Numerous exemplary domains are identified herein.

In certain embodiments of any of the foregoing or following aspects or embodiments described herein, the size of the first portion of a complex of the disclosure can be described. For example, the first portion may be less than or about 500, 450, 400, 350, 300, 250, 200, 175, 150, 140, 130, 125, 120, 110, or less than 100 amino acid residues. In other embodiments, the first portion may be less than or about 90, 80, 75, 70, 65, 60, 55, 50, or 45 amino acid residues. Of course, the first portion of the complex comprises a Surf+ Penetrating Polypeptide. Thus, although additional amino acid residues may be present, a region of the first portion will have the characteristics of a Surf+ Penetrating Polypeptide—even if those characteristics are not applicable when considered over the entire first portion (e.g., the Surf+ Penetrating Polypeptide region of the first portion has a charge/molecular weight ratio of at least 0.75, but the entire first portion does not). It should be noted that the foregoing sizes are exemplary, and Surf+ Penetrating Polypeptides or first portions that are larger are also contemplated.

In certain embodiments of any of the foregoing or following aspects or embodiments described herein, the Surf+ Penetrating Polypeptide has an endogenous function. For example, in certain embodiments, the Surf+ Penetrating Polypeptide is a polypeptide having endogenous function as a DNA binding protein or is a domain of a full length polypeptide that has endogenous function as a DNA binding protein. In other embodiments, the Surf+ Penetrating Polypeptide is a polypeptide having endogenous function as an RNA binding protein or is a domain of a full length polypeptide, which full length polypeptide has endogenous function as an RNA binding protein. In still other embodiments, Surf+ Penetrating Polypeptide is a polypeptide having endogenous function as a heparin binding protein or is a domain of a full length polypeptide, which full length polypeptide has endogenous function as a heparin binding protein. In other embodiments, the Surf+ Penetrating Polypeptide is a polypeptide having endogenous function as a C-C or C-X-C class of chemokine or is a domain of a full length polypeptide, which full length polypeptide has endogenous function as a C-C or C-X-C class of chemokine.

In certain embodiments of any of the foregoing or following aspects or embodiments described herein, complexes do not include Surf+ Penetrating Polypeptides having certain characteristics, as described in detail herein. For example, in certain embodiments, the Surf+ Penetrating Polypeptide is not an antibody or an antigen binding fragment of an antibody.

In certain embodiments of any of the foregoing or following aspects or embodiments described herein, the AAM for use in a complex is a full length antibody molecule or an antigen binding fragment thereof, or a bispecific antibody or antibody fragment. In other embodiments, the AAM moiety is a camelid antibody, an IgNAR, or an antibody like molecule comprising a target binding domain engineered into an Fc domain of the antibody like molecule. In certain embodiment, the AAM moiety comprises an antibody-mimic comprising a protein scaffold, such as a fibronectin-based scaffold. In certain embodiments, the AAM moiety comprises a DARPin polypeptide, an Adnectin® polypeptide or an Anticalin® polypeptide. In other embodiments, the AAM moiety comprises: a target binding scaffold from Src homology domains (e.g. SH2 or SH3 domains), PDZ domains, beta-lactamase, high affinity protease inhibitors, an EGF-like domain, a Kringle-domain, a PAN domain, a Gla domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, a C-type lectin domain, a MAM domain, a von Willebrand factor type A domain, a Somatomedin B domain, a WAP-type four disulfide core domain, a F5/8 type C domain, a Hemopexin domain, a Laminin-type EGF-like domain, or a C2 domain.

In certain embodiments of any of the foregoing or following aspects or embodiments described herein, the two portions or components of the complex are associated non-covalently. In other embodiments, they are associated covalently. Associations may be direct or via a linker, including via a cleavable linker. The two portions of the complex may be associated via both covalent and non-covalent interactions.

In certain embodiments of any of the foregoing or following aspects or embodiments described herein, the complex is a fusion protein (e.g., the Surf+ Penetrating Polypeptide or portion comprising the Surf+ Penetrating Polypeptide is fused, directly or via a linker, to the AAM moiety or portion comprising the AAM moiety). Suitable fusion proteins include, for example, fusion as a single polypeptide chain.

In certain embodiments of any of the foregoing or following aspects or embodiments described herein, the Surf+ Penetrating Polypeptide has an overall net positive charge of +3, +4, +5, +6, +7, +8, +9, +10, +11, +12, +13, +14, +15, +16, +17, +18, +19, +20, or greater than +20. In other embodiments, the Surf+ Penetrating Polypeptide has an overall net charge of +5 to +17, +4-+10, +3-+8, +5-+14, +7-+15, and the like. Similarly, Surf+ Penetrating Polypeptides with a range of charge/molecular weight ratios, as well as a range of mass are also contemplated. For example, in certain embodiments, the Surf+ Penetrating Polypeptide has a mass of about 4, 5, 6, 7, 8, 9, 10, 11, 12, 13, 14, or about 15 kDa. However, larger Surf+ Penetrating Polypeptides are also contemplated and described herein.

In certain embodiments of any of the foregoing or following aspects or embodiments described herein, the Surf+ Penetrating Polypeptide is a domain of naturally occurring ataxin-7 isoform a, C-C motif chemokine 24 precursor or cytochrome c, which domain has surface positive charge and a charge/molecular weight ratio greater than that of its corresponding naturally occurring, full length polypeptide. An exemplary domain is provided in FIGS. 1 and 2. However, other suitable domains include a small domain of any of those described in FIG. 1 or 2 having a mass of 4 kDa, surface positive charge, and charge/molecular weight ratio of at least 0.75.

In certain embodiments of any of the foregoing or following aspects or embodiments described herein, the Surf+ Penetrating Polypeptide is a naturally occurring protein selected from C-C motif chemokine 24 precursor, beta-defensin 103 precursor, cytochrome c, fibroblast growth factor 10 precursor, signal recognition particle 14 kDa protein, C-X-C chemokine 14 precursor or fibroblast growth factor 8 isoform B precursor, or a domain of any of the foregoing, which domain has surface positive charge and a charge/molecular weight ratio of at least 0.75. An exemplary domain is provided in FIGS. 1 and 2. However, other suitable domains include a small domain of any of those described in FIG. 1 or 2 having a mass of 4 kDa, surface positive charge, and charge/molecular weight ratio of at least 0.75.

In certain embodiments of any of the foregoing or following aspects or embodiments described herein, the Surf+ Penetrating Polypeptide is: a full length polypeptide or a domain of C-C motif chemokine 26 precursor; a domain of HB-EGF (proheparin-binding EGF-like growth factor precursor); a domain of protein DEK isoform 1; a domain of hepatocyte growth factor isoform 1 preprotein; a full length polypeptide or a domain of cytochrome c; a full length polypeptide or domain of C-X-C motif chemokine 24 precursor; or a domain of ataxin 7 isoform a.

In certain embodiments of any of the foregoing or following aspects or embodiments described herein, the Surf+ Penetrating Polypeptide is a domain of any of the following, which domain has a charge per molecular weight ratio of at least 0.75 but for which the corresponding full length naturally occurring polypeptide has a charge/molecular weight ratio of less than 0.75:histone-lysine N-methyltransferase MLL isoform 1 precursor; transcription factor AP-1; proheparin-binding EGF-like growth factor precursor; protein DEK isoform 1; hepatocyte growth factor isoform 1 preprotein; epidermal growth factor receptor isoform a precursor; forkhead box protein K2; pre-mRNA-processing factor 40 homolog A; ataxin-7 isoform a, E3 SUMO-protein ligase PIAS1; platelet factor 4 precursor; advanced glycosylation end product-specific receptor isoform 2 precursor; serol regulatory element-binding protein 2; histone acetyltransferase p300; U1 small nuclear ribonucleoprotein A; pre-B-cell leukemia transcription factor 1 isoform 2; homeobox protein Nkx 3.1; homeobox protein Hox-A9; B-cell lymphoma 6 protein isoform 1; ETS domain-containing protein Elk-4 isoform a; pituitary homeobox 3; granulysin isoform NKG5; general transcription factor IIF subunit 1; histone deacetylase complex subunit SAP30; heterochromatin protein 1-binding protein 3; lethal(3)malignant brain tumor-like protein 2; CCAAT/enhancer-binding protein beta; troponin T, cardiac muscle isoform 2; CREB-binding protein isoform B; cyclic AMP-dependent transcription factor ATF-2; cathepsin E isoform a preprotein; glycine receptior subunit alpha-1 isoform 1 precursor; CREB-binding protein isoform b; pituitary adenylate cyclase-activating polypeptide precursor; mastermind-like protein 1; BCL2/adenovirus E1B 19 kDa protein-interacting protein 3; cathelicidin antimicrobial peptide; epidermal growth factor receptor isoform a precursor; transcription factor NF-E2 45 kDa subunit isoform 2; integrin beta-1 isoform 1D precursor.

In certain embodiments of any of the foregoing or following aspects or embodiments described herein, the Surf+ Penetrating Polypeptide is a domain of charged multivesicular body protein 6; homeobox protein Nkx3.1; B-cell lymphoma 6 protein isoform 1; lethal(3)malignant brain tumor-like protein 2; cathepsin E isoform a preprotein; BCL2/adenovirus E1B 19 kDa protein-interacting protein 3; cathelicidin antimicrobial peptide.

In certain embodiments of any of the foregoing or following aspects or embodiments described herein, the Surf+ Penetrating Polypeptide is a domain of heparin-binding EGF-like growth factor precursor (HBEGF), which domain has surface positive charge and a molecular weight of about 8.9 kDa.

Numerous exemplary domains and full length polypeptides having the structural and functional attributes of a Surf+ Penetrating Polypeptide are provided herein. Similarly, fragments of the expressly exemplified domains having the appropriate functional and structural characteristics of a Surf+ Penetrating Polypeptide are also domains within the scope of the disclosure and suitable for use in a complex of the disclosure.

In certain embodiments of any of the foregoing or following aspects or embodiments described herein, the Surf+ Penetrating Polypeptide is a naturally occurring human polypeptide that is modified to increase its overall net charge (e.g., it is supercharged). For example, the Surf+ Penetrating Polypeptide may be a polypeptide engineered to comprise an overall charge from about +10 to about +40. Supercharging can also be described as the change in charge relative to what it was prior to supercharging. Thus, the disclosure contemplates embodiments in which a polypeptide was supercharged by increasing its net charge from negative to positive, such as by increasing by +3, +4, +5, +6, +7, +8, +9, +10, +11, +12, +13, +14, +15, +20, etc. Alternatively, the disclosure contemplates embodiments in which a polypeptide is supercharged to increase the net charge on an already positively charged polypeptide. For example, supercharging may increase the net charge by +3, +4, +5, +6, +7, +8, +9, +10, +11, +12, +13, +14, +15, +20, etc.

In certain embodiments of any of the foregoing or following aspects or embodiments described herein, the AAM moiety binds to a target and the target is a kinase, a transcription factor, or an oncoprotein. In other embodiments, the AAM moiety binds to a target and the target is NFAT-2, calcineurin, JAK-1, JAK-2, SOCS1, SOCS3, ras or Erk. In certain embodiments, the AAM moiety binds to a target which localizes to a subcompartment of a cell (e.g., nucleus, mitochondria, cytoplasm, or cytoplasmic face of cell membrane.

In certain embodiments of any of the foregoing or following aspects or embodiments described herein, the complex is a fusion protein comprising the Surf+ Penetrating Polypeptide and the AAM moiety, and wherein the Surf+ Penetrating Polypeptide is N-terminal to the AAM moiety. In other embodiments, the complex is a fusion protein comprising the Surf+ Penetrating Polypeptide and the AAM moiety, and wherein the Surf+ Penetrating Polypeptide is C-terminal to the AAM moiety.

In another aspect, the disclosure provides a nucleic acid comprising a nucleotide sequence encoding any of the Surf+ Penetrating Polypeptides disclosed herein, or a nucleotide sequence encoding a polypeptide portion comprising a Surf+ Penetratng Polypeptide disclosed herein. Similarly, the disclosure provides a nucleic acid comprising a nucleotide sequence encoding any of the AAM moieties disclosed herein. Moreover, the disclosure provides a nucleic acid comprising a nucleotide sequence encoding a fusion protein comprising a complex of the disclosure.

In another aspect, the disclosure provides vectors comprising any of the nucleic acids of the disclosure, as well as host cells comprising such vectors, and methods of making polypeptides and complexes.

In another aspect, the disclosure provides methods of delivering an AAM moiety into a cell. The method is applicable to any of the complexes discussed herein. Such a complex is provided, and cells are contacted with the complex. Following such contact, the AAM moiety is delivered into the cell.

Similarly, the disclosure provides methods of inhibiting the activity of an intracellular target in a cell and methods of binding an intracellular target in a cell. Any of the complexes described herein, including complexes formed from any combination of Surf+ Penetrating Polypeptide portions and AAM moiety portions are suitable for use in such methods.

In another aspect, the disclosure provides a composition comprising a complex of the disclosure and a pharmaceutically acceptable carrier. Any of the complexes described herein, including complexes formed from any combination of Surf+ Penetrating Polypeptide portions and AAM moiety portions are suitable for use in such a composition.

In certain embodiments of any of the foregoing or following, a complex of the disclosure can penetrate a cell. Similarly, in certain embodiments, a complex of the disclosure binds to the target via the AAM moiety.

The disclosure contemplates all combinations of any of the foregoing aspects and embodiments, as well as combinations with any of the embodiments set forth in the detailed description and examples.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described herein. The materials, methods, and examples are illustrative only and not intended to be limiting. Other features of the disclosure are apparent from the following detailed description and the claims.

### DESCRIPTION OF THE DRAWINGS

FIG. 1 is table of human polypeptides.

FIG. 2 is a table of a subset of the human polypeptides presented in FIG. 1.

### DETAILED DESCRIPTION OF THE DISCLOSURE

Provided herein are complexes comprising (i) a cell penetrating polypeptide having surface positive charge, called a Surf+ Penetrating Polypeptide, and (ii) an antibody or antibody-mimic molecule, such as a polypeptide comprising a protein scaffold, called an AAM moiety that binds to an intracellular target. Also provided are nucleic acid molecules encoding such protein complexes or encoding the Surf+ Penetrating Polypeptide or AAM moiety portion of such protein complexes, as well as methods of making and using such complexes. Without being bound by theory, the Surf+ Penetrating Polypeptide penetrates cells and, when complexed with the AAM moiety, promotes delivery of the AAM moiety into a cell (e.g., promotes internalization of the AAM moiety into cells). Once inside a cell (e.g., in the cytosol, nucleus, or other cellular compartment), the AAM moiety can bind its intracellularly expressed or localized target molecule and impact cellular activity based on its affect on the target molecule. By way of example, an AAM moiety may bind to an intracellular target, such as a polypeptide or peptide, and alter the activity of the target and/or the activity of the cell via one or more of the following mechanisms (i) inhibit one or more functions of the target; (ii) activate one or more functions of the target; (iii) increase or decrease the activity of the target; (iv) promote or inhibit degradation of the target; (v) change the localization of the target; and (vi) prevent binding between the target and another protein (e.g., prevent binding between the target and a binding partner). Thus, the proteins and complexes described herein are provided for delivery of AAM moieties, e.g., therapeutic, diagnostic and research agents, to cells in vivo, ex vivo, or in vitro.

As described in greater detail herein, the portions of the complexes of the disclosure may be associated via covalent or non-covalent interactions. Exemplary interconnections include fusions (direct or via a linker) via a peptide bond and fusions via chemical methods (direct or via a linker). Moreover, as described in greater detail herein, the association between the two portions of the molecule may persist following internalization into a cell or may be transient. For example, if the two portions of a complex are covalently linked via a cleavable linker, the association may be disrupted after the Surf+ Penetrating Polypeptide portion successfully delivers the AAM moiety into a cell (e.g., once inside the cell, the complex may optionally be disrupted).

This disclosure provides an exemplary application of Intraphilin™ technology in which a member of a class of Surf+ Penetrating Polypeptides is delivered into a cell or is used to deliver a cargo molecule into a cell. In the present application, certain Surf+ Penetrating Polypeptides are complexed with an AAM moiety, and these complexes are useful for delivering the AAM moiety into cells.

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The numbering of amino acids in the variable domain, complementarity determining region (CDRs) and framework regions (FR), of an antibody follow, unless otherwise indicated, the Kabat definition as set forth in Kabat et al. Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insertion (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence. Maximal alignment of framework residues frequently requires the insertion of “spacer” residues in the numbering system, to be used for the Fv region. In addition, the identity of certain individual residues at any given Kabat site number may vary from antibody chain to antibody chain due to interspecies or allelic divergence.

The term “complex of the disclosure” is used to refer to a complex comprising a Surf+ Penetrating Polypeptide portion, such as any of the Surf+ Penetrating Polypeptides described herein, associated with at least one AAM moiety portion. The AAM moiety, which may be an antibody or an antibody-mimic, binds a target expressed or otherwise present in a cell, and the Surf+ Penetrating Polypeptide functions to deliver the AAM moiety into a cell.

As used herein, the terms “antibody” and “antibodies”, also known as immunoglobulins, encompass monoclonal antibodies (including full-length monoclonal antibodies), polyclonal antibodies, multispecific antibodies formed from at least two different epitope binding fragments (e.g., bispecific antibodies), human antibodies, humanized antibodies, camelised antibodies, chimeric antibodies, murine or other non-human antibodies, single-chain Fvs (scFv), Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies of the disclosure), intrabodies, and epitope-binding fragments of any of the above. Immunoglobulins include functional fragments accepted in the art, such as Fc, Fab, scFv, Fv, or other derivatives or combinations of the immunoglobulins, domains of the heavy and light chains of the variable region (such as Fd, Vl, Vk, Vh) and the constant region of an intact antibody such as CH1, CH2, CH3, CH4, Cl and Ck, as well as mini-domains consisting of two beta-strands of an immunoglobulin domain connected by a structural loop. In particular, antibodies include immunoglobulin molecules and immunologically active or other functional fragments of immunoglobulin molecules, i.e., molecules that contain at least one antigen-binding. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m(f, z, a or x), G2m(n), G3m(g, b, or c), Am, Em, and Km(1, 2 or 3)). Antibodies may be derived from any mammal, including, but not limited to, humans, monkeys, pigs, horses, rabbits, dogs, cats, mice, etc., or other animals such as birds (e.g. chickens).

As used herein, the term “about” in the context of a given value or range refers to a value or range that is within 20%, preferably within 10%, and more preferably within 5% of the given value or range.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, the terms “associated with,” or “associate by” when used with respect to the Surf+ Penetrating Polypeptide and AAM moiety portions of a complex of the disclosure, means that these portions are physically associated or connected with one another, either directly or via one or more additional moieties, including moieties that serve as a linking agent, to form a structure that is sufficiently stable so that the AAM moiety is delivered into a cell. The association may be via non-covalent interactions (e.g., electrostatic interactions; affinity or avidity; etc.) and/or via covalent interconnections. In either case, the association may be direct or via a linker moiety or via additional polypeptide sequence. Moreover, the association may be disruptable, such as by cleavage of a linker that interconnects the portions of the complex. The complex may be a fusion protein in which the Surf+ Penetrating Polypeptide portion and the AAM moiety portion are connected by a peptide bond as a fusion protein, either directly or via a linker or other additional polypeptide sequence. In certain embodiments, the fusion protein is a single polypeptide chain. In certain embodiments, the AAM moiety binds to an intracellular target (e.g., a target expressed or present intracellularly) that is distinct from the Surf+ Penetrating Polypeptide present in the complex. In other words, although human Surf+ Penetrating Polypeptides may be expressed endogenously inside a cell, in certain embodiments, the target molecule for the AAM moiety is not a Surf+ Penetrating Polypeptide and/or is not the same Surf+ Penetrating Polypeptide as present in that complex. In certain embodiments, the Surf+ Penetrating Polypeptide portion of a complex of the disclosure is not an antibody or antigen-binding fragment of an antibody. In certain embodiments, the Surf+ Penetrating Polypeptide portion of a complex of the disclosure is not an antibody mimic molecule.

As used herein, the term “supercharge” refers to any modification of a protein, the primary purpose of which is to increase the net charge or the surface charge of the protein to make that protein suitable for or to improve its suitability for use as a Surf+ Penetrating Polypeptide. Modifications include, but are not limited to, alterations in amino acid sequence or addition of positively charged moieties.

### Surf+ Penetrating Polypeptides

A “Surf+ Penetrating Polypeptide”, as used herein, is a polypeptide capable of promoting entry into a cell and having, at least, the following characteristics: mass of at least 4 kDa, charge/molecular weight ratio of at least 0.75, and presence of surface positive charge such that the polypeptide is capable of promoting entry into a cell. The Surf+ Penetrating Polypeptide can itself enter into a cell and/or can be associated with an agent, such as an antibody or antibody mimic, such that it also promotes entry into the cell of the agent. In addition to having surface positive charge, the Surf+ Penetrating Polypeptide has a net positive charge. In certain embodiments, Surf+ Penetrating Polypeptides have a mass of at least 4 kDa and a charge/molecular weight ratio of greater than 0.75. A Surf+ Penetrating Polypeptide may be a human polypeptide, including a full length, naturally occurring human polypeptide or a variant of a full length, naturally occurring human polypeptide having one or more amino acid additions, deletions, or substitutions. Moreover, such human polypeptides include domains of full length naturally occurring human polypeptides or a variant of such a domain having one or more amino acid additions, deletions, or substitutions. For the avoidance of doubt, the term “human polypeptide” includes domains (e.g., structural and functional fragments) unless otherwise specified. Further, Surf+ Penetrating Polypeptides include human or non-human proteins engineered to have one or more regions of surface positive charge and a charge/molecular weight ratio of at least 0.75, including supercharged polypeptides. The present disclosure provides numerous examples of Surf+ Penetrating Polypeptides, as well as numerous examples of sub-categories of Surf+ Penetrating Polypeptides. The disclosure contemplates that any of the sub-categories of Surf+ Penetrating Polypeptides, as well as any of the specific polypeptides described herein may be provided as part of a complex comprising an AAM moiety. Moreover, any such complexes may be used to deliver an AAM moiety into a cell.

In the present context, a “variant of a human polypeptide” is a polypeptide that differs from a naturally occurring (full length or domain) human polypeptide by one or more amino acid substitutions, additions or deletions. In certain embodiments, these changes in amino acid sequence may be to increase the overall net charge of the polypeptide and/or to increase the surface charge of the polypeptide (e.g., to supercharge a polypeptide). Alternatively, changes in amino acid sequence may be for other purposes, such as to provide a suitable site for pegylation or to facilitate production. Regardless of the specific changes made, the variant of the human polypeptide will be sufficiently similar based on sequence and/or structure to its naturally occurring human polypeptide such that the variant is more closely related to the naturally occurring human protein than it is to a protein from a non-human organism. In certain embodiments, the amino acid sequence of the variant is at least 80%, 85%, 90%, 95%, 97%, 98%, or 99% identical to a naturally occurring human protein. In certain embodiments, the variant of the naturally occurring human polypeptide is a Surf+ Penetrating Polypeptide having cell penetrating activity and a charge/molecular weight ratio of at least 0.75 or of greater than 0.75, but the naturally occurring human polypeptide from which the variant is derived does not have cell penetrating activity and/or has a charge/molecular weight ratio of less than 0.75. In certain embodiments, the variant does not result in further supercharging of the polypeptide. For example, the variant results in a change in amino acid sequence but not a change in the net charge, surface charge and/or charge/molecular weight ratio of the polypeptide.

In certain embodiments, the Surf+ Penetrating Polypeptide is a human polypeptide having surface positive charge, mass of at least 4 kDa and charge/molecular weight ratio of at least 0.75 or of greater than 0.75. Such a human polypeptide may be a naturally occurring human polypeptide (which may also be a fragment of a naturally occurring human polypeptide), or a variant thereof having one or more amino acid additions, substitutions, deletions, such as additions, substitutions or deletions that increase (or that do not change) surface positive charge, charge/molecular weight ratio or net positive charge.

In certain embodiments, the Surf+ Penetrating Polypeptide is a human polypeptide that is a domain of a naturally occurring human polypeptide. In addition to having surface positive charge and the ability to penetrate cells, the domain of a naturally occurring human polypeptide has a mass of at least 4 kDa and a charge/molecular weight ratio of at least 0.75 or of greater than 0.75. In certain embodiments, the Surf+ Penetrating Polypeptide for use in the disclosure is a domain of a naturally occurring human polypeptide that has a charge/molecular weight ratio of at least 0.75 or of greater than 0.75, but the corresponding, full length, naturally occurring human protein has a charge/molecular weight ratio of less than 0.75. Additionally or alternatively, in certain embodiments, such a domain has an overall net positive charge greater than that of the corresponding, full length, naturally occurring human protein.

In certain embodiments, a Surf+ Penetrating Polypeptide has a mass of at least 4, 5, 6, 10, 20, 50, 100, 200 kDa or 250 kDa. For example, a Surf+ Penetrating Polypeptide may have a mass of about 4, 5, 6, 7, 8, 9, 10, 11, 12, 13, 14, 15, 16, 17, 18, 19, 20, 21, 22, 23, 24, 25, 26, 27 or 28 kDa. By way of another example, a Surf+ Penetrating Polypeptide may have a mass of about 4-30 kDa, about 5-25 kDa, about 4-20 kDa, about 5-18 kDa, about 5-15 kDa, about 4-12 kDa, about 5-10 kDa, and the like. In still other embodiments, the molecular weight of a Surf+ Penetrating Polypeptide (e.g., a naturally occurring or modified Surf+ Penetrating Polypeptide protein) ranges from approximately 5 kDa to approximately 250 kDa, such as 10 to 250 kDa, 50 to 250 kDa, or 50 to 100 kDa. For example, in certain embodiments, the molecular weight of the Surf+ Penetrating Polypeptide ranges from approximately 4 kDa to approximately 100 kDa. In certain embodiments, the molecular weight of the Surf+ Penetrating Polypeptide ranges from approximately 10 kDa to approximately 45 kDa. In certain embodiments, the molecular weight of the Surf+ Penetrating Polypeptide ranges from approximately 5 kDa to approximately 50 kDa. In certain embodiments, the molecular weight of the Surf+ Penetrating Polypeptide ranges from approximately 5 kDa to approximately 27 kDa. In certain embodiments, the molecular weight of the Surf+ Penetrating Polypeptide ranges from approximately 10 kDa to approximately 60 kDa. In certain embodiments, the molecular weight of the Surf+ Penetrating Polypeptide is about 5 kD, about 7.5 kDa, about 10 kDa, about 12.5 kDa, about 15 kDa, about 17.5 kDa, about 20 kDa, about 22.5 kDa, about 25 kDa, about 27.5 kDa, about 30 kDa, about 32.5 kDa, or about 35 kDa. It should be understood that the mass of the Surf+ Penetrating Polypeptide, including the minimal mass of 4 kDa, refers to monomer mass. However, in certain embodiments, a Surf+ Penetrating Polypeptide for use as part of a complex is a dimer, trimer, tetramer, or a higher order multimer.

In certain embodiments, a Surf+ Penetrating Polypeptide for use in the present disclosure is selected to minimize the number of disulfide bonds. In other words, the Surf+ Penetrating Polypeptide may have not more than 2 or 3 or 4 disulfide bonds (e.g., the polypeptide has 0, 1, 2, 3 or 4 disulfide bonds). A Surf+ Penetrating Polypeptide for use in the present disclosure may also be selected to minimize the number of cysteines. In other words, the Surf+ Penetrating Polypeptide may have not more than 2 cysteines, or not more than 4 cysteines, not more than 6 cysteines or not more than 8 cysteines (e.g., 0, 1, 2, 3, 4, 5, 6, 7, 8 cysteines). A Surf+ Penetrating Polypeptide for use in the present disclosure may also be selected to minimize glycosylation sites. In other words, the polypeptide may have not more than 1 or 2 or 3 glycosylation sites (e.g., N-linked or O-linked glycosylation; 0, 1, 2 or 3 sites).

As defined above, a Surf+ Penetrating Polypeptide has surface positive charge. The Surf+ Penetrating Polypeptide also has an overall net positive charge under physiological conditions. Note that when the Surf+ Penetrating Polypeptide is a domain of a naturally occurring polypeptide, the overall net positive charge is that of the domain. For example, in certain embodiments, the Surf+ Penetrating Polypeptide has an overall net positive charge of at least +4, +5, +10, +15, +20, +25, +30, +35, +40, or +50. By way of further example, a Surf+ Penetrating Polypeptide may have an overall net positive charge of about +4, +5, +6, +7, +8, +9, +10, +11, +12, +13, +14, +15, +16, +17, +18, +19, +20, +21, +22, +23, +24, +25, or greater than +25. In certain embodiments, under physiological conditions, the Surf+ Penetrating Polypeptide has a pI greater than or equal to 9, such as a pI of about 9 to about 13 or a pI of between 9 and 13 (inclusive or exclusive). In other embodiments, under physiological conditions, the Surf+ Penetrating Polypeptide has a pI greater than 9 or greater than 9.5, but less than 10. In other embodiments, under physiological conditions, the Surf+ Penetrating Polypeptide has a pI of about 9-9.5, or about 9-10, or about 9.5-10, or about 10-10.5, or about 10-10.3. In other embodiments, under physiological conditions, the Surf+ Penetrating Polypeptide has a pI of about 10-11, about 10.5-11, about 11-12, about 11.5-12, about 12-13, or about 12.5-13. Note that a Surf+ Penetrating Polypeptide may be a polypeptide that has been modified, such as to increase surface charge and/or overall net positive charge as compared to the unmodified protein, and the modified polypeptide may have increased stability and/or increased cell penetrating ability in comparison to the unmodified polypeptide. In some cases, the modified polypeptide may have cell penetrating ability where the unmodified polypeptide did not.

Theoretical net charge serves as a convenient short hand. In certain embodiments, the theoretical net charge on the Surf+ Penetrating Polypeptide (e.g., the naturally occurring Surf+ Penetrating Polypeptide or the modified Surf+ Penetrating Polypeptide) is at least +1, +2, +3, +4, +5, +6, +7, +8, +9, +10, +11, +12, +13, +14, +15, +16, +17, +18, +19, +20, +21, +22, +23, +24, +25, +30, +35, +40 or +50. In other embodiments, the theoretical net charge on the Surf+ Penetrating Polypeptide (e.g., the naturally occurring Surf+ Penetrating Polypeptide or the modified Surf+ Penetrating Polypeptide) is about +1, +2, +3, +4, +5, +6, +7, +8, +9, +10, +11, +12, +13, +14, +15, +16, +17, +18, +19, +20, +21, +22, +23, +24, +25, +30, +35, +40 or +50. For example, the theoretical net charge on the naturally occurring Surf+ Penetrating Polypeptide can be, e.g., at least +1, at least +2, at least +3, at least +4, at least +5, at least +10, at least +15, at least +20, at least +25, at least +30, at least +35, at least +40 or at least +50 or about +1 to +5, +1 to +10, +5 to +10, +5 to +15, +10 to +20, +15 to +20, +20 to +30, +30 to +40, or +40 to +50 and the like. Note that a Surf+ Penetrating Polypeptide may be a polypeptide that has been modified, such as to increase surface charge and/or overall net positive charge as compared to the unmodified protein, and the modified polypeptide may have increased stability and/or increased cell penetrating ability in comparison to the unmodified polypeptide. In some cases, the modified polypeptide may have cell penetrating ability where the unmodified polypeptide did not.

In certain embodiments, the Surf+ Penetrating Polypeptide has a charge:molecular weight ratio (e.g., also referred to as charge/MW or charge/molecular weight) of at least approximately 0.75, 0.8, 1.0, 1.1, 1.2, 1.3, 1.4, 1.5, 1.6, 1.7, 1.8, 1.9, 2.0, 2.5, or 3.0. This ratio is the ratio of the theoretical net charge of the Surf+ Penetrating Polypeptide to its molecular weight in kilodaltons. In certain embodiments, the charge/molecular weight is about 0.75-2.0. In certain embodiments, the charge/molecular weight ratio of the Surf+ Penetrating Polypeptide is greater than 0.75. In certain embodiments, the Surf+ Penetrating Polypeptide is a domain of a naturally occurring human polypeptide where the domain has a charge/molecular weight ratio of at least 0.75 or of greater than 0.75, but the corresponding full length, naturally occurring human polypeptide has a charge/molecular weight of less than 0.75.

For example, in certain embodiments, the Surf+ Penetrating Polypeptide has a charge:molecular weight ratio of at least approximately 0.75 or of greater than 0.75. In certain embodiments, the Surf+ Penetrating Polypeptide has a charge:molecular weight ratio of at least approximately 0.8. In certain embodiments, the Surf+ Penetrating Polypeptide has a charge:molecular weight ratio of at least approximately 1.0. In certain embodiments, the Surf+ Penetrating Polypeptide has a charge:molecular weight ratio of at least approximately 1.2. In certain embodiments, the Surf+ Penetrating Polypeptide has a charge:molecular weight ratio of at least approximately 1.4. In certain embodiments, the Surf+ Penetrating Polypeptide has a charge:molecular weight ratio of at least approximately 1.5. In certain embodiments, the Surf+ Penetrating Polypeptide has a charge:molecular weight ratio of at least approximately 1.6. In certain embodiments, the Surf+ Penetrating Polypeptide has a charge:molecular weight ratio of at least approximately 1.7. In certain embodiments, the Surf+ Penetrating Polypeptide has a charge:molecular weight ratio of at least approximately 1.8. In certain embodiments, the Surf+ Penetrating Polypeptide has a charge:molecular weight ratio of at least approximately 1.9. In certain embodiments, the Surf+ Penetrating Polypeptide has a charge:molecular weight ratio of at least approximately 2.0. In certain embodiments, the Surf+ Penetrating Polypeptide has a charge:molecular weight ratio of at least approximately 2.5. In certain embodiments, the Surf+ Penetrating Polypeptide has a charge:molecular weight ratio of at least approximately 3.0.

In certain embodiments, the Surf+ Penetrating Polypeptide is a naturally occurring human polypeptide or a domain of a naturally occurring human polypeptide, and it is selected based on the endogenous function of the full length, naturally occurring human polypeptide. By way of example, a Surf+ Penetrating Polypeptide for use in this disclosure may have an endogenous function as, for example, a DNA binding protein, an RNA binding protein or a heparin binding protein. Accordingly, in certain embodiments, the disclosure provides complexes in which the Surf+ Penetrating Polypeptide Portion is (i) a domain of a naturally occurring human polypeptide having a charge/molecular weight ratio of at least 0.75 or of greater than 0.75 but for which its naturally occurring, full length human polypeptide does not have a charge/molecular weight ratio of at least 0.75 and (ii) the domain is from a naturally occurring human polypeptide having an endogenous, natural function as a DNA binding protein, an RNA binding protein or a heparin binding protein. In other embodiments, the Surf+ Penetrating Polypeptide does not have an endogenous function as, for example, a DNA binding protein, an RNA binding protein or a heparin binding protein. In certain embodiments, the Surf+ Penetrating Polypeptide does not have an endogenous function as a histone or histone-like protein. In certain embodiments, the Surf+ Penetrating Polypeptide does not have an endogenous function as a homeodomain containing protein.

In certain embodiments, the Surf+ Penetrating Polypeptide has tertiary structure. The presence of such tertiary structure distinguishes Surf+ Penetrating Polypeptides from unstructured, short cell penetrating peptides (CPPs) such as poly-arginine and poly-lysine and also distinguishes Surf+ Penetrating Polypeptides from cell penetrating peptides that have some secondary structure but no tertiary structure, such as penetratin and antenapedia.

In certain embodiments, the Surf+ Penetrating Polypeptide is not an antibody or an antigen-binding fragment of an antibody. As noted above, Surf+ Penetrating Polypeptides are distinguishable based on numerous characteristics from various short cell penetrating peptides known in the art. For example, Surf+ Penetrating Polypeptides are distinguishable based on size, shape and structure, charge distribution and the like. Moreover, in certain embodiments, Surf+ Penetrating Polypeptides and complexes comprising a Surf+ Penetrating Polypeptide have improved cell penetration characteristics compared to short CPPs or complexes comprises short CPPs. Nevertheless, to provide further clarity, in certain embodiments, complexes of the disclosure do not further include a short CPP. Additional exemplary support is provided herein.

In certain embodiments, a complex of the disclosure and/or the Surf+ Penetrating Polypeptide portion of a complex of the disclosure does not include a full length sequence for HIV-Tat, or the portion thereof known in the art as imparting cell penetration activity. In certain embodiments, a complex of the disclosure and/or the Surf+ Penetrating Polypeptide portion of a complex of the disclosure does not contain the protein transduction domain of HIV-Tat, for example, does not contain the contiguous amino acid sequence YGRKKRRQRRR (SEQ ID NO: 612). In certain embodiments, a complex of the disclosure comprising a Surf+ Penetrating Polypeptide penetrates cells more efficiently than a complex comprising all or a portion of HIV-Tat fused to the same cargo.

In certain embodiments, a complex of the disclosure and/or the Surf+ Penetrating Polypeptide portion of a complex of the disclosure does not include the protein transduction domain of an antennapedia protein, such as the Drosophilia antennapedia protein or a mammalian ortholog thereof. In certain embodiments, a complex of the disclosure and/or the Surf+ Penetrating Polypeptide portion of a complex of the disclosure does not include the protein transduction domain of the h-region of fibroblast growth factor 4 (FGF-4). In other embodiments, a complex of the disclosure and/or the Surf+ Penetrating Polypeptide portion of a complex of the disclosure does not include an FGF polypeptide or a 16 residue cell penetrating polypeptide fragment thereof.

In certain embodiments, a complex of the disclosure and/or the Surf+ Penetrating Polypeptide portion of a complex of the disclosure does not include the 16 amino acid residue sequence referred to as penetratin: RQIKIWFQNRRMKWKK (SEQ ID NO: 613). In certain embodiments, a complex of the disclosure and/or the Surf+ Penetrating Polypeptide portion of a complex of the disclosure does not include the 19 amino acid residue sequence referred to as SynB1: RGGRLSYSRRRFSTSTGRA. In certain embodiments, a complex of the disclosure and/or the Surf+ Penetrating Polypeptide portion of a complex of the disclosure does not include the following amino acid sequence referred to as transportan: GWTLNSAGYLLGKINLKALAALAKKIL (SEQ ID NO: 614).

In certain embodiments, a complex of the disclosure and/or the Surf+ Penetrating Polypeptide portion of a complex of the disclosure does not include the following amino acid sequence RKMLKSTRRQRR. In certain embodiments, a complex of the disclosure and/or the Surf+ Penetrating Polypeptide portion of a complex of the disclosure does not include the amino acid sequence selected from one or more of the following amino acid sequences: YGRKKRRQRRR (SEQ ID NO: 615); WLRRIKAWLRRIKA (SEQ ID NO: 616); WLRRIKAWLRRIKAWLRRIKA (SEQ ID NO: 617); KLALKLALKALKAALKLA (SEQ ID NO: 618); KETWWETWWTEWSQPKKKRKV (SEQ ID NO: 619); AGGGGYGRKKRRQRRR (SEQ ID NO: 620); KETWWETWWTEWSQPKKKRKV (SEQ ID NO: 621); GLWRALWRLLRSLWRLLWKA (SEQ ID NO: 622); GLWRALWRALWRSLWKLKRKV (SEQ ID NO: 623); GLWRALWRALRSLWKLKRKV (SEQ ID NO: 624); GLWRALWRGLRSLWKLKRKV (SEQ ID NO: 625); GLWRALWRGLRSLWKKKRKV (SEQ ID NO: 626); GLWRALWRLLRSLWRLLWKA (SEQ ID NO: 627); GLWRALWRALWRSLWKLKWKV (SEQ ID NO: 628); GLWRALWRALWRSLWKSKRKV (SEQ ID NO: 629); GLWRALWRALWRSLWKKKRKV (SEQ ID NO: 630); GLWRALWRLLRSLWRLLWSQ (SEQ ID NO: 631); TRSSRAGLQFPVGRVHRLLRK (SEQ ID NO: 632); RKKRRRESRKKRRRES (SEQ ID NO: 633); GRPRESGKKRKRKRLKP (SEQ ID NO: 634); GKRKKKGKLGKKRDP (SEQ ID NO: 635); GKRKKKGKLGKKRPRSR (SEQ ID NO: 636); RKKRRRESRRARRSPRHL (SEQ ID NO: 637); SRRARRSPRESGKKRKRKR (SEQ ID NO: 638); VKRGLKLRHVRPRVTRMDV (SEQ ID NO: 639); VKRGLKLRHVRPRVTRDV (SEQ ID NO: 640); SRRARRSPRHLGSG (SEQ ID NO: 641); LRRERQSRLRRERQSR GAYDLRRRERQSRLRRRERQSR (SEQ ID NO: 642); WEAALAEALAEALAEHLAEALAEALEALAA KGSWYSMRKMSMKIRPFFPQQ (SEQ ID NO: 643); KTRYYSMKKTTMKIIPFNRL (SEQ ID NO: 644); RGADYSLRAVRMKIRPLVTQ (SEQ ID NO: 645); LGTYTQDFNKFHTFPQTAIGVGAP (SEQ ID NO: 646); TSPLNIHNGQKL (SEQ ID NO: 647); and NSAAFEDLRVLS (SEQ ID NO: 648)

In certain embodiments, a complex of the disclosure and/or the Surf+ Penetrating Polypeptide portion of a complex of the disclosure does not include HSV-1 structural protein Vp22 (DAATATRGRSAASRPTERPRAPARSASRPRRPVE) (SEQ ID NO: 649). In certain embodiments, a complex of the disclosure and/or the Surf+ Penetrating Polypeptide portion of a complex of the disclosure does not include 9 (or, optionally, does not include 7 or 8) consecutive arginine residues (e.g., poly-Arg9). In other embodiments, a complex of the disclosure and/or the Surf+ Penetrating Polypeptide portion of a complex of the disclosure does not include 9 (or, optionally, does not include 7 or 8) consecutive lysine residues (e.g., poly-Lys9). In certain embodiments, a complex of the disclosure and/or the Surf+ Penetrating Polypeptide portion of a complex of the disclosure does not include the PTD of mouse transcription factor Mph-1 (YARVRRRGPRR) (SEQ ID NO: 650), Sim-2 (AKAARQAAR) (SEQ ID NO: 651), HIV-1 viral protein Tat (YGRKKRRQRRR) (SEQ ID NO: 652), Antennapedia protein (Antp) of Drosophila (RQIKIWFQNRRMKWKK) (SEQ ID NO: 653), MTS (AAVALLPAVLLALLAPAAADQNQLMP) (SEQ ID NO: 654), and short amphipathic peptide carriers Pep-1 (KETWWETWWTEWSQPKKKRKV) (SEQ ID NO: 655) and Pep-2 (KETWFETWFTEWSQPKKKRKV) (SEQ ID NO: 656).

In certain embodiments, the Surf+ Penetrating Polypeptide is not a toxin. In certain embodiments, the Surf+ Penetrating Polypeptide is not a homeodomain. In certain embodiments, a complex of the disclosure and/or the Surf+ Penetrating Polypeptide portion of a complex of the disclosure does not include a homeodomain.

The foregoing provides description for characteristics of Surf+ Penetrating Polypeptides and sub-categories of Surf+ Penetrating Polypeptides. The disclosure contemplates that any Surf+ Penetrating Polypeptide for use in the present disclosure may be described based on presence or absence of any one or any combination of any of the foregoing features. Additional features and specific examples of polypeptides having such features are described in greater detail below. Such features and combinations of features (including combinations with features set forth above) may also be used to describe the Surf+ Penetrating Polypeptide for use in accordance with the claimed disclosure. Any such polypeptides or categories or sub-categories may be used as part of a complex of the disclosure (e.g., the disclosure provides complexes comprising any such polypeptides).

Exemplary Surf+ Penetrating Polypeptides

This section provides examples of Surf+ Penetrating Polypeptides and categories of Surf+ Penetrating Polypeptides.

Surf+ Penetrating Polypeptides that may be used, e.g., in a complex with an AAM moiety and/or to deliver an AAM moiety into a cell as described herein, include nucleic acid binding proteins, e.g., DNA binding proteins, RNA binding proteins or heparin binding proteins. In other words, naturally occurring proteins that can function as Surf+ Penetrating Polypeptides may have a natural, endogenous function, such as an endogenous function as a DNA, RNA or heparin binding protein. In some embodiments, Surf+ Penetrating Polypeptides that may be used in the delivery of an AAM moiety, such as a non-antibody protein scaffold (e.g., an antibody mimic or an antibody-like molecule) or an antibody molecule, can be a DNA binding protein, such as a histone component or a histone-like protein. In certain embodiments, the Surf+ Penetrating Polypeptide portion comprises the histone component is histone linker H1. In certain embodiments, the Surf+ Penetrating Polypeptide portion comprises the histone component is core histone H2A. In certain embodiments, the Surf+ Penetrating Polypeptide portion comprises the histone component is core histone H2B. In certain embodiments, the Surf+ Penetrating Polypeptide portion comprises the histone component is core histone H3. In certain embodiments, the Surf+ Penetrating Polypeptide portion comprises the histone component is core histone H4. In certain embodiments, the the Surf+ Penetrating Polypeptide portion comprises the archael histone-like protein, HPhA. In certain embodiments, the the Surf+ Penetrating Polypeptide portion comprises the bacterial histone-like protein, TmHU. In other embodiments, the Surf+ Penetrating Polypeptide portion does not comprise a protein select from any of the foregoing histone components or histone-like proteins. It should be noted that the foregoing proteins have endogenous, natural function as DNA binding proteins. When used as a Surf+ Penetrating Polypeptide according to the disclosure, the disclosure contemplates the use of human polypeptides, including full length polypeptides and domains of full length polypeptides, regardless of whether the domain with cell penetration function is also a domain that modulates DNA binding activity.

In some embodiments, a Surf+ Penetrating Polypeptide that is used to deliver an AAM moiety, such as a non-antibody protein scaffold (e.g., an antibody mimic or an antibody-like molecule) or an antibody molecule, is an RNA binding protein, such as a ribosomal protein (e.g., L11, S7, S9, or a small nucleolar protein (snoRNP), such as nucleolin, fibrillarin, NOP77P), an RNA polymerase (e.g., RNA polymerase I or II), an RNAse, a transcription factor (e.g., a transcriptional U protein (tUTP)), a histone acetyl transferase (hALP), an upstream binding factor (UBF), a splicing protein (e.g., a snRNP (e.g., U1 or U2) or an SR factor), a La protein, or an hnRNP (heterogeneous ribonuclear protein) (e.g., hnRNP Al, hnRNP M or hnRNP L). In other words, in certain embodiments, the Surf+ Penetrating Polypeptide portion comprises any of the foregoing RNA binding proteins. In other embodiments, the Surf+ Penetrating Polypeptide portion does not comprise a protein select from any of the foregoing RNA binding proteins. It should be noted that the foregoing proteins have endogenous, natural function as RNA binding proteins. When used as a Surf+ Penetrating Polypeptide according to the disclosure, the disclosure contemplates the use of human polypeptides, including full length polypeptides and domains of full length polypeptides, regardless of whether the domain with cell penetration function is also a domain that modulates RNA binding activity.

In certain embodiments, the Surf+ Penetrating Polypeptide portion comprises a naturally occurring polypeptide, such as a naturally occurring human polypeptide. Examples of such naturally occurring polypeptides (and UniProt identification numbers) include, but are not limited to, DEK (ID No.: P35659), HB-EGF (ID No.: Q99075), or c-Jun (ID No.: P05412); HGF (ID No.: P14210); cyclon (ID No.: Q9H6F5); PNRC1 (ID No.: Q12796); RNPS1 (ID No.: Q15287); SURF6 (ID No.: 075683); AR6P (ID No.: Q66PJ3); NKAP (ID No.: Q8N5F7); EBP2 (ID No.: Q99848); LSM11 (ID No.: P83369); RL4 (ID No.: P36578); KRR1 (ID No.: Q13601); RY-1 (ID No.: Q8WVK2); BriX (ID No.: Q8TDN6); MNDA (ID No.: P41218); H1b (ID No.: P16401); cyclin (ID No.: Q9UK58); MDK (ID No.: P21741); Midkine (ID No.: P21741); PROK (ID No.: Q9HC23); FGF5 (ID No.: P12034); SFRS (ID No.: Q8N9Q2); AKIP (ID No.: Q9NWT8); CDK (ID No.: Q8N726); beta-defensin (ID No.: P81534); Defensin 3 (ID No.: P81534); PAVAC (ID No.: P18509); PACAP (ID No.: P18509); eotaxin-3 (ID No.: Q9Y258); histone H2A (ID No.: Q7L7L0); HMGB1 (ID No.: P09429); TERF 1 (ID No.: P54274); PIAS 1 (ID No.: 075925); Ku70 (ID No.: P12956); HRX (ID No: Q03164). In certain embodiments, the complex comprises a Surf+ Penetrating Polypeptide portion comprising one of the following: U4/U6.U5 tri-snRNP-associated protein 3 (ID No.: Q8WVK2); beta-defensin (ID No.: P81534); Protein SFRS121P1 (ID No.: Q8N9Q2); midkine (ID No.: P21741); C-C motif chemokine 26 (ID No.: Q9Y258); surfeit locus protein 6 (ID No.: 075683); Aurora kinase A-interacting protein (ID No.: Q9NWT8); NF-kappa-B-activating protein (ID No.: Q8N5F7); histone H1.5 (ID No.: P16401); histone H2A type 3 (ID No.: Q7L7L0); 60S ribosomal protein L4 (ID No.: P36578); isoform 1 of RNA-binding protein with serine-rich domain 1 (ID No.: Q15287-1); isoform 4 of cyclin-dependent kinase inhibitor 2A (ID No.: Q8N726-1); isoform 1 of prokineticin-2 (ID No.: Q9HC23-1); isoform 1 of ADP-ribosylation factor-like protein 6-interacting protein 4 (ID No.: Q66PJ3-1); isoform long of fibroblast growth factor 5 (ID No.: P12034-1); or isoform 1 of cyclin-L1 (ID No.: Q9UK58-1).

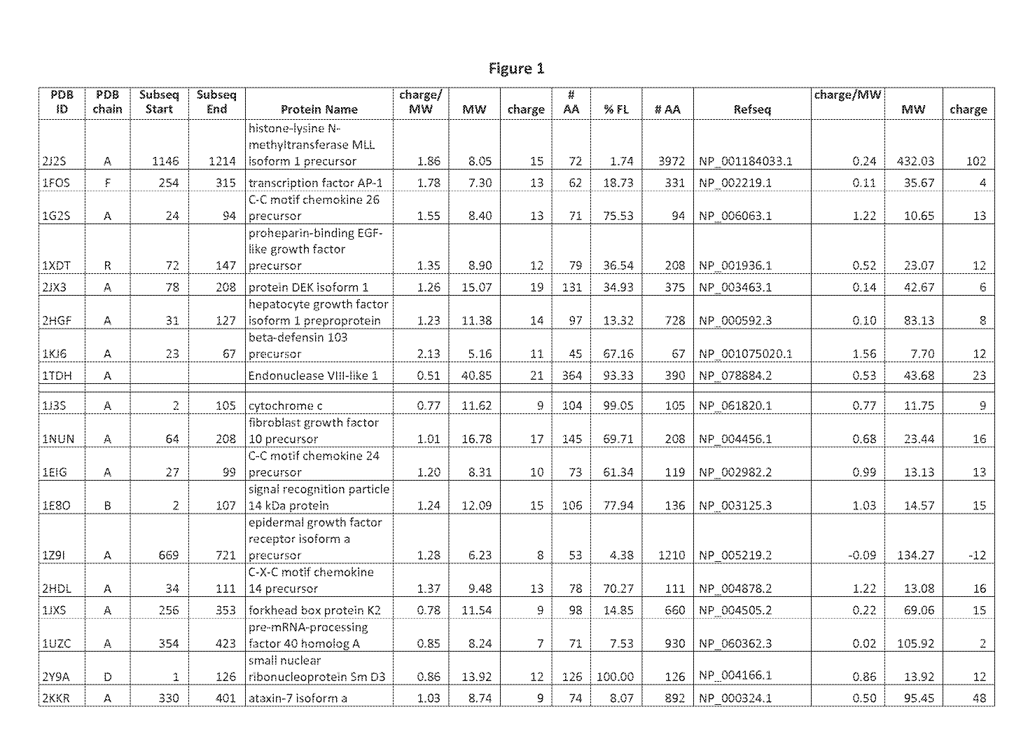

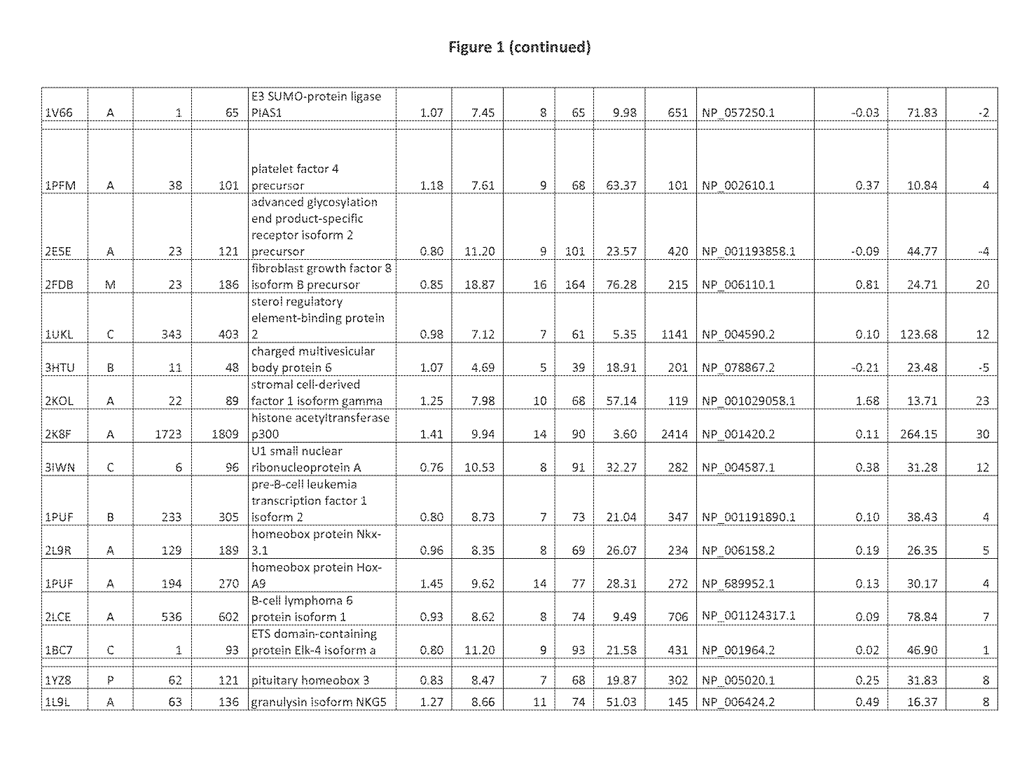

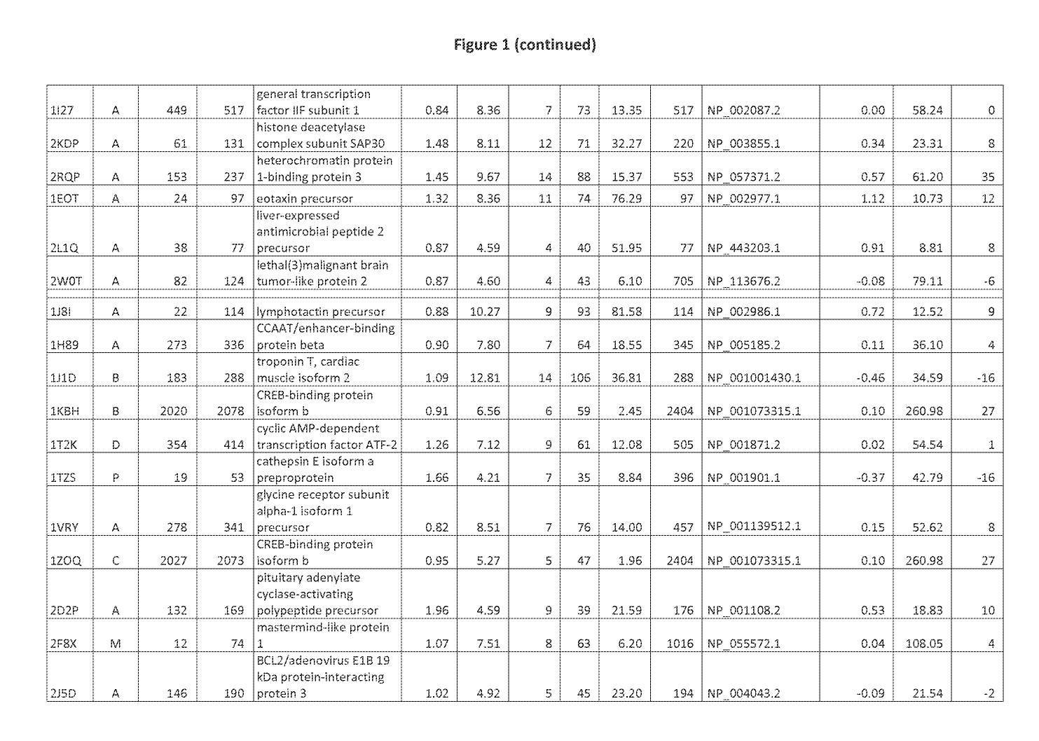

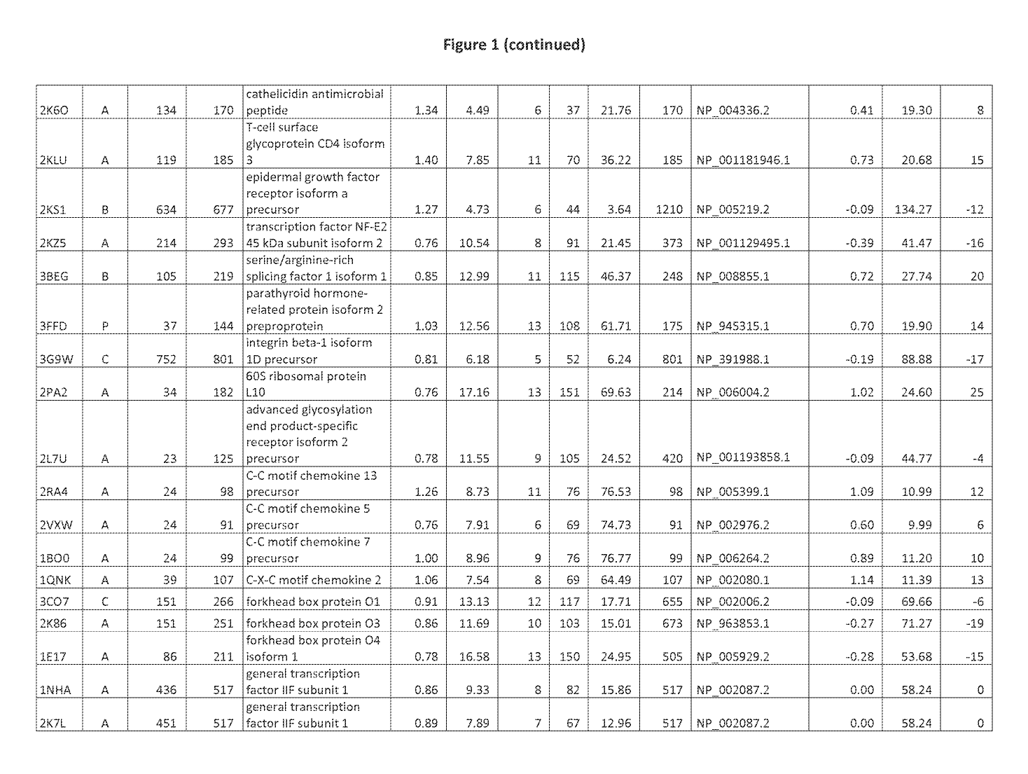

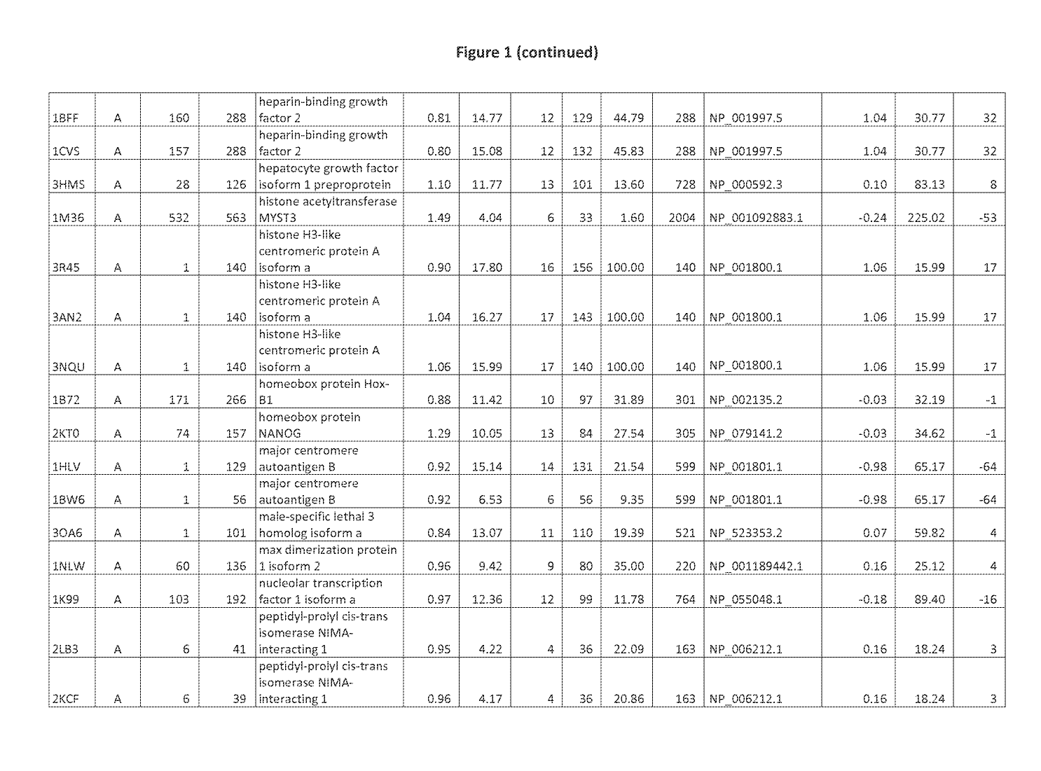

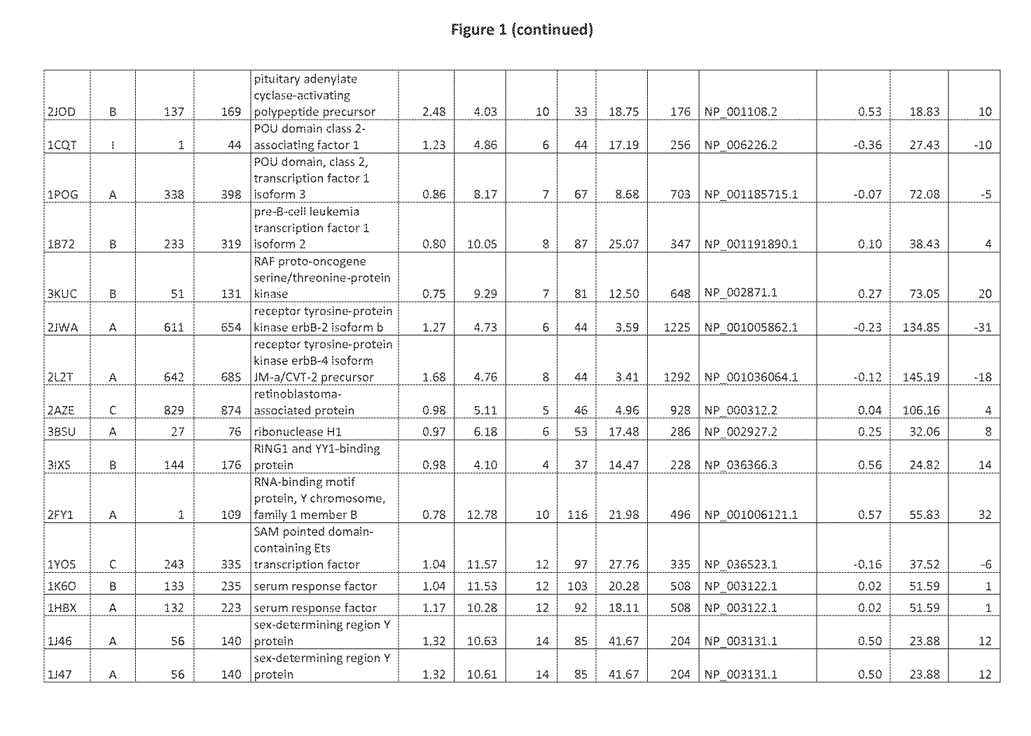

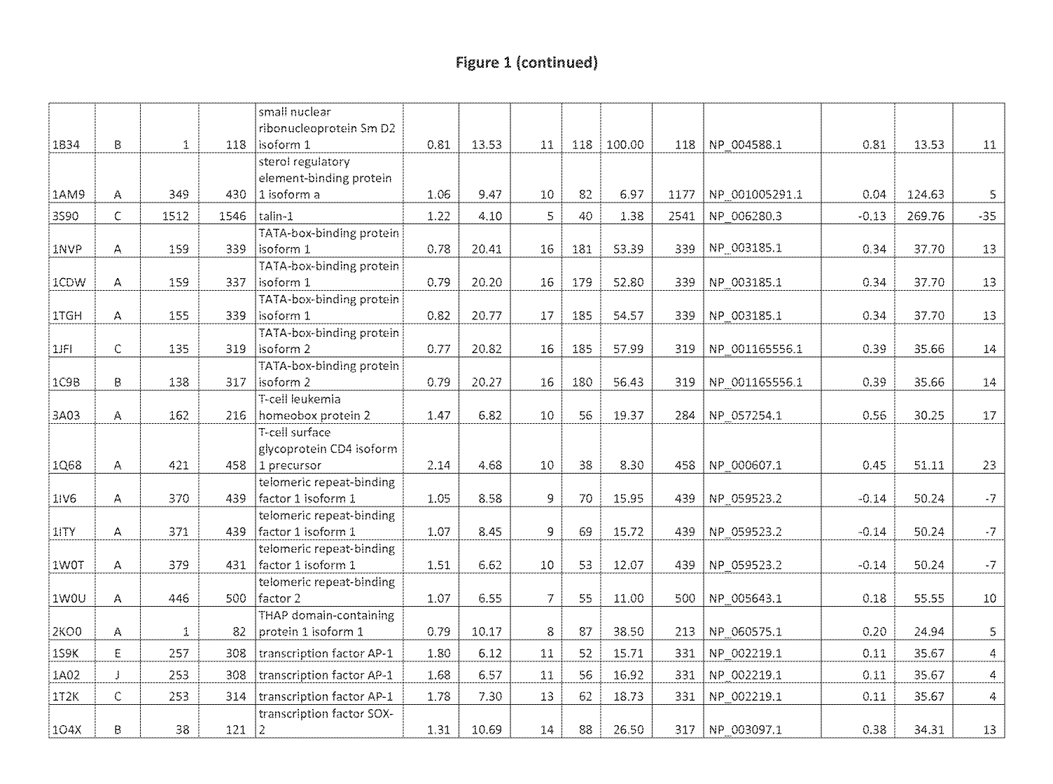

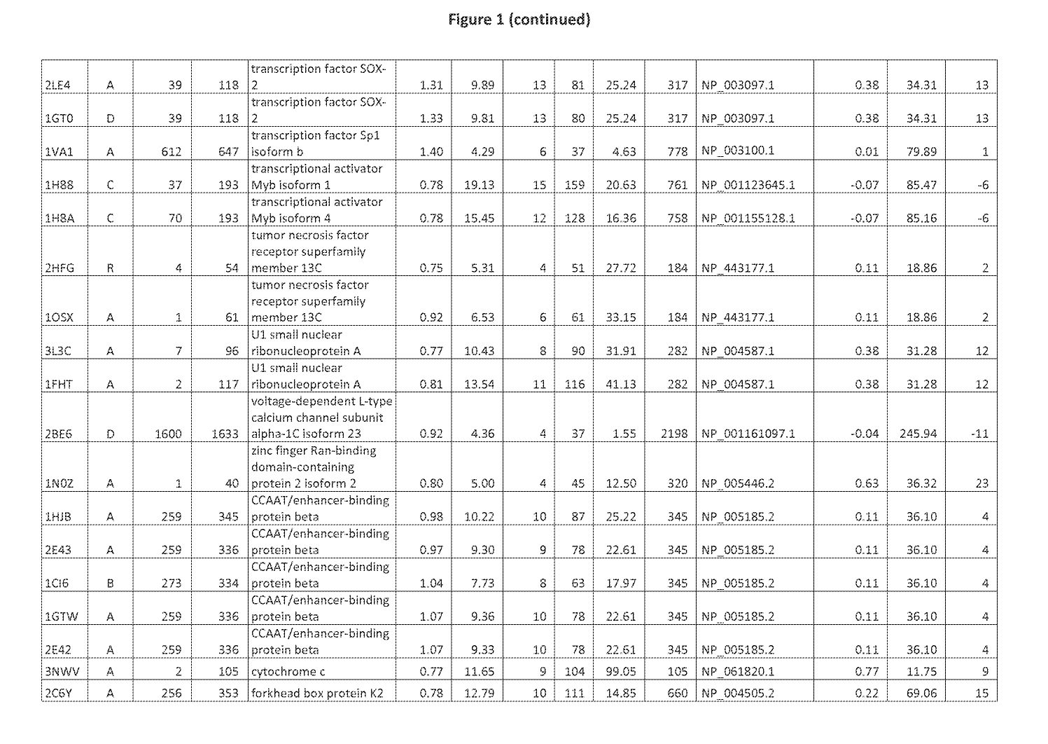

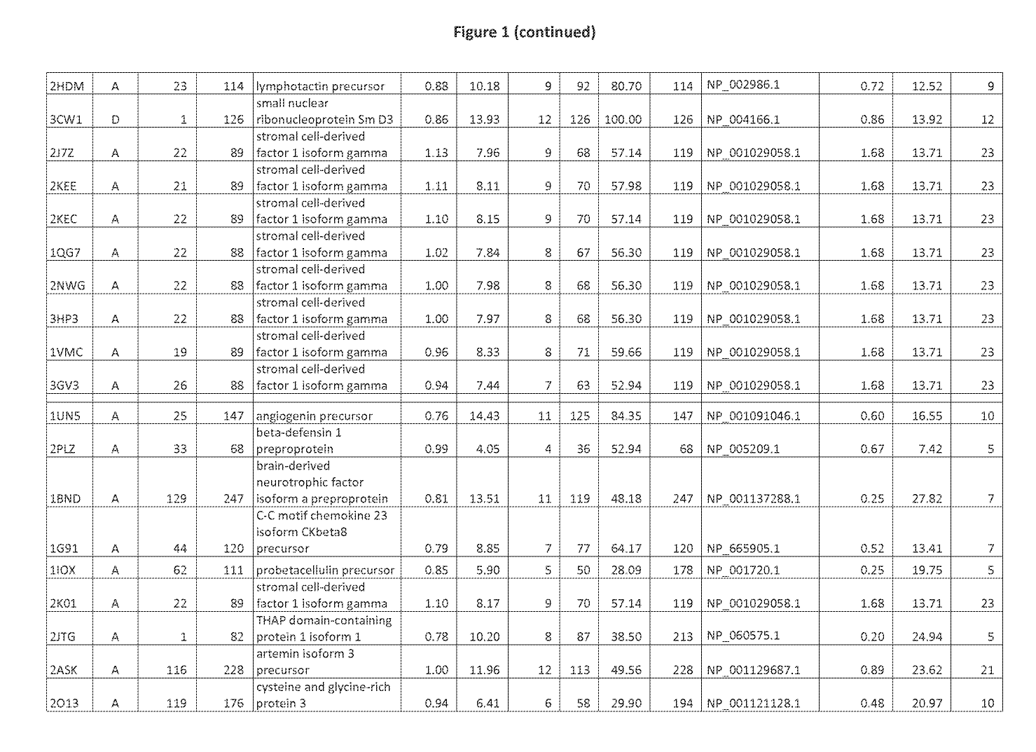

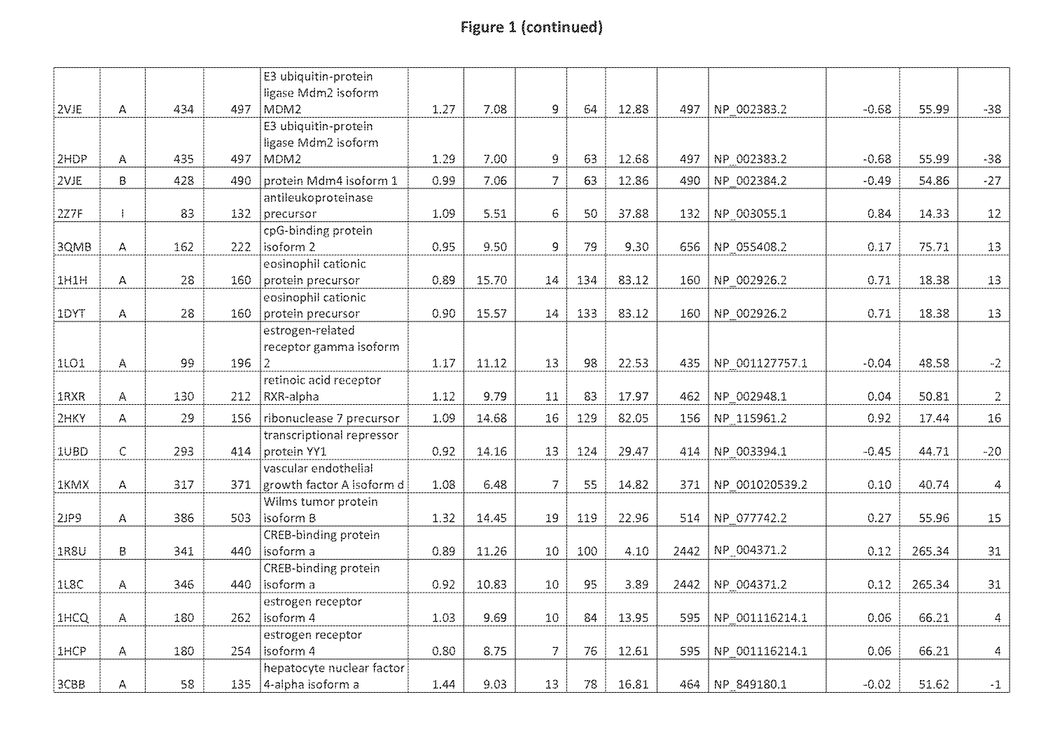

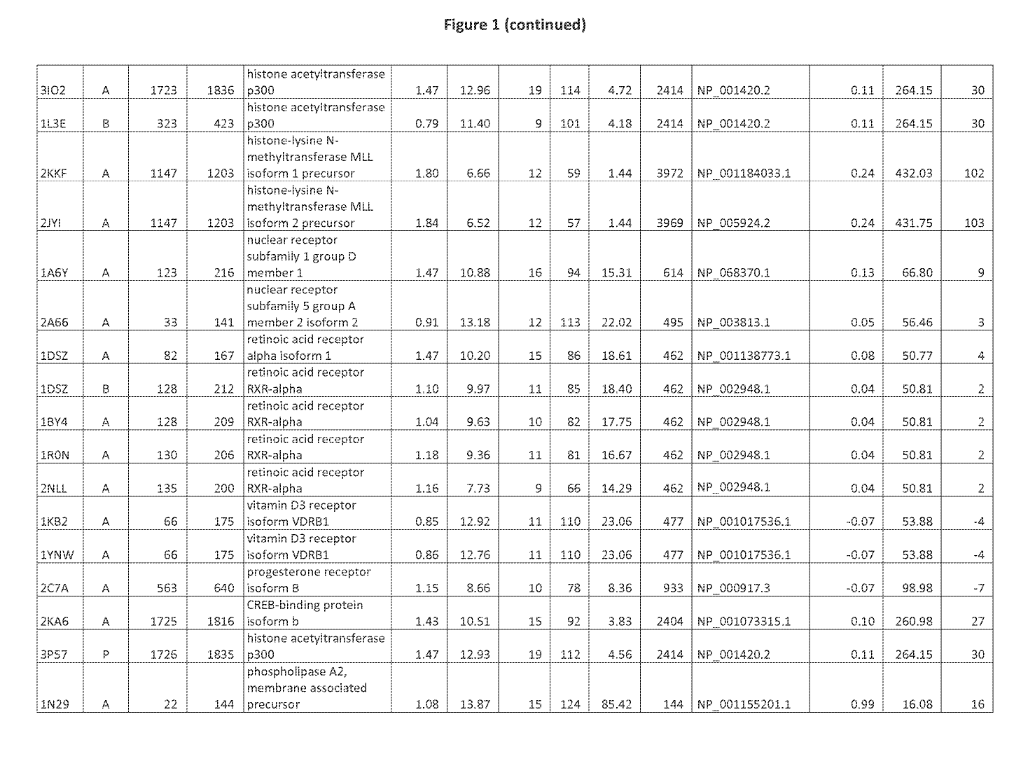

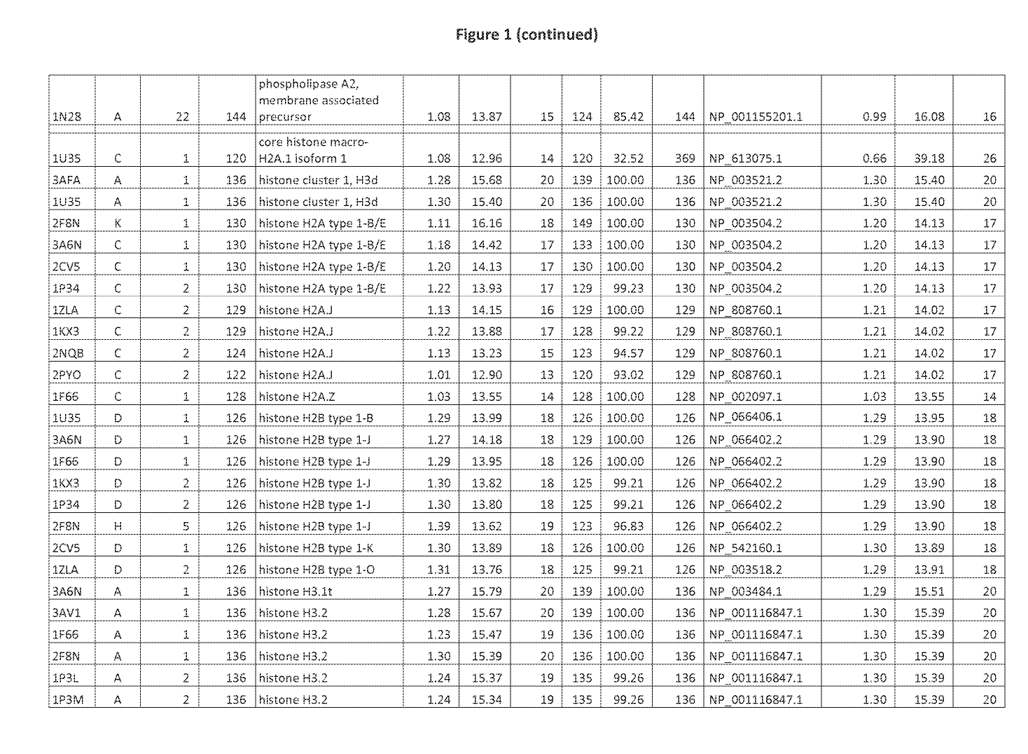

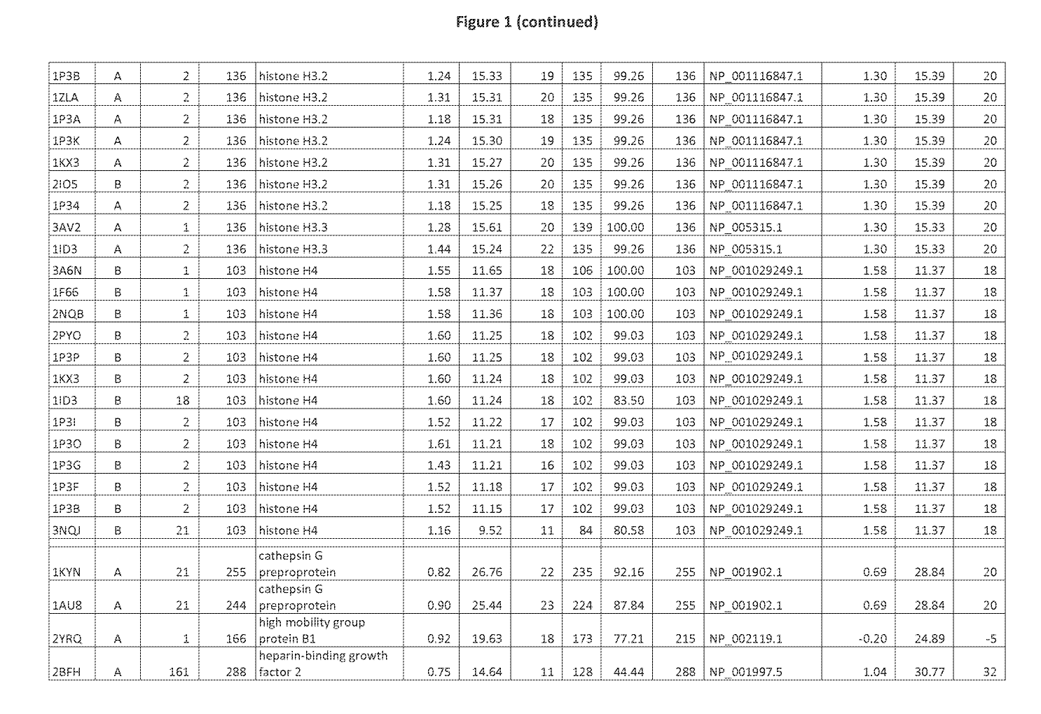

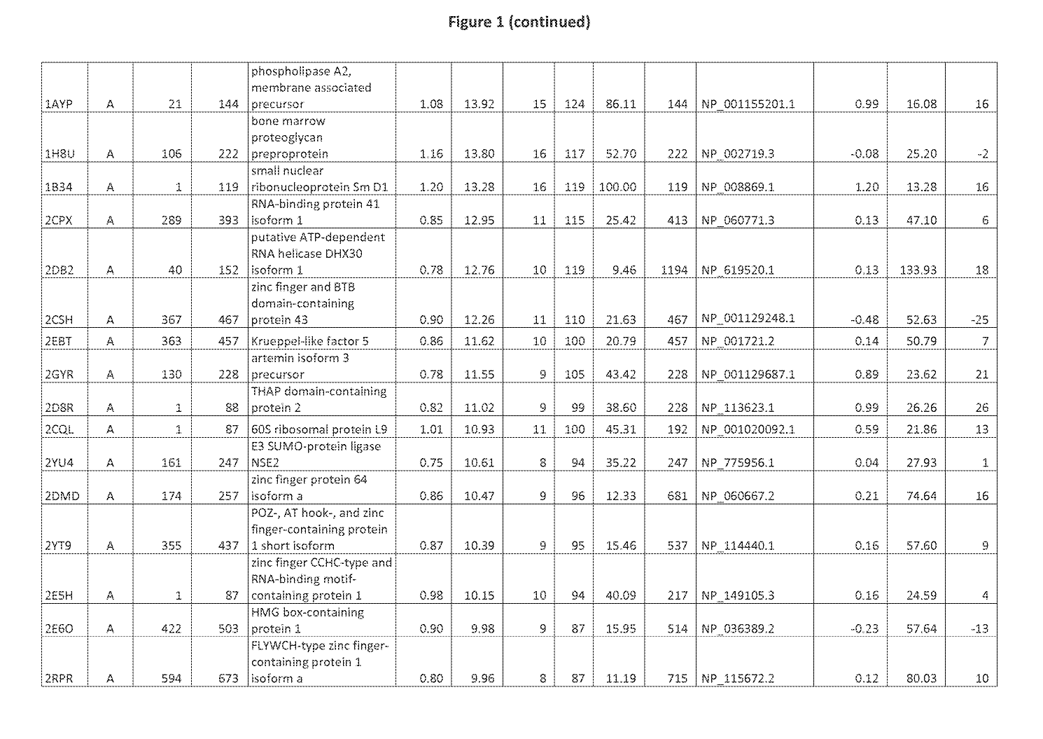

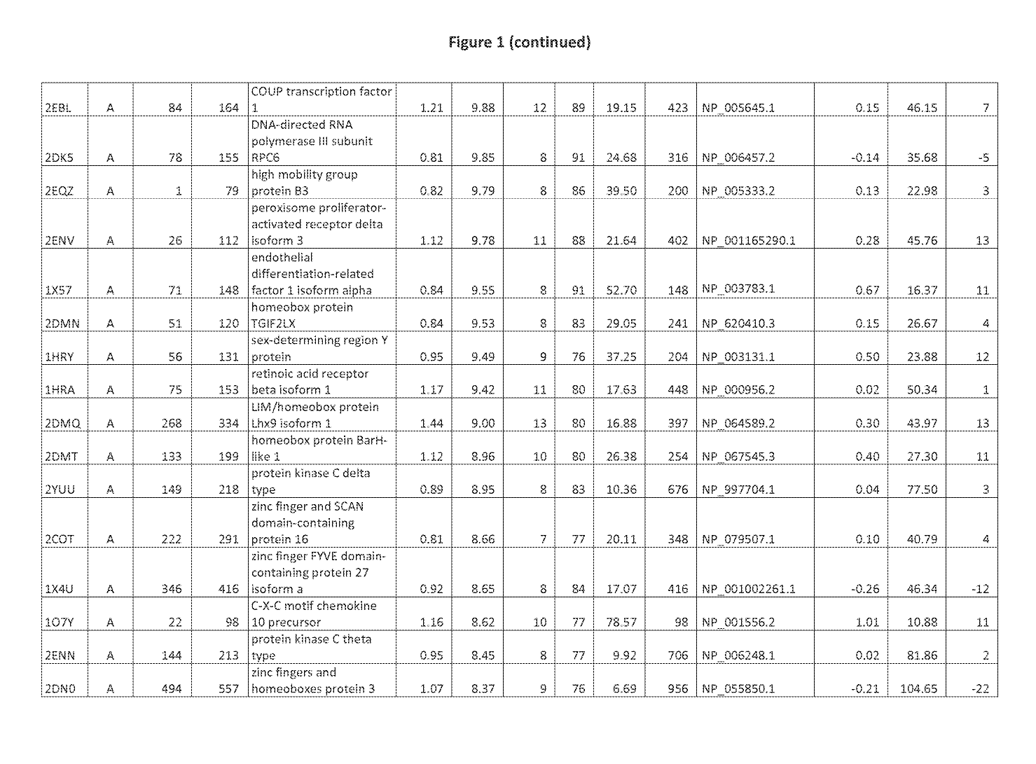

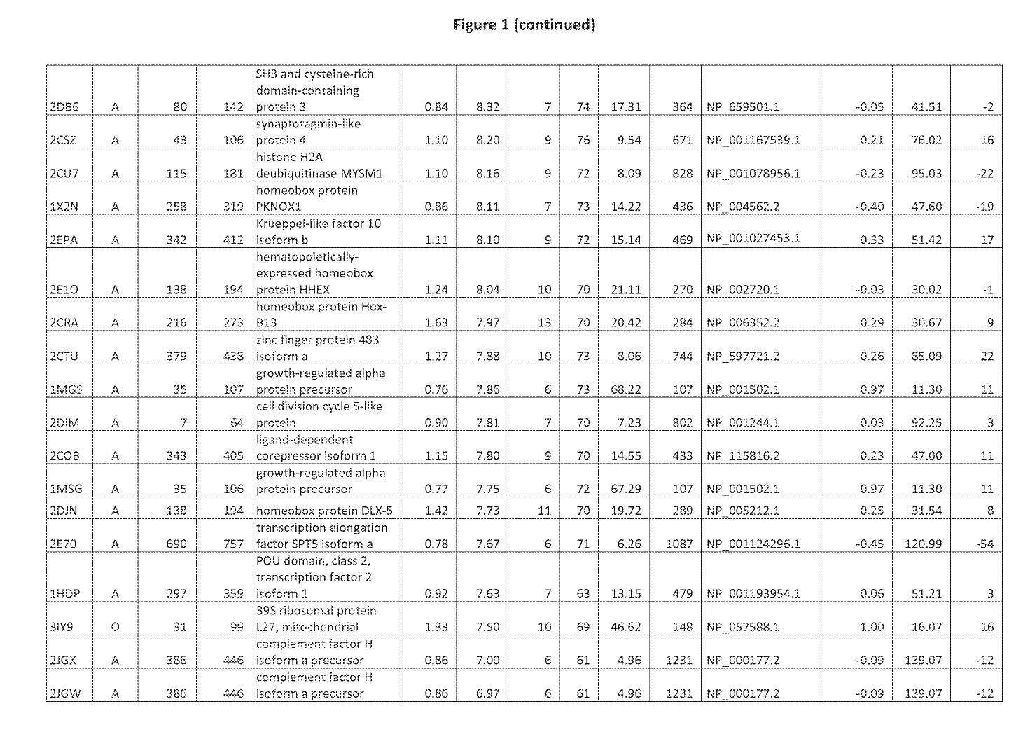

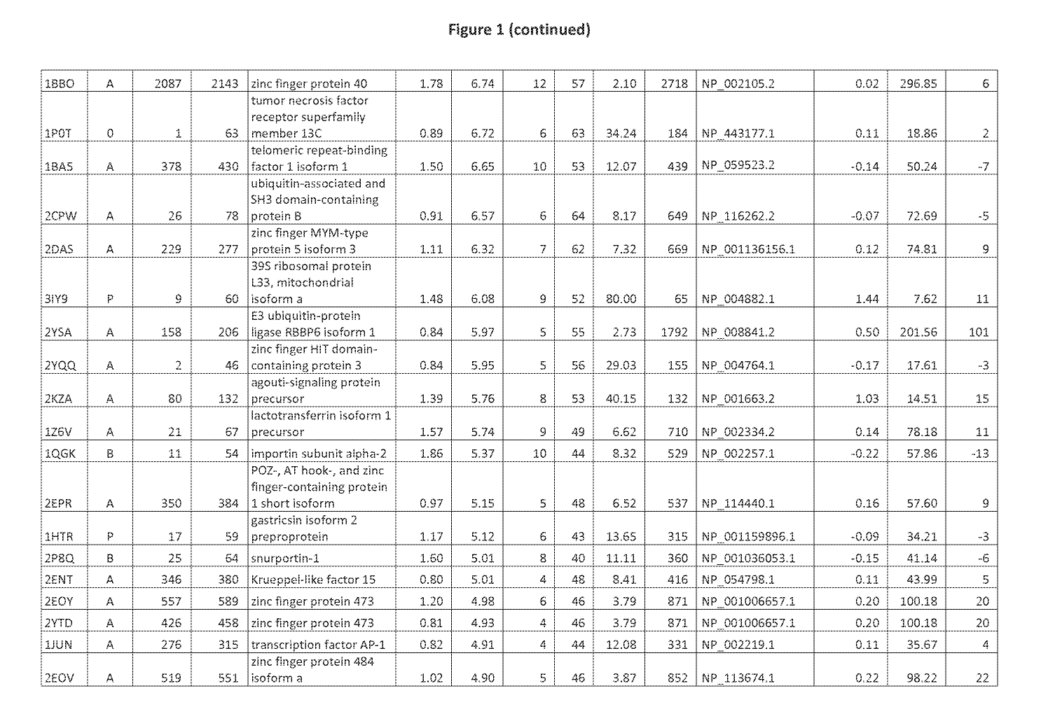

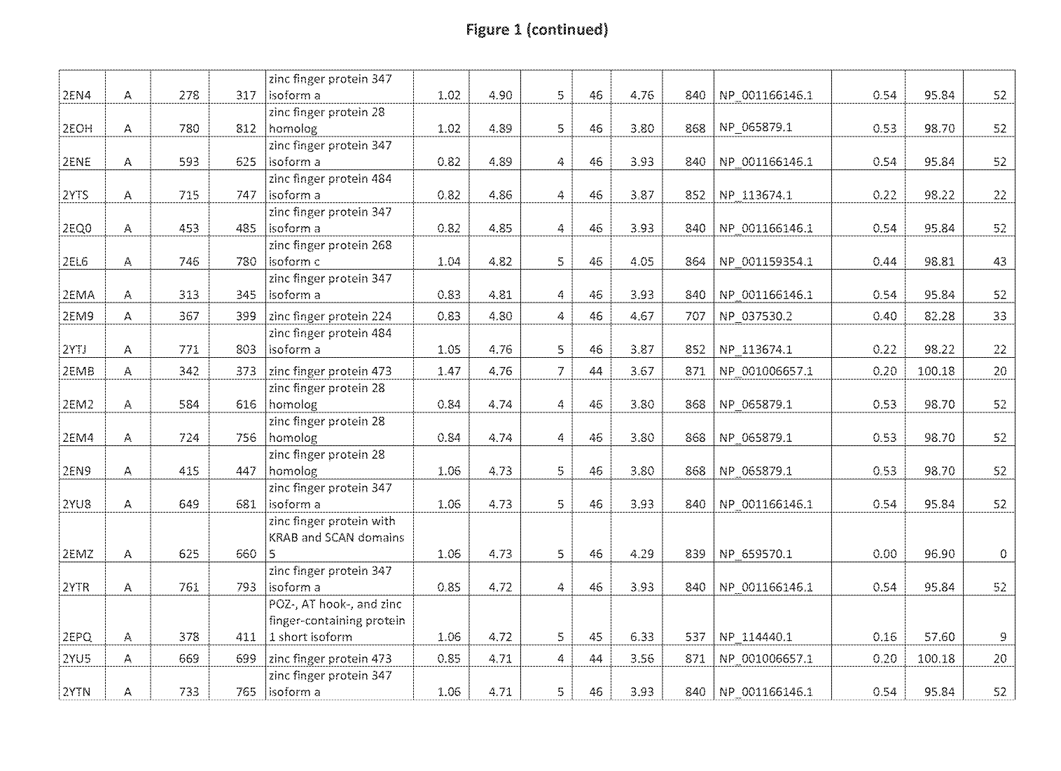

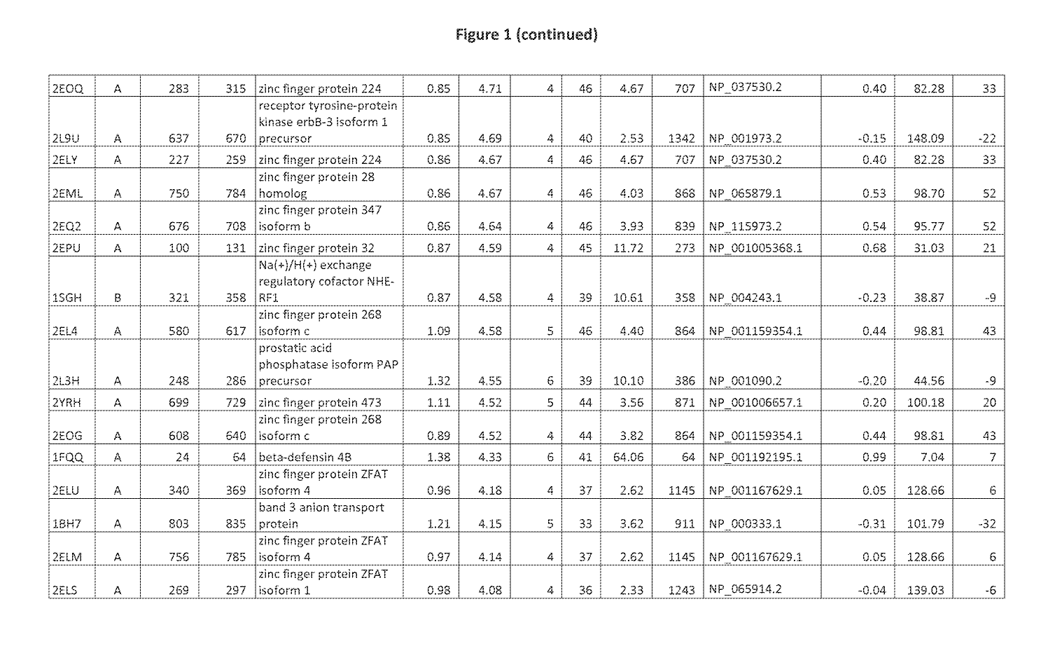

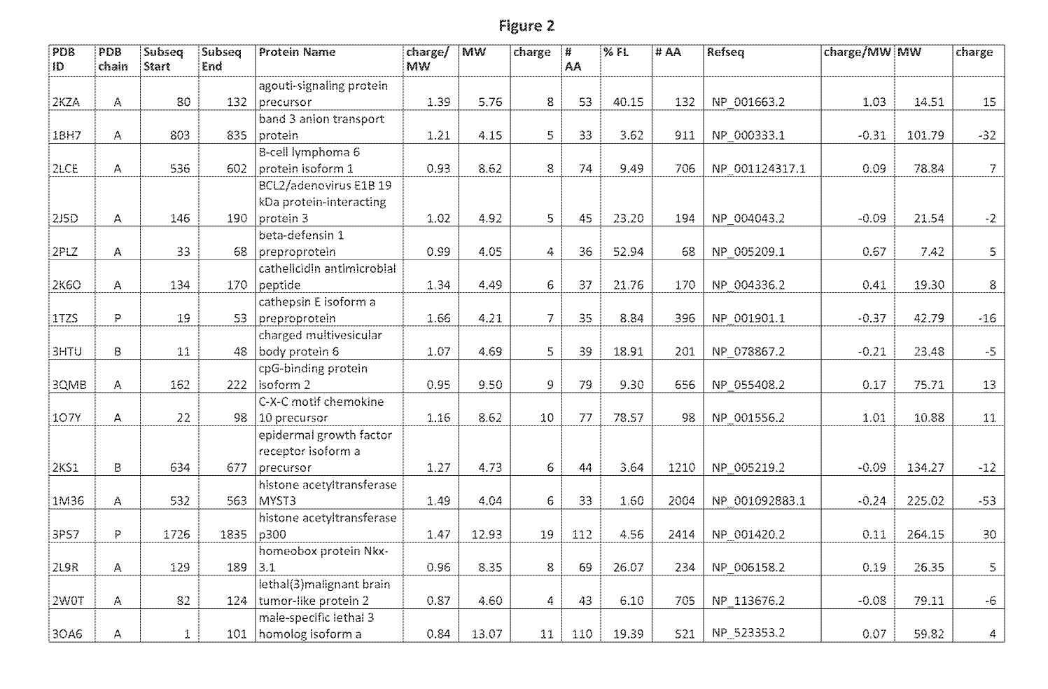

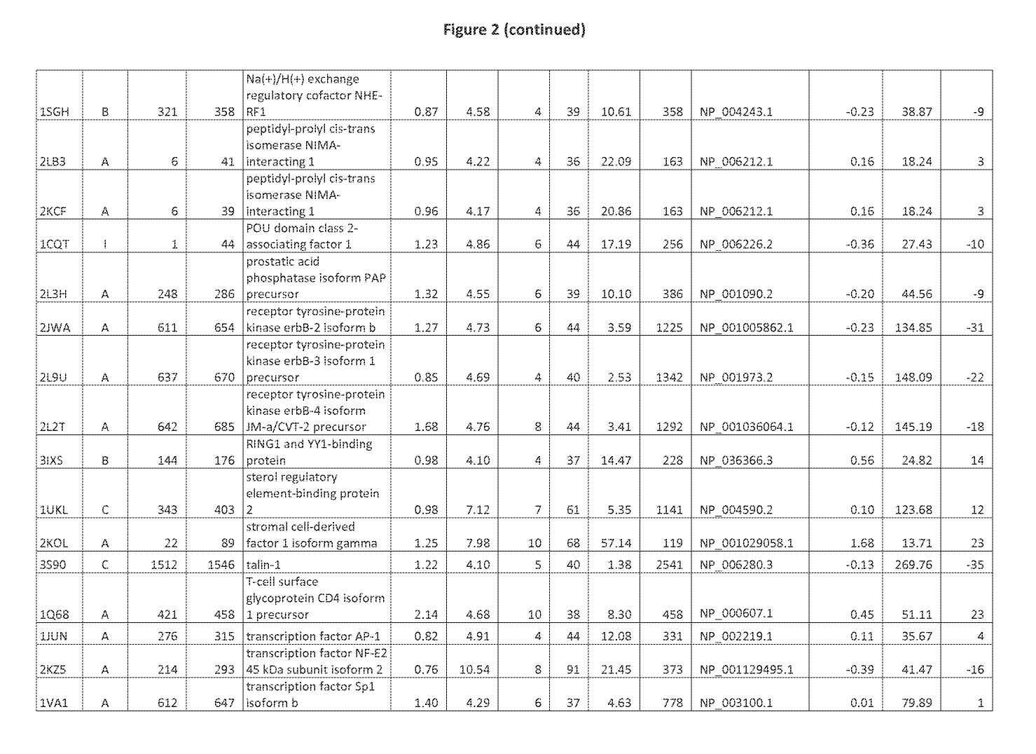

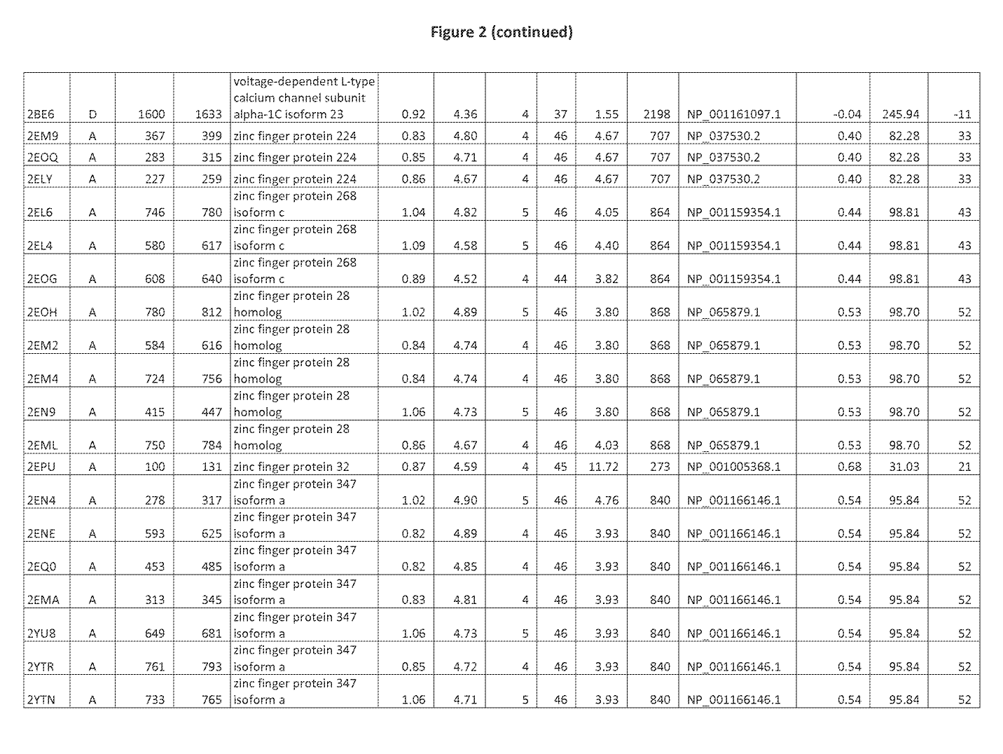

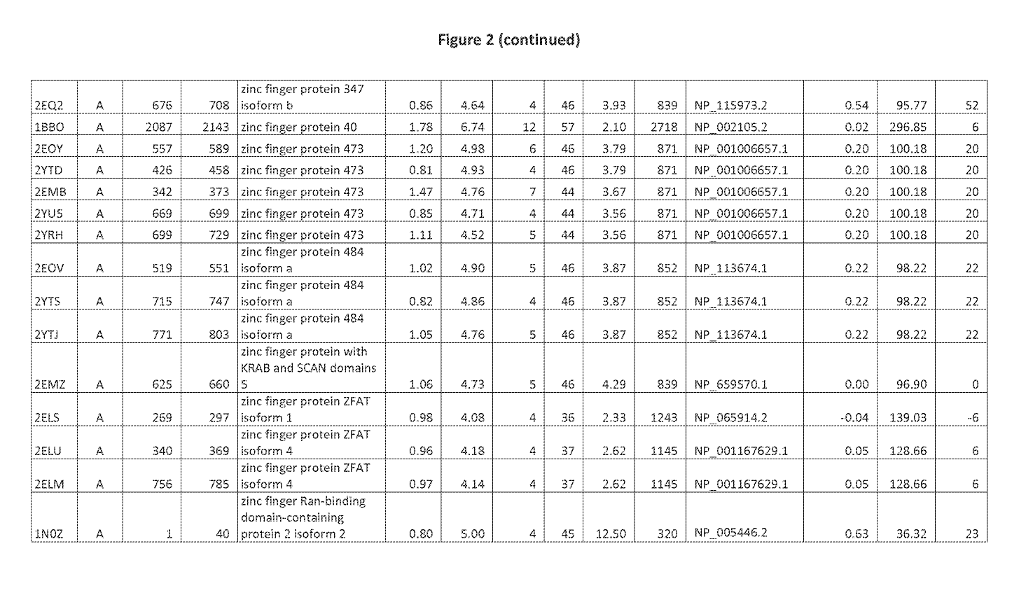

Additional exemplary Surf+ Penetrating Polypeptides are provided in FIGS. 1 and 2. The disclosure contemplates that any of the polypeptides, or fragments thereof, may be used in a complex of the disclosure. Moreover, additional suitable domains are described herein. Thus, the disclosure contemplates complexes comprising a Surf+ Penetrating Polypeptide-containing portion. This portion of the complex may comprise any of the Surf+ Penetrating Polypeptides provided in FIG. 1 or 2, or a full length or near full length naturally occurring polypeptide provided in FIG. 1 or 2, or a domain of any of the foregoing having a mass of at least 4 kDa, surface positive charge, and a charge/molecular weight ratio of at least 0.75. FIG. 1 provides information for exemplary domains of naturally occurring human proteins that are Surf+ Penetrating Polypeptides and can be used in the instant disclosure (e.g., in a complex and/or to deliver an AAM moiety into a cell). FIG. 2 provides similar information for a subset of the proteins provided in FIG. 1. For each entry, a PDB ID number (and chain) is provided, as well as the terminal residues of the fragment, relative to the full length sequence provided in GenBank (e.g., the subsequence start and subsequence end entries). The amino acid sequence for the full length protein sequences provided in GenBank are reproduced herein below in Section 1 of the sequence listing. The amino acid sequence for the particular domains identified by PDB ID number and chain are reproduced below in Section 2 of the sequence listing. The five columns to the right of the protein name provide information for the exemplified fragment (e.g., for the fragment of a naturally occurring human polypeptide, which fragment is a Surf+ Penetrating Polypeptide). For example, these columns indicate the charge/molecular weight, mass, net positive charge, length (# of amino acid residues) of the fragment, and the size of the fragment relative to its corresponding full length protein (% FL). The next column, just to the left of the Gen Bank accession number for the full length protein, indicates the size of the full length protein. The four columns to the right of the Ref seq column (the accession number for the full length protein) provide information for the full length, naturally occurring protein from which the fragment is derived. This information includes the charge/molecular weight of the full length protein, the molecular weight of the full length protein, the net charge (which, in some cases, may be negative) for the full length protein. As is clear from FIG. 1, for several proteins, non-overlapping domains that may be used as a Surf+ Penetrating Polypeptide were identified for a given naturally occurring human protein.

As can be seen upon review of FIG. 1, in some cases, both the full length, naturally occurring protein and a domain have characteristics indicative of a Surf+ Penetrating Polypeptide (e.g., surface positive charge, charge/molecular weight ratio of at least 0.75, etc.). However, in other cases, the full length protein does not have such characteristics, while a domain of the protein does. In certain embodiments, the disclosure provides complexes in which the Surf+ Penetrating Polypeptide has at least the following characteristics: surface positive charge, mass of at least 4 kDa, charge/molecular weight ratio of at least 0.75 or of greater than 0.75, and is a domain of a naturally occurring human polypeptide. In certain embodiments, the selected domain has a charge per molecular weight ratio greater than that of the corresponding full length, naturally occurring human polypeptide. In other embodiments, the selected domain has a charge per molecular weight ratio of at least 0.75 or greater than 0.75, but the full length, naturally occurring human polypeptide has a charge per molecular weight ratio of less than 0.75. In other embodiments, the selected domain has a net theoretical charge greater than that of the corresponding full length, naturally occurring human polypeptide. In other embodiments, the selected domain has a net positive charge and the corresponding, full length, naturally occurring human polypeptide has a net negative charge. The disclosure contemplates the use of any of the specified domains of full length, naturally occurring human proteins, as well as other domains having the charge and molecular weight characteristics of a Surf+ Penetrating Polypeptide. Moreover, the disclosure contemplates the use of full length, naturally occurring human polypeptides having the charge and molecular weight characteristics of a Surf+ Penetrating Polypeptide. Further, the disclosure contemplates that complexes may comprise a full length naturally occurring human polypeptide, even though only a domain of said human polypeptide functions as a Surf+ Penetrating Polypeptide. In such cases, the additional polypeptide sequence can optionally be used to interconnect the Surf+ Penetrating Polypeptide to the AAM moiety. Thus, in certain embodiments, the disclosure provides complexes comprising a first polypeptide portion that comprises a Surf+ Penetrating Polypeptide. Such a Surf+ Penetrating Polypeptide may optionally be provided with additional sequence endogenously present in, for example, the naturally occurring polypeptide from which the Surf+ Penetrating Polypeptide is a domain or may be present without additional sequence endogenously present in the naturally occurring polypeptide from which the Surf+ Penetrating Polypeptide is a domain. In certain embodiments, the presence of additional sequence from the same naturally occurring polypeptide does not result in the portion comprising the Surf+ Penetrating Polypeptide having a charge/molecular weight ratio of less than 0.75. However, in certain embodiments, the presence of additional sequence from the same naturally occurring polypeptide results in the portion comprising the Surf+ Penetrating Polypeptide having a charge/molecular weight ratio of less than 0.75. For the avoidance of doubt, the “portion comprising a Surf+ Penetrating Polypeptide” refers to the Surf+ Penetrating Polypeptide and additional sequence from the same or similar naturally or non-naturally occurring polypeptide. This portion does not include heterologous linker sequence, nuclear localization signals, or additional portions intended to have an independent and distinct biological function (e.g., a moiety to increase the half life of the complex).

The foregoing are exemplary of sub-categories of Surf+ Penetrating Polypeptides that can be used as part of the complexes of the disclosure. For the avoidance of doubt, it should be understood that domains of the naturally occurring human proteins may be modified, such as by introducing one or more amino acid substitutions, deletions or additions. The resulting domain will still be considered a domain of a naturally occurring human polypeptide as long as the domain is readily identifiable based on sequence and/or structure as a domain of that naturally occurring human protein.

In certain embodiments, the Surf+ Penetrating Polypeptide portion comprises (or consists of) a full length naturally occurring polypeptide or a domain of a full length polypeptide presented in FIG. 2. In certain embodiments, the disclosure provides a complex comprising an AAM moiety associated with a human polypeptide (full length or domain) presented in FIG. 2. However, as should be noted, the domains depicted in the figures are merely exemplary. Having identified a suitable domain, such as the domains identified by PDB in FIGS. 1 and 2, suitable sub-domains or non-overlaping domains can be readily identified. Thus, in certain embodiments, the disclosure contemplates the use of any of the domains set forth in FIG. 1 or 2, as well as a fragment (sub-domain; also considered a domain) thereof having a mass of at least 4 kDa, surface positive charge and charge/molecular weight ratio of at least 0.75.

To further illustrate, in certain embodiments, the Surf+ Penetrating Polypeptide is a full length or a domain of C-C motif chemokine 26 precursor (e.g., such as a fragment of about 71 amino acid residues beginning at position 24 of the full length protein, a net charge of +13, and having a charge/MW of 1.55), a domain of HB-EGF (proheparin-binding EGF-like growth factor precursor, such as, a fragment of about 79 amino acid residues beginning at position 72 of the full length protein, a net positive charge of +12, and a charge/molecular weight of 1.35), a domain of protein DEK isoform 1 (e.g., such as a fragment of about 131 amino acid residues beginning at position 78 of the full length protein, a net positive charge of +19, and a charge/molecular weight of 1.26), a domain of hepatocyte growth factor isoform 1 preprotein (e.g., such as a fragment of about 131 amino acid residues beginning at position 31 of the full length protein, a net positive charge of +14, and a charge/molecular weight of 1.23), a full length or a domain of cytochrome c (e.g., such as a fragment of about 104 amino acid residues beginning at position 2 of the full length protein, a net positive charge of +9, and a charge per molecular weight of 0.77), a full length or domain of C-X-C motif chemokine 24 precursor (e.g., such as a fragment of about 78 amino acid residues beginning at position 34 of the full length protein, a net positive charge of +13, and a charge per molecular weight of 1.37), or a domain of ataxin 7 isoform a (e.g., such as a fragment of about 74 amino acid residues beginning at position 330, a net positive charge of +9, and a chare/molecular weight of 1.03). In certain embodiments, the disclosure provides a complex comprising an AAM moiety and any of the foregoing full length, naturally occurring human polypeptides, or a domain thereof, which domain has the charge and charge/molecular weight characteristics of a Surf+ Penetrating Polypeptide. In certain embodiments, the complex (a complex of the disclosure) comprises a domain of the full length, naturally occurring human polypeptide, but the complex does not comprise the full length, naturally occurring human polypeptide.

To further illustrate, in other embodiments, the Surf+ Penetrating Polypeptide is a domain of any of the following, which domain has a charge per molecular weight ratio of at least 0.75 but for which the corresponding full length naturally occurring polypeptide has a charge/molecular weight ratio of less than 0.75:histone-lysine N-methyltransferase MLL isoform 1 precursor; transcription factor AP-1; proheparin-binding EGF-like growth factor precursor; protein DEK isoform 1; hepatocyte growth factor isoform 1 preprotein; epidermal growth factor receptor isoform a precursor; forkhead box protein K2; pre-mRNA-processing factor 40 homolog A; ataxin-7 isoform a, E3 SUMO-protein ligase PIAS1; platelet factor 4 precursor; advanced glycosylation end product-specific receptor isoform 2 precursor; serol regulatory element-binding protein 2; histone acetyltransferase p300; U1 small nuclear ribonucleoprotein A; pre-B-cell leukemia transcription factor 1 isoform 2; homeobox protein Nkx 3.1; homeobox protein Hox-A9; B-cell lymphoma 6 protein isoform 1; ETS domain-containing protein Elk-4 isoform a; pituitary homeobox 3; granulysin isoform NKG5; general transcription factor IIF subunit 1; histone deacetylase complex subunit SAP30; heterochromatin protein 1-binding protein 3; lethal(3)malignant brain tumor-like protein 2; CCAAT/enhancer-binding protein beta; troponin T, cardiac muscle isoform 2; CREB-binding protein isoform B; cyclic AMP-dependent transcription factor ATF-2; cathepsin E isoform a preprotein; glycine receptior subunit alpha-1 isoform 1 precursor; CREB-binding protein isoform b; pituitary adenylate cyclase-activating polypeptide precursor; mastermind-like protein 1; BCL2/adenovirus E1B 19 kDa protein-interacting protein 3; cathelicidin antimicrobial peptide; epidermal growth factor receptor isoform a precursor; transcription factor NF-E2 45 kDa subunit isoform 2; integrin beta-1 isoform 1D precursor; C-C motif chemokine 5 precursor; forkhead box protein 01, 03 or 04; talin 1; TATA-box binding protein isoform 1 or 2; telomeric repeat-binding factor 1 or 2; or lactotransferrin isoform 1 precursor. For each of the foregoing, a suitable fragment is provided in FIG. 1. Moreover, other examples of this sub-category of Surf+ Penetrating Polypeptides are provided in and are immediately apparent from FIG. 1. In certain embodiments, the disclosure provides a complex comprising an AAM moiety and any of the foregoing full length, naturally occurring human polypeptides, or a domain thereof, which domain has the charge and charge/molecular weight characteristics of a Surf+ Penetrating Polypeptide. In certain embodiments, the complex (a complex of the disclosure) comprises a domain of the full length, naturally occurring human polypeptide, but the complex does not comprise the full length, naturally occurring human polypeptide. In certain embodiments, the complex and/or the Surf+ Penetrating Polypeptide portion does not include one of the polypeptides or specific fragments provided in FIG. 1. In certain embodiments, the complex and/or the Surf+ Penetrating Polypeptide portion does not include HRX (Uniprot number Q03164 or fragment identified at PDB 2J2S. In certain embodiments, the complex and/or the Surf+ Penetrating Polypeptide portion does not include c-Jun (Uniprot number P05412 or fragment identified at PDB 1JNM. In certain embodiments, the complex and/or the Surf+ Penetrating Polypeptide portion does not include defensin 3 (Uniprot number P81534 or fragment identified at PDB 1KJ6. In certain embodiments, the complex and/or the Surf+ Penetrating Polypeptide portion does not include HBEGF (Uniprot number Q99075 or fragment identified at PDB 1×DT. In certain embodiments, the complex and/or the Surf+ Penetrating Polypeptide portion does not include N-Dek (Uniprot number P35659 or fragment identified at PDB 2JX3. In certain embodiments, the complex and/or the Surf+Penetrating Polypeptide portion does not include HGF (Uniprot number P14210 or fragment identified at PDB 2HGF. In certain embodiments, the complex and/or the Surf+ Penetrating Polypeptide portion does not include HIST4 (Uniprot number P62805 or fragment identified at PDB 2CV5.

To further illustrate, in other embodiments, the Surf+ Penetrating Polypeptide is a domain of: charged multivesicular body protein 6 (e.g., a fragment of about 39 amino acid residues having a charge/molecular weight of 1.07); homeobox protein Nkx3.1 (e.g., a fragment of about 69 amino acid residue having a charge/molecular weight of 0.96); B-cell lymphoma 6 protein isoform 1 (e.g., a fragment of about 74 amino acid residues having a charge per molecular weight of 0.93); lethal(3)malignant brain tumor-like protein 2 (e.g., a fragment of about 43 amino acid residues having a charge/molecular weight of 0.87); cathepsin E isoform a preprotein (e.g., a fragment of about 35 amino acid residues having a charge/molecular weight of 1.66); BCL2/adenovirus E1B 19 kDa protein-interacting protein 3 (e.g., a fragment of about 45 amino acid residues have a charge/molecular weight of 1.02); cathelicidin antimicrobial peptide (e.g., a fragment of about 37 amino acid residues having a charge/molecular weight of 1.34). In certain embodiments, the disclosure provides a complex comprising an AAM moiety and any of the foregoing full length, naturally occurring human polypeptides, or a domain thereof, which domain has the charge and charge/molecular weight characteristics of a Surf+ Penetrating Polypeptide. In certain embodiments, the complex (a complex of the disclosure) comprises a domain of the full length, naturally occurring human polypeptide, but the complex does not comprise the full length, naturally occurring human polypeptide.

To further illustrate, in other embodiments, the Surf+ Penetrating Polypeptide is selected from a domain of any of: agouti-signaling protein precursor, band 3 anion transport protein, B-cell lymphoma 6 protein isoform 1, BCL2/adenovirus E1B 19 kDa protein-interacting protein 3, beta-defensin 1 preproprotein, cathepsin E isoform a preproprotein, charged multivesicular body protein 6, cpG-binding protein isoform 2, C-X-C motif chemokine 10 precursor, epidermal growth factor receptor isoform a precursor, histone acetyltransferase MYST3, histone acetyltransferase p300, homeobox protein Nkx-3.1, lethal(3)malignant brain tumor-like protein 2, male-specific lethal 3 homolog isoform a, Na(+)/H(+) exchange regulatory cofactor NHE-RF1, peptidyl-prolyl cis-trans isomerase NIMA-interacting 1, peptidyl-prolyl cis-trans isomerase NIMA-interacting 1, POU domain class 2-associating factor 1, prostatic acid phosphatase isoform PAP precursor, receptor tyrosine-protein kinase erbB-2 isoform b, receptor tyrosine-protein kinase erbB-3 isoform 1 precursor, receptor tyrosine-protein kinase erbB-4 isoform JM-a/CVT-2 precursor, RING1 and YY1-binding protein, sterol regulatory element-binding protein 2, stromal cell-derived factor 1 isoform gamma, talin-1, T-cell surface glycoprotein CD4 isoform 1 precursor, transcription factor AP-1, transcription factor NF-E2 45 kDa subunit isoform 2, transcription factor Sp1 isoform b, voltage-dependent L-type calcium channel subunit alpha-1C isoform 23, zinc finger protein 224, zinc finger protein 268 isoform c, zinc finger protein 28 homolog, zinc finger protein 32, zinc finger protein 347 isoform a, zinc finger protein 347 isoform b, or zinc finger protein 40. In certain embodiments, the selected domain is a domain presented in FIG. 2, or a variant thereof. In certain embodiments, the complex (a complex of the disclosure) comprises a domain of the full length, naturally occurring human polypeptide, but the complex does not comprise the full length, naturally occurring human polypeptide.

In certain embodiments, the disclosure provides a complex comprising an AAM moiety and any of the following full length (or substantially full length), naturally occurring human polypeptides: agouti-signaling protein precursor, band 3 anion transport protein, B-cell lymphoma 6 protein isoform 1, BCL2/adenovirus E1B 19 kDa protein-interacting protein 3, beta-defensin 1 preproprotein, cathepsin E isoform a preproprotein, charged multivesicular body protein 6, cpG-binding protein isoform 2, C-X-C motif chemokine 10 precursor, epidermal growth factor receptor isoform a precursor, histone acetyltransferase MYST3, histone acetyltransferase p300, homeobox protein Nkx-3.1, lethal(3)malignant brain tumor-like protein 2, male-specific lethal 3 homolog isoform a, Na(+)/H(+) exchange regulatory cofactor NHE-RF1, peptidyl-prolyl cis-trans isomerase NIMA-interacting 1, peptidyl-prolyl cis-trans isomerase NIMA-interacting 1, POU domain class 2-associating factor 1, prostatic acid phosphatase isoform PAP precursor, receptor tyrosine-protein kinase erbB-2 isoform b, receptor tyrosine-protein kinase erbB-3 isoform 1 precursor, receptor tyrosine-protein kinase erbB-4 isoform JM-a/CVT-2 precursor, RING1 and YY1-binding protein, sterol regulatory element-binding protein 2, stromal cell-derived factor 1 isoform gamma, talin-1, T-cell surface glycoprotein CD4 isoform 1 precursor, transcription factor AP-1, transcription factor NF-E2 45 kDa subunit isoform 2, transcription factor Sp1 isoform b, voltage-dependent L-type calcium channel subunit alpha-1C isoform 23, zinc finger protein 224, zinc finger protein 268 isoform c, zinc finger protein 28 homolog, zinc finger protein 32, zinc finger protein 347 isoform a, zinc finger protein 347 isoform b, or zinc finger protein 40.

FIG. 1 provides specific examples of domains that are Surf+ Penetrating Polypeptides. It should be appreciated that other fragments of the corresponding naturally occurring human proteins may also be suitable, such as an overlapping fragment that retains the surface positive charge of the recited fragment but is shorter or longer (e.g., the starting or ending residue is different but the functional core of surface positive charge is retained; the fragment retains the essential structure of the recited fragment). Fragments that retain the essential structure but differ in length may differ in mass, length, and/or charge/molecular weight. However, essential structure, surface charge and charge/molecular weight of at least 0.75 are maintained. Additionally, FIG. 1 provides examples for several human polypeptides of more than one non-overlapping domain that may be used as a Surf+ Penetrating Polypeptide.

In certain embodiments, the Surf+ Penetrating Polypeptide portion of a complex of the disclosure is or comprises a domain of a human polypeptide, such as a domain of a naturally occurring human polypeptide. A complex may comprise the domain outside of its context in its full length, naturally occurring protein (e.g., the complex does not include the full length human polypeptide from which the domain is a portion). Alternatively, the domain may be provided in the context of its full length polypeptide or in the context of additional polypeptide sequence (but less than all) from the naturally occurring protein from which the Surf+ Penetrating Polypeptide is a domain (e.g., the complex does include the full length human polypeptide from which the domain is an identified portion).

In some embodiments, a complex of the disclosure (e.g., a complex comprising an AAM moiety associated with the polypeptide) comprises a polypeptide listed in Table 1 below. In other words, in some embodiments, a complex comprises a portion comprising a Surf+ Penetrating Polypeptide and the portion comprising a Surf+ Penetrating Polypeptide is selected from a polypeptide listed in Table 1. In certain embodiments, the complex includes at least about 10%, 15%, 20%, 25%, 30%, 35%, 40%, 45%, 50%, 55%, 60%, 65%, 70%, 75%, 80%, 85%, 90%, 95%, 96%, 97%, 98%, or 100% of the full length polypeptide, provided as contiguous amino acid residues.

TABLE 1
Exemplary naturally occurring molecules that
may be used in a complex of the disclosure

| Protein Name | Refseq^{a} |
|---|---|
| 60S ribosomal protein L10 | NP_006004.2 |
| advanced glycosylation end product-specific receptor | NP_001193858.1 |
| isoform 2 precursor | |
| ataxin-7 isoform a | NP_000324.1 |
| B-cell lymphoma 6 protein isoform 1 | NP_001124317.1 |
| BCL2/adenovirus E1B 19 kDa protein-interacting protein 3 | NP_004043.2 |
| cathelicidin antimicrobial peptide | NP_004336.2 |
| cathepsin E isoform a preproprotein | NP_001901.1 |
| C-C motif chemokine 13 precursor | NP_005399.1 |
| C-C motif chemokine 24 precursor | NP_002982.2 |
| C-C motif chemokine 5 precursor | NP_002976.2 |
| C-C motif chemokine 7 precursor | NP_006264.2 |
| CCAAT/enhancer-binding protein beta | NP_005185.2 |
| charged multivesicular body protein 6 | NP_078867.2 |
| CREB-binding protein isoform b | NP_001073315.1 |
| C-X-C motif chemokine 14 precursor | NP_004878.2 |
| C-X-C motif chemokine 2 | NP_002080.1 |
| cyclic AMP-dependent transcription factor ATF-2 | NP_001871.2 |
| cytochrome c | NP_061820.1 |
| E3 SUMO-protein ligase PIAS1 | NP_057250.1 |
| eotaxin precursor | NP_002977.1 |
| epidermal growth factor receptor isoform a precursor | NP_005219.2 |
| epidermal growth factor receptor isoform a precursor | NP_005219.2 |
| ETS domain-containing protein Elk-4 isoform a | NP_001964.2 |
| fibroblast growth factor 10 precursor | NP_004456.1 |
| fibroblast growth factor 8 isoform B precursor | NP_006110.1 |
| forkhead box protein K2 | NP_004505.2 |
| forkhead box protein O1 | NP_002006.2 |
| forkhead box protein O3 | NP_963853.1 |
| forkhead box protein O4 isoform 1 | NP_005929.2 |
| general transcription factor IIF subunit 1 | NP_002087.2 |
| glycine receptor subunit alpha-1 isoform 1 precursor | NP_001139512.1 |
| granulysin isoform NKG5 | NP_006424.2 |
| heparin-binding growth factor 2 | NP_001997.5 |
| hepatocyte growth factor isoform 1 preproprotein | NP_000592.3 |
| heterochromatin protein 1-binding protein 3 | NP_057371.2 |
| histone acetyltransferase MYST3 | NP_001092883.1 |
| histone acetyltransferase p300 | NP_001420.2 |
| histone deacetylase complex subunit SAP30 | NP_003855.1 |
| histone H3-like centromeric protein A isoform a | NP_001800.1 |
| homeobox protein Hox-A9 | NP_689952.1 |
| homeobox protein Hox-B1 | NP_002135.2 |
| homeobox protein NANOG | NP_079141.2 |
| homeobox protein Nkx-3.1 | NP_006158.2 |
| integrin beta-1 isoform 1D precursor | NP_391988.1 |
| lethal(3)malignant brain tumor-like protein 2 | NP_113676.2 |
| liver-expressed antimicrobial peptide 2 precursor | NP_443203.1 |
| lymphotactin precursor | NP_002986.1 |
| major centromere autoantigen B | NP_001801.1 |
| male-specific lethal 3 homolog isoform a | NP_523353.2 |
| mastermind-like protein 1 | NP_055572.1 |
| max dimerization protein 1 isoform 2 | NP_001189442.1 |
| nucleolar transcription factor 1 isoform a | NP_055048.1 |
| parathyroid hormone-related protein isoform 2 preproprotein | NP_945315.1 |
| peptidyl-prolyl cis-trans isomerase NIMA-interacting 1 | NP_006212.1 |
| pituitary adenylate cyclase-activating polypeptide precursor | NP_001108.2 |
| pituitary homeobox 3 | NP_005020.1 |
| platelet factor 4 precursor | NP_002610.1 |
| POU domain class 2-associating factor 1 | NP_006226.2 |
| POU domain, class 2, transcription factor 1 isoform 3 | NP_001185715.1 |
| pre-B-cell leukemia transcription factor 1 isoform 2 | NP_001191890.1 |
| pre-mRNA-processing factor 40 homolog A | NP_060362.3 |
| RAF proto-oncogene serine/threonine-protein kinase | NP_002871.1 |
| receptor tyrosine-protein kinase erbB-2 isoform b | NP_001005862.1 |
| receptor tyrosine-protein kinase erbB-4 isoform JM-a/CVT-2 | NP_001036064.1 |
| precursor | |
| retinoblastoma-associated protein | NP_000312.2 |
| ribonuclease H1 | NP_002927.2 |
| RING1 and YY1-binding protein | NP_036366.3 |
| RNA-binding motif protein, Y chromosome, family 1 | NP_001006121.1 |
| member B | |
| SAM pointed domain-containing Ets transcription factor | NP_036523.1 |
| serine/arginine-rich splicing factor 1 isoform 1 | NP_008855.1 |
| serum response factor | NP_003122.1 |
| sex-determining region Y protein | NP_003131.1 |
| signal recognition particle 14 kDa protein | NP_003125.3 |
| small nuclear ribonucleoprotein Sm D2 isoform 1 | NP_004588.1 |
| small nuclear ribonucleoprotein Sm D3 | NP_004166.1 |
| sterol regulatory element-binding protein 1 isoform a | NP_001005291.1 |
| sterol regulatory element-binding protein 2 | NP_004590.2 |
| stromal cell-derived factor 1 isoform gamma | NP_001029058.1 |
| talin-1 | NP_006280.3 |
| TATA-box-binding protein isoform 1 | NP_003185.1 |
| TATA-box-binding protein isoform 2 | NP_001165556.1 |
| T-cell leukemia homeobox protein 2 | NP_057254.1 |
| T-cell surface glycoprotein CD4 isoform 1 precursor | NP_000607.1 |
| T-cell surface glycoprotein CD4 isoform 3 | NP_001181946.1 |
| telomeric repeat-binding factor 1 isoform 1 | NP_059523.2 |
| telomeric repeat-binding factor 2 | NP_005643.1 |
| THAP domain-containing protein 1 isoform 1 | NP_060575.1 |
| transcription factor AP-1 | NP_002219.1 |
| transcription factor NF-E2 45 kDa subunit isoform 2 | NP_001129495.1 |
| transcription factor SOX-2 | NP_003097.1 |
| transcription factor Sp 1 isoform b | NP_003100.1 |
| transcriptional activator Myb isoform 1 | NP_001123645.1 |
| transcriptional activator Myb isoform 4 | NP_001155128.1 |
| troponin T, cardiac muscle isoform 2 | NP_001001430.1 |
| tumor necrosis factor receptor superfamily member 13C | NP_443177.1 |
| U1 small nuclear ribonucleoprotein A | NP_004587.1 |
| voltage-dependent L-type calcium channel subunit alpha-1C | NP_001161097.1 |
| isoform 23 | |
| zinc finger Ran-binding domain-containing protein 2 isoform 2 | NP_005446.2 |

^{a}“Refseq” is the NCBI Reference Sequence ID on the web at ncbi.nlm.nih.gov/RefSeq/RSfaq.html#background.

Regardless of the specific Surf+ Penetrating Polypeptide or category of Surf+ Penetrating Polypeptide used in a complex, the disclosure contemplates embodiments in which the complex comprises a domain of a full length, naturally occurring human protein, but does not include the full length, naturally occurring human protein as a contiguous amino acid sequence. However, even when a domain of a full length, naturally occurring human protein is providing the Surf+ Penetrating Polypeptide function for a complex, the disclosure contemplates embodiments in which that domain is provided in the context of the full length (or substantially full length), naturally occurring protein—such that the complex comprises the full length, naturally occurring human protein, or when the Surf+ Polypeptide portion includes additional polypeptide sequence (more sequence than is necessary or sufficient to achieve cell penetration).

In some embodiments, a complex comprises a portion comprising a Surf+ Penetrating Polypeptide and the portion comprising a Surf+ Penetrating Polypeptide is selected from a polypeptide listed in FIG. 1 or 2. In certain embodiments, the complex includes at least about 10%, 15%, 20%, 25%, 30%, 35%, 40%, 45%, 50%, 55%, 60%, 65%, 70%, 75%, 80%, 85%, 90%, 95%, 96%, 97%, 98%, or 100% of the full length polypeptide from which the Surf+ Penetrating polypeptide is a domain, provided as contiguous amino acid residues.

For illustrative purposes, the disclosure has provided numerous exemplary Surf+ Penetrating Polypeptides, including numerous human polypeptides. However, Surf+ Penetrating Polypeptides suitable for use also include polypeptides from other species, such as mouse, rat, monkey, etc. Accordingly, the disclosure contemplates use of naturally occurring polypeptides (and domains thereof having characteristics of Surf+ Penetrating Polypeptides) from these other organisms. Accordingly, in one embodiment, the disclosure provides a complex comprising a Surf+ Penetrating Polypeptide, which is a naturally occurring mammalian polypeptide (such as mouse, rat, monkey, etc.) or domain thereof associated with an AAM moiety.

Supercharging

In addition, in certain embodiments, Surf+ Penetrating Polypeptides include naturally occurring or non-human proteins that may be or have been further modified to increase positive charge (e.g., supercharged). These include polypeptides that, prior to supercharging, have a charge/molecular weight ratio of at least 0.75 or of greater than 0.75, as well as polypeptides that do not have a charge/molecular weight ratio of at least 0.75 prior to supercharging. An example is the +52 streptavidin described in the Examples in which streptavidin has been supercharged to have a net positive charge of +52. Another example is the +36 GFP described in the Examples in which GFP has been supercharged to have a net positive charge of +36.

Surf+ Penetrating Polypeptides can be naturally-occurring, or can be produced by changing one or more conserved or non-conserved amino acids on or near the surface of a protein to more polar or charged amino acid residues. The amino acid residues to be modified may be hydrophobic, hydrophilic, charged, or a combination thereof. Surf+ Penetrating Polypeptides can also be produced by the attachment of charged moieties to the protein in order to supercharge the protein.

Natural as well as unnatural proteins (e.g., engineered proteins) may be modified, e.g., to increase the net charge of the protein. Examples of proteins that may be modified include receptors, membrane bound proteins, transmembrane proteins, enzymes, transcription factors, extracellular proteins, therapeutic proteins, cytokines, messenger proteins, DNA-binding proteins, RNA-binding proteins, proteins involved in signal transduction, structural proteins, cytoplasmic proteins, nuclear proteins, hydrophobic proteins, hydrophilic proteins, etc.

A naturally occurring Surf+ Penetrating Polypeptides, or a protein to be modified for supercharging, may be derived from any species of plant, animal, and/or microorganism. In certain embodiments, the protein is a mammalian protein. In certain embodiments, the protein is a human protein. In certain embodiments, the naturally occurring Surf+ Penetrating Polypeptide, or the protein to be modified, is derived from an organism typically used in research. For example, the naturally occurring Surf+ Penetrating Polypeptide, or the protein to be modified, may be from a primate (e.g., ape, monkey), rodent (e.g., rabbit, hamster, gerbil), pig, dog, cat, fish (e.g., Danio rerio), nematode (e.g., C. elegans), yeast (e.g., Saccharomyces cerevisiae), or bacteria (e.g., E. coli). In certain embodiments, the protein is non-immunogenic. In other certain embodiments, the protein is non-antigenic. In certain embodiments, the protein does not have inherent biological activity or has been modified to have no biological activity. In certain embodiments, the protein is chosen based on its targeting ability.

In certain embodiments of the disclosure, the term supercharging is used to refer to changes made to the Surf+ Penetrating Polypeptide or changes made to a polypeptide such that it functions as and meets the definition of a Surf+ Penetrating Polypeptide, but do not include changes in charge or charge density that result from association with the AAM moiety.

In some embodiments, the naturally occurring Surf+ Penetrating Polypeptides, or the protein to be modified is one whose structure has been characterized, for example, by NMR or X-ray crystallography. In some embodiments, the naturally occurring Surf+ Penetrating Polypeptides, or the protein to be modified, is one whose structure has been predicted, for example, by threading homology modeling or de novo structure prediction. In some embodiments, the naturally occurring Surf+ Penetrating Polypeptides, or the protein to be modified, is one whose structure has been correlated and/or related to biochemical activity (e.g., enzymatic activity, protein-protein interactions, etc.). In certain embodiments, the inherent biological activity of a modified protein is reduced or eliminated to reduce the risk of deleterious and/or undesired effects. Alternatively, the biological activity of the modified protein can be increased or potentiated, or a non-naturally occurring biological activity of the protein may be generated as a result of the charge modification concomitant with the creation of the charged-modified Surf+ Penetrating Polypeptides.

For embodiments in which a protein is modified to generate a Surf+ Penetrating Polypeptides, the surface residues of a protein to be modified may be identified using any method known in the art. In certain embodiments, surface residues are identified by computer modeling of the protein. In certain embodiments, the three-dimensional structure of the protein is known and/or determined, and surface residues are identified by visualizing the structure of the protein. Homology modeling and de novo structure prediction are two methods for modeling the 3-D structure of a protein; such methods are particularly useful in the absence of an NMR or crystal structure. In some embodiments, surface residues are predicted using computer software. In certain particular embodiments, an Accessible Surface Area (ASA) is used to predict surface exposure. A high ASA value indicates a surface exposed residue, whereas a low ASA value indicates the exclusion of solvent interactions with the residue. In certain particular embodiments, an Average Neighbor Atoms per Sidechain Atom (AvNAPSA) value is used to predict surface exposure. AvNAPSA is an automated measure of surface exposure which has been implemented as a computer program. A low AvNAPSA value indicates a surface exposed residue, whereas a high value indicates a residue in the interior of the protein. In certain embodiments, the software is used to predict the secondary structure and/or tertiary structure of a protein, and surface residues or near-surface residues are identified based on this prediction. In some embodiments, the prediction of surface residues is based on hydrophobicity and hydrophilicity of the residues and their clustering in the primary sequence of the protein. Besides in silico methods, surface residues of the protein may also be identified using various biochemical techniques, for example, protease cleavage, surface modification, derivatization, labeling, hydrogen-deuterium exchange experiments, etc. We note that such modeling is also useful for identifying domains of a full length protein that possess characteristics of s Surf+ Penetrating Polypeptide.

Optionally, of the surface residues, it is then determined which are conserved or important to the functioning of the protein. However, conserved amino acids may be modified even if the underlying biological activity of the protein is to be retained, reduced, enhanced or augmented by one or more non-naturally occurring biological activities. Identification of conserved residues can be determined using any method known in the art. In certain embodiments, conserved residues are identified by aligning the primary sequence of the protein of interest with related proteins. These related proteins may be from the same family of proteins. Related proteins may also be the same protein from a different species. For example, conserved residues may be identified by aligning the sequences of the same protein from different species. For example, proteins of similar function or biological activity may be aligned. Preferably, 2, 3, 4, 5, 6, 7, 8, 9, 10 or more than 10 different sequences are used to determine the conserved amino acids in the protein. In certain embodiments, a residue is considered conserved if over 50%, over 60%, over 70%, over 75%, over 80%, over 90%, or over 95% of the sequences have the same amino acid in a particular position. In other embodiments, the residue is considered conserved if over 50%, over 60%, over 70%, over 75%, over 80%, over 90%, or over 95% of the sequences have the same or a similar (e.g., valine, leucine, and isoleucine; glycine and alanine; glutamine and asparagine; or aspartate and glutamate) amino acid in a particular position. Many software packages are available for aligning and comparing protein sequences as described herein. As would be appreciated by one of skill in the art, either the conserved residues may be determined first or the surface residues may be determined first. The order does not matter. In certain embodiments, a computer software package may determine surface residues and/or conserved residues, and may optionally do so simultaneously. Important residues in the protein may also be identified by mutagenesis of the protein. For example, alanine scanning of the protein can be used to determine the important amino acid residues in the protein. In some embodiments, site-directed mutagenesis may be used. In certain embodiments, conserving the original biological activity of the protein is not important, and therefore, the steps of identifying the conserved residues and preserving them are not performed.

Each of the surface residues is identified as hydrophobic or hydrophilic. In certain embodiments, residues are assigned a hydrophobicity score. For example, each surface residue may be assigned an octanol/water log P value. Other hydrophobicity parameters may also be used. Such scales for amino acids have been discussed in: Janin, 1979, Nature, 277:491; Wolfenden et al., 1981, Biochemistry, 20:849; Kyte et al., 1982, J. Mol. Biol., 157:105; Rose et al., 1985, Science, 229:834; Corvette et al., 1987, J. Mol. Biol., 195:659; Charton and Charton, 1982, J. Theon. Biol., 99:629; each of which is incorporated by reference. Any of these hydrophobicity parameters may be used in the inventive method to determine which residues to modify. In certain embodiments, hydrophilic or charged residues are identified for modification. Near-surface residues are residues that are either a) not surface residues but immediately adjacent in primary amino acid sequence or within a three-dimensional structure or b) not surface residues that can become surface residues upon the alteration of a polypeptide's tertiary structure. The contribution of near-surface residues in a Surf+ Penetrating Polypeptideis determined using the methods described herein.

In certain embodiments, for generation of Surf+ Penetrating Polypeptides, at least one identified surface residue or near-surface residue is chosen for modification. In certain embodiments, hydrophobic residue(s) are chosen for modification. In other embodiments, hydrophilic and/or charged residue(s) are chosen for modification. In certain embodiments, more than one residue is chosen for modification. In certain embodiments, 1, 2, 3, 4, 5, 6, 7, 8, 9, 10 or more than 10 of the identified residues are chosen for modification. In certain embodiments, over 10, over 15, over 20, or over 25 residues are chosen for modification.

In certain embodiments, multiple variants of a protein, each with different modifications, are produced and tested to determine the best variant in terms of delivery of a biological moiety to a cell, pharmacokinetics, stability, biocompatibility, and/or biological activity, or a biophysical property such as expression level. In some embodiments, a library of protein variants is generated in an in vivo system containing an expression host such as phage, bacteria, yeast or mammalian cells, or in an in vitro system such as mRNA display, ribosome display, or polysome display. Such a library may contain 10, 10², 10³, 10⁴, 10⁵, 10⁶, 10⁷, 10⁸, 10⁹, or over 10⁹, possible variants (including substitutions, deletions of one or more residues, and insertion of one or more residues). By testing the variants resulting from this library, Surf+ Penetrating Polypeptides may be created from polypeptides for which no structural information such as crystal structure is known or available.

In certain embodiments, residues chosen for modification are mutated into more hydrophilic residues (including positively charged residues). Typically, residues are mutated into more hydrophilic natural amino acids. In certain embodiments, residues are mutated into amino acids that are positively charged at physiological pH. For example, a residue may be changed to an arginine, or lysine, or histidine. In certain embodiments, all the residues to be modified are changed into the same alternate residue. For example, all the chosen residues are changed to an arginine residue, a lysine residue or a histidine residue. In other embodiments, the chosen residues are changed into different residues; however, all the final residues are positively charged at physiological pH. In certain embodiments, to create a positively charged protein, all the residues to be mutated are converted to arginine or lysine or histidine residues, or a combination thereof. To give but another example, all the chosen residues for modification are aspartate, glutamate, asparagine, and/or glutamine, and these residues are mutated into arginine, lysine or histidine.

In some embodiments, a protein may be modified to increase the overall net charge on the protein. In certain embodiments, the theoretical net charge is increased, relative to its unmodified protein, by at least +1, at least +2, at least +3, at least +4, at least +5, at least +10, at least +15, at least +20, at least +25, at least +30, at least +35, or at least +40. In certain embodiments, the chosen amino acids are changed into non-ionic, polar residues (e.g., cysteine, serine, threonine, tyrosine, glutamine, and asparagine). In some embodiments, increasing the overall net charge comprises increasing the total number of positively charged residues on or near the surface.

In certain embodiments, the amino acid residues mutated to charged amino acids residues are separated from each other by at least 1, at least 2, at least 3, at least 4, at least 5, at least 6, at least 7, at least 8, at least 9, at least 10, at least 15, at least 20, or at least 25 amino acid residues in the primary amino acid sequence. In certain embodiments, the amino acid residues mutated to positively charged amino acids residues (e.g., arginine, lysine or histidine) are separated from each other by at least 1, at least 2, at least 3, at least 4, at least 5, at least 6, at least 7, at least 8, at least 9, at least 10, at least 15, at least 20, or at least 25 amino acid residues in the primary amino acid sequence. In certain embodiments, fewer than two or only two, three, four or five consecutive amino acids are modified to generate a charge-modified Surf+ Penetrating Polypeptide. Alternatively, wherein a surface projection is present in the polypeptide, more than two, three, four, five, six, seven, eight, nine, or ten consecutive amino acids are modified to generate a charged-modified Surf+ Penetrating Polypeptide.

In certain embodiments, a surface exposed loop, helix, turn, or other secondary structure may contain only 1, 2, 3, 4, 5, 6, 7, 8, 9, 10, 15, 20, 25, 30 or more than 30 charged residues. Distributing the charged residues over the surface of the protein may allow for more stable proteins. In certain embodiments, only 1, 2, 3, 4, or 5 residues per 15-20 amino acids of the primary sequence are mutated to charged amino acids (e.g., arginine, lysine or histidine). In certain embodiments, on average only 1, 2, 3, 4, or 5 residues per 10 amino acids of the primary sequence are mutated to charged amino acids (e.g., arginine, lysine or histidine). In certain embodiments, on average only 1, 2, 3, 4, or 5 residues per 15 amino acids of the primary sequence are mutated to charged amino acids (e.g., arginine, lysine or histidine). In certain embodiments, on average only 1, 2, 3, 4, or 5 residues per 20 amino acids of the primary sequence are mutated to charged amino acids (e.g., arginine, lysine or histidine). In certain embodiments, on average only 1, 2, 3, 4, or 5 residues per 25 amino acids of the primary sequence are mutated to charged amino acids (e.g., arginine, lysine or histidine). In certain embodiments, on average only 1, 2, 3, 4, or 5 residues per 30 amino acids of the primary sequence are mutated to charged amino acids (e.g., arginine, lysine or histidine).

In certain embodiments, at least 50%, at least 60%, at least 70%, at least 80%, or at least 90% of the mutated charged amino acid residues of a charge-modified Surf+ Penetrating Polypeptide are solvent exposed. In certain embodiments, at least 50%, at least 60%, at least 70%, at least 80%, or at least 90% of the mutated charged amino acids residues of the charge-modified Surf+ Penetrating Polypeptide are on the surface of the protein. In certain embodiments, less than 5%, less than 10%, less than 20%, less than 30%, less than 40%, less than 50% of the mutated charged amino acid residues are not solvent exposed. In certain embodiments, less than 5%, less than 10%, less than 20%, less than 30%, less than 40%, less than 50% of the mutated charged amino acid residues are internal amino acid residues.

In some embodiments, amino acids are selected for modification using one or more predetermined criteria. For example, to generate a superpositively charged protein, ASA or AvNAPSA values may be used to identify aspartic acid, glutamic acid, asparagine, and/or glutamine residues with ASA values above a certain threshold value or AvNAPSA values below a certain threshold value, and one or more (e.g., all) of these residues may be changed to arginine, lysine or histidine. In some embodiments, to generate a superpositively charged protein, ASA calculations are used to identify aspartic acid, glutamic acid, asparagine, and/or glutamine residues with ASA above a certain threshold value, and one or more (e.g., all) of these are changed to arginine, lysine or histidine. In some embodiments, to generate a superpositively charged protein, AvNAPSA is used to identify aspartic acid, glutamic acid, asparagine, and/or glutamine residues with AvNAPSA below a certain threshold value, and one or more (e.g., all) of these are changed to arginines. In some embodiments, to generate a superpositively charged protein, AvNAPSA is used to identify aspartic acid, glutamic acid, asparagine, and/or glutamine residues with AvNAPSA below a certain threshold value, and one or more (e.g., all) of these are changed to lysines. In other embodiments, to generate a superpositively charged protein, AvNAPSA is used to identify aspartic acid, glutamic acid, asparagine, and/or glutamine residues with AvNAPSA below a certain threshold value, and one or more (e.g., all) of these are changed to histidines.

In some embodiments, solvent-exposed residues are identified by the number of neighbors. In general, residues that have more neighbors are less solvent-exposed than residues that have fewer neighbors. In some embodiments, solvent-exposed residues are identified by half sphere exposure, which accounts for the direction of the amino acid side chain (Hamelryck, 2005, Proteins, 59:8-48; incorporated herein by reference). In some embodiments, solvent-exposed residues are identified by computing the solvent exposed surface area, accessible surface area, and/or solvent excluded surface of each residue. See, e.g., Lee et al., J. Mol. Biol. 55(3):379-400, 1971; Richmond, J. Mol. Biol. 178:63-89, 1984; each of which is incorporated herein by reference.

The desired modifications or mutations in the protein may be accomplished using any techniques known in the art. Recombinant DNA techniques for introducing such changes in a protein sequence are well known in the art. In certain embodiments, the modifications are made by site-directed mutagenesis of the polynucleotide encoding the protein. Other techniques for introducing mutations are discussed in Molecular Cloning: A Laboratory Manual, 2nd Ed., ed. by Sambrook, Fritsch, and Maniatis (Cold Spring Harbor Laboratory Press: 1989); the treatise, Methods in Enzymology (Academic Press, Inc., N.Y.); Ausubel et al. Current Protocols in Molecular Biology (John Wiley & Sons, Inc., New York, 1999); each of which is incorporated herein by reference. The modified protein is expressed and tested. In certain embodiments, a series of variants is prepared, and each variant is tested to determine its biological activity and its stability. The variant chosen for subsequent use may be the most stable one, the most active one, or the one with the greatest overall combination of activity and stability. After a first set of variants is prepared an additional set of variants may be prepared based on what is learned from the first set. Variants are typically created and over-expressed using recombinant techniques known in the art.

As would be appreciated by one of skill in the art, protein fragments, functional protein domains, and homologous proteins are also considered to be within the scope of this disclosure. For example, provided herein is any protein fragment of a reference protein (meaning a polypeptide sequence at least one amino acid residue shorter than a reference polypeptide sequence but otherwise identical) 10, 20, 30, 40, 50, 60, 70, 80, 90, 100 or greater than 100 amino acids in length. In another example, any protein that includes a stretch of about 20, about 30, about 40, about 50, or about 100 amino acids which are about 40%, about 50%, about 60%, about 70%, about 80%, about 90%, about 95%, or about 100% identical to any of the sequences described herein can be utilized in accordance with the disclosure. In certain embodiments, a protein sequence to be utilized in accordance with the disclosure includes 2, 3, 4, 5, 6, 7, 8, 9, 10, or more mutations as shown in any of the sequences provided or referenced herein.

### Antibody or Antibody-Mimic Moiety (AAM Moiety)

The disclosure provides complexes comprising a Surf+ Penetrating Polypeptide portion, as described above, and an antibody or antibody-mimic moiety (AAM moiety) portion that is associated with the Surf+ Penetrating Polypeptide portion. This section of the application describes the AAM moiety portion of complexes of the disclosure and provides numerous representative examples. The disclosure contemplates that any such AAM moiety may be associated with any Surf+ Penetrating Polypeptide or category of Surf+ Penetrating Polypeptide to form a complex (e.g., may be associated to a portion comprising or consisting of a Surf+ Penetrating Polypeptide). Such a complex has cell penetrating ability (e.g., cell penetrating ability provided by the Surf+ Penetrating Polypeptide portion) and promotes delivery of the AAM moiety into a cell. As described in greater detail below, AAM moieties for use in the context of the present disclosure bind to intraceullar targets (e.g., bind to targets expressed or otherwise present inside a cell). Accordingly, the present disclosure provides complexes and methods for delivering the AAM moiety into a cell where it can bind its target molecule.

As used herein, an “AAM moiety” is an antibody or an antibody mimic molecule that specifically binds to a target molecule expressed or otherwise present intracellularly (an intracellular target). An antibody-mimic molecule is also referred to as an antibody-like molecule. An antibody-mimic binds to a target molecule, but binding is mediated by binding units other than antigen binding portions comprising at least a variable heavy or variable light chain of an antibody. Thus, in an antibody mimic, binding to target is mediated by a different antigen-binding unit, such as a protein scaffold or other engineered binding unit. Numerous categories of antibody-mimics are well known in the art and are described in further detail below.

The term “target” refers to a molecule expressed or otherwise present inside a cell to which an AAM moiety specifically binds (e.g., binds with affinity and specificity distinct from non-specific interactions). In certain embodiments, the target is a peptide or polypeptide, including peptides or polypeptides that are glycosylated, phosphorylated or otherwise post-translationally modified. The term “intracellular target” refers to molecules expressed or otherwise present in a cell so that the target can be contacted while inside the cell by an AAM moiety. For example, a secreted polypeptide that is taken up by a cell is, for some period of time, present inside a cell. Thus, while present inside a cell, such a secreted polypeptide may be an intracellular target available to be contacted by an AAM moiety. In certain embodiments, the intracellular target is a target whose endogenous localization is inside a cell (e.g., the target is not secreted).

In certain embodiments, the AAM moiety binds to a target expressed or otherwise present intracellularly, and that target is distinct from the Surf+ Penetrating Polypeptide to which the AAM moiety is complexed. In other words, the Surf+ Penetrating Polypeptide or Surf+ Penetrating Polypeptide portion to which the AAM moiety is complexed is not also the endogenous target of the AAM moiety. However, in certain embodiments, it is possible that the Surf+ Penetrating Polypeptide may itself bind to or have some affinity for the same target. This, however, is permissible and is not intended to be excluded by the foregoing description.

In certain embodiments, a complex of the disclosure comprises an AAM moiety, wherein the AAM moiety is an antibody that binds to a target molecule expressed inside a cell. In certain embodiments, a complex of the disclosure comprises an AAM moiety, wherein the AAM moiety is an antibody-mimic (e.g., a protein comprising a protein scaffold or other binding unit that binds to a target expressed inside a cell). In certain embodiments, the AAM moiety binds to its target, and that target is a polypeptide expressed in a cell. In certain embodiments, the AAM moiety binds its target molecule, such as a polypeptide, with high affinity (e.g., with an affinity of at least 10⁻⁶, 10⁻⁷, 10⁻⁸, 10⁻⁹, 10⁻¹⁰, or 10⁻¹¹M, or with an affinity in the range of 10⁻⁶ to 10⁻⁸, 10⁻⁷ to 10⁻¹⁰, or 10⁻⁹ to 10⁻¹¹M). In certain embodiments, the AAM moiety binds to its target with an affinity at least 100, at least 1000, or at least 10000 times tighter than its affinity for another polypeptide. Regardless of the affinity with which an AAM moiety binds its target, binding is understood to not include nonspecific binding (e.g., binding due to background or general stickiness of polypeptides).

It should be appreciated that the target may also be expressed extracellularly. However, in the context of the present disclosure, the primary aim is to facilitate delivery of the AAM moiety into a cell to promote binding of the AAM moiety to target expressed inside a cell. Nevertheless, the fact that the target moiety, such as a polypeptide, is also expressed extracellularly does not limit its suitability as a target. Non-limiting examples of target polypeptides are described in greater detail in the portion of the disclosure entitled “Applications”. However, these serve only as examples.

Binding of an AAM moiety to a target is generally intended to have one or more biological consequences or utilities. For example, binding of an AAM moiety may be useful for inhibiting the activity of the target, such as by preventing binding to another protein, by promoting degradation of the target, or by sequestering the target away from its necessary site of action. Binding of an AAM moiety may also be useful for labeling a target to facilitate visualization or monitoring of cells expressing the target. Given a particular known target polypeptide, numerous methods exist for identifying AAM moieties that bind to the target and that have a desired function, e.g., that inhibit activity of the target or that bind to the target without altering activity (so as to serve as a suitable labeling agent). Exemplary methods of making and testing AAM moieties that bind a target are described herein.

In certain embodiments, an AAM moiety is an antibody-mimic comprising a protein scaffold. Scaffold-based AAM moieties have positioning or structural components and target-contacting components in which the target contacting residues are largely concentrated. Thus, in an embodiment, a scaffold-based AAM moiety comprises a scaffold comprising two types of regions, structural and target contacting. The target contacting region shows more variability than does the structural region when a scaffold-based AAM moiety to a first target is compared with a scaffold-based AAM moiety of a second target (where both AAM moieties are of the same category, e.g., both are Adnectins or both are Anticalins®). The structural region tends to be more conserved across AAM moieties that bind different targets. This is analogous to the CDRs and framework regions of antibodies. In the case of an Anticalin®, the first class corresponds to the loops, and the second class corresponds to the anti-parallel strands.

In certain embodiments the AAM moiety is a subunit-based AAM moiety. These AAM moieties are based on an assembly of subunits which provide distributed points of contact with the target that form a domain that binds with high affinity to the target (e.g. as seen with DARPins).

In certain embodiments an AAM moiety for use as part of a complex of the disclosure has a molecular weight of 5-250, 10-200, 5-15, 10-30, 15-30, 20-25 kD. AAM moieties can comprise one or more polypeptide chains.

AAM moieties can be antibody-based or non-antibody-based.

AAM moieties suitable for use in the compositions and methods featured in the disclosure include antibody molecules, such as full-length antibodies and antigen-binding fragments thereof, and single domain antibodies, such as camelids. For example, an antibody molecule is complexed with an Surf+ Penetrating Polypeptide for delivery of the antibody molecule into a cell. The antibody molecule binds an intracellular target, e.g., an intracellular polypeptide, such as to inhibit, label or activate the target, e.g., for treatment of a disorder, for labeling to monitor expression or as a diagnostic, for research or clinical purposes.

Other suitable AAM moieties include polypeptides engineered to contain a scaffold protein, such as a DARPin, an Adnectin®, or an Anticalin®. These are exemplary of antibody-mimic moieties that, in the context of the disclosure, may be complexed with a Surf+ Penetrating Polypeptide to promote delivery of the AAM moiety into a cell. The scaffold protein (e.g., the AAM moiety portion of the complex) binds an intracellular target, e.g., an intracellular polypeptide, such as to inhibit, label or activate the target, e.g., for treatment of a disorder, for labeling to monitor expression or as a diagnostic, for research purposes. Inhibition can be, e.g., by steric inhibition, e.g., by blocking protein interaction with a substrate, or inhibition can be, e.g., by causing target protein degradation.

An AAM moiety for delivery into a cell can be, e.g., an agent for treatment, prophylaxis, diagnosis, imaging, or labeling. In some embodiments, the AAM moiety has a desirable activity in a target cell, but the Surf+ Penetrating Polypeptide that delivers the AAM moiety is inert, i.e., the Surf+ Penetrating Polypeptide has no observable biological function in the cell other than to deliver the agent to the interior of the cell. In other embodiments, the Surf+ Penetrating Polypeptide has at least one desired biological activity, e.g., the polypeptide modifies (e.g., enhances) the effect of the AAM moiety on a target molecule, or the Surf+ Penetrating Polypeptide binds to and affects the activity of a second target molecule that is separate from the first molecule targeted by the high affinity binding ligand.

Before describing exemplary AAM moieties and sub-categories of AAM moieties in greater detail, in should be understood that the AAM moiety itself has charge, size and charge distribution characteristics. However, such charge or charge distribution characteristics are not considered when describing the charge characteristics of the Surf+ Penetrating Polypeptide portion or when evaluating whether the Surf+ Penetrating Polypeptide portion has been supercharged or modified. Rather, supercharging refers to changes to Surf+ Penetrating Polypeptide—other than occur simply by complexing to an AAM moiety.

Antibody Molecules

As used herein, the term “antibody” or “antibody molecule” refers to a protein that includes sufficient sequence (e.g., antibody variable region sequence) to mediate binding to a target, and in embodiments, includes at least one immunoglobulin variable region or an antigen binding fragment thereof.

An antibody molecule can be, for example, a full-length, mature antibody, or an antigen binding fragment thereof. An antibody molecule, also known as an antibody or an immunoglobulin, encompass monoclonal antibodies (including full-length monoclonal antibodies), polyclonal antibodies, multispecific antibodies formed from at least two different epitope binding fragments (e.g., bispecific antibodies), human antibodies, humanized antibodies, camelised antibodies, chimeric antibodies, single-chain Fvs (scFv), Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies of the disclosure), intrabodies, and epitope-binding fragments of any of the above. In particular, antibodies include immunoglobulin molecules and immunologically active fragments of immunoglobulin molecules, i.e., molecules that contain at least one antigen-binding site. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m(f, z, a or x), G2m(n), G3m(g, b, or c), Am, Em, and Km(1, 2 or 3)). Antibodies may be derived from any mammal, including, but not limited to, humans, monkeys, pigs, horses, rabbits, dogs, cats, mice, etc., or other animals such as birds (e.g. chickens). The antibody molecule can be a single domain antibody, e.g., a nanobody, such as a camelid, or a llama- or alpaca-derived single domain antibody, or a shark antibody (IgNAR). The single domain antibody comprises, e.g., only a variable heavy domain (VHH). An antibody molecule can also be a genetically engineered single domain antibody. Typically, the antibody molecule is a human, humanized, chimeric, camelid, shark or in vitro generated antibody.

Examples of fragments include (i) an Fab fragment having a VL, VH, constant light chain domain (CL) and constant heavy chain domain 1 (CH1) domains; (ii) an Fd fragment having VH and CH1 domains; (iii) an Fv fragment having VL and VH domains of a single antibody; (iv) a dAb fragment (Ward, E. S. et al., Nature 341, 544-546 (1989); McCafferty et al (1990) Nature, 348, 552-55; and Holt et al (2003) Trends in Biotechnology 21, 484-490), having a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988 and Huston et al, PNAS USA, 85, 5879-5883, 1988) (viii) bispecific single chain Fv dimers (for example as disclosed in WO 1993/011161) and (ix) “diabodies”, multivalent or multispecific fragments constructed by gene fusion (for example as disclosed in WO94/13804 and Holliger, P. et al, Proc. Natl. Acad. Sci. USA 90 6444-6448, 1993). Fv, scFv or diabody molecules may be stabilized by the incorporation of disulphide bridges linking the VH and VL domains (Reiter, Y. et al, Nature Biotech, 14, 1239-1245, 1996). Minibodies comprising a scFv joined to a CH3 domain may also be made (Hu, S. et al, Cancer Res., 56, 3055-3061, 1996). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group. These antibody fragments are obtained using conventional techniques known to those with skill in the art, and the fragments are screened for utility in the same manner as are intact antibodies. Suitable fragments may, in certain embodiments, be obtained from human or rodent antibodies.

The term “antibody molecule” includes intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function). In certain embodiments, antibodies for use in the present disclosure are labelled, modified to increase half-life, and the like. For example, in certain embodiments, the antibody is chemically modified, such as by PEGylation, or by incorporation in a liposome.

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, light chains devoid of heavy chains, single domain antibodies derived from conventional 4-chain antibodies, and engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. In one aspect of the disclosure, a single domain antibody can be derived from a variable region of the immunoglobulin found in fish, such as, for example, that which is derived from the immunoglobulin isotype known as Novel Antigen Receptor (NAR) found in the serum of shark. Methods of producing single domain antibodies derived from a variable region of NAR (“IgNARs”) are described in WO 03/014161 and Streltsov (2005) Protein Sci. 14:2901-2909. According to another aspect, a single domain antibody is a naturally occurring single domain antibody known as a heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; and such VHHs are within the scope of the disclosure.

The VH and VL regions can be subdivided into regions of hypervariability, termed “complementarity determining regions” (CDR), interspersed with regions that are more conserved, termed “framework regions” (FR). The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Each VH and VL typically includes three CDRs and four FRs, arranged from amino-terminus to carboxy-terminus in the following order: FR1, CDR1, FR2, CDR2, FR3, CDR3, FR4.

The VH or VL chain of the antibody molecule can further include all or part of a heavy or light chain constant region, to thereby form a heavy or light immunoglobulin chain, respectively. In one embodiment, the antibody molecule is a tetramer of two heavy immunoglobulin chains and two light immunoglobulin chains. The heavy and light immunoglobulin chains can be connected by disulfide bonds. The heavy chain constant region typically includes three constant domains, CH1, CH2 and CH3. The light chain constant region typically includes a CL domain. The variable region of the heavy and light chains contains a binding domain that interacts with an antigen. The constant regions of the antibody molecules typically mediate the binding of the antibody to host tissues or factors, including various cells of the immune system (e.g., effector cells) and the first component (C1q) of the classical complement system.

The term “immunoglobulin” comprises various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgD, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the present disclosure. All immunoglobulin classes are also within the scope of the present disclosure. Light chains are classified as either kappa or lambda (κ, λ). Each heavy chain class may be bound with either a kappa or lambda light chain.

The term “antigen-binding fragment” refers to one or more fragments of a full-length antibody that retain the ability to specifically bind to a target of interest. Examples of binding fragments encompassed within the term “antigen-binding fragment” of a full length antibody include (i) a Fab fragment, a monovalent fragment having VL, VH, CL and CH1 domains; (ii) a F(ab′)₂ fragment, a bivalent fragment including two Fab fragments linked by a disulfide bridge at the hinge region; (iii) an Fd fragment having VH and CH1 domains; (iv) an Fv fragment having VL and VH domains of a single arm of an antibody, (v) a dAb fragment (Ward et al., (1989) Nature 341:544-546), which has a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The term “antigen-binding site” refers to the part of an antibody molecule that comprises determinants that form an interface that binds to a target antigen, or an epitope thereof. With respect to proteins (or protein mimetics), the antigen-binding site typically includes one or more loops (of at least four amino acids or amino acid mimics) that form an interface that binds to the target antigen or epitope thereof. Typically, the antigen-binding site of an antibody molecule includes at least one or two CDRs, or more typically at least three, four, five or six CDRs.

Regardless of the type of antibody used, in certain embodiments, the antibody may comprise replacing one or more amino acid residue(s) with a non-naturally occurring or non-standard amino acid, modifying one or more amino acid residue into a non-naturally occurring or non-standard form, or inserting one or more non-naturally occurring or non-standard amino acid into the sequence. Examples of numbers and locations of alterations in sequences are described elsewhere herein. Naturally occurring amino acids include the 20 “standard” L-amino acids identified as G, A, V, L, I, M, P, F, W, S, T, N, Q, Y, C, K, R, H, D, E by their standard single-letter codes. Non-standard amino acids include any other residue that may be incorporated into a polypeptide backbone or result from modification of an existing amino acid residue. Non-standard amino acids may be naturally occurring or non-naturally occurring. Several naturally occurring non-standard amino acids are known in the art, such as 4-hydroxyproline, 5-hydroxylysine, 3-methylhistidine, N-acetylserine, etc. (Voet & Voet, Biochemistry, 2nd Edition, (Wiley) 1995). Those amino acid residues that are derivatised at their N-alpha position will only be located at the N-terminus of an amino-acid sequence. Normally, an amino acid is an L-amino acid, but it may be a D-amino acid. Alteration may therefore comprise modifying an L-amino acid into, or replacing it with, a D-amino acid. Methylated, acetylated and/or phosphorylated forms of amino acids are also known, and amino acids in the present disclosure may be subject to such modification.

In certain embodiments, the antibodies used in the claimed methods are generated using random mutagenesis of one or more selected VH and/or VL genes to generate mutations within the entire variable domain. Such a technique is described by Gram et al., 1992, Proc. Natl. Acad. Sci., USA, 89:3576-3580 who used error-prone PCR. In some embodiments one or two amino acid substitutions are made within an entire variable domain or set of CDRs.

Another method that may be used is to direct mutagenesis to CDR regions of VH or VL genes. Such techniques are disclosed by Barbas et al., 1994, Proc. Natl. Acad. Sci., USA, 91:3809-3813 and Schier et al., 1996, J. Mol. Biol. 263:551-567.

Preparation of Antibodies

Suitable antibodies for use as an AAM moiety can be prepared using methods well known in the art. For example, antibodies can be generated recombinantly, made using phage display, produced using hybridoma technology, etc. Non-limiting examples of techniques are described briefly below.

In general, for the preparation of monoclonal antibodies or their functional fragments, especially of murine origin, it is possible to refer to techniques which are described in particular in the manual “Antibodies” (Harlow and Lane, Antibodies: A Laboratory Manual, Cold Spring Harbor Laboratory, Cold Spring Harbor N.Y., pp. 726, 1988) or to the technique of preparation from hybridomas described by Köhler and Milstein, Nature, 256:495-497, 1975.

Monoclonal antibodies can be obtained, for example, from a cell obtained from an animal immunized against the target antigen, or one of its fragments. Suitable fragments and peptides or polypeptides comprising them may be used to immunise animals to generate antibodies against the target antigen.

The monoclonal antibodies can, for example, be purified on an affinity column on which the target antigen or one of its fragments containing the epitope recognized by said monoclonal antibodies, has previously been immobilized. More particularly, the monoclonal antibodies can be purified by chromatography on protein A and/or G, followed or not followed by ion-exchange chromatography aimed at eliminating the residual protein contaminants as well as the DNA and the lipopolysaccaride (LPS), in itself, followed or not followed by exclusion chromatography on Sepharose™ gel in order to eliminate the potential aggregates due to the presence of dimers or of other multimers. In one embodiment, the whole of these techniques can be used simultaneously or successively.

It is possible to take monoclonal and other antibodies and use techniques of recombinant DNA technology to produce other antibodies or chimeric molecules that bind the target antigen. Such techniques may involve introducing DNA encoding the immunoglobulin variable region, or the CDRs, of an antibody to the constant regions, or constant regions plus framework regions, of a different immunoglobulin. See, for instance, EP-A-184187, GB 2188638A or EP-A-239400, and a large body of subsequent literature. A hybridoma or other cell producing an antibody may be subject to genetic mutation or other changes, which may or may not alter the binding specificity of antibodies produced.

Further techniques available in the art of antibody engineering have made it possible to isolate human and humanised antibodies. For example, human hybridomas can be made as described by Kontermann, R & Dubel, S, Antibody Engineering, Springer-Verlag New York, LLC; 2001, ISBN: 3540413545. Phage display, another established technique for generating antagonists has been described in detail in many publications, such as Kontermann & Dubel, supra and WO92/01047 (discussed further below), and U.S. Pat. No. 5,969,108, U.S. Pat. No. 5,565,332, U.S. Pat. No. 5,733,743, U.S. Pat. No. 5,858,657, U.S. Pat. No. 5,871,907, U.S. Pat. No. 5,872,215, U.S. Pat. No. 5,885,793, U.S. Pat. No. 5,962,255, U.S. Pat. No. 6,140,471, U.S. Pat. No. 6,172,197, U.S. Pat. No. 6,225,447, U.S. Pat. No. 6,291,650, U.S. Pat. No. 6,492,160 and U.S. Pat. No. 6,521,404.

Transgenic mice in which the mouse antibody genes are inactivated and functionally replaced with human antibody genes while leaving intact other components of the mouse immune system, can be used for isolating human antibodies Mendez, M. et al. (1997) Nature Genet, 15(2): 146-156. Humanised antibodies can be produced using techniques known in the art such as those disclosed in, for example, WO91/09967, U.S. Pat. No. 5,585,089, EP592106, U.S. Pat. No. 5,565,332 and WO93/17105. Further, WO2004/006955 describes methods for humanising antibodies, based on selecting variable region framework sequences from human antibody genes by comparing canonical CDR structure types for CDR sequences of the variable region of a non-human antibody to canonical CDR structure types for corresponding CDRs from a library of human antibody sequences, e.g. germline antibody gene segments. Human antibody variable regions having similar canonical CDR structure types to the non-human CDRs form a subset of member human antibody sequences from which to select human framework sequences. The subset members may be further ranked by amino acid similarity between the human and the non-human CDR sequences. In the method of WO2004/006955, top ranking human sequences are selected to provide the framework sequences for constructing a chimeric antibody that functionally replaces human CDR sequences with the non-human CDR counterparts using the selected subset member human frameworks, thereby providing a humanized antibody of high affinity and low immunogenicity without need for comparing framework sequences between the non-human and human antibodies. Chimeric antibodies made according to the method are also disclosed.

Synthetic antibody molecules may be created by expression from genes generated by means of oligonucleotides synthesized and assembled within suitable expression vectors, for example as described by Knappik et al. J. Mol. Biol. (2000) 296, 57-86 or Krebs et al. Journal of Immunological Methods 254 2001 67-84.

Note that regardless of how an antibody of interest is initially identified or made, any such antibody can be subsequently produced using recombinant techniques. For example, a nucleic acid sequence encoding the antibody may be expressed in a host cell. Such methods include expressing nucleic acid sequence encoding the heavy chain and light chain from separate vectors, as well as expressing the nucleic acid sequences from the same vector. These and other techniques using a variety of cell types are well known in the art.

Using these and other techniques known in the art, antibodies that specifically bind to any target can be made. Once made, antibodies can be tested to confirm that they bind to the desired target antigen and to select antibodies having desired properties. Such desired properties include, but are not limited to, selecting antibodies having the desired affinity and cross-reactivity profile. Given that large numbers of candidate antibodies can be made, one of skill in the art can readily screen a large number of candidate antibodies to select those antibodies suitable for the intended use. Moreover, the antibodies can be screened using functional assays to identify antibodies that bind the target and have a particular function, such as the ability to inhibit an activity of the target or the ability to bind to the target without inhibiting its activity. Thus, one can readily make antibodies that bind to a target and are suitable for an intended purpose.

The nucleic acid (e.g., the gene) encoding an antibody can be cloned into a vector that expresses all or part of the nucleic acid. For example, the nucleic acid can include a fragment of the gene encoding the antibody, such as a single chain antibody (scFv), a F(ab′)₂ fragment, a Fab fragment, or an Fd fragment.

Antibodies may also include modifications, e.g., modifications that alter Fc function, e.g., to decrease or remove interaction with an Fc receptor or with Clq, or both. For example, the human IgG4 constant region can have a Ser to Pro mutation at residue 228 to fix the hinge region.

In another example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some antibodies that include an Fc domain, the antibody production system may be designed to synthesize antibodies in which the Fc region is glycosylated. In another example, the Fc domain of IgG molecules is glycosylated at asparagine 297 in the CH2 domain. This asparagine is the site for modification with biantennary-type oligosaccharides. This glycosylation participates in effector functions mediated by Fcγ receptors and complement C1q (Burton and Woof (1992) Adv. Immunol. 51:1-84; Jefferis et al. (1998) Immunol. Rev. 163:59-76). The Fc domain can be produced in a mammalian expression system that appropriately glycosylates the residue corresponding to asparagine 297. The Fc domain can also include other eukaryotic post-translational modifications.

Antibodies can be modified, e.g., with a moiety that improves its stabilization and/or retention in circulation, e.g., in blood, serum, lymph, bronchoalveolar lavage, or other tissues, e.g., by at least 1.5, 2, 5, 10, or 50 fold.

For example, an antibody generated by a method described herein can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 daltons (or about 1,000 to about 15,000, and 2,000 to about 12,500) can be used.

For example, an antibody generated by a method described herein can be conjugated to a water soluble polymer, e.g., a hydrophilic polyvinyl polymer, e.g. polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides that comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon.

Antibody-Mimic Molecules

Antibody-mimic molecules are antibody-like molecules comprising a protein scaffold or other non-antibody target binding region with a structure that facilitates binding with target molecules, e.g., polypeptides. When an antibody mimic comprises a scaffold, the scaffold structure of an antibody-mimic is reminiscent of antibodies, but antibody-mimics do not include the CDR and framework structure of immunoglobulins. Like antibodies, however, a pool of scaffold proteins having different amino acid sequence (but having the same basic scaffold structure) can be made and screened to identify the antibody-mimic molecule having the desired features (e.g., ability to bind a particular target; ability to bind a particular target with a certain affinity; ability to bind a particular target to produce a certain result, such as to inhibit activity of the target). In this way, antibody-mimics molecules that bind a target and that have a desired function can be readily made and tested in much the same way that antibodies can be. There are numerous examples of classes of antibody-mimic molecules; each of which is characterized by a unique scaffold structure. Any of these classes of antibody-mimic molecules may be used as the AAM moiety portion of a complex of the disclosure. Exemplary classes are described below and include, but are not limited to, DARPin polypeptides, Adnectins® polypeptides, and Anticalins® polypeptides.

In certain embodiments, an antibody-mimic moiety molecule can comprise binding site portions that are derived from a member of the immunoglobulin superfamily that is not an immunoglobulin (e.g., a T-cell receptor or a cell-adhesion protein such as CTLA-4, N-CAM, and telokin) Such molecules comprise a binding site portion which retains the conformation of an immunoglobulin fold and is capable of specifically binding to the target antigen or epitope. In some embodiments, antibody-mimic moiety molecules of the disclosure also comprise a binding site with a protein topology that is not based on the immunoglobulin fold (e.g., such as ankyrin repeat proteins or fibronectins) but which nonetheless are capable of specifically binding to a target antigen or epitope.

Antibody-mimic moiety molecules may be identified by selection or isolation of a target-binding variant from a library of binding molecules having artificially diversified binding sites. Diversified libraries can be generated using completely random approaches (e.g., error-prone PCR, exon shuffling, or directed evolution) or aided by art-recognized design strategies. For example, amino acid positions that are usually involved when the binding site interacts with its cognate target molecule can be randomized by insertion of degenerate codons, trinucleotides, random peptides, or entire loops at corresponding positions within the nucleic acid which encodes the binding site (see e.g., U.S. Pub. No. 20040132028). The location of the amino acid positions can be identified by investigation of the crystal structure of the binding site in complex with the target molecule. Candidate positions for randomization include loops, flat surfaces, helices, and binding cavities of the binding site. In certain embodiments, amino acids within the binding site that are likely candidates for diversification can be identified by their homology with the immunoglobulin fold. For example, residues within the CDR-like loops of fibronectin may be randomized to generate a library of fibronectin binding molecules (see, e.g., Koide et al., J. Mol. Biol., 284: 1141-1151 (1998)). Other portions of the binding site which may be randomized include flat surfaces. Following randomization, the diversified library may then be subjected to a selection or screening procedure to obtain binding molecules with the desired binding characteristics. For example, selection can be achieved by art-recognized methods such as phage display, yeast display, or ribosome display.

In one embodiment, an antibody-mimic molecule of the disclosure comprises a binding site from a fibronectin binding molecule. Fibronectin binding molecules (e.g., molecules comprising the Fibronectin type I, II, or III domains) display CDR-like loops which, in contrast to immunoglobulins, do not rely on intra-chain disulfide bonds. The FnIII loops comprise regions that may be subjected to random mutation and directed evolutionary schemes of iterative rounds of target binding, selection, and further mutation in order to develop useful therapeutic tools. Fibronectin-based “addressable” therapeutic binding molecules (“FATBIM”) may be developed to specifically or preferentially bind the target antigen or epitope. Methods for making fibronectin binding polypeptides are described, for example, in WO 01/64942 and in U.S. Pat. Nos. 6,673,901, 6,703,199, 7,078,490, and 7,119,171, which are incorporated herein by reference.

FATBIMs include, for example, the species of fibronectin-based binding molecules termed Adnectins®. As used herein “Adnectins®,” also called “monobodies,” are genetically engineered proteins that functionally mimic antibodies and that typically exhibit highly specific and high-affinity target protein binding. In some embodiments, an Adnectin® comprises far fewer amino acid residues than does an antibody, and in other embodiments, the Adnectin® is approximately the size as a single variable domain of an antibody. In one embodiment, the Adnectin® comprises approximately 90 amino acids, e.g., 94 amino acids, and has a molecular mass of about 10 kDa, which is fifteen times smaller than an IgG type antibody, and comparable to the size of a single variable domain of an antibody. In certain embodiments the structure of an Adnectin® is based on the structure of human fibronectin, and more specifically on the structure of the tenth extracellular type III domain of human fibronectin. This domain has a structure analogous to antibody variable domains, with seven beta sheets forming a barrel and three exposed loops on each side, which are analogous to the three complementarity determining regions. Unlike antibodies, however, Adnectins® typically lack binding sites for metal ions and a central disulfide bond. Adnectins® can be engineered to have specificity for different target proteins by modifying the loops between the second and third beta sheets, and between the sixth and seventh beta sheets (i.e., by modifying loops BC and FG of the tenth extracellular type III domain of fibronectin). Adnectins® are described in, e.g., U.S. Pat. No. 7,115,396. In certain embodiments, the disclosure provides a complex comprising a Surf+ Penetrating Polypeptide associated with an Adnectin (e.g., a antibody-mimic based on the structure of human fibronectin), wherein the Adnectin binds to an intracellularly expressed target. In other words, in certain embodiments, complexes of the disclosure comprise an AAM moiety portion comprising a scaffold structure based on fibronectin, such as the tenth extracellular type III domain of fibronectin.

In another embodiment, an antibody-mimic molecule of the disclosure comprises a binding site from an affibody. As used herein Affibody® molecules are derived from the immunoglobulin binding domains of staphylococcal Protein A (SPA) (see e.g., Nord et al., Nat. Biotechnol., 15: 772-777 (1997)). An Affibody® is an antibody mimic that has unique binding sites that bind specific targets. Affibody® molecules can be small (e.g., consisting of three alpha helices with 58 amino acids and having a molar mass of about 6 kDa), have an inert format (no Fc function), and have been successfully tested in humans as targeting moieties. Affibody® molecules have been shown to withstand high temperatures (90° C.) or acidic and alkaline conditions (pH 2.5 or pH 11, respectively). Affibody® binding sites employed in the disclosure may be synthesized by mutagenizing an SPA-related protein (e.g., Protein Z) derived from a domain of SPA (e.g., domain B) and selecting for mutant SPA-related polypeptides having binding affinity for a target antigen or epitope. Other methods for making affibody binding sites are described in U.S. Pat. Nos. 6,740,734 and 6,602,977 and in WO 00/63243, each of which is incorporated herein by reference. In certain embodiments, the disclosure provides a complex comprising a Surf+ Penetrating Polypeptide associated with an Affibody, wherein the Affibody binds to an intraceullarly expressed target.

In another embodiment, an antibody-mimic molecule of the disclosure comprises a binding site from an anticalin. As used herein, Anticalins® are antibody functional mimetics derived from human lipocalins. Lipocalins are a family of naturally-occurring binding proteins that bind and transport small hydrophobic molecules such as steroids, bilins, retinoids, and lipids. The main structure of Anticalins® is similar to wild type lipocalins. The central element of this protein architecture is a beta-barrel structure of eight antiparallel strands, which supports four loops at its open end. These loops form the natural binding site of the lipocalins and can be reshaped in vitro by extensive amino acid replacement, thus creating novel binding specificities.

Anticalins® possess high affinity and specificity for their prescribed ligands as well as fast binding kinetics, so that their functional properties are similar to those of antibodies. Anticalins® however, have several advantages over antibodies, including smaller size, composition of a single polypeptide chain, and a simple set of four hypervariable loops that can be easily manipulated at the genetic level. Anticalins®, for example, are about eight times smaller than antibodies with a size of about 180 amino acids and a mass of about 20 kDa. Anticalins® have better tissue penetration than antibodies and are stable at temperatures up to 70° C., and also unlike antibodies, Anticalins® can be produced in bacterial cells (e.g., E. coli cells) in large amounts. Further, while antibodies and most other antibody mimetics can only be directed at macromolecules like proteins, Anticalins® are able to selectively bind to small molecules as well. Anticalins® are described in, e.g., U.S. Pat. No. 7,723,476. In certain embodiments, the disclosure provides a complex comprising a Surf+ Penetrating Polypeptide associated with an Affibody, wherein the Affibody binds to an intraceullarly expressed target.

In another embodiment, an antibody-mimic molecule of the disclosure comprises a binding site from a cysteine-rich polypeptide. Cysteine-rich domains employed in the practice of the present disclosure typically do not form an alpha-helix, a beta-sheet, or a beta-barrel structure. Typically, the disulfide bonds promote folding of the domain into a three-dimensional structure. Usually, cysteine-rich domains have at least two disulfide bonds, more typically at least three disulfide bonds. An exemplary cysteine-rich polypeptide is an A domain protein. A-domains (sometimes called “complement-type repeats”) contain about 30-50 or 30-65 amino acids. In some embodiments, the domains comprise about 35-45 amino acids and in some cases about 40 amino acids. Within the 30-50 amino acids, there are about 6 cysteine residues. Of the six cysteines, disulfide bonds typically are found between the following cysteines: Cl and C3, C2 and C5, C4 and C6. The A domain constitutes a ligand binding moiety. The cysteine residues of the domain are disulfide linked to form a compact, stable, functionally independent moiety. Clusters of these repeats make up a ligand binding domain, and differential clustering can impart specificity with respect to the ligand binding. Exemplary proteins containing A-domains include, e.g., complement components (e.g., C6, C7, C8, C9, and Factor I), serine proteases (e.g., enteropeptidase, matriptase, and corin), transmembrane proteins (e.g., ST7, LRP3, LRP5 and LRP6) and endocytic receptors (e.g. Sortilin-related receptor, LDL-receptor, VLDLR, LRP1, LRP2, and ApoER2). Methods for making A-domain proteins of a desired binding specificity are disclosed, for example, in WO 02/088171 and WO 04/044011, each of which is incorporated herein by reference.

In another embodiment, an antibody-mimic molecule of the disclosure comprises a binding site from a repeat protein. Repeat proteins are proteins that contain consecutive copies of small (e.g., about 20 to about 40 amino acid residues) structural units or repeats that stack together to form contiguous domains. Repeat proteins can be modified to suit a particular target binding site by adjusting the number of repeats in the protein. Exemplary repeat proteins include designed ankyrin repeat proteins (i.e., a DARPins) (see e.g., Binz et al., Nat. Biotechnol., 22: 575-582 (2004)) or leucine-rich repeat proteins (i.e., LRRPs) (see e.g., Pancer et al., Nature, 430: 174-180 (2004)).

As used here, “DARPins” are genetically engineered antibody mimetic proteins that typically exhibit highly specific and high-affinity target protein binding. DARPins were first derived from natural ankyrin proteins. In certain embodiments, DARPins comprise three, four or five repeat motifs of an ankyrin protein. In certain embodiments, a unit of an ankyrin repeat consists of 30-34 amino acid residues and functions to mediate protein-protein interactions. In certain embodiments, each ankyrin repeat exhibits a helix-turn-helix conformation, and strings of such tandem repeats are packed in a nearly linear array to form helix-turn-helix bundles connected by relatively flexible loops. In certain embodiments, the global structure of an ankyrin repeat protein is stabilized by intra- and inter-repeat hydrophobic and hydrogen bonding interactions. The repetitive and elongated nature of the ankyrin repeats provides the molecular bases for the unique characteristics of ankyrin repeat proteins in protein stability, folding and unfolding, and binding specificity. While not wishing to be bound by theory, it is believed that the ankyrin repeat proteins do not recognize specific sequences, and interacting residues are discontinuously dispersed into the whole molecules of both the ankyrin repeat protein and its target protein. In addition, the availability of thousands of ankyrin repeat sequences has made it feasible to use rational design to modify the specificity and stability of an ankyrin repeat domain for use as a DARPin to target any number of proteins. The molecular mass of a DARPin domain is typically about 14 or 18 kDa for four- or five-repeat DARPins, respectively. DARPins are described in, e.g., U.S. Pat. No. 7,417,130. All so far determined tertiary structures of ankyrin repeat units share a characteristic composed of a beta-hairpin followed by two antiparallel alpha-helices and ending with a loop connecting the repeat unit with the next one. Domains built of ankyrin repeat units are formed by stacking the repeat units to an extended and curved structure. LRRP binding sites from part of the adaptive immune system of sea lampreys and other jawless fishes and resemble antibodies in that they are formed by recombination of a suite of leucine-rich repeat genes during lymphocyte maturation. Methods for making DARpin or LRRP binding sites are described in WO 02/20565 and WO 06/083275, each of which is incorporated herein by reference.

Another example of an AAM moiety suitable for use in the present disclosure is based on technology in which binding regions are engineered into the Fc domain of an antibody molecule. These antibody-like molecules are another example of AAM moieties for use in the present disclosure. In certain embodiments, antibody mimics include all or a portion of an antibody like molecule, comprising the CH2 and CH3 domains of an immunoglulin, engineered with non-CDR loops of constant and/or variable domains, thereby mediating binding to an epitope via the non-CDR loops. Exemplary technology includes technology from F-Star, such as antigen binding Fc molecules (termed Fcab™) or full length antibody like molecules with dual functionality (mAb² ™). Fcab™ (antigen binding Fc) are a “compressed” version of these antibody like molecules. These molecules include the CH2 and CH3 domains of the Fc portion of an antibody, naturally folded as a homodimer (50 kDa). Antigen binding sites are engineered into the CH3 domains, but the molecules lack traditional antibody variable regions.

Similar antibody like molecules are referred to as mAb² ™ molecules. Full length IgG antibodies with additional binding domains (such as two) engineered into the CH3 domains. Depending on the type of additional binding sites engineered into the CH3 domains, these molecules may be bispecific or multispecific or otherwise facilitate tissue targeting.

This technology is described in, for example, WO08/003103, WO12/007167, and US application 20090298195, the disclosures of which are hereby incorporated by reference.

In other embodiments, an antibody-mimic molecule of the disclosure comprises binding sites derived from Src homology domains (e.g. SH2 or SH3 domains), PDZ domains, beta-lactamase, high affinity protease inhibitors, or small disulfide binding protein scaffolds such as scorpion toxins. Methods for making binding sites derived from these molecules have been disclosed in the art, see e.g., Panni et al., J. Biol. Chem., 277: 21666-21674 (2002), Schneider et al., Nat. Biotechnol., 17: 170-175 (1999); Legendre et al., Protein Sci., 11:1506-1518 (2002); Stoop et al., Nat. Biotechnol., 21: 1063-1068 (2003); and Vita et al., PNAS, 92: 6404-6408 (1995). Yet other binding sites may be derived from a binding domain selected from the group consisting of an EGF-like domain, a Kringle-domain, a PAN domain, a Gla domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an Immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, a Laminin-type EGF-like domain, a C2 domain, and other such domains known to those of ordinary skill in the art, as well as derivatives and/or variants thereof. Exemplary antibody-mimic moiety molecules, and methods of making the same, can also be found in Stemmer et al., “Protein scaffolds and uses thereof”, U.S. Patent Publication No. 20060234299 (Oct. 19, 2006) and Hey, et al., Artificial, Non-Antibody Binding Proteins for Pharmaceutical and Industrial Applications, TRENDS in Biotechnology, vol. 23, No. 10, Table 2 and pp. 514-522 (October 2005).

In one embodiment, an antibody-mimic molecule comprises a Kunitz domain. “Kunitz domains” as used herein, are conserved protein domains that inhibit certain proteases, e.g., serine proteases. Kunitz domains are relatively small, typically being about 50 to 60 amino acids long and having a molecular weight of about 6 kDa. Kunitz domains typically carry a basic charge and are characterized by the placement of two, four, six or eight or more that form disulfide linkages that contribute to the compact and stable nature of the folded peptide. For example, many Kunitz domains have six conserved cysteine residues that form three disulfide linkages. The disulfide-rich α/β fold of a Kunitz domain can include two, three (typically), or four or more disulfide bonds.

Kunitz domains have a pear-shaped structure that is stabilized the, e.g., three disulfide bonds, and that contains a reactive site region featuring the principal determinant P1 residue in a rigid confirmation. These inhibitors competitively prevent access of a target protein (e.g., a serine protease) for its physiologically relevant macromolecular substrate through insertion of the P1 residue into the active site cleft. The P1 residue in the proteinase-inhibitory loop provides the primary specificity determinant and dictates much of the inhibitory activity that particular Kunitz protein has toward a targeted proteinase. Typically, the N-terminal side of the reactive site (P) is energetically more important that the P′ C-terminal side. In most cases, lysine or arginine occupy the P1 position to inhibit proteinases that cleave adjacent to those residues in the protein substrate. Other residues, particularly in the inhibitor loop region, contribute to the strength of binding. Generally, about 10-12 amino acid residues in the target protein and 20-25 residues in the proteinase are in direct contact in the formation of a stable proteinase-inhibitor complex and provide a buried area of about 600 to 900 A. By modifying the residues in the P site and surrounding residues Kunitz domains can be designed to target and inhibit or activate a protein of choice, e.g., an intracellular protein of choice. Kunitz domains are described in, e.g., U.S. Pat. No. 6,057,287.

In another embodiment, an antibody-mimic molecule of the disclosure is an Affilin®. As used herein “Affilin®” molecules are small antibody-mimic proteins which are designed for specific affinities towards proteins and small compounds. New Affilin® molecules can be very quickly selected from two libraries, each of which is based on a different human derived scaffold protein. Affilin® molecules do not show any structural homology to immunoglobulin proteins. There are two commonly-used Affilin® scaffolds, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

In another embodiment, an antibody-mimic moiety molecule of the disclosure is an Avimer. Avimers are evolved from a large family of human extracellular receptor domains by in vitro exon shuffling and phage display, generating multidomain proteins with binding and inhibitory properties Linking multiple independent binding domains has been shown to create avidity and results in improved affinity and specificity compared with conventional single-epitope binding proteins. In certain embodiments, Avimers consist of two or more peptide sequences of 30 to 35 amino acids each, connected by linker peptides. The individual sequences are derived from A domains of various membrane receptors and have a rigid structure, stabilised by disulfide bonds and calcium. Each A domain can bind to a certain epitope of the target protein. The combination of domains binding to different epitopes of the same protein increases affinity to this protein, an effect known as avidity (hence the name). Other potential advantages include simple and efficient production of multitarget-specific molecules in Escherichia coli, improved thermostability and resistance to proteases. Avimers with sub-nanomolar affinities have been obtained against a variety of targets. Alternatively, the domains can be directed against epitopes on different target proteins. This approach is similar to the one taken in the development of bispecific monoclonal antibodies. In a study, the plasma half-life of an anti-interleukin 6 avimer could be increased by extending it with an anti-immunoglobulin G domain. Additional information regarding Avimers can be found in U.S. patent application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

The foregoing provides numerous examples of classes of antibody-mimics. In certain embodiments, the disclosure provides complexes in which the AAM moiety portion is an antibody-mimic that binds to an intracellular target, such as any of the foregoing classes antibody-mimics. Any of these antibody-mimics may be complexed with a Surf+ Penetrating Polypeptide or a portion comprising a Surf+ Penetrating Polypeptide, including any of the sub-categories or specific examples of Surf+ Penetrating Polypeptides.

### Formation of Complexes

The present disclosure provides complexes comprising (i) a Surf+ Penetrating Polypeptide portion and (ii) an AAM moiety portion (e.g., at least one AAM moiety) associated with the Surf+ Penetrating Polypeptide portion. The complexes are useful, for example, for delivery into a cell, and thus facilitate delivery of the AAM moiety into a cell where it can bind its intracellular target. Below are provided examples of complexes of the disclosure and how the portions of the complexes are associated and/or made. The present disclosure provides complexes comprising (i) a Surf+ Penetrating Polypeptide portion and (ii) an AAM moiety portion (e.g., at least one AAM moiety) associated with the Surf+ Penetrating Polypeptide portion. The AAM moiety portion binds to an intracellular target and the Surf+ Penetrating Polypeptide portion facilitates entry of the complex, and thus entry of the AAM moiety, into cells. Once inside the cell, the AAM moiety portion can bind the intracellularly expressed target. In certain embodiments, the association between the AAM moiety and the Surf+ Penetrating Polypeptide is disruptable. Thus, in certain embodiments, once the complex enters the cell, the association can be disrupted and the AAM moiety alone can bind or continue binding to the target. However, the association need not be disrupted, and the complex may remain intact after entry into the cell.

Complexes of the disclosure may, in certain embodiments, include portions in addition to the Surf+ Penetrating Polypeptide portion and the AAM moiety portion. For example, the complexes may include one or more linkers, the complexes may include sequence that helps localize the complex to a sub-cellular location, and/or the complex may include tags to facilitate detection and/or purification of the complex or a portion of the complex. These additional sequences may be located at the N-terminus, at the C-terminus or internally. Moreover, additional portions may be interconnected to the Surf+ Polypeptide portion to the AAM moiety portion or to both.

Complexes of the disclosure, including fusion proteins, comprises a Surf+ Penetrating Polypeptide that penetrates cells associated with an AAM moiety that binds to an intraceelular target. When provided as a complex, such as a fusion protein, these complexes penetrate cells and bind to the intracellular target via the AAM moiety. When provided as a complex or fusion protein (e.g., when the Surf+ Penetrating Polypeptide and the AAM moiety are associate), the complex penetrates cells and the AAM moiety is able to bind to its intracellular target. By way of example, an AAM moiety may bind to an intracellular target, such as a polypeptide or peptide, and alter the activity of the target and/or the activity of the cell via one or more of the following mechanisms (i) inhibit one or more functions of the target; (ii) activate one or more functions of the target; (iii) increase or decrease the activity of the target; (iv) promote or inhibit degradation of the target; (v) change the localization of the target; and (vi) prevent binding between the target and another protein.

In certain embodiments, the Surf+ Penetrating Polypeptide and AAM moiety portions of the complex are associated covalently. For example, these two portions may be fused (e.g., the complex comprises a fusion protein). Covalent interactions may be direct or indirect (via a linker). Additional interactions, such as non-covalent interactions, may also be involved in the association between the two portions. Thus, in some embodiments, such covalent interactions are mediated by one or more linkers. In some embodiments, the linker is a cleavable linker. In certain embodiments, the cleavable linker comprises an amide, an ester, or a disulfide bond. For example, the linker may be an amino acid sequence that is cleavable by a cellular enzyme. In certain embodiments, the enzyme is a protease. In other embodiments, the enzyme is an esterase. In some embodiments, the enzyme is one that is more highly expressed in certain cell types than in other cell types. For example, the enzyme may be one that is more highly expressed in tumor cells than in non-tumor cells. Exemplary sequences that can be used in linkers and enzymes that cleave those linkers are presented in Table 2.

TABLE2
Exemplary cleavable linker sequences.

| Cleavable | SEQ ID | |
|---|---|---|
| sequencer | NO: | Enzymes that Target the Linker |
| X-AGVF-X | 670 | Lysosomal thiol proteinases (see, e.g., Duncan et al., |
| | | Biosci. Rep., 2: 1041-46, 1982; incorporated herein by |
| | | reference) |
| X-GFLG-X | 671 | Lysosomal cysteine proteinases (see, e.g., Vasey et al., |
| | | Clin. Canc. Res., 5: 83-94, 1999; incorporated herein by |
| | | reference) |
| X-FK-X | 672 | Cathepsin B-ubiquitous, overexpressed in many solid |
| | | tumors, such as breast cancer (see, e.g., Dubowchik et |
| | | al., Bioconjugate Chem., 13: 855-69, 2002; |
| | | incorporated herein by reference) |
| X-A*L-X | 673 | Lysosomal hydrolases (see, e.g., Trouet et al., Proc. |
| | | Natl. Acad. Sci., USA, 79: 626-29, 1982; incorporated |
| | | herein by reference) |
| X-A*LA*L-X | 674 | Cathepsin B-ubiquitous, overexpressed in many solid |
| | | tumors, such as breast cancer (see, e.g., Schmid et al., |
| | | Bioconjugate Chemistry, 18: 702-16, 2007; |
| | | incorporated herein by reference) |
| X-AL*AL*A-X | 675 | Cathepsin D-ubiquitous (see, e.g., Czerwinski et al., |
| | | Proc. Natl. Acad. Sci. USA, 95: 11520-25, 1998; |
| | | incorporated herein by reference) |

“X” denotes the Surf+ Penetrating Polypeptide or AAM moiety.
“*” refers to observed cleavage site.

Other exemplary linkers include flexible linkers, such as one or more repeats of glycine and serine (Gly/Ser linkers). In certain embodiments, the flexible linker comprises glycine, alanine and/or serine amino acid residues. Simple amino acids (e.g., amino acids with simple side chains (e.g., H, CH₃ or CH₂OH) and/or unbranched) provide greater flexibility (e.g., two-dimensional or three-dimensional flexibility) within the linker. Further, alternating the glycine, alanine and/or serine residues may provide even greater flexibility with in the linker. The amino acids can alternate/repeat in any manner consistent with the linker remaining functional (e.g., resulting in expressed and/or active fusion protein). Exemplary flexible linkers include linkers comprising repeats of gly-gly-gly-gly-ser, gly-ser, ala-ser, and ala-gly. Other combinations are also possible.

In certain embodiments, the Surf+ Penetrating Polypeptide and the AAM moiety are fused by using a construct that comprises an intein, which is self-spliced out to join the Surf+ Penetrating Polypeptide and the AAM moiety via a peptide bond.

In another embodiment, e.g., where expression of a fusion construction is not practical (e.g., is inefficient) or not possible, the Surf+ Penetrating Polypeptide and the AAM moiety are synthesized by using a viral 2A peptide construct that comprises the Surf+ Penetrating Polypeptide and the AAM moiety for bicistronic expression. In this embodiment, the Surf+ Penetrating Polypeptide and the AAM moiety genes may be expressed on the bicistronic construct, and the 2A peptide results in cotranslational “cleavage” of the two proteins (Trichas et al., BMC Biology 6:40, 2008).

The disclosure contemplates complexes in which the Surf+ Penetrating Polypeptide and the AAM moiety portions are associated by a covalent or non-covalent linkage. In either case, the association may be direct or via one or more additional intervening liners or moieties.

In some embodiments, a Surf+ Penetrating Polypeptide and an AAM moiety are associated through chemical or proteinaceous linkers or spacers. Exemplary linkers and spacers include, but are not restricted to, substituted or unsubstituted alkyl chains, polyethylene glycol derivatives, amino acid spacers, sugars, or aliphatic or aromatic spacers common in the art.

Suitable linkers include, for example, homobifunctional and heterobifunctional cross-linking molecules. The homobifunctional molecules have at least two reactive functional groups, which are the same. The reactive functional groups on a homobifunctional molecule include, for example, aldehyde groups and active ester groups. Homobifunctional molecules having aldehyde groups include, for example, glutaraldehyde and subaraldehyde.

Homobifunctional linker molecules having at least two active ester units include esters of dicarboxylic acids and N-hydroxysuccinimide. Some examples of such N-succinimidyl esters include disuccinimidyl suberate and dithio-bis-(succinimidyl propionate), and their soluble bis-sulfonic acid and bis-sulfonate salts such as their sodium and potassium salts.

Heterobifunctional linker molecules have at least two different reactive groups. Examples of heterobifunctional reagents containing reactive disulfide bonds include N-succinimidyl 3-(2-pyridyl-dithio)propionate (Carlsson et al., 1978. Biochem. J., 173:723-737), sodium S-4-succinimidyloxycarbonyl-alpha-methylbenzylthiosulfate, and 4-succinimidyloxycarbonyl-alpha-methyl-(2-pyridyldithio)toluene. Examples of heterobifunctional reagents comprising reactive groups having a double bond that reacts with a thiol group include succinimidyl 4-(N-maleimidomethyl)cyclohexahe-1-carboxylate and succinimidyl m-maleimidobenzoate. Other heterobifunctional molecules include succinimidyl 3-(maleimido)propionate, sulfosuccinimidyl 4-(p-maleimido-phenyl)butyrate, sulfosuccinimidyl 4-(N-maleimidomethyl-cyclohexane)-1-carboxylate, maleimidobenzoyl-5N-hydroxy-succinimide ester.

Other means of cross-linking proteins utilize affinity molecule binding pairs, which selectively interact with acceptor groups. One entity of the binding pair can be fused or otherwise linked to the Surf+ Penetrating Polypeptide and the other entity of the binding pair can be fused or otherwise linked to the AAM moiety. Exemplary affinity molecule binding pairs include biotin and streptavidin, and derivatives thereof; metal binding molecules; and fragments and combinations of these molecules. Exemplary affinity binding pairs include StreptTag (WSHPQFEK) (SEQ ID NO: 657)/SBP (streptavidin binding protein), cellulose binding domain/cellulose, chitin binding domain/chitin, S-peptide/S-fragment of RNAseA, calmodulin binding peptide/calmodulin, and maltose binding protein/amylose.

In one embodiment, the Surf+ Penetrating Polypeptide and the AAM moiety are linked by ubiquitin (and ubiquitin-like) conjugation.

The disclosure also provides nucleic acids encoding a Surf+ Penetrating Polypeptide and an AAM moiety, such as an antibody molecule, or a non-antibody molecule scaffold, such as a DARPin, an Adnectin®, an Anticalin®, or a Kunitz domain polypeptide. The complex of a Surf+ Penetrating Polypeptide and an AAM moiety can be expressed as a fusion protein, optionally separated by a peptide linker. The peptide linker can be cleavable or not cleavable. A nucleic acid encoding a fusion protein can express the fusion in any orientation. For example, the nucleic acid can express an N-terminal Surf+ Penetrating Polypeptide fused to a C-terminal AAM moiety (e.g., antibody), or can express an N-terminal AAM moiety fused to a C-terminal Surf+ Penetrating Polypeptide.

A nucleic acid encoding an Surf+ Penetrating Polypeptide can be on a vector that is separate from a vector that carries a nucleic acid encoding a AAM moiety. The Surf+ Penetrating Polypeptide and the AAM moiety can be expressed separately, and complexed (including chemically linked) prior to introduction to a cell for intracellular delivery. The isolated complex can be formulated for administration to a subject, as a pharmaceutical composition.

The disclosure also provides host cells comprising a nucleic acid encoding the Surf+ Penetrating Polypeptide or the AAM moiety, or comprising the complex as a fusion protein. The host cells can be, for example, prokaryotic cells (e.g., E. coli) or eukaryotic cells.

In certain embodiments, the recombinant nucleic acids encoding an complex, or the portions thereof, may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used. In certain aspects, this disclosure relates to an expression vector comprising a nucleotide sequence encoding a complex of the disclosure (e.g., a complex comprising a Surf+ Penetrating Polypeptide portion and an AAM moiety portion) polypeptide and operably linked to at least one regulatory sequence. Regulatory sequences are art-recognized and are selected to direct expression of the encoded polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

The disclosure also provides host cells comprising or transfected with a nucleic acid encoding the complex as a fusion protein. The host cells can be, for example, prokaryotic cells (e.g., E. coli) or eukaryotic cells. Other suitable host cells are known to those skilled in the art.

In addition to the nucleic acid sequence encoding the complex or portions of the complex, a recombinant expression vector may carry additional nucleic acid sequences, such as sequences that regulate replication of the vector in a host cells (e.g., origins of replication) and selectable marker genes. The selectable marker gene facilitates selection of host cells into which the vector has been introduced.

Exemplary selectable marker genes include the ampicillin and the kanamycin resistance genes for use in E. coli.

The present disclosure further pertains to methods of producing fusion proteins of the disclosure. For example, a host cell transfected with an expression vector can be cultured under appropriate conditions to allow expression of the polypeptide to occur. The polypeptide may be secreted and isolated from a mixture of cells and medium containing the polypeptides. Alternatively, the polypeptides may be retained in the cytoplasm or in a membrane fraction and the cells harvested, lysed and the protein isolated. A cell culture includes host cells, media and other byproducts. Suitable media for cell culture are well known in the art. The polypeptides can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptides. In a preferred embodiment, the polypeptide is a fusion protein containing a domain which facilitates its purification.

A nucleic acid encoding a Surf+ Penetrating Polypeptide can be on a vector that is separate from a vector that carries a nucleic acid encoding an AAM moiety. The portions of the complex can be expressed separately, and complexed prior to introduction to a cell for intracellular delivery. The isolated complex can be formulated for administration to a subject, as a pharmaceutical composition.

Recombinant nucleic acids of the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli. The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

It should be understood that fusion polypeptides or protein of the present disclosure can be made in numerous ways. For example, a Surf+ Penetrating Polypeptide and an AAM moiety can be made separately, such as recombinantly produced in two separate cell cultures from nucleic acid constructs encoding their respective proteins. Once made, the proteins can be chemically conjugated directly or via a linker. By way of another example, the fusion polypeptide can be made as an inframe fusion in which the entire fusion polypeptide, optionally including one or more linker, tag or other moiety, is made from a nucleic acid construct that includes nucleotide sequence encoding both a Surf+ Penetrating Polypeptide portion and an AAM moiety portion of the complex.

In certain embodiments, a complex of the disclosure is formed under conditions where the linkage (e.g., by a covalent or non-covalent linkage) is formed, while the activity of the AAM moiety is maintained.

To minimize the effect of linkage on AAM moiety activity (e.g., target binding), any linkage to the AAM moiety can be at a site on the protein that is distant from the target-interacting region of the AAM moiety.

Further, in the case of a cleavable linker, an enzyme that cleaves a linker between the a Surf+ Penetrating Polypeptide and an AAM moiety does not have an effect on the AAM moiety, such that the structure of the AAM moiety remains intact and the AAM moiety retains its target binding activity.

In other embodiments, the Surf+ Penetrating Polypeptide and AAM moiety portions of the complex are separated, e.g., within the cell, under conditions where the linkage (e.g., a covalent or non-covalent linkage) is dissociated, while the activity of the AAM moiety is maintained. For example, the Surf+ Penetrating Polypeptide and AAM moiety can be joined by a cleavable peptide linker that is subject to a protease that does not interfere with activity of the AAM moiety.

In some embodiments the Surf+ Penetrating Polypeptide portion and AAM moiety portion are separated in the endosome due to the lower pH of the endosome. Thus in these embodiments, the linker is cleaved or broken in response to the lower pH, but the activity of the AAM moiety is not affected.

In some embodiments, the AAM moiety binds and inhibits (or activates) activity of the intracellular target while the AAM moiety is still complexed with the Surf+ Penetrating Polypeptide. Thus the complex does not dissociate in the cell, prior to the activity of the AAM moiety on the target protein. While in other embodiments, the Surf+ Penetrating Polypeptide and AAM moiety dissociate following delivery into the cell and, for example, the AAM moiety may interact with its intracellular target after dissociation from the Surf+ Penetrating Polypeptide.

It should be noted that the disclosure contemplates that the foregoing description of complexes is applicable to any of the embodiments and combinations of embodiments described herein. For example, the description is applicable in the context of complexes in which the AAM moiety portion is associated with a portion comprising a Surf+ Penetrating Polypeptid presented in the context of additional sequence, such as additional sequence from its own naturally occurring polypeptide. In this context, any interconnection is via the two portions of the complex (the AAM portion and the Surf+ Penetrating Polypeptide portion), but the interconnection may not be directly between the Surf+ Penetrating Polypeptide and the AAM moiety.

Modifications

As detailed above, the disclosure contemplates that Surf+ Penetrating Polypeptides (naturally occurring or generated by protein modification) may be modified chemically or biologically. For example one or more amino acids may be added, deleted, or changed from the primary sequence. This includes changes intended to supercharge a polypeptide (e.g., to increase surface positive charge, net charge or charge/molecular weight). However, modifications to the Surf+ Penetrating Polypeptides also include variation that is not intended to supercharge the protein.

In this section, additional modifications are described. The modifications may be modifications to a complex of the disclosure, and the modification may be appended directly or indirectly to either or both of the Surf+ Penetrating Polypeptide portion or the AAM moiety portion. For example, a polyhistidine tag or other tag may be added to the complex or to either polypeptide portion of the complex to aid in the purification of the complex or of either portion of the complex. Other peptides, protein or small molecules may be added onto the complex to alter the biological, biochemical, and/or biophysical properties of the complex. For example, a targeting peptide may be added to the primary sequence of the Surf+ Penetrating Polypeptides or complex.

Other modifications of the Surf+ Penetrating Polypeptides or complex include, but are not limited to, post-translational or post-production modifications (e.g., glycosylation, phosphorylation, acylation, lipidation, farnesylation, acetylation, proteolysis, etc.). In certain embodiments, the Surf+ Penetrating Polypeptides or complex may be modified to reduce its immunogenicity. In certain embodiments, the Surf+ Penetrating Polypeptides or complex may be modified to improve half-life or bioavailability.

In certain embodiments, the complex or either portion of the complex may be conjugated to a soluble polymer or carbohydrate, e.g., to increase serum half life of the Surf+ Penetrating Polypeptide, AAM moiety and/or complex. For example, the Surf+ Penetrating Polypeptides, AAM moiety or complex may be conjugated to a polyethylene glycol (PEG) polymer, e.g., a monomethoxy PEG. Other polymers useful as stabilizing materials may be of natural, semi-synthetic (modified natural) or synthetic origin. Exemplary natural polymers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrin, dextrose, glucose, polyglucose, polydextrose, pustulan, chitin, agarose, keratin, chondroitin, dermatan, hyaluronic acid, alginic acid, xanthin gum, starch and various other natural homopolymer or heteropolymers, such as those containing one or more of the following aldoses, ketoses, acids or amines: erythose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Accordingly, suitable polymers include, for example, proteins, such as albumin, polyalginates, and polylactide-coglycolide polymers. Exemplary semi-synthetic polymers include carboxymethylcellulose, hydroxymethylcellulose, hydroxypropylmethylcellulose, methylcellulose, and methoxycellulose. Exemplary synthetic polymers include polyphosphazenes, hydroxyapatites, fluoroapatite polymers, polyethylenes (such as, for example, polyethylene glycol (including for example, the class of compounds referred to as PLURONIC™, commercially available from BASF, Parsippany, N.J.), polyoxyethylene, and polyethylene terephthalate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof.

One of skill in the art can envision a multitude of ways of modifying the Surf+ Penetrating Polypeptides, AAM moieties or complexes of the disclosure without departing from the scope of the present disclosure. In certain embodiments, the primary purpose of the modification is a purpose other than to further supercharge the complex versus that of the unmodified complex. The disclosure contemplates that any of the foregoing modifications may be to the Surf+ Penetrating Polypeptide portion of a complex or to the AAM moiety portion of a complex. Moreover, the modification may be made prior to complex formation, concurrently with complex, such as fusion protein formation, or as a post-production step following complex (such as fusion protein) formation.

Additional examples of modifications include localization domains to facilitate localization of the complex to the intended intracellular location. Once again, the localization domain may be appended directly or indirectly to the Surf+ Penetrating Polypeptide portion or to the AAM moiety portion. Exemplary localization domains include, for example, nuclear localization signal, a mitochondrial matrix localization signal, and the like. In certain embodiments, it may be preferable to append the localization domain to the AAM moiety so that, in the event that the association between the Surf+ Penetrating Polypeptide and the AAM moiety is disrupted (such as by cleavage of a cleavable linker) after entry into the cell, the AAM moiety will still include the localization domain.

The foregoing are merely exemplary of modification of the complexes of the disclosure whose primary purpose is other than to further supercharge the complex, relative to the unmodified complex.

Detectable Moieties

It is further contemplated that complexes of the disclosure can be modified to comprise a detectable moiety. Detectable moieties include fluorescent or otherwise detectable polypeptides, peptide, radioactive or other moieties which allow for detection of the complex or the portions of the complex. Such detectable moieties can be included in the polypeptide sequence of the complex, or operably linked thereto, such as in a fusion protein, or by covalent or non-covalent linkages. The disclosure contemplates that the detectable moiety may be appended directly or indirectly to the Surf+ Penetrating Polypeptide portion of the complex and/or the AAM moiety portion of the complex and/or to any linker portion.

Exemplary fluorescent proteins include green fluorescent protein, blue fluorescent protein, cyan fluorescent protein or yellow fluorescent protein. Other exemplary fluorescent proteins include, but are not limited to, enhanced green fluorescent protein (EGFP), split GFP, AcGFP, TurboGFP, Emerald, Azami Green, ZsGreen, EBFP, Sapphire, T-Sapphire, ECFP, mCFP, Cerulean, CyPet, AmCyanl, Midori-Ishi Cyan, mTFP1 (Teal), enhanced yellow fluorescent protein (EYFP), Topaz, Venus, mCitrine, YPet, PhiYFP, ZsYellowl, mBanana, Kusabira Orange, mOrange, dTomato, dTomato-Tandem, DsRed, DsRed2, DsRed-Express (T1), DsRed-Monomer, mTangerine, mStrawberry, AsRed2, mRFP1, JRed, mCherry, HcRed1, mRaspberry, HcRed1, HcRed-Tandem, mPlum, and AQ143.

Additional suitable labels that can be used in accordance with the disclosure include, but are not limited to, fluorescent, chemiluminescent, chromogenic, phosphorescent, and/or radioactive labels. In addition, when an epitope tag is included in a complex, the complex is detectable using an antibody that is immunoreactive with the epitope tag.

Any complex of the disclosure can be readily tested to confirm that, following complex formation, the complex retains the ability to penetrate cells and the AAM moiety retains the ability to specifically bind its target. This testing can be done regardless of whether the complex is a fusion protein (directly or via a linker) or a chemical fusion or otherwise associated. By way of example, the Surf+ Penetrating Polypeptide may be tested for cell penetration activity alone and the AAM moiety may be tested for specific binding (in vitro or ex vivo) to its target. After confirming that the selected Surf+ Penetrating Polypeptide does penetrate cells and the AAM moiety does bind its target, a complex is generated using any suitable method. Following complex formation, cell penetration activity is again assessed to confirm that complex formation did not interfere with cell penetration activity, and that the Surf+ Penetrating Polypeptide penetrates cells in association with this cargo. Additionally, following complex formation, specific binding of the AAM moiety (present in the complex) is tested to confirm complex formation does not interfere with the ability of the AAM moiety to specifically bind its target.

### Applications

The present disclosure provides complexes comprising (i) a Surf+ Penetrating Polypeptide portion and (ii) an AAM moiety portion, wherein the Surf+ Penetrating Polypeptide portion is associated with the AAM moiety portion. The present disclosure also provides methods for using such complexes. As detailed throughout, the AAM moiety binds to a target expressed in a cell and providing the AAM moiety as a complex promotes delivery of the AAM moiety into the cell (e.g., due to the cell penetrating ability of the Surf+ Penetrating Polypeptide). Once inside the cell, the AAM moiety can bind to its target. Such binding may occur while the AAM moiety remained complexed to the Surf+ Penetrating Polypeptide portion, or such binding may occur after cleavage or dissociation of the two portions of the complex. Additionally, binding may initially occur while the AAM moiety is complexed to the Surf+ Penetrating Polypeptide, but the complex may then be disrupted or cleaved so that, subsequently, the AAM moiety alone is bound to the target (e.g., the target polypeptide or peptide expressed in the cell).

Any AAM moiety may be provided as a complex with a Surf+ Penetrating Polypeptide and delivered to a cell using the inventive system. Given the ability to readily make and test antibodies and antibody-mimics, and thus, to generate AAM moieties capable of binding to a target and having a desired activity (e g, inhibiting the function of the target, promoting the function of the target, binding without interfering or altering the function of the target), the present system may be used in combination with virtually any target, such as a polypeptide or peptide, expressed in a cell. Accordingly, the complexes of the disclosure have numerous applications, including research uses, therapeutic uses, diagnostic uses, imaging uses, and the like, and such uses are applicable over a wide range of targets and disease indications.

The following provides specific examples, including examples of specific targets. However, the potential uses of complexes of the disclosure are not limited to specific target polypeptides or peptides. Rather, the generally uses include, at least, the following. Complexes of the disclosure are useful for delivering AAM moieties into cells where they are useful for labeling a target protein, such as for imaging cells, tissues and whole organisms. Labeling may be useful when performing research studies of protein expression, disease progression, cell fate, protein localization and the like. Labeling may be useful diagnostically or prognostically, such as in cases where target expression correlates with a particular condition. In certain embodiments, an AAM moiety intended for labeling may be selected such that it does not interfere with the function of the target (e.g., a moiety that binds to a target but does not alter the activity of the target).

In addition, complexes of the disclosure may be used in research setting to study target expression, presence/absence of target in a disease state, impact of inhibiting or promoting target activity, etc. Complexes of the disclosure are suitable for these studies in vitro or in vivo. By promoting delivery of the AAM moiety into cells, complexes of the disclosure help avoid false negative results obtained when an AAM moiety is unable to penetrate a cell (e.g., a non-experiment because the AAM moiety cannot contact a target expressed inside the cell).

Further, complexes of the disclosure have therapeutic uses by promoting delivery of therapeutic AAM moieties into cells in humans or animals (including animal models of a disease or condition). Once again, the use of complexes of the disclosure decrease failure of an AAM moiety due to inability to effectively penetrate cells or due to the inability to effectively penetrate cells at concentrations that are not otherwise toxic to the organism.

Regardless of whether a complex of the disclosure is used in a research, diagnostic, prognostic or therapeutic context, the result is that the AAM moiety is delivered into a cell following contacting the cell with the complex (e.g., either contacting a cell in culture or administrated to a subject). Once inside the cells, the AAM moiety binds its intracellular target.

In certain embodiments, the AAM moiety binds a target expressed in the nucleus or in the cytosol of a cell. In some embodiments, AAM moiety binds a membrane associated target, e.g., a target localized on the cytosolic side of the cell membrane, the cytosolic side of the nuclear membrane, or the cytosolic side of the mitochondrial membrane.

In certain embodiment, a Surf+ Penetrating Polypeptide is complexed with an AAM moiety that binds an intracllular target in the nucleus of a cell, such as an NFAT (Nuclear Factor of Activated T cells) (e.g., NFAT-2), a STAT (Signal Transducer and Activator of Transcription) (e.g., STAT-3, STAT-5, or STAT-6) or RORgammaT (retinoic acid-related orphan receptor).

In certain embodiments, a Surf+ Penetrating Polypeptide is complexed with an AAM moiety that binds an intracellular target in the cytosol of the cell, such as FK506, calcineurin, or a Janus Kinase (e.g., JAK-1 or JAK-2.

In another embodiment, a Surf+ Penetrating Polypeptide is complexed with an AAM moiety that binds an intracellular target localized on the cytosoloic side of the cell membrane, such as ras, a PI3K (phosphoinositide-3-kinase), or fms-related tyrosine kinase 1 (vascular endothelial growth factor/vascular permeability factor receptor).

In yet other embodiments, a Surf+ Penetrating Polypeptide is complexed with an AAM moiety that binds an intracellular target localized on the cytosoloic side of the mitochondrial membrane, such as Bcl-2.

In some embodiments, the AAM moiety binds a kinase, a transcription factor or an oncoprotein. For example, the AAM moiety can bind a kinase, such as a JAK kinase (e.g., JAK-1 or JAK-2) or b-raf (v-raf murine sarcoma viral oncogene homolog B1) or Erk (mitogen-activated protein kinase 1). By way of further example, the AAM moiety can bind a transcription factor, such as Hif1-alpha, a STAT (e.g., STAT-3, STAT-5 or STAT-6), or IRF-1 (Interferon Regulatory Factor 1). In some embodiments, the AAM moiety binds an oncogene, such as ras, b-raf or Akt (v-akt murine thymoma viral oncogene homolog 1).

In some embodiments, a complex comprising (i) a Surf+ Penetrating Polypeptide portion and (ii) an AAM moiety portion in accordance with the present disclosure may be used for therapeutic purposes, or may be used for diagnostic purposes. The disease or condition that may be treated depends on the target (e.g., the target is one for which binding by an AAM moiety has a therapeutic benefit).

For example, a complex in accordance with the present disclosure may be used for treatment of any of a variety of diseases, disorders, and/or conditions, including but not limited to one or more of the following: autoimmune disorders; inflammatory disorders; and proliferative disorders, including cancers. In one embodiment, the disease treated by the complex is a cardiovascular disorder, or an angiogenic disorder such as macular degeneration. In another embodiment, the disease treated by the complex is an eye disease, such as age-related macular degeneration (AMD), diabetic macular edema (DME), retinitis pigmentosa, or uveitis.

In some embodiments, a complex is useful for treating one or more of the following: an infectious disease; a neurological disorder; a respiratory disorder; a digestive disorder; a musculoskeletal disorder; an endocrine, metabolic, or nutritional disorders; a urological disorder; psychological disorder; a skin disorder; a blood and lymphatic disorder; etc.

In certain embodiments, the complex of the disclosure binds, via the AAM moiety, a protein set forth in Table 3 (each, an intracellular target). In other words, the AAM moiety portion of the complex binds (e.g., specifically binds) to the target expressed or otherwise located inside the cell (the intracellular target). In certain embodiments, targeting the protein may be useful in the research, diagnosis, prognosis, monitoring or treatment of the listed disease.

TABLE 3
Exemplary intracellular target proteins.

| | | | Intracellular |
|---|---|---|---|
| Diseases | Target | Protein class | Location of Target |
| cancer, age-related | Hifl -alpha | Txn factor | nuclear |
| macular | | | |
| degeneration, | | | |
| ischemia, rheumtoid | | | |
| arthritis | | | |
| dry eye, psoriasis | Calcineurin | phosphatase | cytosol |
| psoriasis | peptidylprolyl isomerase | peptidylprolyl | cytosol |
| | A (cyclophilin A) | isomerase | |
| psoriasis | peptidylprolyl isomerase | peptidylprolyl | cytosol |
| | A (FK506 binding | isomerase | |
| | protein/immunophilin) | | |
| dry eye, psoriasis | NFATs (NFAT-2) | Txn factor | nuclear |
| cancer, Transplant | mechanistic/mammalian | serine/threonine | cytosol |
| Rejection, | target of rapamycin | kinase | |
| Restenosis, | mTOR, FRAP1; | | |
| glycogen storage | (serine/threonine kinase) | | |
| disease | | | |
| myelofibrosis, | Janus Kinases (such as | non-receptor | cytosol |
| cancer, | JAK-1 and JAK-2) | tyrosine kinase | |
| inflammation | | | |
| inflammatory | SOCS1, SOCS3 | STAT binding | cytosol |
| diseases | (suppressor of cytokine | protein | |
| (rheumatoid | signaling) | | |
| arthritis, gout, | | | |
| crohn's disease), | | | |
| epilespy, | | | |
| Huntington Disease | | | |
| autoimmune | STAT-3 (signal | Txn factor | nuclear |
| diseases such as | transducer and activator | | |
| multiple sclerosis | of transcription) | | |
| and cancer, age- | | | |
| related macular | | | |
| degeneration, | | | |
| uveitis | | | |
| cancer (Sezary | STAT-5 | Txn factor | nuclear |
| disease) | | | |
| autoimmune | STAT-6 | Txn factor | nuclear |
| diseases such as | | | |
| atopic dermatitis | | | |
| and emphysema, | | | |
| COPD, lung | | | |
| fibrosis, acute | | | |
| asthma | | | |
| cancer | Ras | GTPase, signal | cytosolic-side of |
| | | transducing protein | cell membrane |
| cancer such as | b-raf | serine/threonine | cytosol |
| melanoma | | kinase | |
| cancer, prion | Erk | Txn factor | multiple locations |
| diseases such as | | | depending on cell- |
| Creutzfeldt-Jakob | | | type and disease |
| Disease | | | |
| cancer | MAP Kinases (mitogen | serine/threonine | cytosol |
| | activated kinases) | kinase | |
| cancer | Jnk (C-Jun N-terminal | serine/threonine | cytosol |
| | kinase) | kinase | |
| cancer | MEK (MAP/Erk kinase) | serine/threonine | cytosol |
| | | kinase | |
| cancer | PI3K (phosphatidyl | lipid kinase | cytosolic-side of |
| | inositol 3 kinase) | | cell membrane |
| cancer | AKT | serine/threonine | cytosol |
| | | kinase | |
| inflammatory | Caspase-1 (cysteine- | protease | cytosol |
| diseases (arthritis, | aspartic proteases) | | |
| gout, inflammatory | | | |
| bowel disease), | | | |
| neurodiseases | | | |
| (Huntington | | | |
| Disease, epilepsy) | | | |
| and metabolic | | | |
| diseases such as | | | |
| diabetes type 2 and | | | |
| obesity, cryopyrin- | | | |
| associated periodic | | | |
| syndromes, chronic | | | |
| obstructive | | | |
| pulmonary disease | | | |
| inflammatory | NEMO also known as | regulatory binding | cytosol |
| diseases such as | IKKγ (IKK gamma) | protein/adaptor | |
| psoriasis, | | scaffold protein | |
| rheumatoid arthritis, | | | |
| age-related macular | | | |
| degeneration, | | | |
| cancer, duchene | | | |
| muscular dystrophy, | | | |
| ALS, and cachexia- | | | |
| induced cardiac | | | |
| atrophy | | | |
| inflammatory | MyD88 (Myeloid | regulatory binding | cytosol |
| diseases | differentiation primary | protein/adaptor | |
| (rhuematoid | response) | scaffold protein | |
| arthritis, gout, | | | |
| crohn's disease), | | | |
| epilespy, | | | |
| Huntington Disease; | | | |
| pyogenic bacterial | | | |
| infections | | | |
| inflammatory | ASC | regulatory binding | cytosol |
| diseases (arthritis, | | protein/adaptor | |
| gout, inflammatory | | scaffold protein | |
| bowel disease), | | | |
| neurodiseases | | | |
| (Huntington | | | |
| Disease, epilepsy) | | | |
| and metabolic | | | |
| diseases such as | | | |
| diabetes type 2 and | | | |
| obesity, cryopyrin- | | | |
| associated periodic | | | |
| syndromes, chronic | | | |
| obstructive | | | |
| pulmonary disease | | | |
| inflammatory | NLRP3 (inflammasome | regulatory binding | cytosol |
| diseases (arthritis, | component) | protein/adaptor | |
| gout, inflammatory | | scaffold protein | |
| bowel disease), | | | |
| neurodiseases | | | |
| (Huntington | | | |
| Disease, epilepsy) | | | |
| and metabolic | | | |
| diseases such as | | | |
| diabetes type 2 and | | | |
| obesity, cryopyrin- | | | |
| associated periodic | | | |
| syndromes, chronic | | | |
| obstructive | | | |
| pulmonary disease | | | |
| inflammatory and | retinoic acid-related | Txn factor | nuclear |
| autoimmune | orphan receptor (RORγT) | | |
| diseases such as | (RORgammaT) | | |
| inflammatory bowel | | | |
| disease, multiple | | | |
| sclerosis, Gout, | | | |
| Arthritis, psoriasis | | | |
| cancer | Thymidylate synthase | metabolic enzyme | cytosol & nucleus |
| cancer | abl tyrosine kinase; bcr- | tyrosine kinase | cytosol |
| | abl (product of | | |
| | chromosomal | | |
| | translocation) | | |
| | Interferon Regulatory | Txn factor | nucleus |
| | Factor 1 (IRF-1) - | | |
| | transcription factor | | |
| cancer | fms-related tyrosine | tyrosine kinase | cytosolic-side of |
| | kinase 1 (vascular | | cell membrane |
| | endothelial growth | | |
| | factor/vascular | | |
| | permeability factor | | |
| | receptor) | | |
| cancer | fms-related tyrosine | tyrosine kinase | cytosolic-side of |
| | kinase 3 | | cell membrane |
| cancer | kinase insert domain | tyrosine kinase | cytosolic-side of |
| | receptor (a type III | | cell membrane |
| | receptor tyrosine kinase) | | |
| cancer | macrophage stimulating 1 | tyrosine kinase | cytosolic-side of |
| | receptor (c-met-related | | cell membrane |
| | tyrosine kinase) | | |
| cancer, diabetic | protein kinase C family | serine/threonine | multiple locations |
| retinopathy | (alpha, beta) | kinase | depending on cell- |
| | | | type and disease |
| | | | (cytosolic, |
| | | | associated with cell |
| | | | membrane |
| Cancer | beta tubulin/microtubule | cytoskeletal | cytosol |
| | | structural protein | |
| Cancer, Charcot- | kinesins and | microtubule | cytosol |
| Marie-Tooth, | chromosome-associated | associated motor | |
| neurogenerative | KIF | protein | |
| diseases, eye | | | |
| disorder | | | |
| Cancer, kidney | Dynein | microtubule | cytosol |
| diseases, respiratory | | associated motor | |
| diseases, hearing | | protein | |
| loss | | | |
| inflammation, pain | prostaglandin- | cyclooxygenase | cytosolic face of |
| | endoperoxide synthase 2 | | membranes |
| | (prostaglandin G/H | | |
| | synthase and | | |
| | cyclooxygenase) COX-2 | | |
| cancer | Rho associated protein | serine/threonine | cytosol |
| | kinases | kinase | |
| cancer | Aurora protein kinases | serine/threonine | nucleus-cytosol |
| | | kinase | (functions before |
| | | | and during nuclear |
| | | | envelope |
| | | | breakdown) |
| | Insulin receptor substrates | regulatory binding | cytosolic face of |
| | (IRS) | protein/adaptor | plasma membrane |
| | | scaffold protein | |
| cancer | focal adhesion kinases | tyrosine kinase | cytosol |
| | (PTK2) | | |
| cancer | cyclin dependent kinases | serine/threonine | nucleus |
| | | kinase | |
| Cancer | Bcl-2 | regulatory binding | outer mitochondrial |
| | | protein/adaptor | membrane |
| | | scaffold protein | |
| cancer | Telomerase | reverse | nuclear |
| | | transcriptase | |
| cancer | cytochrome c | electron transport | cytosol (when |
| | | pathway component, | released from |
| | | regulatory binding | mitochondria) |
| | | protein/adaptor | |
| | | scaffold protein | |
| | | (only in context of | |
| | | stimulating apoptosis) | |

The foregoing are merely exemplary of intracellular targets. The present disclosure is application to any target (e.g., generating complexes comprising an AAM moiety that binds to any intracellular target).

Regardless of the target or the particular use, in certain embodiments, a complex is administered to a cell or organism in an effective amount. The term “effective amount” means an amount of an agent to be delivered that is sufficient, when administered to a cell or a subject to have the desired effect. In the context of the present disclosure, an effective amount may be the amount sufficient to promote delivery of the complex into a cell and to promote binding of the AAM moiety to its target. In a therapeutic setting, an effective amount is the amount sufficient to treat (e.g., alleviate, improve or delay onset of one or more symptoms of) a disease, disorder, and/or condition.

In one embodiment, the AAM moiety is bispecific, e.g., is a bispecific antibody, or bispecific fragment thereof. A complex comprising a bispecific antibody can bind two different target polypeptides at the same time, or at different times.

A complex of the disclosure may be used in a clinical setting, such as for therapeutic purposes. Therapeutic complexes may include an AAM moiety that binds to and reduces the activity of one or more targets (e.g., polypeptide targets). Such AAM moieties are particularly useful for treating a disease, disorder, and/or condition associated with high levels of one or more particular targets, or high activity levels of one or more particular targets.

In some embodiments, the complex is detectable (e.g., one or both of the Surf+ Penetrating Polypeptide portion and the AAM moiety portion are modified with a detectable label). For example, one or both portions of the complex may include at least one fluorescent moiety. In some embodiments, the Surf+ Penetrating Polypeptide portion has inherent fluorescent qualities. In some embodiments, one or both portions of the complex may be associated with at least one fluorescent moiety (e.g., conjugated to a fluorophore, fluorescent dye, etc.). Alternatively or additionally, one or both portions of the complex may include at least one radioactive moiety (e.g., protein may comprise iodine-131 or Yttrium-90; etc.). Such detectable moieties may be useful for detecting and/or monitoring delivery of the complex to a target site.

A complex associated with a detectable label can be used in detection, imaging, disease staging, diagnosis, or patient selection. Suitable labels include fluorescent, chemiluminescent, enzymatic labels, colorimetric, phosphorescent, density-based labels, e.g., labels based on electron density, and in general contrast agents, and/or radioactive labels.

In some embodiments, the complexes featured in the disclosure may be used for research purposes, e.g., to efficiently deliver AAM moieties to cells in a research context. In some embodiments, the complexes may be used as research tools to efficiently transduce cells with antibody molecules or with other AAM moieties. In some embodiments, complexes may be used as research tools to efficiently introduce an AAM moiety into cells for purposes of studying the effect of the AAM moiety on cellular activity. In certain embodiments, a complex can be used to deliver an AAM moiety into a cell for the purpose of studying the biological activity of the target peptide or protein (e.g., what happens if the target is inhibited or agonized, etc.). In certain embodiments, a complex may be introduced into a cell for the purpose of studying the biological activity of the AAM moiety (e.g., does it inhibit target activity, does it promote target activity, etc.).

### Pharmaceutical Compositions

The present disclosure provides complexes of the disclosure (e.g., a Surf+ Penetrating Polypeptide portions-associated with an AAM moiety portion). This section describes exemplary compositions, such as compositions of a complex of the disclosure formulated in a pharmaceutically acceptable carrier. Any of the complexes comprising any of the Surf+ Penetrating Polypeptides amd any of the AAM moieties described herein may be formulated in accordance with this section of the disclosure.

Thus, in certain aspects, the present disclosure provides compositions, such as pharmaceutical compositions, comprising one or more such complexes, and one or more pharmaceutically acceptable excipients. Pharmaceutical compositions may optionally include one or more additional therapeutically active substances. In accordance with some embodiments, a method of administering pharmaceutical compositions comprising one or more Surf+ Penetrating Polypeptide or one or more complexes of the disclosure (e.g., a complex comprising a Surf+ Penetrating Polypeptide associated with at least one AAM moiety) to be delivered to a subject in need thereof is provided. In some embodiments, compositions are administered to humans. For the purposes of the present disclosure, the phrase “active ingredient” generally refers to an AAM moiety portion complexed with a Surf+ Penetrating Polypeptide portion to be delivered as described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts, as well as suitable or adaptable for research use. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects or patients to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the disclosure may be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may include between 0.1% and 100% (w/w) active ingredient.

Pharmaceutical formulations may additionally include a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, an active ingredient is mixed with at least one inert, pharmaceutically acceptable excipient. In the case of capsules, tablets and pills, the dosage form may comprise buffering agents.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of active ingredient may be as high as the solubility limit of the active ingredient in the solvent.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 um to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other opthalmically-administrable formulations which are useful include those which comprise the active ingredient in microcrystalline form and/or in a liposomal preparation. Ear drops and/or eye drops are contemplated as being within the scope of this disclosure.

In certain embodiments, complexes of the disclosure and compositions of the disclosure, including pharmaceutical preparations, are non-pyrogenic. In other words, in certain embodiments, the compositions are substantially pyrogen free. In one embodiment, the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in relatively large dosages and/or over an extended period of time (e.g., such as for the patient's entire life), even small amounts of harmful and dangerous endotoxin could be dangerous. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

### Administration

The present disclosure provides methods for delivering an AAM moiety into a cell. Cells or tissues are contacted with a complex comprising an AAM moiety and a Surf+ Penetrating Polypeptide, thereby promoting delivery of the AAM moiety into the cell.

The present disclosure provides methods comprising administering Surf+ Penetrating Polypeptide/AAM moiety complexes to a subject in need thereof, as well as methods of contacting cells or cells in culture with such complexes. The disclosure contemplates that any of the complexes of the disclosure (e.g., complexes including a Surf+ Penetrating Polypeptide Portion and a AAM moiety portion) may be administrated, such as described herein. Complexes of the disclosure, including as pharmaceutical compositions, may be administered or otherwise used for research, diagnostic, imaging, prognostic, or therapeutic purposes, and may be used or administered using any amount and any route of administration effective for preventing, treating, diagnosing, researching or imaging a disease, disorder, and/or condition. The exact amount required will vary from subject to subject, depending on the species, age, and general condition of the subject, the severity of the disease, the particular composition, its mode of administration, its mode of activity, and the like. Compositions in accordance with the disclosure are typically formulated in dosage unit form for ease of administration and uniformity of dosage. It will be understood, however, that the total daily usage of the compositions of the present disclosure will be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Surf+ Penetrating Polypeptide/AAM moiety complexes (e.g., complexes of the disclosure) comprising at least one agent to be delivered and/or pharmaceutical, prophylactic, diagnostic, research or imaging compositions thereof may be administered to animals, such as mammals (e.g., humans, domesticated animals, cats, dogs, mice, rats, etc.). In some embodiments, complexes of the disclosure comprising at least one agent to be delivered, and/or pharmaceutical, prophylactic, diagnostic, research or imaging compositions thereof are administered to humans.

Complexes of the disclosure comprising at least one agent to be delivered and/or pharmaceutical, prophylactic, research diagnostic, or imaging compositions thereof in accordance with the present disclosure may be administered by any route and may be formulated in a manner suitable for the selected route of administration or in vitro application. In some embodiments, complexes of the disclosure, and/or pharmaceutical, prophylactic, diagnostic, or imaging compositions thereof, are administered by one or more of a variety of routes, including oral, intravenous, intramuscular, intra-arterial, intramedullary, intrathecal, subcutaneous, intraventricular, transdermal, intradermal, rectal, intravaginal, intraperitoneal, topical (e.g. by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter. Other devices suitable for administration include, e.g., microneedles, intradermal specific needles, Foley's catheters (e.g., for bladder instillation), and pumps, e.g., for continuous release.

In some embodiments, complexes of the disclosure, and/or pharmaceutical, prophylactic, diagnostic, research or imaging compositions thereof, are administered by systemic intravenous injection. In specific embodiments, complexes of the disclosure and/or pharmaceutical, prophylactic, research diagnostic, or imaging compositions thereof may be administered intravenously and/or orally. In specific embodiments, complexes of the disclosure, and/or pharmaceutical, prophylactic, research diagnostic, or imaging compositions thereof, may be administered in a way which allows the complex to cross the blood-brain barrier, vascular barrier, or other epithelial barrier.

Complexes of the disclosure comprising at least one AAM moiety to be delivered may be used in combination with one or more other therapeutic, prophylactic, diagnostic, research or imaging agents. By “in combination with,” it is not intended to imply that the agents must be administered at the same time and/or formulated for delivery together, although these methods of delivery are within the scope of the disclosure. Compositions of the disclosure can be administered concurrently with, prior to, or subsequent to, one or more other desired therapeutics, other reagents or medical procedures. In general, each agent will be administered at a dose and/or on a time schedule determined for that agent. In some embodiments, the disclosure encompasses the delivery of pharmaceutical, prophylactic, diagnostic, research or imaging compositions in combination with agents that improve their bioavailability, reduce and/or modify their metabolism, inhibit their excretion, and/or modify their distribution within the body.

It will further be appreciated that therapeutic, prophylactic, diagnostic, research or imaging active agents utilized in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer in accordance with the disclosure may be administered concurrently with a chemotherapeutic agent), or they may achieve different effects (e.g., control of any adverse effects).

### Kits

The disclosure provides a variety of kits (or pharmaceutical packages) for conveniently and/or effectively carrying out methods of the present disclosure. Typically kits will comprise sufficient amounts and/or numbers of components to allow a user to perform multiple treatments of a subject(s) and/or to perform multiple experiments for desired uses (e.g., laboratory or diagnostic uses). Alternatively, a kit may be designed and intended for a single use. Components of a kit may be disposable or reusable.

In some embodiments, kits include one or more of (i) a Surf+ Penetrating Polypeptide as described herein and an AAM moiety to be delivered; and (ii) instructions (or labels) for forming complexes comprising the Surf+ Penetrating Polypeptide associated with the AAM moiety (e.g., with at least one AAM moiety). Optionally, such kits may further include instructions for using the complex in a research, diagnostic or therapeutic setting.

In some embodiments, a kit includes one or more of (i) a Surf+ Penetrating Polypeptide portion as described herein and an AAM moiety portion to be delivered or a complex of such Surf+ Penetrating Polypeptide associated with such AAM moiety; (ii) at least one pharmaceutically acceptable excipient; (iii) a syringe, needle, applicator, etc. for administration of a pharmaceutical, prophylactic, diagnostic, or imaging composition to a subject; and (iv) instructions and/or a label for preparing the pharmaceutical composition and/or for administration of the composition to the subject.

In some embodiments, a kit includes one or more of (i) a pharmaceutical composition comprising a complex of the disclosure (e.g., a Surf+ Penetrating Polypeptide portion as described herein associated with an AAM moiety portion to be delivered); (ii) a syringe, needle, applicator, etc. for administration of the pharmaceutical, prophylactic, diagnostic, or imaging composition to a subject; and (iii) instructions and/or a label for administration of the pharmaceutical, prophylactic, diagnostic, or imaging composition to the subject. Optionally, the kit need not include the syringe, needle, or applicator, but instead provides the composition in a vial, tube or other container suitable for long or short term storage until use.

In some embodiments, a kit includes one or more components useful for modifying proteins of interest, such as by supercharging the protein, to produce a Surf+ Penetrating Polypeptide. These kits typically include all or most of the reagents needed. In certain embodiments, such a kit includes computer software to aid a researcher in designing the engineered or otherwise modified Surf+ Penetrating Polypeptide an in accordance with the disclosure. In certain embodiments, such a kit includes reagents necessary for performing site-directed mutagenesis.

In some embodiments, a kit may include additional components or reagents. For example, a kit may include buffers, reagents, primers, oligonucleotides, nucleotides, enzymes, buffers, cells, media, plates, tubes, instructions, vectors, etc.

In some embodiments, a kit comprises two or more containers. In certain embodiments, a kit may include one or more first containers which comprise a Surf+ Penetrating Polypeptide, and optionally, at least one AAM moiety molecule to be delivered, or a complex comprising a Surf+ Penetrating Polypeptide and at least one AAM moiety to be delivered for diagnosing or prognosing a disease, disorder or condition or for research use; and the kit also includes one or more second containers which comprise one or more other prophylactic or therapeutic agents useful for the prevention, management or treatment of the same disease, disorder or condition, or useful for the same research application.

In some embodiments, a kit includes a number of unit dosages of a pharmaceutical, prophylactic, diagnostic, or imaging composition comprising a complex of the disclosure or comprising a Surf+ Penetrating Polypeptide, and optionally, at least one AAM moiety to be delivered. In some embodiments, the unit dosage form is suitable for intravenous, intramuscular, intranasal, oral, topical or subcutaneous delivery. Thus, the disclosure herein encompasses solutions, preferably sterile solutions, suitable for each delivery route. A memory aid may be provided, for example in the form of numbers, letters, and/or other markings and/or with a calendar insert, designating the days/times in the treatment schedule in which dosages can be administered. Placebo dosages, and/or calcium dietary supplements, either in a form similar to or distinct from the dosages of the pharmaceutical, prophylactic, diagnostic, or imaging compositions, may be included to provide a kit in which a dosage is taken every day.

In some embodiments, the kit may further include a device suitable for administering the composition according to a specific route of administration or for practicing a screening assay.

Kits may include one or more vessels or containers so that certain of the individual components or reagents may be separately housed. Exemplary containers include, but are not limited to, vials, bottles, pre-filled syringes, IV bags, blister packs (comprising one or more pills). A kit may include a means for enclosing individual containers in relatively close confinement for commercial sale (e.g., a plastic box in which instructions, packaging materials such as styrofoam, etc., may be enclosed). Kit contents can be packaged for convenient use in a laboratory.

In the case of kits sold for laboratory and/or diagnostic use, the kit may optionally contain a notice indicating appropriate use, safety considerations, and any limitations on use. Moreover, in the case of kits sold for laboratory and/or diagnostic use, the kit may optionally comprise one or more other reagents, such as positive or negative control reagents, useful for the particular diagnostic or laboratory use.

In the case of kits sold for therapeutic and/or diagnostic use, a kit may also contain a notice in the form prescribed by a governmental agency regulating the manufacture, use or sale of pharmaceuticals or biological products, which notice reflects (a) approval by the agency of manufacture, use or sale for human administration, (b) directions for use, or both.

These and other aspects of the present disclosure will be further appreciated upon consideration of the following Examples, which are intended to illustrate certain particular embodiments of the disclosure but are not intended to limit its scope, as defined by the claims.

### EXAMPLE

The disclosure now being generally described, it will be more readily understood by reference to the following examples, which are included merely for purposes of illustration of certain aspects and embodiments of the present disclosure, and are not intended to limit the disclosure.

### Example 1

### A Microtubule Localizing Complex

In one exemplification, an antibody to tubulin is biotinylated at the sulfhydryl groups on one or more cysteines and conjugated to a supercharged streptavidin (+52SAV). +52SAV is an example of a Surf+ Penetrating Polypeptide. It has high net positive charge, surface positive charge and penetrates cells. +52SAV is a tetramer of four monomers, each of which has a net charge of +13. The mass of each monomer is 16.54 kDa and the charge/molecular weight ratio of the tetramer is 0.79.

Each monomer of the +52SAV tetramer has the following amino acid sequence: DPSKDSKAQVSAAKAGITGTWYNQLGSTFIVTAGAKGALTGTYESAVGNAK SRYVLTGRYDSAPATKGSGTALGWTVAWKNKYRNAHSATTWSGQYVGGA KARINTQWLLTSGTTKAKAWKSTLVGHDTFTKVKPSAASIDAAKKAGVNNG NPLDAVQQ (SEQ ID NO: 658).

For in vitro analysis of this complex, cells in culture are contacted with the +52SAV-tubulin antibody complex. The complex is internalized by the cells. Once inside a cell, the tubulin antibody binds its target (e.g., tubulin expressed by microtubules in the cell), which is detected by immunofluorescence with antibodies to the tubulin antibody after cell fixation and permeabilization.

For in vivo studies, the +52SAV-tubulin antibody complex is injected subcutaneously into rats and, following a punch biopsy and/or harvest of various tissue samples, immunohistochemistry is performed with antibodies to the tubulin antibody to detect tissue penetration and biodistribution.

Suitable controls are conducted and include the use of an anti-tubulin antibody alone to confirm that the AAM moiety alone does not efficiently penetrate non-permeabilized cells or does so at levels substantially less than that of the complex, as well as the use of the Surf+ Penetrating Polypeptide alone to confirm that it does not independently bind specifically to the intracellular target.

+52SAV expression and purification: His6×-tagged+52SAV was expressed in BL21(DE3) cells, grown in Terrific Broth media (Boston Bioproducts, Ashland, Mass.), and induced with 1 mM IPTG for 4 hours at 37° C. Cells were lysed with 5 mL of lysis buffer (1× Bugbuster® (EMD Chemicals, Rockland, MA), 20 mM Hepes pH 7.5, 150 mM NaCl, 25 U/mL Benzonase (EMD Chemicals, Rockland, MA), 0.1 mg/mL lysozyme and EDTA-free 1×protease inhibitors (Roche, South San Francisco, Calif.)) per gram of cell paste. The resulting inclusion body pellet from centrifugation of the lysate was washed three times with lysis buffer, then resuspended in 6M guanidinium hydrochloride, pH 1.5 and dialyzed against the same buffer overnight. The denatured protein was refolded by dialysis against 50 mM Hepes pH 7.5, 150 mM NaCl, and 0.3M guanidinium hydrochloride. Affinity purification of refolded +52SAV was carried out using Iminobiotin Agarose according to the manufacturer's instructions (Pierce®, Thermo Fisher Scientific Inc., Rockford, Ill.).

Biotinylation of antibody: Disulfide bonds of commercially available anti-tubulin antibody (sheep polyclonal; Cytoskeleton, Inc., Denver, Colo.) were reduced by 1 hour incubation with 10 mM beta-mercaptoethanol at 37° C. Residual beta-mercaptoethanol was removed from the antibody using Zeba™ Spin Desalting Columns (Pierce®, Thermo Fisher Scientific Inc., Rockford, Ill.) according to the manufacturer's instructions. The resulting reduced antibody was biotinylated on the free sulfhydryl groups using EZ-Link® BMCC-Biotin (Pierce®, Thermo Fisher Scientific Inc., Rockford, Ill.) according to the manufacturer's instructions. The level of biotinylation (usually 1-2 biotin molecules per antibody) was determined using a Fluorescence Biotin Quantitation kit (Pierce®, Thermo Fisher Scientific Inc., Rockford, Ill.).

Generation of the antibody/+52SAV complex: +52SAV was incubated with biotinylated antibody and free biotin to generate a 1:1 molar ratio of antibody bound to +52SAV. This complex was then purified using a cation exchange resin (SP sepharose, fast flow; GE Healthcare).

Cell uptake and visualization: HeLa cells (ATCC, Manassas, Va.) were plated at a density of 10⁴ cells per well of a 96-well dish one day prior to treatment with protein. Uptake and binding of tubulin antibody to intracellular microtubules will be assessed by dose ranging (0.05 to 2 μM) and time course incubation of the antibody/+52SAV complex with cells. After treatment, cells are fixed with 4% paraformaldehyde followed by permeabilization with 0.5% saponin. The fixed and permeabilized cells are incubated with a fluorescent labeled secondary antibody and visualized by fluorescence microscopy.

In the foregoing example, +52SAV may be replaced by a human Surf+ Penetrating Polypeptide, such as a fragment of a naturally occurring polypeptide set forth in FIG. 1 or FIG. 2, including specific domains identified by PDB number, or fragments thereof having surface positive charge, a mass of at least 4 kDa, and a charge/molecular weight ratio of at least 0.75. Amino acid sequence information for full length proteins identified in FIGS. 1 and 2 by GenBank Accession number are provided in Section 1 of the Sequence Listing. Amino acid sequence information for domains of protein identified in FIGS. 1 and 2 by PDB identifier are provided in Section 2 of the Sequence Listing.

Moreover, the commercially available anti-tubulin antibody may be replaced by a recombinantly produced anti-tubulin antibody. Use of a recombinantly produced antibody facilitates generating complexes as fusion proteins comprising a Surf+ Penetrating Polypeptide portion and an AAM moiety portion. Such replacement of the specific embodiments set forth in these examples with other suitable embodiments is specifically contemplated.

### Example 2

### A Nuclear Pore Localizing Complex

In another exemplification, antibody to nucleoporin (mouse monoclonal [QE5]; Abcam, Cambridge, Mass.) is biotinylated at the sulfhydryl groups at one or more cysteines and conjugated to a supercharged streptavidin (+52SAV).

For in vitro analysis of this complex, cells in culture are contacted with the +52SAV-nucleoporin antibody complex. The complex is internalized by the cells. Once inside a cell, the nucleoporin antibody binds to the nuclear pore in the cell (e.g., binds to its target nucleoporin expressed by the nuclear pore), which is detected by immunofluorescence with antibodies to the nucleoporin antibody after cell fixation and permeabilization.

For in vivo studies, the +52SAV-nucleoporin antibody complex is injected subcutaneously into rats and, following a punch biopsy and/or harvest of various tissue samples, immunohistochemistry is performed with antibodies to the nucleoporin antibody to detect tissue penetration and biodistribution. Methods for preparation and testing of the +52SAV-antibody complex will be followed as described above.

In the foregoing example, +52SAV may be replaced by a human Surf+ Penetrating Polypeptide, such as a fragment of a naturally occurring polypeptide set forth in FIG. 1 or FIG. 2, including specific domains identified by PDB number, or fragments thereof having surface positive charge, a mass of at least 4 kDa, and a charge/molecular weight ratio of at least 0.75. Amino acid sequence information for full length proteins identified in FIGS. 1 and 2 by GenBank Accession number are provided in Section 1 of the Sequence Listing. Amino acid sequence information for domains of protein identified in FIGS. 1 and 2 by PDB identifier are provided in Section 2 of the Sequence Listing.

Moreover, the commercially available antibody may be replaced by a recombinantly produced antibody. Use of a recombinantly produced antibody facilitates generating complexes as fusion proteins comprising a Surf+ Penetrating Polypeptide portion and an AAM moiety portion. Such replacement of the specific embodiments set forth in these examples with other suitable embodiments is specifically contemplated.

### Example 3

### A Golgi Localizing Complex

In another exemplification, antibody to p58 Golgi protein (mouse monoclonal [58K-9]; Abcam) is biotinylated at the sulfhydryl groups at one or more cysteines and conjugated to a supercharged streptavidin (+52SAV).

For in vitro analysis of this complex, cells in culture are contacted with the +52SAV-p58 Golgi antibody complex. The complex is internalized by the cells. Once inside a cell, the p58 Golgi antibody binds to the perinuclear Golgi apparatus in the cell, which is detected by immunofluorescence with antibodies to the p58 Golgi antibody after cell fixation and permeabilization.

For in vivo studies, the +52SAV-p58 Golgi antibody complex is injected subcutaneously into rats and, following a punch biopsy and/or harvest of various tissue samples, immunohistochemistry is performed with antibodies to the p58 Golgi antibody to detect tissue penetration and biodistribution. Methods for preparation and testing of the +52SAV-antibody complex will be followed as described above.

In the foregoing example, +52SAV may be replaced by a human Surf+ Penetrating Polypeptide, such as a fragment of a naturally occurring polypeptide set forth in FIG. 1 or FIG. 2, including specific domains identified by PDB number, or fragments thereof having surface positive charge, a mass of at least 4 kDa, and a charge/molecular weight ratio of at least 0.75. Amino acid sequence information for full length proteins identified in FIGS. 1 and 2 by GenBank Accession number are provided in Section 1 of the Sequence Listing. Amino acid sequence information for domains of protein identified in FIGS. 1 and 2 by PDB identifier are provided in Section 2 of the Sequence Listing.

Moreover, the commercially available antibody may be replaced by a recombinantly produced antibody. Use of a recombinantly produced antibody facilitates generating complexes as fusion proteins comprising a Surf+ Penetrating Polypeptide portion and an AAM moiety portion. Such replacement of the specific embodiments set forth in these examples with other suitable embodiments is specifically contemplated.

### Example 4

### An Inhibitory Complex

In another exemplification, a neutralizing antibody to caspasel (mouse monoclonal [D57A2]; Cell Signaling Technology, Inc.®, Danvers, Mass.) is biotinylated at the sulfhydryl groups at one or more cysteines and conjugated to a supercharged streptavidin (+52SAV).

For in vitro analysis of this complex, cells in culture are contacted with the +52SAV-caspase antibody complex. The complex is internalized by the cells. Internalization is confirmed, as described above, using immunofluorescence with secondary antibodies to the caspase 1 antibody. The functional activity of the caspasel antibody inside the cell is assayed by, for example, measuring the effect on inhibition of pro-IL-1β processing and reduction in levels of secreted active IL-1β, which can be monitored by an immunoassay of the cell supernatant such as an ELISA assay, for which a commercially available kit is available (Pierce®, Thermo Fisher Scientific Inc., Rockford, Ill.). Such an assay is used to confirm that once delivered into cells, the neutralizing antibody to caspasel maintains its function (e.g., the antibody inhibits an activity of caspasel).

For in vivo studies, mice are injected intraarticularly with monosodium urate crystals plus C18 free fatty acids to induce joint swelling. Such joint swelling may be monitored by macroscopic scoring, by ^{99m}Tc uptake, by local IL-1β levels and/or by quantifying immune cell influx into the joint, and each of these methods have been previously described (Joosten L A, et al. (2010) Arthritis & Rheumatism 62:3237-3248). Given that the neutralizing caspasel antibody reduces IL-1β levels, the complex is evaluated for its ability to alleviate symptoms caused, in whole or in part, by elevated local IL-1β levels. The +52SAV-caspase 1 antibody complex is injected intraarticularly with dose ranging and time course (including prior to, concomitant with and post injection of urate crystals plus C18 free fatty acids) studies. Following injection, treated mice are evaluated for inhibition of joint swelling in comparison to untreated mice.

In the foregoing example, +52SAV may be replaced by a human Surf+ Penetrating Polypeptide, such as a fragment of a naturally occurring polypeptide set forth in FIG. 1 or FIG. 2, including specific domains identified by PDB number, or fragments thereof having surface positive charge, a mass of at least 4 kDa, and a charge/molecular weight ratio of at least 0.75. Amino acid sequence information for full length proteins identified in FIGS. 1 and 2 by GenBank Accession number are provided in Section 1 of the Sequence Listing. Amino acid sequence information for domains of protein identified in FIGS. 1 and 2 by PDB identifier are provided in Section 2 of the Sequence Listing.

Moreover, the commercially available antibody may be replaced by a recombinantly produced antibody. Use of a recombinantly produced antibody facilitates generating complexes as fusion proteins comprising a Surf+ Penetrating Polypeptide portion and an AAM moiety portion. Such replacement of the specific embodiments set forth in these examples with other suitable embodiments is specifically contemplated.

### Example 5

### Complexes Comprising Naturally Occurring Surf+ Penetrating Polypeptides

In another exemplification, a naturally occurring human Surf+ Penetrating Polypeptide, such as a cell penetrating fragment of HBEGF, are fused in frame for expression of a chimeric fusion protein to an AAM moiety, such as an Adnectin®, DARPin, nanobody, scFv or single VH or VL domain antibody. Although HBEGF and the AAM moiety can be directly linked, in this example the two moieties are interconnected via a linker, such as a (G_{4S)}₃ (i.e., a (Gly-Gly-Gly-Gly-Ser)₃) linker. A suitable HBEGF fragment is set forth in PDB ID 1×DT and is a polypeptide of about 79 amino acid residues (e.g., includes about amino acid residues 72-147 of the full length HBEGF protein). This HBEGF domain is an example of a naturally occurring human Surf+ Penetrating Polypeptide. It has surface positive charge, charge/molecular weight of at least 0.75, and a molecular weight of at least 4 kDa. Specifically, this polypeptide has a molecular weight of about 8.9 kDa, a net charge of +12, and a charge/molecular weight of 1.35. Moreover, this HBEGF fragment is exemplary of Surf+ Penetrating Polypeptides having a charge/molecular weight of at least 0.75, but for which the charge/molecular weight of the full length naturally occurring protein is less than 0.75 (e.g., charge/molecular weight of full length HBEGF is about 0.52). Subdomains (e.g., smaller functional fragments) of HBEGF having surface positive charge, a mass of at least 4 kDa, a charge/molecular weight ratio of at least 0.75, and cell penetrating capability may also be used.

Optionally, the complex includes one or more tags to facilitate detection and/or purification. In one example, a 10 amino acid sequence including the 6×His tag is appended to the N-terminus of the fusion protein (MGHHHHHHGG) (SEQ ID NO: 659) and a 9 amino acid myc epitope tag plus two glycines as a linker sequence (GGEQKLISEEDL) (SEQ ID NO: 660) is appended to the C-terminus of the fusion protein.

For in vitro analysis, this His-HBEGF-linker-AAM moiety-myc fusion protein is contacted with and internalized by a cell. Accumulation in the cell is monitored by immunofluorescence with an anti-myc antibody (mouse monoclonal [9E10]; Abcam, Cambridge, Mass.).

The AAM moiety may be an scFv that binds tubulin. The His-HBEGF-linker-tubulin scFv-myc fusion protein is contacted with and internalized by a cell and the myc-tagged tubulin scFv binds to microtubules in the cell, which can be subsequently detected by immunofluorescence with anti-myc tag antibody following fixation, permeabilization.

For this and other examples, the order of the fusion protein may be altered so that the Surf+ Penetrating Polypeptide portion of the complex is located C-terminally to the AAM moiety portion of the complex, e.g. myc-tubulin scFv-linker-HBEGF-His.

HBEGF expression and purification: the His-HBEGF-tubulin scFv-myc fusion protein was expressed in SHuffle® cells (New England Biolabs, Ipswich, Mass.), grown in Progro™ media (Expression Technologies, San Diego, Calif.), and induced with 0.5 mM IPTG for 19 hours at 22° C. Cells were lysed in lysis buffer as described above. The lysate supernatant was subjected to fractionationg on a HiTrap™ IMAC column (GE Healthcare, Piscataway, N.J.), followed by a SP-HP cation exchange column (GE Healthcare, Piscataway, N.J.), and finally a Superdex™ 75 10/300 GL gel filtration column (GE Healthcare, Piscataway, N.J.) to purify the fusion protein. The fusion protein is stored in high salt PBS buffer (8 mM sodium phosphate, 2 mM potassium phosphate, 2.7 mM KCl, 0.5 M NaCl, pH 7.4)

Cell uptake and visualization: HeLa cells are plated as above and subjected to dose ranging (0.05 to 2 μM) and time course studies for uptake of the His-HBEGF-tubulin scFv-myc fusion protein. After incubation with the fusion protein, cells are fixed and permeabilized as described above. The fixed and permeabilized cells are incubated with a fluorescent labeled secondary antibody and visualized by fluorescent microscopy.

In the foregoing example, the Surf+ Penetrating Polypeptide may be replaced by a human Surf+ Penetrating Polypeptide, such as a fragment of a naturally occurring polypeptide set forth in FIG. 1 or FIG. 2, including specific domains identified by PDB number, or fragments thereof having surface positive charge, a mass of at least 4 kDa, and a charge/molecular weight ratio of at least 0.75. Amino acid sequence information for full length proteins identified in FIGS. 1 and 2 by GenBank Accession number are provided in Section 1 of the Sequence Listing. Amino acid sequence information for domains of protein identified in FIGS. 1 and 2 by PDB identifier are provided in Section 2 of the Sequence Listing.

### Example 6

### Complexes Comprising an Antibody-Mimic Moiety

In some embodiments, the AAM moiety in the complex is an Adnectin® sequence, such as the naïve, wild type Fn3 Adnectin®, which has no target binding protein in the cells, but is studied for biophysical and biochemical properties in fusion with a Surf+ Penetrating Polypeptide of the disclosure and for monitoring uptake into cells.

Alternatively, a complex of a Surf+ Penetrating Polypeptide and the HA4 or 7c12 Adnectin® sequence is made and studied. These particular AAM moieties bind to the SH2 domain of the Abelson kinase, as described by Grebien, F et at (2011) Cell 147:306-319. The resulting complex is internalized by cells and binds (via the AAM moiety) to the cytoplasmic Bcr-Abl kinase fusion protein. Either complex is studied in vitro and/or in vivo, such as using assays described above. Additionally, such complexes will be evaluated in dose ranging and time course studies for ability to inhibit Abl kinase activity and leukemogenesis in mouse BaF3 cells harboring Bcr-Abl kinase, as previously described (Grebien, F et at (2011) Cell 147:306-319).

### Example 7

### Complexes Comprising an Antibody-Mimic Moiety

In some embodiments, the AAM moiety complexed to a Surf+ Penetrating Polypeptide (e.g., chemically conjugated or complexed as a fusion protein) is a designed ankyrin repeat protein, or DARPin, such as a naïve DARPin or the 2A1 and 2F6 DARPins that bind to the CC2-LZ domain of IKKγ/NEMO, as previously described (Wyler, E. et at (2007) Protein Science 16:2013-2022). For any of these complexes, a His tag is optionally appended to the fusion protein to facilitate purification from E. coli, and a myc epitope tag is optionally appended to the DARPin sequence to monitor intracellular uptake, localization and persistence of the myc tagged DARPin protein inside the cells.

HEK293T cells are transiently transfected with an NF-kB reporter plasmid, such as pIgκ-luc, and co-transfected with a β-galactosidase expressing reporter plasmid. After 24 hours, cells are stimulated with 10 ng/mL TNF-α and cell lysates are assayed for both reporter protein activities, where the β-galactosidase activity is used to normalize transfection and reporter protein activity. The His-Surf+ Penetrating Polypeptide-linker-DARPin-myc fusion protein is contacted with the cells for dose ranging and time course studies of inhibition of NEMO activity and reduced NF-kB activation following TNF-α stimulation, as previously described (Wyler, E. et at (2007) Protein Science 16:2013-2022).

### Example 8

### Target Subcellular Localization

The present disclosure provides complexes and methods for delivering AAM moieties into cells. The target of the particular AAM moiety may itself be localized in, for example, the nucleus, peroxisome, cytoplasm, mitochondria, cytoplasmic face of the cell membrane, etc.

In some embodiments, the target of the particular AAM moiety is localized in the nucleus. Optionally, a nuclear localization sequence (NLS), for instance the peptide sequence DPKKKRKV (SEQ ID NO: 661), is included in the complex, such that the complex has any of the following exemplary structures to facilitate its targeting to the nucleus: His-Surf+ Penetrating Polypeptide-linker-NLS-AAM moiety-myc; His-Surf+ Penetrating Polypeptide-linker-AAM moiety-NLS-myc; NLS-AAM moiety-linker-Surf+ Penetrating Polypeptide; AAM moiety-NLS-linker-Surf+Penetrating Polypeptide. As detailed throughout, His and/or myc tags may be present, absent or replaced with another tag. Moreover, additional linkers may be present or absent. After contacting and penetration into the cell, the AAM moiety will transit to and accumulate inside the nucleus. Accumulation in the cell nucleus is monitored by immunofluorescence with an anti-myc antibody and is detected by fluorescence microscopy of live or fixed cells.

In some embodiments, the target is localized in the peroxisome. Optionally, a peroxisomal targeting sequence (PTS) is appended to the C-terminus of the AAM moiety (His-Surf+ Penetrating Polypeptide-linker-myc-AAM moiety-PTS). After contacting and penetration into the cell, the AAM moiety portion will transit to and accumulate inside peroxisomes. Accumulation in the cell is monitored by immunofluorescence with an anti-myc antibody. Alternatively, the PTS may be appended to another portion of the complex, such as to the Surf+ Penetrating Polypeptide portion.

In some embodiments, the target is localized to the cytosolic face of the plasma membrane. Optionally, a plasma membrane localization signal sequence (KLNPPDESGPGCMSCKCVLS) (SEQ ID NO: 662) is appended to the C-terminus of the AAM moiety (His-Surf+ Penetrating Polypeptide-linker-AAM moiety-myc-membrane localization signal) to facilitate its targeting and binding to the cytosolic face of the plasma membrane. After contacting and penetration into the cell, the AAM moiety will transit to and accumulates at the cytosolic face of the plasma membrane, which is monitored by immunofluorescence with an anti-myc antibody and detected by fluorescence microscopy of live or fixed cells. Alternatively, the plasma membrane localization signal may be appended to another portion of the complex, such as to the Surf+ Penetrating Polypeptide portion.

In some embodiments, the target is localized in the mitochondrial matrix. Optionally, a mitochondrial matrix localization signal sequence (MLS) is appended to the N-terminus of the AAM moiety, which is followed by the linker sequence and then the Surf+ Penetrating Polypeptide (MLS-AAM moiety-myc-linker-Surf+ Penetrating Polypeptide). After contacting and penetration into the cell, the AAM moiety will transit to and accumulate inside the mitochondrial matrix. Accumulation in the cell is monitored by immunofluorescence with an anti-myc antibody and detected by fluorescence microscopy of live or fixed cells. Alternatively, the MLS may be appended to another portion of the complex, such as to the Surf+ Penetrating Polypeptide portion.

### Example 9

### Surface-Charged Fusion Proteins with Single Chain Antibody (scFv) to Huntingtin Protein

A complex comprising a supercharged GFP protein (another example of a Surf+ Penetrating Polypeptide, in this case a charge engineered protein) fused via a glycine-serine linker to an AAM moiety (in this case, an scFv that specifically binds huntingtin protein; an intracellular target) was expressed and purified. The complex was also tagged on the N-terminus with a Myc tag and on the C-terminus with a Hisx6 tag. A control lacking the AAM moiety was also expressed and purified. The complexes are fusion protein and can be represented as:
Myc-+36GFP-(G₄5)₂-C4-His₆;
Myc-+36GFP-His₆;
   where “+36GFP” denotes the supercharged GFP portion; “C4” denotes the particular AAM moiety used in this example; and (G₄5)₂ denotes the linker used to link the supercharged GFP portion to the AAM moiety (this linker is also referred to as GS10). In this particular example, the supercharged GFP portion has a net charge of +36. The amino acid sequence of +36GFP is set forth in SEQ ID NO: 663. The AAM moiety in this example is an scFv that specifically binds huntingtin protein; an intracellular target. This single chain Fv, also known as an intrabody because it is an scFv that binds an intracellular target, is denoted “C4”. The C4 scFv binds to the first 17 amino acids of huntingtin protein and has been demonstrated to delay the aggregation phenotype when the gene is delivered in adeno-associated viral vectors (AAV2/1) in mice (J Neuopathol Exp Neurol. 2010. 69(10):1078-1085). In other words, the scFv binds to the intracellular protein and prevents the bound protein from binding to another protein, in this case, another huntington protein molecule. This is an example of one mechanism by which an AAM might impact the activity of an intracellular target. In this example, the AAM is preventing the bound protein from binding its binding partner (a protein) which may be a different protein or another molecule of the same protein.

Inability to get penetration of the protein has limited its use to such a viral-based approach.

In this example, the complex is a fusion protein and the GFP and scFv portion are interconnected via a peptide linker. This fusion protein is a single polypeptide chain (e.g., the portions are connected to form a single polypeptide chain). Here, the peptide linker is a ten amino acid linker, specifically (GGGGS)₂. In this particular example, the GFP portion is N-terminal to the scFv. However, in other embodiments, the GFP portion may be C-terminal to the scFv portion. Moreover, the linker sequence and/or length can be varied, and the fusion protein may or may not have a tag. The amino acid sequence for the GFP-scFv fusion protein (Myc-+36GFP-(G₄S)₂-C4-His₆) is set forth in SEQ ID NO: 664. The amino acid sequence of the control complex (Myc-+36GFP-His₆) is set forth in SEQ ID NO: 665.

### Example 10

### The Binding of AMM Moiety to its Target is Maintained when Delivered into Cells as a Fusion Protein with a Surf+ Penetrating Polypeptide

Experiments were conducted to demonstrate that the complex described in Example 9 (Myc-+36GFP-(G₄5)₂-C4-His₆) can be effectively delivered into cells and disrupt aggregation of mHTT. In other words, does the fusion protein have the ability to penetrate cells and yet retain the ability of the C4 (scFv; AAM moiety) to bind its intracellular target and disrupt the binding of this target to its binding partners (e.g., disrupt binding to another protein—whether that other protein be the same or different).

C4 has been previously shown to block HTT aggregation when delivered by transient transfection using a viral system (Butler and Messer, PLosOne 2011, 6;e29199). This assay was employed to assess whether C4 maintains its activity when delivered into cels via a Surf+ Penetrating Polypeptide. In this assay a HTT exon 1 protein fragment containing 46 glutamine repeats and a red fluorescence protein tag (HDex1-RFP) was expressed in ST14A cells by transient transfection. ST14A cell are immortalized rat neuron progenitor cells, a cell line representative of immature CNS cells. If left untreated the protein forms punctate aggregates in the cells, which can be visualized by fluorescence microscopy. The assay is as followed:
1. Transfect cells using jetPEI™ with a plasmid encoding HDex1-46Q-RFP
2. Change media 4 hours post transfection
3. Add purified Myc-+36GFP-(G₄S)₂—C4-His₆ or Myc-+36GFP-His₆ 6 hours post transfection at a concentration of 2 uM.
4. Perform live cell imaging at 48 hours post transfection in green fluorescence, red fluorescence and phase contrast.
5. Fix a sample of each group for HA labeling.

6. Count the number of aggregates in each sample.

The results indicated that +36GFP-linker-C4 fusion protein reduces aggregation of HDex1-46QRFP (HTT46Q-RFP) by 30% at 48 hours relative to +36GFP alone at 2 micromolar. The number of aggregates formed by HTT46Q-RFP in the cells was determined by counting the number of aggregates seen when imaging for red fluorescence. Visual counting indicated 30% less aggregates in the +36GFP-linker-C4-treated cells, as compared to the +36GFP-treated cells. These results indicate that +36GFP efficiently delivers C4 to the cytoplasm of ST14A cells, where it is able to bind to and prevent aggregation of HTT.

The 30% decrease in aggregation observed in this Example is significant. In an experiment performed by Butler and Messer, where C4 was expressed via viral transfection as an intrabody with a PEST sequence that targets for proteosomal degradation, aggregation was reduced 51% for HDex1-25Q and 78% for HDex1-72Q at 48 hours post-transfection (Butler and Messer, PLosOne 2011, 6;e29199). In such an experiment however, the intrabody is likely continuously expressed over the time course and the PEST sequence may further decrease aggregation by targeting HTT for proteosomal degradation. The 30% decrease observed in this Example is notable with a singular administration of protein in which the C4 scFv is fused to a Surf+ Penetrating Polypeptide. The use of a human Surf+ Penetrating Polypeptide is described below.

### Sequences:

Myc-(+36)GFP-His6 (where the underlined
sequence depicts +36 GFP.
(SEQ ID NO: 665)
MEQKLISEEDLGSASKGERLFRGKVPILVELKGDVNGHKFSVRGKGKGDA
TRGKLTLKFICTTGKLPVPWPTLVTTLTYGVQCFSRYPKHMKRHDFFKSA
MPKGYVQERTISFKKDGKYKTRAEVKFEGRTLVNRIKLKGRDFKEKGNIL
GHKLRYNFNSHKVYITADKRKNGIKAKFKIRHNVKDGSVQLADHYQQNTP
IGRGPVLLPRNHYLSTRSKLSKDPKEKRDHMVLLEFVTAAGIKHGRDERY
KGHGHHHHHH
Myc-(+36)GFP-(G₄S)₂-C4_scFv-His6 (where the
underlined sequence is, from N- to C- terminus,
GFP, linker, C4 scFv).
(SEQ ID NO: 664)
MEQKLISEEDLGSASKGERLFRGKVPILVELKGDVNGHKFSVRGKGKGDA
TRGKLTLKFICTTGKLPVPWPTLVTTLTYGVQCFSRYPKHMKRHDFFKSA
MPKGYVQERTISFKKDGKYKTRAEVKFEGRTLVNRIKLKGRDFKEKGNIL
GHKLRYNFNSHKVYITADKRKNGIKAKFKIRHNVKDGSVQLADHYQQNTP
IGRGPVLLPRNHYLSTRSKLSKDPKEKRDHMVLLEFVTAAGIKHGRDERY
KGHGGGGSGGGGSQVQLQESGGGLVQPGGSLRLSCAASGFTFSSYSMSWV
RQAPGKGLEWVAVISYDGSNKYYADSVKGRFTISRDNSKNTLYLQMNSLR
AEDTAVYYCARDRYFDLWGRGTLVTVSSGGGGSGGGGSGGGGSQSALTQP
ASVSGSPGQSITISCTGTSSDIGAYNYVSWYQQYPGKAPKLLIYDVSNRP
SGISNRFSGSKSGDTASLTISGLQAEDEADYYCSSFANSGPLFGGGTKVT
VLGGHGHHHHHH

### Example 11

### Fusion Protein Comprising a Domain of FGF10 Fused to an AAM Moiety

In some embodiments, the Surf+ Penetrating Polypeptide is a domain of FGF10 having surface positive charge, an overall net positive charge, and a charge/molecular weight ratio greater than that of full length, unprocessed, naturally occurring FGF10. An exemplary AAM moiety which can be fused to the Surf+ Penetrating Polypeptide is an scFv. In such a fusion protein, the FGF10 portion may be N- or C-terminal to the AAM moiety.

The fusion proteins optionally include a linker that interconnects the FGF10 portion to the AAM moiety. Suitable linkers include a glycine/serine rich linker. When present, the linker may also include a serum-stable proteolytic cleavage site, such as a site cleavable by cathepsin class proteases. Cleavable linkers permit the separation of the AAM moiety from the FGF10 portion following internalization.

The following exemplary fusion protein is generated:

Myc-FGF10 portion-GS₁₀-AAM-His₆

Where, for example:

FGF10 portion is a domain of full length, naturally occurring human FGF10;

AAM is the AMM moiety and can be an scFv;

(GS)₁₀ is the linker amino acid sequence “GGGGSGGGGS”;

His₆ is the tag “HHHHHH”; and

Myc is the tag “EQKLISEEDL”.

The fusion protein is internalized by cells and binds (via the AAM moiety) to the target of interest. The fusion protein is studied in vitro and/or in vivo, such as using assays described herein.

An exemplary fusion protein is a fusion protein made by fusing a domain of FGF10 to a scFv specific for huntingtin protein. The fusion protein is tagged on the N-terminus with a Myc tag and on the C-terminus with a Hisx6 tag. A control lacking the AAM moiety is also made. The complexes can be represented as:
Myc-FGF10-His₆
Myc-FGF10-GS₁₀-C4-His₆
   where “FGF10” denotes the domain of FGF10, “C4” denotes the particular AAM moiety used in this example, as described above; and GS₁₀ denotes the linker (also known as (G₄S)₂ used to link the FGF10 portion to the AAM moiety.

In this particular example, the FGF10 portion has the amino acid sequence set forth in SEQ ID NO: 666.

The AAM moiety in this example is an scFv specific for huntingtin protein. This scFv, denoted “C4”, targets the first 17 amino acids of huntingtin protein and has been demonstrated to delay the aggregation phenotype when the gene is delivered in adeno-associated viral vectors (AAV2/1) in mice (J Neuopathol Exp Neurol. 2010. 69(10):1078-1085).

Experiments are conducted to demonstrate that the complex Myc-FGF10-GS₁₀-C4-His₆ can be effectively delivered into cells and disrupt aggregation of mHTT. The experimental procedure is as outlined above.

### Example 12

### Fusion Protein Comprising a Variant Domain of FGF10 Fused to an AAM Moiety

A fusion protein is made by fusing a variant domain of FGF10 having one or more amino acid additions, deletions, or substitutions relative to the naturally occurring domain, to an AAM moiety. The complex is tagged on the N-terminus with a Myc tag and on the C-terminus with a Hisx6 tag. A control lacking the AAM moiety is also made. The complexes can be represented as:
Myc-FGF10(mut4)-His₆
Myc-FGF10(mut4)-GS₁₀-C4-His₆
   where “FGF10(mut4)” denotes the variant domain of FGF10, “C4” denotes the particular AAM moiety used in this example; and GS₁₀ denotes the linker used to link the variant FGF10 portion to the AAM moiety.

In this particular example, the variant FGF10 portion has the amino acid sequence set forth in SEQ ID NO: 667. This variant FGF10 portion has been modified to minimize mitogenic effects and includes the following mutations: R78A/T114R/E158A/K195A. See e.g., Yeh et al. PNAS (2003) 100:2266-71; Ibrahimi et al. Mol Cell Biol. (2005) 25:671-84; and Wang et al. Cytokine (2010) 49:338-43. The amino acid sequence for the FGF10(mut4)-scFv fusion protein (Myc-FGF10(mut4)-GS₁₀-C4-His₆) is set forth in SEQ ID NO: 668. The amino acid sequence of the control complex (Myc-FGF10(mut4)-His₆) is set forth in SEQ ID NO: 669.

The AAM moiety in this example is an scFv specific for huntingtin protein. This scFv, denoted “C4”, targets the first 17 amino acids of huntingtin protein and has been demonstrated to delay the aggregation phenotype when the gene is delivered in adeno-associated viral vectors (AAV2/1) in mice (J Neuopathol Exp Neurol. 2010. 69(10):1078-1085).

Experiments are conducted to demonstrate that the complex Myc-FGF10(mut4)-GS₁₀-C4-His₆ can be effectively delivered into cells and disrupt aggregation of mHTT. Experiments for evaluating activity of the fusion protein are as outlined above.

Myc-FGF10(mut4)-His6
(SEQ ID NO: 669)
MEQKLISEEDLGSGRHVRSYNHLQGDVAWRKLFSFTKYFLKIEKNGKVSG
TKKENCPYSILEIRSVEIGVVAVKAINSNYYLAMNKKGKLYGSKEFNNDC
KLKERIEANGYNTYASFNWQHNGRQMYVALNGKGAPRRGQKTRRANTSAH
FLPMVVHSGHGHHHHHH
Myc-FGF10(mut4)-GS10-C4_scFv-His6
(SEQ ID NO: 668)
MEQKLISEEDLGSGRHVRSYNHLQGDVAWRKLFSFTKYFLKIEKNGKVSG
TKKENCPYSILEIRSVEIGVVAVKAINSNYYLAMNKKGKLYGSKEFNNDC
KLKERIEANGYNTYASFNWQHNGRQMYVALNGKGAPRRGQKTRRANTSAH
FLPMVVHSGHGGGGSGGGGSQVQLQESGGGLVQPGGSLRLSCAASGFTFS
SYSMSWVRQAPGKGLEWVAVISYDGSNKYYADSVKGRFTISRDNSKNTLY
LQMNSLRAEDTAVYYCARDRYFDLWGRGTLVTVSSGGGGSGGGGSGGGGS
QSALTQPASVSGSPGQSITISCTGTSSDIGAYNYVSWYQQYPGKAPKLLI
YDVSNRPSGISNRFSGSKSGDTASLTISGLQAEDEADYYCSSFANSGPLF
GGGTKVTVLGGHGHHHHHH

Sequence Listing Information

The following sequence information is intended to provide a detailed description for the amino acid sequences referenced in FIGS. 1 and 2 by GenBank accession number and/or PDB identifier. As such, all such sequence information should be considered part of the detailed description of the invention and provides additional description for Surf+ Penetrating Polypeptides, as well as polypeptides suitable for use as a portion of a complex comprising a Surf+ Penetrating Polypeptide.

The disclosure contemplates complexes comprising an amino acid sequence selected from amongst any of the amino acid sequences provided in this sequence listing, as well as functional fragments thereof (e.g., domains thereof having surface positive charge, a mass of at least 4 kDa, a charge/molecular weight ratio of at least 0.75). Such polypeptides are suitable for use in complexes of the disclosure. Moreover, in certain embodiments, complexes of the disclosure comprise an amino acid sequence at least 85%, 90%, 95%, 96%, 97%, 98%, or 99% identical to any of the foregoing.

Section 1 of Sequence Listing: Amino acid sequence information for full length sequences referenced by GenBank accession number in FIGS. 1 and 2.

### Amino Acid Sequence Information Disclosed in Genbank for Naturally Occurring Proteins Identified by Genbank Accession Number in FIG. 1

NP_001184033.1 histone-lysine N-methyltransferase MLL isoform 1 precursor
(SEQ ID NO: 1)
MAHSCRWRFPARPGTTGGGGGGGRRGLGGAPRQRVPALLLPPGPPVGGGGPGAPPSPPAVA
AAAAAAGSSGAGVPGGAAAASAASSSSASSSSSSSSSASSGPALLRVGPGFDAALQVSAAIGT
NLRRFRAVFGESGGGGGSGEDEQFLGFGSDEEVRVRSPTRSPSVKTSPRKPRGRPRSGSDRNS
AILSDPSVFSPLNKSETKSGDKIKKKDSKSIEKKRGRPPTFPGVKIKITHGKDISELPKGNKEDS
LKKIKRTPSATFQQATKIKKLRAGKLSPLKSKFKTGKLQIGRKGVQIVRRRGRPPSTERIKTPS
GLLINSELEKPQKVRKDKEGTPPLTKEDKTVVRQSPRRIKPVRIIPSSKRTDATIAKQLLQRAK
KGAQKKIEKEAAQLQGRKVKTQVKNIRQFIMPVVSAISSRIIKTPRRFIEDEDYDPPIKIARLES
TPNSRFSAPSCGSSEKSSAASQHSSQMSSDSSRSSSPSVDTSTDSQASEEIQVLPEERSDTPEVH
PPLPISQSPENESNDRRSRRYSVSERSFGSRTTKKLSTLQSAPQQQTSSSPPPPLLTPPPPLQPAS
SISDHTPWLMPPTIPLASPFLPASTAPMQGKRKSILREPTFRWTSLKHSRSEPQYFSSAKYAKE
GLIRKPIFDNFRPPPLTPEDVGFASGFSASGTAASARLFSPLHSGTRFDMHKRSPLLRAPRFTPS
EAHSRIFESVTLPSNRTSAGTSSSGVSNRKRKRKVFSPIRSEPRSPSHSMRTRSGRLSSSELSPLT
PPSSVSSSLSISVSPLATSALNPTFTFPSHSLTQSGESAEKNQRPRKQTSAPAEPFSSSSPTPLFP
WFTPGSQTERGRNKDKAPEELSKDRDADKSVEKDKSRERDREREKENKRESRKEKRKKGSE
IQSSSALYPVGRVSKEKVVGEDVATSSSAKKATGRKKSSSHDSGTDITSVTLGDTTAVKTKIL
IKKGRGNLEKTNLDLGPTAPSLEKEKTLCLSTPSSSTVKHSTSSIGSMLAQADKLPMTDKRVA
SLLKKAKAQLCKIEKSKSLKQTDQPKAQGQESDSSETSVRGPRIKHVCRRAAVALGRKRAVF
PDDMPTLSALPWEEREKILSSMGNDDKSSIAGSEDAEPLAPPIKPIKPVTRNKAPQEPPVKKGR
RSRRCGQCPGCQVPEDCGVCTNCLDKPKFGGRNIKKQCCKMRKCQNLQWMPSKAYLQKQ
AKAVKKKEKKSKTSEKKDSKESSVVKNVVDSSQKPTPSAREDPAPKKSSSEPPPRKPVEEKS
EEGNVSAPGPESKQATTPASRKSSKQVSQPALVIPPQPPTTGPPRKEVPKTTPSEPKKKQPPPP
ESGPEQSKQKKVAPRPSIPVKQKPKEKEKPPPVNKQENAGTLNILSTLSNGNSSKQKIPADGV
HRIRVDFKEDCEAENVWEMGGLGILTSVPITPRVVCFLCASSGHVEFVYCQVCCEPFHKFCLE
ENERPLEDQLENWCCRRCKFCHVCGRQHQATKQLLECNKCRNSYHPECLGPNYPTKPTKKK
KVWICTKCVRCKSCGSTTPGKGWDAQWSHDFSLCHDCAKLFAKGNFCPLCDKCYDDDDYE
SKMMQCGKCDRWVHSKCENLSGTEDEMYEILSNLPESVAYTCVNCTERHPAEWRLALEKE
LQISLKQVLTALLNSRTTSHLLRYRQAAKPPDLNPETEESIPSRSSPEGPDPPVLTEVSKQDDQ
QPLDLEGVKRKMDQGNYTSVLEFSDDIVKIIQAAINSDGGQPEIKKANSMVKSFFIRQMERVF
PWFSVKKSRFWEPNKVSSNSGMLPNAVLPPSLDHNYAQWQEREENSHTEQPPLMKKIIPAPK
PKGPGEPDSPTPLHPPTPPILSTDRSREDSPELNPPPGIEDNRQCALCLTYGDDSANDAGRLLYI
GQNEWTHVNCALWSAEVFEDDDGSLKNVHMAVIRGKQLRCEFCQKPGATVGCCLTSCTSN
YHFMCSRAKNCVFLDDKKVYCQRHRDLIKGEVVPENGFEVFRRVFVDFEGISLRRKFLNGLE
PENIHMMIGSMTIDCLGILNDLSDCEDKLFPIGYQCSRVYWSTTDARKRCVYTCKIVECRPPV
VEPDINSTVEHDENRTIAHSPTSFTESSSKESQNTAEIISPPSPDRPPHSQTSGSCYYHVISKVPRI
RTPSYSPTQRSPGCRPLPSAGSPTPTTHEIVTVGDPLLSSGLRSIGSRRHSTSSLSPQRSKLRIMS
PMRTGNTYSRNNVSSVSTTGTATDLESSAKVVDHVLGPLNSSTSLGQNTSTSSNLQRTVVTV
GNKNSHLDGSSSSEMKQSSASDLVSKSSSLKGEKTKVLSSKSSEGSAHNVAYPGIPKLAPQV
HNTTSRELNVSKIGSFAEPSSVSFSSKEALSFPHLHLRGQRNDRDQHTDSTQSANSSPDEDTEV
KTLKLSGMSNRSSIINEHMGSSSRDRRQKGKKSCKETFKEKHSSKSFLEPGQVTTGEEGNLKP
EFMDEVLTPEYMGQRPCNNVSSDKIGDKGLSMPGVPKAPPMQVEGSAKELQAPRKRTVKVT
LTPLKMENESQSKNALKESSPASPLQIESTSPTEPISASENPGDGPVAQPSPNNTSCQDSQSNN
YQNLPVQDRNLMLPDGPKPQEDGSFKRRYPRRSARARSNMFFGLTPLYGVRSYGEEDIPFYS
SSTGKKRGKRSAEGQVDGADDLSTSDEDDLYYYNFTRTVISSGGEERLASHNLFREEEQCDL
PKISQLDGVDDGTESDTSVTATTRKSSQIPKRNGKENGTENLKIDRPEDAGEKEHVTKSSVGH
KNEPKMDNCHSVSRVKTQGQDSLEAQLSSLESSRRVHTSTPSDKNLLDTYNTELLKSDSDNN
NSDDCGNILPSDIMDFVLKNTPSMQALGESPESSSSELLNLGEGLGLDSNREKDMGLFEVFSQ
QLPTTEPVDSSVSSSISAEEQFELPLELPSDLSVLTTRSPTVPSQNPSRLAVISDSGEKRVTITEK
SVASSESDPALLSPGVDPTPEGHMTPDHFIQGHMDADHISSPPCGSVEQGHGNNQDLTRNSST
PGLQVPVSPTVPIQNQKYVPNSTDSPGPSQISNAAVQTTPPHLKPATEKLIVVNQNMQPLYVL
QTLPNGVTQKIQLTSSVSSTPSVMETNTSVLGPMGGGLTLTTGLNPSLPTSQSLFPSASKGLLP
MSHHQHLHSFPAATQSSFPPNISNPPSGLLIGVQPPPDPQLLVSESSQRTDLSTTVATPSSGLKK
RPISRLQTRKNKKLAPSSTPSNIAPSDVVSNMTLINFTPSQLPNHPSLLDLGSLNTSSHRTVPNII
KRSKSSIMYFEPAPLLPQSVGGTAATAAGTSTISQDTSHLTSGSVSGLASSSSVLNVVSMQTTT
TPTSSASVPGHVTLTNPRLLGTPDIGSISNLLIKASQQSLGIQDQPVALPPSSGMFPQLGTSQTP
STAAITAASSICVLPSTQTTGITAASPSGEADEHYQLQHVNQLLASKTGIHSSQRDLDSASGPQ
VSNFTQTVDAPNSMGLEQNKALSSAVQASPTSPGGSPSSPSSGQRSASPSVPGPTKPKPKTKR
FQLPLDKGNGKKHKVSHLRTSSSEAHIPDQETTSLTSGTGTPGAEAEQQDTASVEQSSQKEC
GQPAGQVAVLPEVQVTQNPANEQESAEPKTVEEEESNFSSPLMLWLQQEQKRKESITEKKPK
KGLVFEISSDDGFQICAESIEDAWKSLTDKVQEARSNARLKQLSFAGVNGLRMLGILHDAVV
FLIEQLSGAKHCRNYKFRFHKPEEANEPPLNPHGSARAEVHLRKSAFDMFNFLASKHRQPPE
YNPNDEEEEEVQLKSARRATSMDLPMPMRFRHLKKTSKEAVGVYRSPIHGRGLFCKRNIDA
GEMVIEYAGNVIRSIQTDKREKYYDSKGIGCYMFRIDDSEVVDATMHGNAARFINHSCEPNC
YSRVINIDGQKHIVIFAMRKIYRGEELTYDYKFPIEDASNKLPCNCGAKKCRKFLN
NP_002219.1 transcription factor AP-1
(SEQ ID NO: 2)
MTAKMETTFYDDALNASFLPSESGPYGYSNPKILKQSMTLNLADPVGSLKPHLRAKNSDLLT
SPDVGLLKLASPELERLIIQSSNGHITTTPTPTQFLCPKNVTDEQEGFAEGFVRALAELHSQNT
LPSVTSAAQPVNGAGMVAPAVASVAGGSGSGGFSASLHSEPPVYANLSNFNPGALSSGGGA
PSYGAAGLAFPAQPQQQQQPPHHLPQQMPVQHPRLQALKEEPQTVPEMPGETPPLSPIDMES
QERIKAERKRMRNRIAASKCRKRKLERIARLEEKVKTLKAQNSELASTANMLREQVAQLKQ
KVMNHVNSGCQLMLTQQLQTF
NP_006063.1 C-C motif chemokine 26 precursor
(SEQ ID NO: 3)
MMGLSLASAVLLASLLSLHLGTATRGSDISKTCCFQYSHKPLPWTWVRSYEFTSNSCSQR
AVIFTTKRGKKVCTHPRKKWVQKYISLLKTPKQL
NP_001936.1 proheparin-binding EGF-like growth factor precursor
(SEQ ID NO: 4)
MKLLPSVVLKLFLAAVLSALVTGESLERLRRGLAAGTSNPDPPTVSTDQLLPLGGGRDRKVR
DLQEADLDLLRVTLSSKPQALATPNKEEHGKRKKKGKGLGKKRDPCLRKYKDFCIHGECKY
VKELRAPSCICHPGYHGERCHGLSLPVENRLYTYDHTTILAVVAVVLSSVCLLVIVGLLMFR
YHRRGGYDVENEEKVKLGMTNSH
NP_003463.1 protein DEK isoform 1
(SEQ ID NO: 5)
MSASAPAAEGEGTPTQPASEKEPEMPGPREESEEEEDEDDEEEEEEEKEKSLIVEGKREKKKV
ERLTMQVSSLQREPFTIAQGKGQKLCEIERIHFFLSKKKTDELRNLHKLLYNRPGTVSSLKKN
VGQFSGFPFEKGSVQYKKKEEMLKKFRNAMLKSICEVLDLERSGVNSELVKRILNFLMHPKP
SGKPLPKSKKTCSKGSKKERNSSGMARKAKRTKCPEILSDESSSDEDEKKNKEESSDDEDKES
EEEPPKKTAKREKPKQKATSKSKKSVKSANVKKADSSTTKKNQNSSKKESESEDSSDDEPLI
KKLKKPPTDEELKETIKKLLASANLEEVTMKQICKKVYENYPTYDLTERKDFIKTTVKELIS
NP_000592.3 hepatocyte growth factor isoform 1 preproprotein
(SEQ ID NO: 6)
MWVTKLLPALLLQHVLLHLLLLPIAIPYAEGQRKRRNTIHEFKKSAKTTLIKIDPALKIK
TKKVNTADQCANRCTRNKGLPFTCKAFVFDKARKQCLWFPFNSMSSGVKKEFGHEFDLYE
NKDYIRNCIIGKGRSYKGTVSITKSGIKCQPWSSMIPHEHSFLPSSYRGKDLQENYCRNPRGEE
GGPWCFTSNPEVRYEVCDIPQCSEVECMTCNGESYRGLMDHTESGKICQRWDHQTPHRHKF
LPERYPDKGFDDNYCRNPDGQPRPWCYTLDPHTRWEYCAIKTCADNTMNDTDVPLETTECI
QGQGEGYRGTVNTIWNGIPCQRWDSQYPHEHDMTPENFKCKDLRENYCRNPDGSESPWCFT
TDPNIRVGYCSQIPNCDMSHGQDCYRGNGKNYMGNLSQTRSGLTCSMWDKNMEDLHRHIF
WEPDASKLNENYCRNPDDDAHGPWCYTGNPLIPWDYCPISRCEGDTTPTIVNLDHPVISCAK
TKQLRVVNGIPTRTNIGWMVSLRYRNKHICGGSLIKESWVLTARQCFPSRDLKDYEAWLGIH
DVHGRGDEKCKQVLNVSQLVYGPEGSDLVLMKLARPAVLDDFVSTIDLPNYGCTIPEKTSCS
VYGWGYTGLINYDGLLRVAHLYIMGNEKCSQHHRGKVTLNESEICAGAEKIGSGPCEGDYG
GPLVCEQHKMRMVLGVIVPGRGCAIPNRPGIFVRVAYYAKWIHKIILTYKVPQS
NP_001075020.1 beta-defensin 103 precursor
(SEQ ID NO: 7)
MRIHYLLFALLFLFLVPVPGHGGIINTLQKYYCRVRGGRCAVLSCLPKEEQIGKCSTRGR
KCCRRKK
NP_078884.2 Endonuclease VIII-like 1
(SEQ ID NO: 8)
MPEGPELHLASQFVNEACRALVFGGCVEKSSVSRNPEVPFESSAYRISASARGKELRLIL
SPLPGAQPQQEPLALVFRFGMSGSFQLVPREELPRHAHLRFYTAPPGPRLALCFVDIRRF
GRWDLGGKWQPGRGPCVLQEYQQFRENVLRNLADKAFDRPICEALLDQRFFNGIGNYLRAE
ILYRLKIPPFEKARSVLEALQQHRPSPELTLSQKIRTKLQNPDLLELCHSVPKEVVQLGGKGY
GSESGEEDFAAFRAWLRCYGMPGMSSLQDRHGRTIWFQGDPGPLAPKGRKSRKKKSKATQL
SPEDRVEDALPPSKAPSRTRRAKRDLPKRTATQRPEGTSLQQDPEAPTVPKKGRRKGRQAAS
GHCRPRKVKADIPSLEPEGTSAS
NP_061820.1 cytochrome c
(SEQ ID NO: 9)
MGDVEKGKKIFIMKCSQCHTVEKGGKHKTGPNLHGLFGRKTGQAPGYSYTAANKNKGIIW
GEDTLMEYLENPKKYIPGTKMIFVGIKKKEERADLIAYLKKATNE
NP_004456.1 fibroblast growth factor 10 precursor
(SEQ ID NO: 10)
MWKWILTHCASAFPHLPGCCCCCFLLLFLVSSVPVTCQALGQDMVSPEATNSSSSSFSSP
SSAGRHVRSYNHLQGDVRWRKLFSFTKYFLKIEKNGKVSGTKKENCPYSILEITSVEIGV
VAVKAINSNYYLAMNKKGKLYGSKEFNNDCKLKERIEENGYNTYASFNWQHNGRQMYVA
LNGKGAPRRGQKTRRKNTSAHFLPMVVHS
NP_002982.2 C-C motif chemokine 24 precursor
(SEQ ID NO: 11)
MAGLMTIVTSLLFLGVCAHHIIPTGSVVIPSPCCMFFVSKRIPENRVVSYQLSSRSTCLK
AGVIFTTKKGQQFCGDPKQEWVQRYMKNLDAKQKKASPRARAVAVKGPVQRYPGNQTTC
NP_003125.3 signal recognition particle 14 kDa protein
(SEQ ID NO: 12)
MVLLESEQFLTELTRLFQKCRTSGSVYITLKKYDGRTKPIPKKGTVEGFEPADNKCLLRA
TDGKKKISTVVSSKEVNKFQMAYSNLLRANMDGLKKRDKKNKTKKTKAAAAAAAAAPAA
AATAPTTAATTAATAAQ
NP_005219.2 epidermal growth factor receptor isoform a precursor
(SEQ ID NO: 13)
MRPSGTAGAALLALLAALCPASRALEEKKVCQGTSNKLTQLGTFEDHFLSLQRMFNNCEVV
LGNLEITYVQRNYDLSFLKTIQEVAGYVLIALNTVERIPLENLQIIRGNMYYENSYALAVLSN
YDANKTGLKELPMRNLQEILHGAVRFSNNPALCNVESIQWRDIVSSDFLSNMSMDFQNHLGS
CQKCDPSCPNGSCWGAGEENCQKLTKIICAQQCSGRCRGKSPSDCCHNQCAAGCTGPRESD
CLVCRKFRDEATCKDTCPPLMLYNPTTYQMDVNPEGKYSFGATCVKKCPRNYVVTDHGSC
VRACGADSYEMEEDGVRKCKKCEGPCRKVCNGIGIGEFKDSLSINATNIKHFKNCTSISGDLH
ILPVAFRGDSFTHTPPLDPQELDILKTVKEITGFLLIQAWPENRTDLHAFENLEIIRGRTKQHGQ
FSLAVVSLNITSLGLRSLKEISDGDVIISGNKNLCYANTINWKKLFGTSGQKTKIISNRGENSC
KATGQVCHALCSPEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYADAGHVCHLCH
PNCTYGCTGPGLEGCPTNGPKIPSIATGMVGALLLLLVVALGIGLFMRRRHIVRKRTLRRLLQ
ERELVEPLTPSGEAPNQALLRILKETEFKKIKVLGSGAFGTVYKGLWIPEGEKVKIPVAIKELR
EATSPKANKEILDEAYVMASVDNPHVCRLLGICLTSTVQLITQLMPFGCLLDYVREHKDNIGS
QYLLNWCVQIAKGMNYLEDRRLVHRDLAARNVLVKTPQHVKITDFGLAKLLGAEEKEYHA
EGGKVPIKWMALESILHRIYTHQSDVWSYGVTVWELMTFGSKPYDGIPASEISSILEKGERLP
QPPICTIDVYMIMVKCWMIDADSRPKFRELIIEFSKMARDPQRYLVIQGDERMHLPSPTDSNF
YRALMDEEDMDDVVDADEYLIPQQGFFSSPSTSRTPLLSSLSATSNNSTVACIDRNGLQSCPI
KEDSFLQRYSSDPTGALTEDSIDDTFLPVPEYINQSVPKRPAGSVQNPVYHNQPLNPAPSRDP
HYQDPHSTAVGNPEYLNTVQPTCVNSTFDSPAHWAQKGSHQISLDNPDYQQDFFPKEAKPN
GIFKGSTAENAEYLRVAPQSSEFIGA
NP_004878.2 C-X-C motif chemokine 14 precursor
(SEQ ID NO: 14)
MSLLPRRAPPVSMRLLAAALLLLLLALYTARVDGSKCKCSRKGPKIRYSDVKKLEMKPKYP
HCEEKMVIITTKSVSRYRGQEHCLHPKLQSTKRFIKWYNAWNEKRRVYEE
NP_004505.2 forkhead box protein K2
(SEQ ID NO: 15)
MAAAAAALSGAGTPPAGGGAGGGGAGGGGSPPGGWAVARLEGREFEYLMKKRSVTIGRNS
SQGSVDVSMGHSSFISRRHLEIFTPPGGGGHGGAAPELPPAQPRPDAGGDFYLRCLGKNGVF
VDGVFQRRGAPPLQLPRVCTFRFPSTNIKITFTALSSEKREKQEASESPVKAVQPHISPLTINIP
DTMAHLISPLPSPTGTISAANSCPSSPRGAGSSGYKVGRVMPSDLNLMADNSQPENEKEASG
GDSPKDDSKPPYSYAQLIVQAITMAPDKQLTLNGIYTHITKNYPYYRTADKGWQNSIRHNLS
LNRYFIKVPRSQEEPGKGSFWRIDPASESKLIEQAFRKRRPRGVPCFRTPLGPLSSRSAPASPN
HAGVLSAHSSGAQTPESLSREGSPAPLEPEPGAAQPKLAVIQEARFAQSAPGSPLSSQPVLITV
QRQLPQAIKPVTYTVATPVTTSTSQPPVVQTVHVVHQIPAVSVTSVAGLAPANTYTVSGQAV
VTPAAVLAPPKAEAQENGDHREVKVKVEPIPAIGHATLGTASRIIQTAQTTPVQTVTIVQQAP
LGQHQLPIKTVTQNGTHVASVPTAVHGQVNNAAASPLHMLATHASASASLPTKRHNGDQPE
QPELKRIKTEDGEGIVIALSVDTPPAAVREKGVQN
NP_060362.3 pre-mRNA-processing factor 40 homolog A
(SEQ ID NO: 16)
MCSGSGRRRSSLSPTMRPGTGAERGGLMMGHPGMHYAPMGMHPMGQRANMPPVPHGMM
PQMMPPMGGPPMGQMPGMMSSVMPGMMMSHMSQASMQPALPPGVNSMDVAAGTASGA
KSMWTEHKSPDGRTYYYNTETKQSTWEKPDDLKTPAEQLLSKCPWKEYKSDSGKPYYYNS
QTKESRWAKPKELEDLEGYQNTIVAGSLITKSNLHAMIKAEESSKQEECTTTSTAPVPTTEIPT
TMSTMAAAEAAAAVVAAAAAAAAAAAAANANASTSASNTVSGTVPVVPEPEVTSIVATVV
DNENTVTISTEEQAQLTSTPAIQDQSVEVSSNTGEETSKQETVADFTPKKEEEESQPAKKTYT
WNTKEEAKQAFKELLKEKRVPSNASWEQAMKMIINDPRYSALAKLSEKKQAFNAYKVQTE
KEEKEEARSKYKEAKESFQRFLENHEKMTSTTRYKKAEQMFGEMEVWNAISERDRLEIYED
VLFFLSKKEKEQAKQLRKRNWEALKNILDNMANVTYSTTWSEAQQYLMDNPTFAEDEELQ
NMDKEDALICFEEHIRALEKEEEEEKQKSLLRERRRQRKNRESFQIFLDELHEHGQLHSMSSW
MELYPTISSDIRFTNMLGQPGSTALDLFKFYVEDLKARYHDEKKIIKDILKDKGFVVEVNTTF
EDFVAIISSTKRSTTLDAGNIKLAFNSLLEKAEAREREREKEEARKMKRKESAFKSMLKQAAP
PIELDAVWEDIRERFVKEPAFEDITLESERKRIFKDFMHVLEHECQHHHSKNKKHSKKSKKH
HRKRSRSRSGSDSDDDDSHSKKKRQRSESRSASEHSSSAESERSYKKSKKHKKKSKKRRHKS
DSPESDAEREKDKKEKDRESEKDRTRQRSESKHKSPKKKTGKDSGNWDTSGSELSEGELEKR
RRTLLEQLDDDQ
NP_004166.1 small nuclear ribonucleoprotein Sm D3
(SEQ ID NO: 17)
MSIGVPIKVLHEAEGHIVTCETNTGEVYRGKLIEAEDNMNCQMSNITVTYRDGRVAQLEQVY
IRGSKIRFLILPDMLKNAPMLKSMKNKNQGSGAGRGKAAILKAQVAARGRGRGMGRGNIFQ
KRR
NP_000324.1 ataxin-7 isoform a
(SEQ ID NO: 18)
MSERAADDVRGEPRRAAAAAGGAAAAAARQQQQQQQQQQPPPPQPQRQQHPPPPPRRTRP
EDGGPGAASTSAAAMATVGERRPLPSPEVMLGQSWNLWVEASKLPGKDGTELDESFKEFGK
NREVMGLCREDMPIFGFCPAHDDFYLVVCNDCNQVVKPQAFQSHYERRHSSSSKPPLAVPPT
SVFSFFPSLSKSKGGSASGSNRSSSGGVLSASSSSSKLLKSPKEKLQLRGNTRPMHPIQQSRVP
HGRIMTPSVKVEKIHPKMDGTLLKSAVGPTCPATVSSLVKPGLNCPSIPKPTLPSPGQILNGKG
LPAPPTLEKKPEDNSNNRKFLNKRLSEREFDPDIHCGVIDLDTKKPCTRSLTCKTHSLTQRRA
VQGRRKRFDVLLAEHKNKTREKELIRHPDSQQPPQPLRDPHPAPPRTSQEPHQNPHGVIPSES
KPFVASKPKPHTPSLPRPPGCPAQQGGSAPIDPPPVHESPHPPLPATEPASRLSSEEGEGDDKEE
SVEKLDCHYSGHHPQPASFCTFGSRQIGRGYYVFDSRWNRLRCALNLMVEKHLNAQLWKKI
PPVPSTTSPISTRIPHRTNSVPTSQCGVSYLAAATVSTSPVLLSSTCISPNSKSVPAHGTTLNAQP
AASGAMDPVCSMQSRQVSSSSSSPSTPSGLSSVPSSPMSRKPQKLKSSKSLRPKESSGNSTNC
QNASSSTSGGSGKKRKNSSPLLVHSSSSSSSSSSSSHSMESFRKNCVAHSGPPYPSTVTSSHSIG
LNCVTNKANAVNVRHDQSGRGPPTGSPAESIKRMSVMVNSSDSTLSLGPFIHQSNELPVNSH
GSFSHSHTPLDKLIGKKRKCSPSSSSINNSSSKPTKVAKVPAVNNVHMKHTGTIPGAQGLMNS
SLLHQPKARP
NP_057250.1 E3 SUMO-protein ligase PIAS1
(SEQ ID NO: 19)
MADSAELKQMVMSLRVSELQVLLGYAGRNKHGRKHELLTKALHLLKAGCSPAVQMKIKEL
YRRRFPQKIMTPADLSIPNVHSSPMPATLSPSTIPQLTYDGHPASSPLLPVSLLGPKHELELPHL
TSALHPVHPDIKLQKLPFYDLLDELIKPTSLASDNSQRFRETCFAFALTPQQVQQISSSMDISGT
KCDFTVQVQLRFCLSETSCPQEDHFPPNLCVKVNTKPCSLPGYLPPTKNGVEPKRPSRPINITS
LVRLSTTVPNTIVVSWTAEIGRNYSMAVYLVKQLSSTVLLQRLRAKGIRNPDHSRALIKEKLT
ADPDSEIATTSLRVSLLCPLGKMRLTIPCRALTCSHLQCFDATLYIQMNEKKPTWVCPVCDK
KAPYEHLIIDGLFMEILKYCTDCDEIQFKEDGTWAPMRSKKEVQEVSASYNGVDGCLSSTLE
HQVASHHQSSNKNKKVEVIDLTIDSSSDEEEEEPSAKRTCPSLSPTSPLNNKGILSLPHQASPV
SRTPSLPAVDTSYINTSLIQDYRHPFHMTPMPYDLQGLDFFPFLSGDNQHYNTSLLAAAAAA
VSDDQDLLHSSRFFPYTSSQMFLDQLSAGGSTSLPTTNGSSSGSNSSLVSSNSLRESHSHTVTN
RSSTDTASIFGIIPDIISLD
NP_002610.1 platelet factor 4 precursor
(SEQ ID NO: 20)
MSSAAGFCASRPGLLFLGLLLLPLVVAFASAEAEEDGDLQCLCVKTTSQVRPRHITSLEV
IKAGPHCPTAQLIATLKNGRKICLDLQAPLYKKIIKKLLES
NP_001193858.1 advanced glycosylation end product-specific receptor
isoform 2 precursor
(SEQ ID NO: 21)
MAAGTAVGAWVLVLSLWGAVVGAQNITARIGEPLVLKCKGAPKKPPQRLEWKLNTGRTEA
WKVLSPQGGGPWDSVARVLPNGSLFLPAVGIQDEGIFRCQAMNRNGKETKSNYRVRVYQIP
GKPEIVDSASELTAGVPNKVVEESRRSRKRPCEQEVGTCVSEGSYPAGTLSWHLDGKPLVPN
EKGVSVKEQTRRHPETGLFTLQSELMVTPARGGDPRPTFSCSFSPGLPRHRALRTAPIQPRVW
EPVPLEEVQLVVEPEGGAVAPGGTVTLTCEVPAQPSPQIHWMKDGVPLPLPPSPVLILPEIGPQ
DQGTYSCVATHSSHGPQESRAVSISIIEPGEEGPTAGSVGGSGLGTLALALGILGGLGTAALLI
GVILWORRQRRGEERKAPENQEEEEERAELNOSEEPEAGESSTGGP
NP_006110.1 fibroblast growth factor 8 isoform B precursor
(SEQ ID NO: 22)
MGSPRSALSCLLLHLLVLCLQAQVTVQSSPNFTQHVREQSLVTDQLSRRLIRTYQLYSRT
SGKHVQVLANKRINAMAEDGDPFAKLIVETDTGSRVRVRGAETGLYICMNKKGKLIAKSN
GKGKDCVFTEIVLENNYTALQNAKYEGWYMAFTRKGRPRKGSKTRQHQREVHFMKRLPRG
HHTTEQSLRFEFLNYPPFTRSLRGSQRTWAPEPR
NP_004590.2 sterol regulatory element-binding protein 2
(SEQ ID NO: 23)
MDDSGELGGLETMETLTELGDELTLGDIDEMLQFVSNQVGEFPDLFSEQLCSSFPGSGGSGSS
SGSSGSSSSSSNGRGSSSGAVDPSVQRSFTQVTLPSFSPSAASPQAPTLQVKVSPTSVPTTPRAT
PILQPRPQPQPQPQTQLQQQTVMITPTFSTTPQTRIIQQPLIYQNAATSFQVLQPQVQSLVTSSQ
VQPVTIQQQVQTVQAQRVLTQTANGTLQTLAPATVQTVAAPQVQQVPVLVQPQIIKTDSLVL
TTLKTDGSPVMAAVQNPALTALTTPIQTAALQVPTLVGSSGTILTTMPVMMGQEKVPIKQVP
GGVKQLEPPKEGERRTTHNIIEKRYRSSINDKIIELKDLVMGTDAKMHKSGVLRKAIDYIKYL
QQVNHKLRQENMVLKLANQKNKLLKGIDLGSLVDNEVDLKIEDFNQNVLLMSPPASDSGSQ
AGFSPYSIDSEPGSPLLDDAKVKDEPDSPPVALGMVDRSRILLCVLTFLCLSFNPLTSLLQWG
GAHDSDQHPHSGSGRSVLSFESGSGGWFDWMMPTLLLWLVNGVIVLSVFVKLLVHGEPVIR
PHSRSSVTFWRHRKQADLDLARGDFAAAAGNLQTCLAVLGRALPTSRLDLACSLSWNVIRY
SLQKLRLVRWLLKKVFQCRRATPATEAGFEDEAKTSARDAALAYHRLHQLHITGKLPAGSA
CSDVHMALCAVNLAECAEEKIPPSTLVEIHLTAAMGLKTRCGGKLGFLASYFLSRAQSLCGP
EHSAVPDSLRWLCHPLGQKFFMERSWSVKSAAKESLYCAQRNPADPIAQVHQAFCKNLLER
AIESLVKPQAKKKAGDQEEESCEFSSALEYLKLLHSFVDSVGVMSPPLSRSSVLKSALGPDIIC
RWWTSAITVAISWLQGDDAAVRSHFTKVERIPKALEVTESPLVKAIFHACRAMHASLPGKAD
GQQSSFCHCERASGHLWSSLNVSGATSDPALNHVVQLLTCDLLLSLRTALWQKQASASQAV
GETYHASGAELAGFQRDLGSLRRLAHSFRPAYRKVFLHEATVRLMAGASPTRTHQLLEHSL
RRRTTQSTKHGEVDAWPGQRERATAILLACRHLPLSFLSSPGQRAVLLAEAARTLEKVGDRR
SCNDCQQMIVKLGGGTAIAAS
NP_078867.2 charged multivesicular body protein 6
(SEQ ID NO: 24)
MGNLFGRKKQSRVTEQDKAILQLKQQRDKLRQYQKRIAQQLERERALARQLLRDGRKERA
KLLLKKKRYQEQLLDRTENQISSLEAMVQSIEFTQIEMKVMEGLQFGNECLNKMHQVMSIEE
VERILDETQEAVEYQRQIDELLAGSFTQEDEDAILEELSAITQEQIELPEVPSEPLPE
KIPENVPVKARPRQAELVAAS
NP_001029058.1 stromal cell-derived factor 1 isoform gamma
(SEQ ID NO: 25)
MNAKVVVVLVLVLTALCLSDGKPVSLSYRCPCRFFESHVARANVKHLKILNTPNCALQIVAR
LKNNNRQVCIDPKLKWIQEYLEKALNKGRREEKVGKKEKIGKKKRQKKRKAAQKRKN
NP_001420.2 histone acetyltransferase p300
(SEQ ID NO: 26)
MAENVVEPGPPSAKRPKLSSPALSASASDGTDFGSLFDLEHDLPDELINSTELGLTNGGD
INQLQTSLGMVQDAASKHKQLSELLRSGSSPNLNMGVGGPGQVMASQAQQSSPGLGLINSM
VKSPMTQAGLTSPNMGMGTSGPNQGPTQSTGMMNSPVNQPAMGMNTGMNAGMNPGMLA
AGNGQGIMPNQVMNGSIGAGRGRQNMQYPNPGMGSAGNLLTEPLQQGSPQMGGQTGLRG
PQPLKMGMMNNPNPYGSPYTQNPGQQIGASGLGLQIQTKTVLSNNLSPFAMDKKAVPGGG
MPNMGQQPAPQVQQPGLVTPVAQGMGSGAHTADPEKRKLIQQQLVLLLHAHKCQRREQA
NGEVRQCNLPHCRTMKNVLNHMTHCQSGKSCQVAHCASSRQIISHWKNCTRHDCPVCLPL
KNAGDKRNQQPILTGAPVGLGNPSSLGVGQQSAPNLSTVSQIDPSSIERAYAALGLPYQVNQ
MPTQPQVQAKNQQNQQPGQSPQGMRPMSNMSASPMGVNGGVGVQTPSLLSDSMLHSAINS
QNPMMSENASVPSLGPMPTAAQPSTTGIRKQWHEDITQDLRNHLVHKLVQAIFPTPDPAALK
DRRMENLVAYARKVEGDMYESANNRAEYYHLLAEKIYKIQKELEEKRRTRLOKQNMLPNA
AGMVPVSMNPGPNMGQPQPGMTSNGPLPDPSM1RGSVPNQMMPRITPQSGLNQFGQMSMA
QPPIVPRQTPPLQHHGQLAQPGALNPPMGYGPRMQQPSNQGQFLPQTQFPSQGMNVTNIPLA
PSSGQAPVSQAQMSSSSCPVNSPIMPPGSQGSHIHCPQLPQPALHQNSPSPVPSRTPTPHHTPPS
IGAQQPPATTIPAPVPTPPAMPPGPQSQALHPPPRQTPTPPTTQLPQQVQPSLPAAPSADQPQQ
QPRSQQSTAASVPTPTAPLLPPQPATPLSQPAVSIEGQVSNPPSTSSTEVNSQAIAEKQPSQEVK
MEAKMEVDQPEPADTQPEDISESKVEDCKMESTETEERSTELKTEIKEEEDQPSTSATQSSPA
PGQSKKKIFKPEELRQALMPTLEALYRQDPESLPFRQPVDPQLLGIPDYFDIVKSPMDLSTIKR
KLDTGQYQEPWQYVDDIWLMFNNAWLYNRKTSRVYKYCSKLSEVFEQEIDPVMQSLGYCC
GRKLEFSPQTLCCYGKQLCTIPRDATYYSYQNRYHFCEKCFNEIQGESVSLGDDPSQPQTTIN
KEQFSKRKNDTLDPELFVECTECGRKMHQICVLHHEIIWPAGFVCDGCLKKSARTRKFNKFS
AKRLPSTRLGTFLENRVNDFLRRQNHPESGEVTVRVVHASDKTVEVKPGMKARFVDSGEMA
ESFPYRTKALFAFEEIDGVDLCFFGMHVQEYGSDCPPPNQRRVYISYLDSVHFFRPKCLRTAV
YHEILIGYLEYVKKLGYTTGHIWACPPSEGDDYIFHCHPPDQKIPKPKRLQEWYKKMLDKAV
SERIVHDYKDIFKQATEDRLTSAKELPYFEGDFWPNVLEESIKELEQEEEERKREENTSNESTD
VTKGDSKNAKKKNNKKTSKNKSSLSRGNKKKPGMPNVSNDLSQKLYATMEKHKEVFFVIR
LIAGPAANSLPPIVDPDPLIPCDLMDGRDAFLTLARDKHLEFSSLRRAQWSTMCMLVELHTQS
QDRFVYTCNECKHHVETRWHCTVCEDYDLCITCYNTKNHDHKMEKLGLGLDDESNNQQA
AATQSPGDSRRLSIQRCIQSLVHACQCRNANCSLPSCQKMKRVVQHTKGCKRKTNGGCPICK
QLIALCCYHAKHCQENKCPVPFCLNIKQKLRQQQLQHRLQQAQMLRRRMASMQRTGVVGQ
QQGLPSPTPATPTTPTGQQPTTPQTPQPTSQPQPTPPNSMPPYLPRTQAAGPVSQGKAAGQVT
PPTPPQTAQPPLPGPPPAAVEMAMQIQRAAETQRQMAHVQIFQRPIQHQMPPMTPMAPMGM
NPPPMTRGPSGHLEPGMGPTGMQQQPPWSQGGLPQPQQLQSGMPRPAMMSVAQHGQPLN
MAPQPGLGQVGISPLKPGTVSQQALQNLLRTLRSPSSPLQQQQVLSILHANPQLLAAFIKQRA
AKYANSNPQPIPGQPGMPQGQPGLQPPTMPGQQGVHSNPAMQNMNPMQAGVQRAGLPQQ
QPQQQLQPPMQQMSPQAQQMNMNHNTMPSQFRDILRRQQMMQQQQQQGAGPGIGPGMA
NHNQFQQPQGVGYPPQQQQRMQHHMQQMQQGNMGQIGQLPQALGAEAGASLQAYQQRL
LQQQMGSPVQPNPMSPQQHMLPNQAQSPHLQGQQIPNSLSNQVRSPQPVPSPRPQSQPPHSSP
SPRMQPQPSPHHVSPQTSSPHPGLVAAQANPMEQGHFASPDQNSMLSQLASNPGMANLHGA
SATDLGLSTDNSDLNSNLSQSTLDIH
NP_004587.1 U1 small nuclear ribonucleoprotein A
(SEQ ID NO: 27)
MAVPETRPNHTIYINNLNEKIKKDELKKSLYAIFSQFGQILDILVSRSLKMRGQAFVIFK
EVSSATNALRSMQGFPFYDKPMRIQYAKTDSDIIAKMKGTFVERDRKREKRKPKSQETPATK
KAVQGGGATPVVGAVQGPVPGMPPMTQAPRIMHMMPGQPPYMPPPGMIPPPGLAPGQIPPG
AMPPQQLMPGQMPPAQPLSENPPNHILFLTNLPEETNELMLSMLFNQFPGFKEVRLVPGRHDI
AFVEFDNEVQAGAARDALQGFKITQNNAMKISFAKK
NP_001191890.1 pre-B-cell leukemia transcription factor I isoform 2
(SEQ ID NO: 28)
MDEQPRLMHSHAGVGMAGHPGLSQHLQDGAGGTEGEGGRKQDIGDILQQIMTITDQSLDEA
QARKHALNCHRMKPALFNVLCE1KEKTVLSIRGAQEEEPTDPQLMRLDNMLLAEGVAGPEK
GGGSAAAAAAAAASGGAGSDNSVEHSDYRAKLSQIRQIYHTELEKYEQACNEFTTHVMNLL
REQSRTRPISPKEIERMVSIIHRKFSSIQMQLKQSTCEAVMILRSRFLDARRKRRNFNKQATEIL
NEYFYSHLSNPYPSEEAKEELAKKCGITVSQVSNWFGNKRIRYKKNIGKFQEEANIYAAKTA
VTATNVSAHGSQANSPSTPNSAGGYPSPCYQPDRRIQ
NP_006158.2 homeobox protein Nkx-3.1
(SEQ ID NO: 29)
MLRVPEPRPGEAKAEGAAPPTPSKPLTSFLIQDILRDGAQRQGGRTSSQRQRDPEPEPEP
EPEGGRSRAGAQNDQLSTGPRAAPEEAETLAETEPERHLGSYLLDSENTSGALPRLPQTP
KQPQKRSRAAFSHTQVIELERKFSHQKYLSAPERAHLAKNLKLTETQVKIWFQNRRYKTKRK
QLSSELGDLEKHSSLPALKEEAFSRASLVSVYNSYPYYPYLYCVGSWSPAFW
NP_689952.1 homeobox protein Hox-A9
(SEQ ID NO: 30)
MATTGALGNYYVDSFLLGADAADELSVGRYAPGTLGQPPRQAATLAEHPDFSPCSFQSKAT
VFGASWNPVHAAGANAVPAAVYHHHHHHPYVHPQAPVAAAAPDGRYMRSWLEPTPGALS
FAGLPSSRPYGIKPEPLSARRGDCPTLDTHTLSLTDYACGSPPVDREKQPSEGAFSENNAENES
GGDKPPIDPNNPAANWLHARSTRKKRCPYTKHQTLELEKEFLFNMYLTRDRRYEVARLLNL
TERQVKIWFQNRRMKMKKINKDRAKDE
NP_001124317.1 B-cell lymphoma 6 protein isoform 1
(SEQ ID NO: 31)
MASPADSCIQFTRHASDVLLNLNRLRSRDILTDVVIVVSREQFRAHKTVLMACSGLFYSIFTD
QLKCNLSVINLDPEINPEGFCILLDFMYTSRLNLREGNIMAVMATAMYLQMEHVVDTCRKFI
KASEAEMVSAIKPPREEFLNSRMLMPQDIMAYRGREVVENNLPLRSAPGCESRAFAPSLYSG
LSTPPASYSMYSHLPVSSLLFSDEEFRDVRMPVANPFPKERALPCDSARPVPGEYSRPTLEVSP
NVCHSNIYSPKETIPEEARSDMHYSVAEGLKPAAPSARNAPYFPCDKASKEEERPSSEDEIAL
HFEPPNAPLNRKGLVSPQSPQKSDCQPNSPTESCSSKNACILQASGSPPAKSPTDPKACNWKK
YKFIVLNSLNQNAKPEGPEQAELGRLSPRAYTAPPACQPPMEPENLDLQSPTKLSASGEDSTIP
QASRLNNIVNRSMTGSPRSSSESHSPLYMHPPKCTSCGSQSPQHAEMCLHTAGPTFPEEMGET
QSEYSDSSCENGAFFCNECDCRFSEEASLKRHTLQTHSDKPYKCDRCQASFRYKGNLASHKT
VHTGEKPYRCNICGAQFNRPANLKTHTRIHSGEKPYKCETCGARFVQVAHLRAHVLIHTGEK
PYPCEICGTRFRHLQTLKSHLRIHTGEKPYHCEKCNLHFRHKSQLRLHLRQKHGAITNTKVQ
YRVSATDLPPELPKAC
NP_001964.2 ETS domain-containing protein Elk-4 isoform a
(SEQ ID NO: 32)
MDSAITLWQFLLQLLQKPQNKHMICWTSNDGQFKLLQAEEVARLWGIRKNKPNMNYDKLS
RALRYYYVKNIIKKVNGQKFVYKFVSYPEILNMDPMTVGRIEGDCESLNFSEVSSSSKDVEN
GGKDKPPQPGAKTSSRNDYIHSGLYSSFTLNSLNSSNVKLFKLIKTENPAEKLAEKKSPQEPTP
SVIKFVTTPSKKPPVEPVAATISIGPSISPSSEETIQALETLVSPKLPSLEAPTSASNVMTAFATTP
PISSIPPLQEPPRTPSPPLSSHPDIDTDIDSVASQPMELPENLSLEPKDQDSVLLEKDKVNNSSRS
KKPKGLELAPTLVITSSDPSPLGILSPSLPTASLTPAFFSQTPIILTPSPLLSSIHFWSTLSPVAPLS
PARLQGANTLFQFPSVLNSHGPFTLSGLDGPSTPGPFSPDLQKT
NP_005020.1 pituitary homeobox 3
(SEQ ID NO: 33)
MEFGLLSEAEARSPALSLSDAGTPHPQLPEHGCKGQEHSDSEKASASLPGGSPEDGSLKK
KQRRQRTHFTSQQLQELEATFQRNRYPDMSTREEIAVWTNLTEARVRVWFKNRRAKWRKR
ERSQQAELCKGSFAAPLGGLVPPYEEVYPGYSYGNWPPKALAPPLAAKTFPFAFNSVNVGPL
ASQPVFSPPSSIAASMVPSAAAAPGTVPGPGALQGLGGGPPGLAPAAVSSGAVSCPYASAAA
AAAAAASSPYVYRDPCNSSLASLRLKAKQHASFSYPAVHGPPPAANLSPCQYAVERPV
NP_006424.2 granulysin isoform NKG5
(SEQ ID NO: 34)
MATWALLLLAAMLLGNPGLVFSRLSPEYYDLARAHLRDEEKSCPCLAQEGPQGDLLTKTQE
LGRDYRTCLTIVQKLKKMVDKPTQRSVSNAATRVCRTGRSRWRDVCRNFMRRYQSRVTQG
LVAGETAQQICEDLRLCIPSTGPL
NP_002087.2 general transcription factor IIF subunit 1
(SEQ ID NO: 35)
MAALGPSSQNVTEYVVRVPKNTTKKYNIMAFNAADKVNFATWNQARLERDLSNKKIYQEE
EMPESGAGSEFNRKLREEARRKKYGIVLKEFRPEDQPWLLRVNGKSGRKFKGIKKGGVTEN
TSYYIFTQCPDGAFEAFPVHNWYNFTPLARHRTLTAEEAEEEWERRNKVLNHFSIMQQRRLK
DQDQDEDEEEKEKRGRRKASELRIHDLEDDLEMSSDASDASGEEGGRVPKAKKKAPLAKGG
RKKKKKKGSDDEAFEDSDDGDFEGQEVDYMSDGSSSSQEEPESKAKAPQQEEGPKGVDEQS
DSSEESEEEKPPEEDKEEEEEKKAPTPQEKKRRKDSSEESDSSEESDIDSEASSALFMAKKKTP
PKRERKPSGGSSRGNSRPGTPSAEGGSTSSTLRAAASKLEQGKRVSEMPAAKRLRLDTGPQS
LSGKSTPQPPSGKTTPNSGDVQVTEDAVRRYLTRKPMTTKDLLKKFQTKKTGLSSEQTVNVL
AQILKRLNPERKMINDKMHFSLKE
NP_003855.1 histone deacetylase complex subunit SAP30
(SEQ ID NO: 36)
MNGFTPDEMSRGGDAAAAVAAVVAAAAAAASAGNGTGAGTGAEVPGAGAVSAAGPPGA
AGPGPGQLCCLREDGERCGRAAGNASFSKRIQKSISQKKVKIELDKSARHLYICDYHKNLIQS
VRNRRKRKGSDDDGGDSPVQDIDTPEVDLYQLQVNTLRRYKRHFKLPTRPGLNKAQLVEIV
GCHFRSIPVNEKDTLTYFIYSVKNDKNKSDLKVDSGVH
NP_057371.2 heterochromatin protein 1-binding protein 3
(SEQ ID NO: 37)
MATDTSQGELVHPKALPLIVGAQLIHADKLGEKVEDSTMPIRRTVNSTRETPPKSKLAEG
EEEKPEPDISSEESVSTVEEQENETPPATSSEAEQPKGEPENEEKEENKSSEETKKDEKD
QSKEKEKKVKKTIPSWATLSASQLARAQKQTPMASSPRPKMDAILTEAIKACFQKSGASVVA
IRKYIIHKYPSLELERRGYLLKQALKRELNRGVIKQVKGKGASGSFVVVQKSRKTPQKSRNR
KNRSSAVDPEPQVKLEDVLPLAFTRLCEPKEASYSLIRKYVSQYYPKLRVDIRPQLLKNALQR
AVERGQLEQITGKGASGTFQLKKSGEKPLLGGSLMEYAILSAIAAMNEPKTCSTTALKKYVL
ENHPGTNSNYQMHLLKKTLQKCEKNGWMEQISGKGFSGTFQLCFPYYPSPGVLFPKKEPDD
SRDEDEDEDESSEEDSEDEEPPPKRRLQKKTPAKSPGKAASVKQRGSKPAPKVSAAQRGKAR
PLPKKAPPKAKTPAKKTRPSSTVIKKPSGGSSKKPATSARKEVKLPGKGKSTMKKSFRVKK
NP_002977.1 eotaxin precursor
(SEQ ID NO: 38)
MKVSAALLWLLLIAAAFSPQGLAGPASVPTTCCFNLANRKIPLQRLESYRRITSGKCPQK
AVIFKTKLAKDICADPKKKWVQDSMKYLDQKSPTPKP
NP_443203.1 liver-expressed antimicrobial peptide 2 precursor
(SEQ ID NO: 38)
MWHLKLCAVLMIFLLLLGQIDGSPIPEVSSAKRRPRRMTPFWRGVSLRPIGASCRDDSEC
ITRLCRKRRCSLSVAQE
NP_113676.2 lethal(3)malignant brain tumor-like protein 2
(SEQ ID NO: 39)
MEKPRSIEETPSSEPMEEEEDDDLELFGGYDSFRSYNSSVGSESSSYLEESSEAENEDRE
AGELPTSPLHLLSPGTPRSLDGSGSEPAVCEMCGIVGTREAFFSKTKRFCSVSCSRSYSS
NSKKASILARLQGKPPTKKAKVLHKAAWSAKIGAFLHSQGTGQLADGTPTGQDALVLGFD
WGKFLKDHSYKAAPVSCFKHVPLYDQWEDVMKGMKVEVLNSDAVLPSRVYWIASVIQTA
GYRVLLRYEGFENDASHDFWCNLGTVDVHPIGWCAINSKILVPPRTIHAKFTDWKGYLMKR
LVGSRTLPVDFHIKMVESMKYPFRQGMRLEVVDKSQVSRTRMAVVDTVIGGRLRLLYEDGD
SDDDFWCHMWSPLIHPVGWSRRVGHGIKMSERRSDMAHHPTFRKIYCDAVPYLFKKVRAV
YTEGGWFEEGMKLEAIDPLNLGNICVATVCKVLLDGYLMICVDGGPSTDGLDWFCYHASSH
AIFPATFCQKNDIELTPPKGYEAQTFNWENYLEKTKSKAAPSRLFNMDCPNHGFKVGMKLE
AVDLMEPRLICVATVKRVVHRLLSIHFDGWDSEYDQWVDCESPDIYPVGWCELTGYQLQPP
VAAEPATPLKAKEATKKKKKQFGKKRKRIPPTKTRPLRQGSKKPLLEDDPQGARKISSEPVP
GEIIAVRVKEEHLDVASPDKASSPELPVSVENIKQETDD
NP_002986.1 lymphotactin precursor
(SEQ ID NO: 40)
MRLLILALLGICSLTAYIVEGVGSEVSDKRTCVSLTTQRLPVSRIKTYTITEGSLRAVIF
ITKRGLKVCADPQATWVRDVVRSMDRKSNTRNNMIQTKPTGTQQSTNTAVTLTG
NP_005185.2 CCAAT/enhancer-binding protein beta
(SEQ ID NO: 41)
MQRLVAWDPACLPLPPPPPAFKSMEVANFYYEADCLAAAYGGKAAPAAPPAARPGPRPPAG
ELGSIGDHERAIDFSPYLEPLGAPQAPAPATATDTFEAAPPAPAPAPASSGQHHDFLSDLFSDD
YGGKNCKKPAEYGYVSLGRLGAAKGALHPGCFAPLHPPPPPPPPPAELKAEPGFEPADCKRK
EEAGAPGCGAGMAAGFPYALRAYLGYQAVPSGSSQSLSTSSSSSPPGTPSPADAKAPPTACY
AGAAPAPSQVKSKAKKTVDKHSDEYKIRRERNNIAVRKSRDKAKMRNLETQHKVLELTAEN
ERLQKKVEQLSRELSTLRNLFKQLPEPLLASSGHC
NP_001001430.1 troponin T, cardiac muscle isoform 2
(SEQ ID NO: 42)
MSDIEEVVEEYEEEEQEEAAVEEQEEAAEEDAEAEAETEETRAEEDEEEEEAKEAEDGPMEE
SKPKPRSFMPNLVPPKIPDGERVDFDDIHRKRMEKDLNELQALIEAHFENRKKEEEELVSLKD
RIERRRAERAEQQRIRNEREKERQNRLAEERARREEEENRRKAEDEARKKKALSNMMHFGG
YIQKQAQTERKSGKRQTEREKKKKILAERRKVLAIDHLNEDQLREKAKELWQSIYNLEAEKF
DLQEKFKQQKYEINVLRNRINDNQKVSKTRGKAKVTGRWK
NP_001073315.1 CREB-binding protein isoform b
(SEQ ID NO: 43)
MAENLLDGPPNPKRAKLSSPGFSANDSTDFGSLFDLENDLPDELIPNGGELGLLNSGNLV
PDAASKHKQLSELLRGGSGSSINPGIGNVSASSPVQQGLGGQAQGQPNSANMASLSAMGKSP
LSQGDSSAPSLPKQAASTSGPTPAASQALNPQAQKQVGLATSSPATSQTGPGICMNANFNQT
HPGLLNSNSGHSLINQASQGQAQVMNGSLGAAGRGRGAGMPYPTPAMQGASSSVLAETLT
QVSPQMTGHAGLNTAQAGGMAKMGITGNTSPFGQPFSQAGGQPMGATGVNPQLASKQSM
VNSLPTFPTDIKNTSVTNVPNMSQMQTSVGIVPTQAIATGPTADPEKRKLIQQQLVLLLHAHK
CQRREQANGEVRACSLPHCRTMKNVLNHMTHCQAGKACQAILGSPASGIQNTIGSVGTGQQ
NATSLSNPNPIDPSSMQRAYAALGLPYMNQPQTQLQPQVPGQQPAQPQTHQQMRTLNPLGN
NPMNIPAGGITTDQQPPNLISESALPTSLGATNPLMNDGSNSGNIGTLSTIPTAAPPSSTGVRKG
WHEHVTQDLRSHLVHKLVQAIFPTPDPAALKDRRMENLVAYAKKVEGDMYESANSRDEYY
HLLAEKIYKIQKELEEKRRSRLHKQGILGNQPALPAPGAQPPVIPQAQPVRPPNGPLSLPVNR
MQVSQGMNSFNPMSLGNVQLPQAPMGPRAASPMNHSVQMNSMGSVPGMAISPSRMPQPPN
MMGAHTNNMMAQAPAQSQFLPQNQFPSSSGAMSVGMGQPPAQTGVSQGQVPGAALPNPL
NMLGPQASQLPCPPVTQSPLHPTPPPASTAAGMPSLQHTTPPGMTPPQPAAPTQPSTPVSSSG
QTPTPTPGSVPSATQTQSTPTVQAAAQAQVTPQPQTPVQPPSVATPQSSQQQPTPVHAQPPGT
PLSQAAASIDNRVPTPSSVASAETNSQQPGPDVPVLEMKTETQAEDTEPDPGESKGEPRSEM
MEEDLQGASQVKEETDIAEQKSEPMEVDEKKPEVKVEVKEEEESSSNGTASQSTSPSQPRKKI
FKPEELRQALMPTLEALYRQDPESLPFRQPVDPQLLGIPDYFDIVKNPMDLSTIKRKLDTGQY
QEPWQYVDDVWLMFNNAWLYNRKTSRVYKFCSKLAEVFEQEIDPVMQSLGYCCGRKYEFS
PQTLCCYGKQLCTIPRDAAYYSYQNRYHFCEKCFTEIQGENVTLGDDPSQPQTTISKDQFEKK
KNDTLDPEPFVDCKECGRKMHQICVLHYDIIWPSGFVCDNCLKKTGRPRKENKFSAKRLQTT
RLGNHLEDRVNKFLRRQNHPEAGEVFVRVVASSDKTVEVKPGMKSRFVDSGEMSESFPYRT
KALFAFEEIDGVDVCFFGMHVQEYGSDCPPPNTRRVYISYLDSIHFFRPRCLRTAVYHEILIGY
LEYVKKLGYVTGHIWACPPSEGDDYIFHCHPPDQKIPKPKRLQEWYKKMLDKAFAERIIHDY
KDIFKQATEDRLTSAKELPYFEGDFWPNVLEESIKELEQEEEERKKEESTAASETTEGSQGDS
KNAKKKNNKKTNKNKSSISRANKKKPSMPNVSNDLSQKLYATMEKHKEVFFVIHLHAGPVI
NTLPPIVDPDPLLSCDLMDGRDAFLTLARDKHWEFSSLRRSKWSTLCMLVELHTQGQDRFV
YTCNECKHHVETRWHCTVCEDYDLCINCYNTKSHAHKMVKWGLGLDDEGSSQGEPQSKSP
QESRRLSIQRCIQSLVHACQCRNANCSLPSCQKMKRVVQHTKGCKRKTNGGCPVCKQLIALC
CYHAKHCQENKCPVPFCLNIKHKLRQWQHRLQQAQLMRRRMATMNTRNVPQQSLPSPTS
APPGTPTQQPSTPQTPQPPAQPQPSPVSMSPAGFPSVARTQPPTTVSTGKPTSQVPAPPPPAQPP
PAAVEAARQIEREAQQQQHLYRVNINNSMPPGRTGMGTPGSQMAPVSLNVPRPNQVSGPVM
PSMPPGQWQQAPLPQQQPMPGLPRPVISMQAQAAVAGPRMPSVQPPRSISPSALQDLLRTLK
SPSSPQQQQQVLNILKSNPQLMAAFIKQRTAKYVANQPGMQPQPGLQSQPGMQPQPGMHQQ
PSLQNLNAMQAGVPRPGVPPQQQAMGGLNPQGQALNIMNPGHNPNMASMNPQYRHMFRR
QLLQQQQQQQQQQQQQQQQQQGSAGMAGGMAGHGQFQQPQGPGGYPPAMQQQQRMQQ
HLPLQGSSMGQMAAQMGQLGQMGQPGLGADSTPNIQQALQQRILQQQQMKQQIGSPGQPN
PMSPQQHMLSGQPQASHLPGQQIATSLSNQVRSPAPVQSPRPQSQPPHSSPSPRIQPQPSPHHV
SPQTGSPHPGLAVTMASSIDQGHLGNPEOSAMLPQLNTPSRSALSSELSLVGDTTGDTLEKFV
EGL
NP_00187l.2 cyclic AMP-dependent transcription factor ATF-2
(SEQ ID NO: 44)
MKFKLHVNSARQYKDLWNMSDDKPFLCTAPGCGQRFTNEDHLAVHKHKHEMTLKFGPAR
NDSVIVADQTPTPTRFLKNCEEVGLFNELASPFENEFKKASEDDIKKMPLDLSPLATPIIRSKIE
EPSVVETTHQDSPLPHPESTTSDEKEVPLAQTAQPTSAIVRPASLQVPNVLLTSSD
SSVIIQQAVPSPTSSTVITQAPSSNRPIVPVPGPFPLLLHLPNGQTMPVAIPASITSSNV
HVPAAVPLVRPVTMVPSVPGIPGPSSPQPVQSEAKMRLKAALTQQHPPVTNGDTVKGHGSG
LVRTQSEESRPQSLQQPATSTTETPASPAHTTPQTQSTSGRRRRAANFDPDFKRRKFLE
RNRAAASRCRQKRKVWVQSLEKKAEDLSSLNGQLQSEVTLLRNEVAQLKQLLLAHKDCPV
TAMQKKSGYHTADKDDSSEDISVPSSPHTEAIQHSSVSTSNGVSSTSKAEAVATSVLTQMAD
QSTEPALSQIVMAPSSQSQPSGS
NP_001901.1 cathepsin E isoform a preproprotein
(SEQ ID NO: 45)
MKTLLLLLLVLLELGEAQGSLHRVPLRRHPSLKKKLRARSQLSEFWKSHNLDMIQFTESC
SMDQSAKEPLINYLDMEYFGTISIGSPPQNFTVIFDTGSSNLWVPSVYCTSPACKTHSRF
QPSQSSTYSQPGQSFSIQYGTGSLSGIIGADQVSVEGLTVVGQQFGESVTEPGQTFVDAE
FDGILGLGYPSLAVGGVTPVFDNMMAQNLVDLPMFSVYMSSNPEGGAGSELIFGGYDHSHF
SGSLNWVPVTKQAYWQIALDNIQVGGTVMFCSEGCQAIVDTGTSLITGPSDKIKQLQNAIGA
APVDGEYAVECANLNVMPDVTFTINGVPYTLSPTAYTLLDFVDGMQFCSSGFQGLDIHPPAG
PLWILGDVFIRQFYSVFDRGNNRVGLAPAVP
NP_001139512.1 glycine receptor subunit alpha-1 isoform 1 precursor
(SEQ ID NO: 46)
MYSFNTLRLYLWETIVFFSLAASKEAEAARSAPKPMSPSDFLDKLMGRTSGYDARIRPNF
KGPPVNVSCNIFINSFGSIAETTMDYRVNIFLRQQWNDPRLAYNEYPDDSLDLDPSMLDS
IWKPDLFFANEKGAHFHEITTDNKLLRISRNGNVLYSIRITLTLACPMDLKNFPMDVQTC
IMQLESFGYTMNDLIFEWQEQGAVQVADGLTLPQFILKEEKDLRYCTKHYNTGKFTCIEARF
HLERQMGYYLIQMYIPSLLIVILSWISFWINMDAAPARVGLGITTVLTMTTQSSGSRA
SLPKVSYVKAIDIWMAVCLLFVFSALLEYAAVNFVSRQHKELLRFRRKRRHHKSPMLNLFQE
DEAGEGRFNFSAYGMGPACLQAKDGISVKGANNSNTTNPPPAPSKSPEEMRKLFIQRAKKID
KISRIGFPMAFLIFNMFYWIIYKIVRREDVHNQ
NP_001108.2 pituitary adenylate cyclasc-activating polypeptide precursor
(SEQ ID NO: 47)
MTMCSGARLALLVYGIIMHSSVYSSPAAAGLRFPGIRPEEEAYGEDGNPLPDFDGSEPPG
AGSPASAPRAAAAWYRPAGRRDVAHGILNEAYRKVLDQLSAGKHLQSLVARGVGGSLGGG
AGDDAEPLSKRHSDGIFTDSYSRYRKQMAVKKYLAAVLGKRYKQRVKNKGRRIAYL
NP_055572.1 mastermind-like protein 1
(SEQ ID NO: 48)
MVLPTCPMAEFALPRHSAVMERLRRRIELCRRHHSTCEARYEAVSPERLELERQHTFALH
QRCIQAKAKRAGKHRQPPAATAPAPAAPAPRLDAADGPEHGRPATHLHDTVKRNLDSATSP
QNGDQQNGYGDLFPGHKKTRREAPLGVAISSNGLPPASPLGQSDKPSGADALQSSGKHSLGL
DSLNKKRLADSSLHLNGGSNPSESFPLSLNKELKQEPVEDLPCMITGTVGSISQSNLMPDLNL
NEQEWKELIEELNRSVPDEDMKDLFNEDFEEKKDPESSGSATQTPLAQDINIKTEFSPAAFEQ
EQLGSPQVRAGSAGQTFLGPSSAPVSTDSPSLGGSQTLFHTSGQPRADNPSPNLMPASAQAQ
NAQRALAGVVLPSQGPGGASELSSAHQLQQIAAKQKREQMLQNPQQATPAPAPGQMSTWQ
QTGPSHSSLDVPYPMEKPASPSSYKQDFTNSKLLMMPSVNKSSPRPGGPYLQPSHVNLLSHQ
PPSNLNQNSANNQGSVLDYGNTKPLSHYKADCGQGSPGSGQSKPALMAYLPQQLSHISHEQ
NSLFLMKPKPGNMPFRSLVPPGQEQNPSSVPVQAQATSVGTQPPAVSVASSHNSSPYLSSQQ
QAAVMKQHQLLLDQQKQREQQQKHLQQQQFLQRQQHLLAEQEKQQFQRHLTRPPPQYQD
PTQGSFPQQVGQFTGSSAAVPGMNTLGPSNSSCPRVFPQAGNLMPMGPGHASVSSLPTNSGQ
QDRGVAQFPGSQNMPQSSLYGMASGITQIVAQPPPQATNGHAHIPRQTNVGQNTSVSAAYG
QNSLGSSGLSQQHNKGTLNPGLTKPPVPRVSPAMGGQNSSWQHQGMPNLSGQTPGNSNVSP
FTAASSFHMQQQAHLKMSSPQFSQAVPNRPMAPMSSAAAVGSLLPPVSAQQRTSAPAPAPPP
TAPQQGLPGLSPAGPELGAFSQSPASQMGGRAGLHCTQAYPVRTAGQELPFAYSGQPGGSGL
SSVAGHTDLIDSLLKNRTSEEWMSDLDDLLGSQ
NP_004043.2 BCL2/adenovirus E1B 19 kDa protein-interacting protein 3
(SEQ ID NO: 49)
MSQNGAPGMQEESLQGSWVELHFSNNGNGGSVPASVSIYNGDMEKILLDAQHESGRSSSKS
SHCDSPPRSQTPQDTNRASETDTHSIGEKNSSQSEEDDIERRKEVESILKKNSDWIWDW
SSRPENIPPKEFLFKHPKRTATLSMRNTSVMKKGGIFSAEFLKVFLPSLLLSHLLAIGLG
IYIGRRLTTSTSTF
NP_004336.2 cathelicidin antimicrobial peptide
(SEQ ID NO: 50)
MKTQRDGHSLGRWSLVLLLLGLVMPLAIIAQVLSYKEAVLRAIDGINQRSSDANLYRLLDLD
PRPTMDGDPDTPKPVSFTVKETVCPRTTQQSPEDCDFKKDGLVKRCMGTVTLNQARGSFDIS
CDKDNKRFALLGDFFRKSKEKIGKEFKRIVQRIKDFLRNLVPRTES
NP_001181946.1 T-cell surface glycoprotein CD4 isoform 3
(SEQ ID NO: 51)
MGKKLPLHLTLPQALPQYAGSGNLTLALEAKTGKLHQEVNLVVMRATQLQKNLTCEVWGP
TSPKLMLSLKLENKEAKVSKREKAVWVLNPEAGMWQCLLSDSGQVLLESNIKVLPTW STPV
QPMALIVLGGVAGLLLFIGLGIFFCVRCRHRRRQAERMSQIKRLLSEKKTCQCPHRFQKTCSPI
NP_005219.2 epidermal growth factor receptor isoform a precursor
(SEQ ID NO: 52)
MRPSGTAGAALLALLAALCPASRALEEKKVCQGTSNKLTQLGTFEDHFLSLQRMFNNCEVV
LGNLEITYVQRNYDLSFLKTIQEVAGYVLIALNTVERIPLENLQIIRGNMYYENSYALAVLSN
YDANKTGLKELPMRNLQEILHGAVRFSNNPALCNVESIQWRDIVSSDFLSNMSMDFQNHLGS
CQKCDPSCPNGSCWGAGEENCQKLTKIICAQQCSGRCRGKSPSDCCHNQCAAGCTGPRESD
CLVCRKFRDEATCKDTCPPLMLYNPTTYQMDVNPEGKYSFGATCVKKCPRNYVVTDHGSC
VRACGADSYEMEEDGVRKCKKCEGPCRKVCNGIGIGEFKDSLSINATNIKHFKNCTSISGDLH
ILPVAFRGDSFTHTPPLDPQELDILKTVKEITGFLLIQAWPENRTDLHAFENLEIIRGRTKQHGQ
FSLAVVSLNITSLGLRSLKEISDGDVIISGNKNLCYANTINWKKLFGTSGQKTKIISNRGENSC
KATGQVCHALCSPEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYADAGHVCHLCH
PNCTYGCTGPGLEGCPTNGPKIPSIATGMVGALLLLLVVALGIGLFMRRRHIVRKRTLRRLLQ
ERELVEPLTPSGEAPNQALLRILKETEFKKIKVLGSGAFGTVYKGLWIPEGEKVKIPVAIKELR
EATSPKANKEILDEAYVMASVDNPHVCRLLGICLTSTVQLITQLMPFGCLLDYVREHKDNIGS
QYLLNWCVQIAKGMNYLEDRRLVHRDLAARNVLVKTPQHVKITDFGLAKLLGAEEKEYHA
EGGKVPIKWMALESILHRIYTHQSDVWSYGVTVWELMTFGSKPYDGIPASEISSILEKGERLP
QPPICTIDVYMIMVKCWMIDADSRPKFRELIIEFSKMARDPQRYLVIQGDERMHLPSPTDSNF
YRALMDEEDMDDVVDADEYLIPQQGFFSSPSTSRTPLLSSLSATSNNSTVACIDRNGLQSCPI
KEDSFLQRYSSDPTGALTEDSIDDTFLPVPEYINQSVPKRPAGSVQNPVYHNQPLNPAPSRDP
HYQDPHSTAVGNPEYLNTVQPTCVNSTFDSPAHWAQKGSHQISLDNPDYQQDFFPKEAKPN
GIFKGSTAENAEYLRVAPQSSEFIGA
NP_001129495.1 transcription factor NF-E2 45 kDa subunit isoform 2
(SEQ ID NO: 53)
MSPCPPQQSRNRVIQLSTSELGEMELTWQEIMSITELQGLNAPSEPSFEPQAPAPYLGPP
PPTTYCPCSIHPDSGFPLPPPPYELPASTSHVPDPPYSYGNMAIPVSKPLSLSGLLSEPL
QDPLALLDIGLPAGPPKPQEDPESDSGLSLNYSDAESLELEGTEAGRRRSEYVEMYPVEY
PYSLMPNSLAHSNYTLPAAETPLALEPSSGPVRAKPTARGEAGSRDERRALAMKIPFPTD
KIVNLPVDDFNELLARYPLTESQLALVRDIRRRGKNKVAAQNCRKRKLETIVQLERELER
LTNERERLLRARGEADRTLEVMRQQLTELYRDIFQHLRDESGNSYSPEEYALQQAADGTI
FLVPRGTKMEATD
NP_008855.1 serine/arginine-rich splicing factor 1 isoform 1
(SEQ ID NO: 54)
MSGGGVIRGPAGNNDCRIYVGNLPPDIRTKDIEDVFYKYGAIRDIDLKNRRGGPPFAFVE
FEDPRDAEDAVYGRDGYDYDGYRLRVEFPRSGRGTGRGGGGGGGGGAPRGRYGPPSRRSE
NRVVVSGLPPSGSWQDLKDHMREAGDVCYADVYRDGTGVVEFVRKEDMTYAVRKLDNTK
FRSHEGETAYIRVKVDGPRSPSYGRSRSRSRSRSRSRSRSNSRSRSYSPRRSRGSPRYSPRHSRS
RSRT
NP_945315.1 parathyroid hormone-related protein isoform 2 preproprotein
(SEQ ID NO: 55)
MQRRLVQQWSVAVFLLSYAVPSCGRSVEGLSRRLKRAVSEHQLLHDKGKSIQDLRRRFFLH
HLIAEIHTAEIRATSEVSPNSKPSPNTKNHPVRFGSDDEGRYLTQETNKVETYKEQPLK
TPGKKKKGKPGKRKEQEKKKRRTRSAWLDSGVTGSGLEGDHLSDTSTTSLELDSR
NP_391988.1 integrin beta-1 isoform 1D
(SEQ ID NO: 56)
precursorMNLQPIFWIGLISSVCCVFAQTDENRCLKANAKSCGECIQAGPNCGWCTNSTFLQE
GMPTSARCDDLEALKKKGCPPDDIENPRGSKDIKKNKNVTNRSKGTAEKLKPEDITQIQPQQ
LVLRLRSGEPQTFTLKFKRAEDYPIDLYYLMDLSYSMKDDLENVKSLGTDLMNEMRRITSDF
RIGFGSFVEKTVMPYISTTPAKLRNPCTSEQNCTSPFSYKNVLSLTNKGEVFNELVGKQRISGN
LDSPEGGFDAIMQVAVCGSLIGWRNVTRLLVFSTDAGFHFAGDGKLGGIVLPNDGQCHLEN
NMYTMSHYYDYPSIAHLVQKLSENNIQTIFAVTEEFQPVYKELKNLIPKSAVGTLSANSSNVI
QLIIDAYNSLSSEVILENGKLSEGVTISYKSYCKNGVNGTGENGRKCSNISIGDEVQFEISITSN
KCPKKDSDSFKIRPLGFTEEVEVILQYICECECQSEGIPESPKCHEGNGTFECGACRCNEGRVG
RHCECSTDEVNSEDMDAYCRKENSSEICSNNGECVCGQCVCRKRDNTNEIYSGKFCECDNF
NCDRSNGLICGGNGVCKCRVCECNPNYTGSACDCSLDTSTCEASNGQICNGRGICECGVCKC
TDPKFQGQTCEMCQTCLGVCAEHKECVQCRAFNKGEKKDTCTQECSYFNITKVESRDKLPQ
PVQPDPVSHCKEKDVDDCWFYFTYSVNGNNEVMVHVVENPECPTGPDIIPIVAGVVAGIVLI
GLALLLIWKLLMIIHDRREFAKFEKEKMNAKWDTQENPIYKSPINNFKNPNYGRKAGL
NP_006004.2 60S ribosomal protein L10
(SEQ ID NO: 57)
MGRRPARCYRYCKNKPYPKSRFCRGVPDAKIRIFDLGRKKAKVDEFPLCGHMVSDEYEQLS
SEALEAARICANKYMVKSCGKDGFHIRVRLHPFHVIRINKMLSCAGADRLQTGMRGAFGKP
QGTVARVHIGQVIMSIRTKLQNKEHVIEALRRAKFKFPGRQKIHISKKWGFTKFNADEFEDM
VAEKRLIPDGCGVKYIPNRGPLDKWRALHS
NP_001193858.1 advanced glycosylation end product-specific receptor
isoform 2 precursor
(SEQ ID NO: 58)
MAAGTAVGAWVLVLSLWGAVVGAQNITARIGEPLVLKCKGAPKKPPQRLEWKLNTGRTEA
WKVLSPQGGGPWDSVARVLPNGSLFLPAVGIQDEGIFRCQAMNRNGKETKSNYRVRVYQIP
GKPEIVDSASELTAGVPNKVVEESRRSRKRPCEQEVGTCVSEGSYPAGTLSWHLDGKPLVPN
EKGVSVKEQTRRHPETGLFTLQSELMVTPARGGDPRPTFSCSFSPGLPRHRALRTAPIQPRVW
EPVPLEEVQLVVEPEGGAVAPGGTVTLTCEVPAQPSPQIHWMKDGVPLPLPPSPVLILPEIGPQ
DQGTYSCVATHSSHGPQESRAVSISIIEPGEEGPTAGSVGGSGLGTLALALGILGGLGTAALLI
GVILWQRRQRRGEERKAPENQEEEEERAELNQSEEPEAGESSTGGP
NP_005399.1 C-C motif chemokine 13 precursor
(SEQ ID NO: 59)
MKVSAVLLCLLLMTAAFNPQGLAQPDALNVPSTCCFTFSSKKISLQRLKSYVITTSRCPQ
KAVIFRTKLGKEICADPKEKWVQNYMKHLGRKAHTLKT
NP_002976.2 C-C motif chemokine 5 precursor
(SEQ ID NO: 60)
MKVSAAALAVILIATALCAPASASPYSSDTTPCCFAYIARPLPRAHIKEYFYTSGKCSNP
AVVFVTRKNRQVCANPEKKWVREYINSLEMS
NP_006264.2 C-C motif chemokine 7 precursor
(SEQ ID NO: 61)
MKASAALLCLLLTAAAFSPQGLAQPVGINTSTTCCYRFINKKIPKQRLESYRRTTSSHCP
REAVIFKTKLDKEICADPTQKWVQDFMKHLDKKTQTPKL
NP_002080.1 C-X-C motif chemokine 2
(SEQ ID NO: 62)
MARATLSAAPSNPRLLRVALLLLLLVAASRRAAGAPLATELRCQCLQTLQGIHLKNIQSV
KVKSPGPHCAQTEVIATLKNGQKACLNPASPMVKKIIEKMLKNGKSN
NP_002006.2 forkhead box protein O1
(SEQ ID NO: 63)
MAEAPQVVEIDPDFEPLPRPRSCTWPLPRPEFSQSNSATSSPAPSGSAAANPDAAAGLPS
ASAAAVSADFMSNLSLLEESEDFPQAPGSVAAAVAAAAAAAATGGLCGDFQGPEAGCLHPA
PPQPPPPGPLSQHPPVPPAAAGPLAGQPRKSSSSRRNAWGNLSYADLITKAIESSAEKRLTLSQ
IYEWMVKSVPYFKDKGDSNSSAGWKNSIRHNLSLHSKFIRVQNEGTGKSSWWMLNPEGGKS
GKSPRRRAASMDNNSKFAKSRSRAAKKKASLQSGQEGAGDSPGSQFSKWPASPGSHSNDDF
DNWSTFRPRTSSNASTISGRLSPIMTEQDDLGEGDVHSMVYPPSAAKMASTLPSLSEISNPEN
MENLLDNLNLLSSPTSLTVSTQSSPGTMMQQTPCYSFAPPNTSLNSPSPNYQKYTYGQSSMSP
LPQMPIQTLQDNKSSYGGMSQYNCAPGLLKELLTSDSPPHNDIMTPVDPGVAQPNSRVLGQN
VMMGPNSVMSTYGSQASHNKMMNPSSHTHPGHAQQTSAVNGRPLPHTVSTMPHTSGMNR
LTQVKTPVQVPLPHPMQMSALGGYSSVSSCNGYGRMGLLHQEKLPSDLDGMFIERLDCDME
SIIRNDLMDGDTLDFNFDNVLPNQSFPHSVKTTTHSWVSG
NP_963853.1 forkhead box protein O3
(SEQ ID NO: 64)
MAEAPASPAPLSPLEVELDPEFEPQSRPRSCTWPLQRPELQASPAKPSGETAADSMIPEE
EDDEDDEDGGGRAGSAMAIGGGGGSGTLGSGLLLEDSARVLAPGGQDPGSGPATAAGGLSG
GTQALLQPQQPLPPPQPGAAGGSGQPRKCSSRRNAWGNLSYADLITRAIESSPDKRLTLSQIY
EWMVRCVPYFKDKGDSNSSAGWKNSIRHNLSLHSRFMRVQNEGTGKSSWWIINPDGGKSG
KAPRRRAVSMDNSNKYTKSRGRAAKKKAALQTAPESADDSPSQLSKWPGSPTSRSSDELDA
WTDFRSRTNSNASTVSGRLSPIMASTELDEVQDDDAPLSPMLYSSSASLSPSVSKPCTVELPR
LTDMAGTMNLNDGLTENLMDDLLDNITLPPSQPSPTGGLMQRSSSFPYTTKGSGLGSPTSSFN
STVFGPSSLNSLRQSPMQTIQENKPATFSSMSHYGNQTLQDLLTSDSLSHSDVMMTQSDPLM
SQASTAVSAQNSRRNVMLRNDPMMSFAAQPNQGSLVNQNLLHHQHQTQGALGGSRALSNS
VSNMGLSESSSLGSAKHQQQSPVSQSMQTLSDSLSGSSLYSTSANLPVMGHEKFPSDLDLDM
FNGSLECDMESIIRSELMDADGLDFNFDSLISTQNVVGLNVGNFTGAKQASSQSWVPG
NP_005929.2 forkhead box protein O4 isoform 1
(SEQ ID NO: 65)
MDPGNENSATEAAAIIDLDPDFEPQSRPRSCTWPLPRPEIANQPSEPPEVEPDLGEKVHT
EGRSEPILLPSRLPEPAGGPQPGILGAVTGPRKGGSRRNAWGNQSYAELISQAIESAPEK
RLTLAQIYEWMVRTVPYFKDKGDSNSSAGWKNSIRHNLSLHSKFIKVHNEATGKSSWWML
NPEGGKSGKAPRRRAASMDSSSKLLRGRSKAPKKKPSVLPAPPEGATPTSPVGHFAKWSGSP
CSRNREEADMWTTFRPRSSSNASSVSTRLSPLRPESEVLAEEIPASVSSYAGGVPPTLNEGLEL
LDGLNLTSSHSLLSRSGLSGFSLQHPGVTGPLHTYSSSLFSPAEGPLSAGEGCF
SSSQALEALLTSDTPPPPADVLMTQVDPILSQAPTLLLLGGLPSSSKLATGVGLCPKPLE
APGPSSLVPTLSMIAPPPVMASAPIPKALGTPVLTPPTEAASQDRMPQDLDLDMYMENLE
CDMDNIISDLMDEGEGLDFNFEPDP
NP_002087.2 general transcription factor IIF subunit 1
(SEQ ID NO: 66)
MAALGPSSQNVTEYVVRVPKNTTKKYNIMAFNAADKVNFATWNQARLERDLSNKKIYQEE
EMPESGAGSEFNRKLREEARRKKYGIVLKEFRPEDQPWLLRVNGKSGRKFKGIKKGGVTEN
TSYYIFTQCPDGAFEAFPVHNWYNFTPLARHRTLTAEEAEEEWERRNKVLNHFSIMQQRRLK
DQDQDEDEEEKEKRGRRKASELRIHDLEDDLEMSSDASDASGEEGGRVPKAKKKAPLAKGG
RKKKKKKGSDDEAFEDSDDGDFEGQEVDYMSDGSSSSQEEPESKAKAPQQEEGPKGVDEQS
DSSEESEEEKPPEEDKEEEEEKKAPTPQEKKRRKDSSEESDSSEESDIDSEASSALFMAKKKTP
PKRERKPSGGSSRGNSRPGTPSAEGGSTSSTLRAAASKLEQGKRVSEMPAAKRLRLDTGPQS
LSGKSTPQPPSGKTTPNSGDVQVTEDAVRRYLTRKPMTTKDLLKKFQTKKTGLSSEQTVNVL
AQILKRLNPERKMINDKMHFSLKE
NP_002087.2 general transcription factor IIF subunit 1
(SEQ ID NO: 67)
MAALGPSSQNVTEYVVRVPKNTTKKYNIMAFNAADKVNFATWNQARLERDLSNKKIYQEE
EMPESGAGSEFNRKLREEARRKKYGIVLKEFRPEDQPWLLRVNGKSGRKFKGIKKGGVTEN
TSYYIFTQCPDGAFEAFPVHNWYNFTPLARHRTLTAEEAEEEWERRNKVLNHFSIMQQRRLK
DQDQDEDEEEKEKRGRRKASELRIHDLEDDLEMSSDASDASGEEGGRVPKAKKKAPLAKGG
RKKKKKKGSDDEAFEDSDDGDFEGQEVDYMSDGSSSSQEEPESKAKAPQQEEGPKGVDEQS
DSSEESEEEKPPEEDKEEEEEKKAPTPQEKKRRKDSSEESDSSEESDIDSEASSALFMAKKKTP
PKRERKPSGGSSRGNSRPGTPSAEGGSTSSTLRAAASKLEQGKRVSEMPAAKRLRLDTGPQS
LSGKSTPQPPSGKTTPNSGDVQVTEDAVRRYLTRKPMTTKDLLKKFQTKKTGLSSEQTVNVL
AQILKRLNPERKMINDKMHFSLKE
NP_001997.5 heparin-binding growth factor 2
(SEQ ID NO: 68)
MVGVGGGDVEDVTPRPGGCQISGRGARGCNGIPGAAAWEAALPRRRPRRHPSVNPRSRAAG
SPRTRGRRTEERPSGSRLGDRGRGRALPGGRLGGRGRGRAPERVGGRGRGRGTAAPRAAPA
ARGSRPGPAGTMAAGSITTLPALPEDGGSGAFPPGHFKDPKRLYCKNGGFFLRIHPDGRVDG
VREKSDPHIKLQLQAEERGVVSIKGVCANRYLAMKEDGRLLASKCVTDECFFFERLESNNYN
TYRSRKYTSWYVALKRTGQYKLGSKTGPGQKAILFLPMSAKS
NP_000592.3 hepatocyte growth factor isoform 1 preproprotein
(SEQ ID NO: 69)
MWVTKLLPALLLQHVLLHLLLLPIAIPYAEGQRKRRNTIHEFKKSAKTTLIKIDPALKIK
TKKVNTADQCANRCTRNKGLPFTCKAFVFDKARKQCLWFPFNSMSSGVKKEFGHEFDLYE
NKDYIRNCIIGKGRSYKGTVSITKSGIKCQPWSSMIPHEHSFLPSSYRGKDLQENYCRNPRGEE
GGPWCFTSNPEVRYEVCDIPQCSEVECMTCNGESYRGLMDHTESGKICQRWDHQTPHRHKF
LPERYPDKGFDDNYCRNPDGQPRPWCYTLDPHTRWEYCAIKTCADNTMNDTDVPLETTECI
QGQGEGYRGTVNTIWNGIPCQRWDSQYPHEHDMTPENFKCKDLRENYCRNPDGSESPWCFT
TDPNIRVGYCSQIPNCDMSHGQDCYRGNGKNYMGNLSQTRSGLTCSMWDKNMEDLHRHIF
WEPDASKLNENYCRNPDDDAHGPWCYTGNPLIPWDYCPISRCEGDTTPTIVNLDHPVISCAK
TKQLRVVNGIPTRTNIGWMVSLRYRNKHICGGSLIKESWVLTARQCFPSRDLKDYEAWLGIH
DVHGRGDEKCKQVLNVSQLVYGPEGSDLVLMKLARPAVLDDFVSTIDLPNYGCTIPEKTSCS
VYGWGYTGLINYDGLLRVAHLYIMGNEKCSQHHRGKVTLNESEICAGAEKIGSGPCEGDYG
GPLVCEQHKMRMVLGVIVPGRGCAIPNRPGIFVRVAYYAKWIHKIILTYKVPQS
NP_001092883.1 histone acetyltransferase MYST3
(SEQ ID NO: 70)
MVKLANPLYTEWILEAIKKVKKQKQRPSEERICNAVSSSHGLDRKTVLEQLELSVKDGTI
LKVSNKGLNSYKDPDNPGRIALPKPRNHGKLDNKQNVDWNKLIKRAVEGLAESGGSTLKSI
ERFLKGQKDVSALFGGSAASGFHQQLRLAIKRAIGHGRLLKDGPLYRLNTKATNVDGKESCE
SLSCLPPVSLLPHEKDKPVAEPIPICSFCLGTKEQNREKKPEELISCADCGNSGHPSCLKFSPEL
TVRVKALRWQCIECKTCSSCRDQGKNADNMLFCDSCDRGFHMECCDPPLTRMPKGMWICQ
ICRPRKKGRKLLQKKAAQIKRRYTNPIGRPKNRLKKQNTVSKGPFSKVRTGPGRGRKRKITLS
SQSASSSSEEGYLERIDGLDFCRDSNVSLKFNKKTKGLIDGLTKFFTPSPDGRKARGEVVDYS
EQYRIRKRGNRKSSTSDWPTDNQDGWDGKQENEERLFGSQEIMTEKDMELFRDIQEQALQK
VGVTGPPDPQVRCPSVIEFGKYEIHTWYSSPYPQEYSRLPKLYLCEFCLKYMKSRTILQQHMK
KCGWFHPPANEIYRKNNISVFEVDGNVSTIYCQNLCLLAKLFLDHKTLYYDVEPFLFYVLTQ
NDVKGCHLVGYFSKEKHCQQKYNVSCIMILPQYQRKGYGRFLIDFSYLLSKREGQAGSPEKP
LSDLGRLSYMAYWKSVILECLYHQNDKQISIKKLSKLTGICPQDITSTLHHLRMLDFRSDQFV
IIRREKLIQDHMAKLQLNLRPVDVDPECLRWTPVIVSNSVVSEEEEEEAEEGENEEPQCQERE
LEISVGKSVSHENKEQDSYSVESEKKPEVMAPVSSTRLSKQVLPHDSLPANSQPSRRGRWGR
KNRKTQERFGDKDSKLLLEETSSAPQEQYGECGEKSEATQEQYTESEEQLVASEEQPSQDGK
PDLPKRRLSEGVEPWRGQLKKSPEALKCRLTEGSERLPRRYSEGDRAVLRGFSESSEEEEEPE
SPRSSSPPILTKPTLKRKKPFLHRRRRVRKRKHHNSSVVTETISETTEVLDEPFEDSDSERPMPR
LEPTFEIDEEEEEEDENELFPREYFRRLSSQDVLRCQSSSKRKSKDEEEDEESDDADDTPILKP
VSLLRKRDVKNSPLEP
DTSTPLKKKKGWPKGKSRKPIHWKKRPGRKPGFKLSREIMPVSTQACVIEPIVSIPKAGR
KPKIQESEETVEPKEDMPLPEERKEEEEMQAEAEEAEEGEEEDAASSEVPAASPADSSNS
PETETKEPEVEEEEEKPRVSEEQRQSEEEQQELEEPEPEEEEDAAAETAQNDDHDADDED
DGHLESTKKKELEEQPTREDVKEEPGVQESFLDANMQKSREKIKDKEETELDSEEEQPSH
DTSVVSEQMAGSEDDHEEDSHTKEELIELKEEEEIPHSELDLETVQAVQSLTQEESSEHE
GAYQDCEETLAACQTLQSYTQADEDPQMSMVEDCHASEHNSPISSVQSHPSQSVRSVSSPNV
PALESGYTQISPEQGSLSAPSMQNMETSPMMDVPSVSDHSQQVVDSGFSDLGSIESTTENYEN
PSSYDSTMGGSICGNSSSQSSCSYGGLSSSSSLTQSSCVVTQQMASMGSSCSMMQQSSVQPA
ANCSIKSPQSCVVERPPSNQQQQPPPPPPQQPQPPPPQPQPAPQPPPPQQQPQ
QQPQPQPQQPPPPPPPQQQPPLSQCSMNNSFTPAPMIMEIPESGSTGNISIYERIPGDFG
AGSYSQPSATFSLAKLQQLTNTIMDPHAMPYSHSPAVTSYATSVSLSNTGLAQLAPSHPL
AGTPQAQATMTPPPNLASTTMNLTSPLLQCNMSATNIGIPHTQRLQGQMPVKGHISIRSK
SAPLPSAAAHQQQLYGRSPSAVAMQAGPRALAVQRGMNMGVNLMPTPAYNVNSMNMNTL
NAMNSYRMTQPMMNSSYHSNPAYMNQTAQYPMQMQMGMMGSQAYTQQPMQPNPHGN
MMYTGPSHHSYMNAAGVPKQSLNGPYMRR
NP_001800.1 histone H3-like centromeric protein A isoform a
(SEQ ID NO: 71)
MGPRRRSRKPEAPRRRSPSPTPTPGPSRRGPSLGASSHQHSRRRQGWLKEIRKLQKSTHL
LIRKLPFSRLAREICVKFTRGVDFNWQAQALLALQEAAEAFLVHLFEDAYLLTLHAGRVTLF
PKDVQLARRIRGLEEGLG
NP_002135.2 homeobox protein Hox-B1
(SEQ ID NO: 72)
MDYNRMNSFLEYPLCNRGPSAYSAHSAPTSFPPSSAQAVDSYASEGRYGGGLSSPAFQQNSG
YPAQQPPSTLGVPFPSSAPSGYAPAACSPSYGPSQYYPLGQSEGDGGYFHPSSYGAQLGGLSD
GYGAGGAGPGPYPPQHPPYGNEQTASFAPAYADLLSEDKETPCPSEPNTPTARTFDWMKVK
RNPPKTAKVSEPGLGSPSGLRTNFTTRQLTELEKEFHFNKYLSRARRVEIAATLELNETQVKI
WFQNRRMKQKKREREEGRVPPAPPGCPKEAAGDASDQSTCTSPEASPSSVTS
NP_079141.2 homeobox protein NANOG
(SEQ ID NO: 73)
MSVDPACPQSLPCFEASDCKESSPMPVICGPEENYPSLQMSSAEMPHTETVSPLPSSMDL
LIQDSPDSSTSPKGKQPTSAEKSVAKKEDKVPVKKQKTRTVFSSTQLCVLNDRFQRQKYL
SLQQMQELSNILNLSYKQVKTWFQNQRMKSKRWQKNNWPKNSNGVTQKASAPTYPSLYSS
YHQGCLVNPTGNLPMWSNQTWNNSTWSNQTQNIQSWSNHSWNTQTWCTQSWNNQAWNS
PFYNCGEESLQSCMQFQPNSPASDLEAALEAAGEGLNVIQQTTRYFSTPQTMDLFLNYSMNM
QPEDV
NP_001801.1 major centromere autoantigen B
(SEQ ID NO: 74)
MGPKRRQLTFREKSRIIQEVEENPDLRKGEIARRFNIPPSTLSTILKNKRAILASERKYG
VASTCRKTNKLSPYDKLEGLLIAWFQQIRAAGLPVKGIILKEKALRIAEELGMDDFTASN
GWLDRFRRRHGVVSCSGVARARARNAAPRTPAAPASPAAVPSEGSGGSTTGWRAREEQPPS
VAEGYASQDVFSATETSLWYDFLPDQAAGLCGGDGRPRQATQRLSVLLCANADGSEKLPPL
VAGKSAKPRAGQAGLPCDYTANSKGGVTTQALAKYLKALDTRMAAESRRVLLLAGRLAAQ
SLDTSGLRHVQLAFFPPGTVHPLERGVVQQVKGHYRQAMLLKAMAALEGQDPSGLQLGLT
EALHFVAAAWQAVEPSDIAACFREAGFGGGPNATITTSLKSEGEEEEEEEEEEEEEEGEGEEE
EEEGEEEEEEGGEGEELGEEEEVEEEGDVDSDEEEEEDEESSSEGLEAEDWAQGVVEAGGSF
GAYGAQEEAQCPTLHFLEGGEDSDSDSEEEDDEEEDDEDEDDDDDEEDGDEVPVPSFGEAM
AYFAMVKRYLTSFPIDDRVQSHILHLEHDLVHVTRKNHARQAGVRGLGHQS
NP_523353.2 male-specific lethal 3 homolog isoform a
(SEQ ID NO: 75)
MSASEGMKFKFHSGEKVLCFEPDPTKARVLYDAKIVDVIVGKDEKGRKIPEYLIHFNGWNRS
WDRWAAEDHVLRDTDENRRLQRKLARKAVARLRSTGRKKKRCRLPGVDSVLKGLPTEEKD
ENDENSLSSSSDCSENKDEEISEESDIEEKTEVKEEPELQTRREMEERTITIEIPEVLKKQLEDD
CYYINRRKRLVKLPCQTNIITILESYVKHFAINAAFSANERPRHHHVMPHANMNVHYIPAEKN
VDLCKEMVDGLRITFDYTLPLVLLYPYEQAQYKKVTSSKFFLPIKESATSTNRSQEELSPSPPL
LNPSTPQSTESQPTTGEPATPKRRKAEPEALQSLRRSTRHSANCDR
LSESSASPQPKRRQQDTSASMPKLFLHLEKKTPVHSRSSSPIPLTPSKEGSAVFAGFEGR
RTNEINEVLSWKLVPDNYPPGDQPPPPSYIYGAQHLLRLFVKLPEILGKMSFSEKNLKAL
LKHFDLFLRFLAEYHDDFFPESAYVAACEAHYSTKNPRAIY
NP_001189442.1 max dimerization protein 1 isoform 2
(SEQ ID NO: 76)
MAAAVRMNIQMLLEAADYLERREREAEHGYASMLPYNNKDRDALKRRNKSKKNNSSSRST
HNEMEKNRRAHLRLCLEKLKGLVPLGPESSRHTTLSLLTKAKLHIKKLEDCDRKAVHQIDQL
QREQRHLKRQLEKLGIERIRMDSIGSTVSSERSDSDREIDVDVESTDYLTGDLDWSSSSVSDS
DERGSMQSLGSDEGYSSTSIKRIKLQDSHKACLGL
NP_055048.1 nucleolar transcription factor 1 isoform a
(SEQ ID NO: 77)
MNGEADCPTDLEMAAPKGQDRWSQEDMLTLLECMKNNLPSNDSSKFKTTESHMDWEKVA
FKDFSGDMCKLKWVEISNEVRKFRTLTELILDAQEHVKNPYKGKKLKKHPDFPKKPLTPYFR
FFMEKRAKYAKLHPEMSNLDLTKILSKKYKELPEKKKMKYIQDFQREKQEFERNLARFRED
HPDLIQNAKKSDIPEKPKTPQQLWYTHEKKVYLKVRPDATTKEVKDSLGKQWSQLSDKKRL
KWIHKALEQRKEYEEIMRDYIQKHPELNISEEGITKSTLTKAERQLKDKFDGRPTKPPPNSYSL
YCAELMANMKDVPSTERMVLCSQQWKLLSQKEKDAYHKKCDQKKKDYEVELLRFLESLPE
EEQQRVLGEEKMLNINKKQATSPASKKPAQEGGKGGSEKPKRPVSAMFIFSEEKRRQLQEER
PELSESELTRLLARMWNDLSEKKKAKYKAREAALKAQSERKPGGEREERGKLPESPKRAEEI
WQQSVIGDYLARFKNDRVKALKAMEMTWNNMEKKEKLMWIKKAAEDQKRYERELSEMR
APPAATNSSKKMKFQGEPKKPPMNGYQKFSQELLSNGELNHLPLKERMVEIGSRWQRISQSQ
KEHYKKLAEEQQKQYKVHLDLWVKSLSPQDRAAYKEYISNKRKSMTKLRGPNPKSSRTTLQ
SKSESEEDDEEDEDDEDEDEEEEDDENGDSSEDGGDSSESSSEDESEDGDENEEDDEDEDDD
EDDDEDEDNESEGSSSSSSSSGDSSDSDSN
NP_006212.1 peptidyl-prolyl cis-trans isomerase NIMA-interacting 1
(SEQ ID NO: 78)
MADEEKLPPGWEKRMSRSSGRVYYFNHITNASQWERPSGNSSSGGKNGQGEPARVRCSHLL
VKHSQSRRPSSWRQEKITRTKEEALELINGYIQKIKSGEEDFESLASQFSDCSSAKARGDLGAF
SRGQMQKPFEDASFALRTGEMSGPVFTDSGIHIILRTE
NP_001108.2 pituitary adenylate cyclase-activating polypeptide precursor
(SEQ ID NO: 79)
MTMCSGARLALLVYGIIMHSSVYSSPAAAGLRFPGIRPEEEAYGEDGNPLPDFDGSEPPG
AGSPASAPRAAAAWYRPAGRRDVAHGILNEAYRKVLDQLSAGKHLQSLVARGVGGSLGGG
AGDDAEPLSKRHSDGIFTDSYSRYRKQMAVKKYLAAVLGKRYKQRVKNKGRRIAYL
NP_006226.2 POU domain class 2-associating factor 1
(SEQ ID NO: 80)
MLWQKPTAPEQAPAPARPYQGVRVKEPVKELLRRKRGHASSGAAPAPTAVVLPHQPLATYT
TVGPSCLDMEGSVSAVTEEAALCAGWLSQPTPATLQPLAPWTPYTEYVPHEAVSCPYSADM
YVQPVCPSYTVVGPSSVLTYASPPLITNVTTRSSATPAVGPPLEGPEHQAPLTYFPWPQPLSTL
PTSTLQYQPPAPALPGPQFVQLPISIPEPVLQDMEDPRRAASSLTIDKLLLEEE
DSDAYALNHTLSVEGF
NP_001185715.1 POU domain, class 2, transcription factor 1 isoform 3
(SEQ ID NO: 81)
MADGGAASQDESSAAAAAAADSRMNNPSETSKPSMESGDGNTGTQTNGLDFQKQPVPVGG
AISTAQAQAFLGHLHQVQLAGTSLQAAAQSLNVQSKSNEESGDSQQPSQPSQQPSVQAAIPQ
TQLMLAGGQITGDLQQLQQLQQQNLNLQQFVLVHPTTNLQPAQFIISQTPQGQQGLLQAQNL
LTQLPQQSQANLLQSQPSITLTSQPATPTRTIAATPIQTLPQSQSTPKRIDTPSLEEPSDLEELEQ
FAKTFKQRRIKLGFTQGDVGLAMGKLYGNDFSQTTISRFEALNLSFKNMCKLKPLLEKWLN
DAENLSSDSSLSSPSALNSPGIEGLSRRRKKRTSIETNIRVALEKSFLE
NQKPTSEEITMIADQLNMEKEVIRVWFCNRRQKEKRINPPSSGGTSSSPIKAIFPSPTSL
VATTPSLVTSSAATTLTVSPVLPLTSAAVTNLSVTGTSDTTSNNTATVISTAPPASSAVT
SPSLSPSPSASASTSEASSASETSTTQTTSTPLSSPLGTSQVMVTASGLQTAAAAALQGA
AQLPANASLAAMAAAAGLNPSLMAPSQFAAGGALLSLNPGTLSGALSPALMSNSTLATIQAL
ASGGSLPITSLDATGNLVFANAGGAPNIVTAPLFLNPQNLSLLTSNPVSLVSAAAASA
GNSAPVASLHATSTSAESIQNSLFTVASASGAASTTTTASKAQ
NP_001191890.1 pre-B-cell leukemia transcription factor 1 isoform 2
(SEQ ID NO: 82)
MDEQPRLMHSHAGVGMAGHPGLSQHLQDGAGGTEGEGGRKQDIGDILQQIMTITDQSLDEA
QARKHALNCHRMKPALFNVLCEIKEKTVLSIRGAQEEEPTDPQLMRLDNMLLAEGVAGPEK
GGGSAAAAAAAAASGGAGSDNSVEHSDYRAKLSQIRQIYHTELEKYEQACNEFTTHVMNLL
REQSRTRPISPKEIERMVSIIHRKFSSIQMQLKQSTCEAVMILRSRFLDARRKRRNFNKQATEIL
NEYFYSHLSNPYPSEEAKEELAKKCGITVSQVSNWFGNKRIRYKKNIGKFQEEANIYAAKTA
VTATNVSAHGSQANSPSTPNSAGGYPSPCYQPDRRIQ
NP_002871.1 RAF proto-oncogene serine/threonine-protein kinase
(SEQ ID NO: 83)
MEHIQGAWKTISNGFGFKDAVFDGSSCISPTIVQQFGYQRRASDDGKLTDPSKTSNTIRVFLP
NKQRTVVNVRNGMSLHDCLMKALKVRGLQPECCAVFRLLHEHKGKKARLDWNTDAASLI
GEELQVDFLDHVPLTTHNFARKTFLKLAFCDICQKFLLNGFRCQTCGYKFHEHCSTKVPTMC
VDWSNIRQLLLFPNSTIGDSGVPALPSLTMRRMRESVSRMPVSSQHRYSTPHAFTFNTSSPSSE
GSLSQRQRSTSTPNVHMVSTTLPVDSRMIEDAIRSHSESASPSALSSSPNNLSPTGWSQPKTPV
PAQRERAPVSGTQEKNKIRPRGQRDSSYYWEIEASEVMLSTRIGSGSFGTVYKGKWHGDVA
VKILKVVDPTPEQFQAFRNEVAVLRKTRHVNILLFMGYMTKDNLAIVTQWCEGSSLYKHLH
VQETKFQMFQLIDIARQTAQGMDYLHAKNIIHRDMKSNNIFLHEGLTVKIGDFGLATVKSRW
SGSQQVEQPTGSVLWMAPEVIRMQDNNPFSFQSDVYSYGIVLYELMTGELPYSHINNRDQIIF
MVGRGYASPDLSKLYKNCPKAMKRLVADCVKKVKEERPLFPQILSSIELLQHSLPKINRSASE
PSLHRAAHTEDINACTLTTSPRLPVF
NP_001005862.1 receptor tyrosine-protein kinase erbB-2 isoform b
(SEQ ID NO: 84)
MKLRLPASPETHLDMLRHLYQGCQVVQGNLELTYLPTNASLSFLQDIQEVQGYVLIAHNQV
RQVPLQRLRIVRGTQLFEDNYALAVLDNGDPLNNTTPVTGASPGGLRELQLRSLTEILKGGV
LIQRNPQLCYQDTILWKDIFHKNNQLALTLIDTNRSRACHPCSPMCKGSRCWGESSEDCQSLT
RTVCAGGCARCKGPLPTDCCHEQCAAGCTGPKHSDCLACLHFNHSGICELHCPALVTYNTD
TFESMPNPEGRYTFGASCVTACPYNYLSTDVGSCTLVCPLHNQEVTAEDGTQRCEKCSKPCA
RVCYGLGMEHLREVRAVTSANIQEFAGCKKIFGSLAFLPESFDGDPASNTAPLQPEQLQVFET
LEEITGYLYISAWPDSLPDLSVFQNLQVIRGRILHNGAYSLTLQGLGISWLGLRSLRELGSGLA
LIHHNTHLCFVHTVPWDQLFRNPHQALLHTANRPEDECVGEGLACHQLCARGHCWGPGPT
QCVNCSQFLRGQECVEECRVLQGLPREYVNARHCLPCHPECQPQNGSVTCFGPEADQCVAC
AHYKDPPFCVARCPSGVKPDLSYMPIWKFPDEEGACQPCPINCTHSCVDLDDKGCPAEQRAS
PLTSIISAVVGILLVVVLGVVFGILIKRRQQKIRKYTMRRLLQETELVEPLTPSGAMPNQAQM
RILKETELRKVKVLGSGAFGTVYKGIWIPDGENVKIPVAIKVLRENTSPKANKEILDEAYVMA
GVGSPYVSRLLGICLTSTVQLVTQLMPYGCLLDHVRENRGRLGSQDLLNWCMQIAKGMSYL
EDVRLVHRDLAARNVLVKSPNHVKITDFGLARLLDIDETEYHADGGKVPIKWMALESILRRR
FTHQSDVWSYGVTVWELMTFGAKPYDGIPAREIPDLLEKGERLPQPPICTIDVYMIMVKCW
MIDSECRPRFRELVSEFSRMARDPQRFVVIQNEDLGPASPLDSTFYRSLLEDDDMGDLVDAEE
YLVPQQGFFCPDPAPGAGGMVHHRHRSSSTRSGGGDLTLGLEPSEEEAPRSPLAPSEGAGSD
VFDGDLGMGAAKGLQSLPTHDPSPLQRYSEDPTVPLPSETDGYVAPLTCSPQPEYVNQPDVR
PQPPSPREGPLPAARPAGATLERPKTLSPGKNGVVKDVFAFGGAVENPEYLTPQGGAAPQPH
PPPAFSPAFDNLYYWDQDPPERGAPPSTFKGTPTAENPEYLGLDVPV
NP_001036064.1 receptor tyrosine-protein kinase erbB-4 isoform JM-a/
CVT-2 precursor
(SEQ ID NO: 85)
MKPATGLWVWVSLLVAAGTVQPSDSQSVCAGTENKLSSLSDLEQQYRALRKYYENCEVVM
GNLEITSIEHNRDLSFLRSVREVTGYVLVALNQFRYLPLENLRIIRGTKLYEDRYALAIFLNYR
KDGNFGLQELGLKNLTEILNGGVYVDQNKFLCYADTIHWQDIVRNPWPSNLTLVSTNGSSG
CGRCHKSCTGRCWGPTENHCQTLTRTVCAEQCDGRCYGPYVSDCCHRECAGGCSGPKDTD
CFACMNFNDSGACVTQCPQTFVYNPTTFQLEHNFNAKYTYGAFCVKKCPHNFVVDSSSCVR
ACPSSKMEVEENGIKMCKPCTDICPKACDGIGTGSLMSAQTVDSSNIDKFINCTKINGNLIFLV
TGIHGDPYNAIEAIDPEKLNVFRTVREITGFLNIQSWPPNMTDFSVFSNLVTIGGRVLYSGLSL
LILKQQGITSLQFQSLKEISAGNIYITDNSNLCYYHTINWTTLFSTINQRIVIRDNRKAENCTAE
GMVCNHLCSSDGCWGPGPDQCLSCRRFSRGRICIESCNLYDGEFREFENGSICVECDPQCEK
MEDGLLTCHGPGPDNCTKCSHFKDGPNCVEKCPDGLQGANSFIFKYADPDRECHPCHPNCT
QGCNGPTSHDCIYYPWTGHSTLPQHARTPLIAAGVIGGLFILVIVGLTFAVYVRRKSIKKKRA
LRRFLETELVEPLTPSGTAPNQAQLRILKETELKRVKVLGSGAFGTVYKGIWVPEGETVKIPV
AIKILNETTGPKANVEFMDEALIMASMDHPHLVRLLGVCLSPTIQLVTQLMPHGCLLEYVHE
HKDNIGSQLLLNWCVQIAKGMMYLEERRLVHRDLAARNVLVKSPNHVKITDFGLARLLEGD
EKEYNADGGKMPIKWMALECIHYRKFTHQSDVWSYGVTIWELMTFGGKPYDGIPTREIPDL
LEKGERLPQPPICTIDVYMVMVKCWMIDADSRPKFKELAAEFSRMARDPQRYLVIQGDDRM
KLPSPNDSKFFQNLLDEEDLEDMMDAEEYLVPQAFNIPPPIYTSRARIDSNRNQFVYRDGGFA
AEQGVSVPYRAPTSTIPEAPVAQGATAEIFDDSCCNGTLRKPVAPHVQEDSSTQRYSADPTVF
APERSPRGELDEEGYMTPMRDKPKQEYLNPVEENPFVSRRKNGDLQALDNPEYHNASNGPP
KAEDEYVNEPLYLNTFANTLGKAEYLKNNILSMPEKAKKAFDNPDYWNHSLPPRSTLQHPD
YLQEYSTKYFYKQNGRIRPIVAENPEYLSEFSLKPGTVLPPPPYRHRNTVV
NP_000312.2 retinoblastoma-associated protein
(SEQ ID NO: 86)
MPPKTPRKTAATAAAAAAEPPAPPPPPPPEEDPEQDSGPEDLPLVRLEFEETEEPDFTALCQKL
KIPDHVRERAWLTWEKVSSVDGVLGGYIQKKKELWGICIFIAAVDLDEMSFTFTELQKNIEIS
VHKFFNLLKEIDTSTKVDNAMSRLLKKYDVLFALFSKLERTCELIYLTQPSSSISTEINSALVL
KVSWITFLLAKGEVLQMEDDLVISFQLMLCVLDYFIKLSPPMLLKEPYKTAVIPINGSPRTPRR
GQNRSARIAKQLENDTRIIEVLCKEHECNIDEVKNVYFKNFIPFMNSLGLVTSNGLPEVENLS
KRYEEIYLKNKDLDARLFLDHDKTLQTDSIDSFETQRTPRKSNLDEEVNVIPPHTPVRTVMNT
IQQLMMILNSASDQPSENLISYFNNCTVNPKESILKRVKDIGYIFKEKFAKAVGQGCVEIGSQR
YKLGVRLYYRVMESMLKSEEERLSIQNFSKLLNDNIFHMSLLACALEVVMATYSRSTSQNLD
SGTDLSFPWILNVLNLKAFDFYKVIESFIKAEGNLTREMIKHLERCEHRIMESLAWLSDSPLFD
LIKQSKDREGPTDHLESACPLNLPLQNNHTAADMYLSPVRSPKKKGSTTRVNSTANAETQAT
SAFQTQKPLKSTSLSLFYKKVYRLAYLRLNTLCERLLSEHPELEHIIWTLFQHTLQNEYELMR
DRHLDQIMMCSMYGICKVKNIDLKFKIIVTAYKDLPHAVQETFKRVLIKEEEYDSIIVFYNSV
FMQRLKTNILQYASTRPPTLSPIPHIPRSPYKFPSSPLRIPGGNIYISPLKSPYKISEGLPTPTKMT
PRSRILVSIGESFGTSEKFQKINQMVCNSDRVLKRSAEGSNPPKPLKKLRFDIEGSDEADGSKH
LPGESKFQQK
LAEMTSTRTRMQKQKMNDSMDTSNKEEK
NP_002927.2 ribonuclease H1
(SEQ ID NO: 87)
MSWLLFLAHRVALAALPCRRGSRGFGMFYAVRRGRKTGVFLTWNECRAQVDRFPAARFKK
FATEDEAWAFVRKSASPEVSEGHENQHGQESEAKASKRLREPLDGDGHESAEPYAKHMKPS
VEPAPPVSRDTFSYMGDFVVVYTDGCCSSNGRRRPRAGIGVYWGPGHPLNVGIRLPGRQTN
QRAEIHAACKAIEQAKTQNINKLVLYTDSMFTINGITNWVQGWKKNGWKTSAGKEVINKED
FVALERLTQGMDIQWMHVPGHSGFIGNEEADRLAREGAKQSED
NP_036366.3 RING1 and YY1-binding protein
(SEQ ID NO: 88)
MTMGDKKSPTRPKRQAKPAADEGFWDCSVCTFRNSAEAFKCSICDVRKGTSTRKPRINSQL
VAQQVAQQYATPPPPKKEKKEKVEKQDKEKPEKDKEISPSVTKKNTNKKTKPKSDILKDPPS
EANSIQSANATTKTSETNHTSRPRLKNVDRSTAQQLAVTVGNVTVIITDFKEKTRSSSTSSSTV
TSSAGSEQQNQSSSGSESTDKGSSRSSTPKGDMSAVNDESF
NP_001006121.1 RNA-binding motif protein, Y chromosome, family 1 member B
(SEQ ID NO: 89)
MVEADHPGKLFIGGLNRETNEKMLKAVFGKHGPISEVLLIKDRTSKSRGFAFITFENPAD
AKNAAKDMNGKSLHGKAIKVEQAKKPSFQSGGRRRPPASSRNRSPSGSLRSARGSRGGTRG
WLPSQEGHLDDGGYTPDLKMSYSRGLIPVKRGPSSRSGGPPPKKSAPSAVARSNSWMGSQG
PMSQRRENYGVPPRRATISSWRNDRMSTRHDGYATNDGNHPSCQETRDYAPPSRGYAYRD
NGHSNRDEHSSRGYRNHRSSRETRDYAPPSRGHAYRDYGHSRRDESYSRGYRNRRSSRETR
EYAPPSRGHGYRDYGHSRRHESYSRGYRNHPSSRETRDYAPPHRDYAYRDYGHSSWDEHSS
RGYSYHDGYGEALGRDHSEHLSGSSYRDALQRYGTSHGAPPARGPRMSYGGSTCHAYSNTR
DRYGRSWESYSSCGDFHYCDREHVCRKDQRNPPSLGRVLPDPREAYGSSSYVASIVDGGESR
SEKGDSSRY
NP_036523.1 SAM pointed domain-containing Ets transcription factor
(SEQ ID NO: 90)
MGSASPGLSSVSPSHLLLPPDTVSRTGLEKAAAGAVGLERRDWSPSPPATPEQGLSAFYL
SYFDMLYPEDSSWAAKAPGASSREEPPEEPEQCPVIDSQAPAGSLDLVPGGLTLEEHSLE
QVQSMVVGEVLKDIETACKLLNITADPMDWSPSNVQKWLLWTEHQYRLPPMGKAFQELAG
KELCAMSEEQFRQRSPLGGDVLHAHLDIWKSAAWMKERTSPGAIHYCASTSEESWTDSEVD
SSCSGQPIHLWQFLKELLLKPHSYGRFIRWLNKEKGIFKIEDSAQVARLWGIRKNRPAMNYD
KLSRSIRQYYKKGIIRKPDISQRLVYQFVHPI
NP_003122.1 serum response factor
(SEQ ID NO: 91)
MLPTQAGAAAALGRGSALGGSLNRTPTGRPGGGGGTRGANGGRVPGNGAGLGPGRLEREA
AAAAATTPAPTAGALYSGSEGDSESGEEEELGAERRGLKRSLSEMEIGMVVGGPEASAAATG
GYGPVSGAVSGAKPGKKTRGRVKIKMEFIDNKLRRYTTFSKRKTGIMKKAYELSTLTGTQVL
LLVASETGHVYTFATRKLQPMITSETGKALIQTCLNSPDSPPRSDPTTDQRMSATGFEETDLT
YQVSESDSSGETKDTLKPAFTVTNLPGTTSTIQTAPSTSTTMQVSSGPSFPITNYLAPVSASVSP
SAVSSANGTVLKSTGSGPVSSGGLMQLPTSFTLMPGGAVAQQVPVQAIQVHQAPQQASPSR
DSSTDLTQTSSSGTVTLPATIMTSSVPTTVGGHMMYPSPHAVMYAPTSGLGDGSLTVLNAFS
QAPSTMQVSHSQVQEPGGVPQVFLTASSGTVQIPVSAVQLHQMAVIGQQAGSSSNLTELQVV
NLDTAHSTKSE
NP_003131.1 sex-determining region Y protein
(SEQ ID NO: 92)
MQSYASAMLSVFNSDDYSPAVQENIPALRRSSSFLCTESCNSKYQCETGENSKGNVQDRVKR
PMNAFIVWSRDQRRKMALENPRMRNSEISKQLGYQWKMLTEAEKWPFFQEAQKLQAMHR
EKYPNYKYRPRRKAKMLPKNCSLLPADPASVLCSEVQLDNRLYRDDCTKATHSRMEHQLG
HLPPINAASSPQQRDRYSHWTKL
NP_004588.1 small nuclear ribonucleoprotein Sm D2 isoform 1
(SEQ ID NO: 93)
MSLLNKPKSEMTPEELQKREEEEFNTGPLSVLTQSVKNNTQVLINCRNNKKLLGRVKAFDRH
CNMVLENVKEMWTEVPKSGKGKKKSKPVNKDRYISKMFLRGDSVIVVLRNPLIAGK
NP001005291.1 sterol regulatory element-binding protein 1 isoform a
(SEQ ID NO: 94)
MDEPPFSEAALEQALGEPCDLDAALLTDIEGEVGAGRGRANGLDAPRAGADRGAMDCTFED
MLQLINNQDSDFPGLFDPPYAGSGAGGTDPASPDTSSPGSLSPPPATLSSSLEAFLSGPQAAPS
PLSPPQPAPTPLKMYPSMPAFSPGPGIKEESVPLSILQTPTPQPLPGALLPQSFPAPAPPQFSSTP
VLGYPSPPGGFSTGSPPGNTQQPLPGLPLASPPGVPPVSLHTQVQSVVPQQLLTVTAAPTAAP
VTTTVTSQIQQVPVLLQPHFIKADSLLLTAMKTDGATVKAAGLSPLVSGTTVQTGPLPTLVSG
GTILATVPLVVDAEKLPINRLAAGSKAPASAQSRGEKRTAHNAIEKRYRSSINDKIIELKDLVV
GTEAKLNKSAVLRKAIDYIRFLQHSNQKLKQENLSLRTAVHKSKSLKDLVSACGSGGNTDVL
MEGVKTEVEDTLTPPPSDAGSPFQSSPLSLGSRGSGSGGSGSDSEPDSPVFEDSKAKPEQRPSL
HSRGMLDRSRLALCTLVFLCLSCNPLASLLGARGLPSPSDTTSVYHSPGRNVLGTESRDGPG
WAQWLLPPVVWLLNGLLVLVSLVLLFVYGEPVTRPHSGPAVYFWRHRKQADLDLARGDFA
QAAQQLWLALRALGRPLPTSHLDLACSLLWNLIRHLLQRLWVGRWLAGRAGGLQQDCALR
VDASASARDAALVYHKLHQLHTMGKHTGGHLTATNLALSALNLAECAGDAVSVATLAEIY
VAAALRVKTSLPRALHFLTRFFLSSARQACLAQSGSVPPAMQWLCHPVGHRFFVDGDWSVL
STPWESLYSLAGNPVDPLAQVTQLFREHLLERALNCVTQPNPSPGSADGDKEFSDALGYLQL
LNSCSDAAGAPAYSFSISSSMATTTGVDPVAKWWASLTAVVIHWLRRDEEAAERLCPLVEH
LPRVLQESERPLPRAALHSFKAARALLGCAKAESGPASLTICEKASGYLQDSLATTPASSSIDK
AVQLFLCDLLLVVRTSLWRQQQPPAPAPAAQGTSSRPQASALELRGFQRDLSSLRRLAQSFR
PAMRRVFLHEATARLMAGASPTRTHQLLDRSLRRRAGPGGKGGAVAELEPRPTRREHAEAL
LLASCYLPPGFLSAPGQRVGMLAEAARTLEKLGDRRLLHDCQQMLMRLGGGTTVTSS
NP006280.3 talin-1
(SEQ ID NO: 95)
MVALSLKISIGNVVKTMQFEPSTMVYDACRIIRERIPEAPAGPPSDFGLFLSDDDPKKGI
WLEAGKALDYYMLRNGDTMEYRKKQRPLKIRMLDGTVKTIMVDDSKTVTDMLMTICARIG
ITNHDEYSLVRELMEEKKEEITGTLRKDKTLLRDEKKMEKLKQKLHTDDELNWLDHGRTLR
EQGVEEHETLLLRRKFFYSDQNVDSRDPVQLNLLYVQARDDILNGSHPVSFDKACEFAGFQC
QIQFGPHNEQKHKAGFLDLKDFLPKEYVKQKGERKIFQAHKNCGQMSEIEAKVRYVKLARS
LKTYGVSFFLVKEKMKGKNKLVPRLLGITKECVMRVDEKTKEVIQEWNLTNIKRWAASPKS
FTLDFGDYQDGYYSVQTTEGEQIAQLIAGYIDIILKKKKSKDHFGLEGDEESTMLEDSVSPKK
STVLQQQYNRVGKVEHGSVALPAIMRSGASGPENFQVGSMPPAQQQITSGQMHRGHMPPLT
SAQQALTGTINSSMQAVQAAQATLDDFDTLPPLGQDAASKAWRKNKMDESKHEIHSQVDAI
TAGTASVVNLTAGDPAETDYTAVGCAVTTISSNLTEMSRGVKLLAALLEDEGGSGRPLLQA
AKGLAGAVSELLRSAQPASAEPRQNLLQAAGNVGQASGELLQQIGESDTDPHFQDALMQLA
KAVASAAAALVLKAKSVAQRTEDSGLQTQVIAAATQCALSTSQLVACTKVVAPTISSPVCQE
QLVEAGRLVAKAVEGCVSASQAATEDGQLLRGVGAAATAVTQALNELLQHVKAHATGAG
PAGRYDQATDTILTVTENIFSSMGDAGEMVRQARILAQATSDLVNAIKADAEGESDLENSRK
LLSAAKILADATAKMVEAAKGAAAHPDSEEQQQRLREAAEGLRMATNAAAQNAIKKKLVQ
RLEHAAKQAAASATQTIAAAQHAASTPKASAGPQPLLVQSCKAVAEQIPLLVQGVRGSQAQ
PDSPSAQLALIAASQSFLQPGGKMVAAAKASVPTIQDQASAMQLSQCAKNLGTALAELRTA
AQKAQEACGPLEMDSALSVVQNLEKDLQEVKAAARDGKLKPLPGETMEKCTQDLGNSTKA
VSSAIAQLLGEVAQGNENYAGIAARDVAGGLRSLAQAARGVAALTSDPAVQAIVLDTASDV
LDKASSLIEEAKKAAGHPGDPESQQRLAQVAKAVTQALNRCVSCLPGQRDVDNALRAVGD
ASKRLLSDSLPPSTGTFQEAQSRLNEAAAGLNQAATELVQASRGTPQDLARASGRFGQDFST
FLEAGVEMAGQAPSQEDRAQVVSNLKGISMSSSKLLLAAKALSTDPAAPNLKSQLAAAARA
VTDSINQLITMCTQQAPGQKECDNALRELETVRELLENPVQPINDMSYFGCLDSVMENSKVL
GEAMTGISQNAKNGNLPEFGDAISTASKALCGFTEAAAQAAYLVGVSDPNSQAGQQGLVEP
TQFARANQAIQMACQSLGEPGCTQAQVLSAATIVAKHTSALCNSCRLASARTTNPTAKRQFV
QSAKEVANSTANLVKTIKALDGAFTEENRAQCRAATAPLLEAVDNLSAFASNPEFSSIPAQIS
PEGRAAMEPIVISAKTMLESAGGLIQTARALAVNPRDPPSWSVLAGHSRTVSDSIKKLITSMR
DKAPGQLECETAIAALNSCLRDLDQASLAAVSQQLAPREGISQEALHTQMLTAVQEISHLIEP
LANAARAEASQLGHKVSQMAQYFEPLTLAAVGAASKTLSHPQQMALLDQTKTLAESALQL
LYTAKEAGGNPKQAAHTQEALEEAVQMMTEAVEDLTTTLNEAASAAGVVGGMVDSITQAI
NQLDEGPMGEPEGSFVDYQTTMVRTAKAIAVTVQEMVTKSNTSPEELGPLANQLTSDYGRL
ASEAKPAAVAAENEEIGSHIKHRVQELGHGCAALVTKAGALQCSPSDAYTKKELIECARRVS
EKVSHVLAALQAGNRGTQACITAASAVSGIIADLDTTIMFATAGTLNREGTETFADHREGILK
TAKVLVEDTKVLVQNAAGSQEKLAQAAQSSVATITRLADVVKLGAASLGAEDPETQVVLIN
AVKDVAKALGDLISATKAAAGKVGDDPAVWQLKNSAKVMVTNVTSLLKTVKAVEDEATK
GTRALEATTEHIRQELAVFCSPEPPAKTSTPEDFIRMTKGITMATAKAVAAGNSCRQEDVIAT
ANLSRRAIADMLRACKEAAYHPEVAPDVRLRALHYGRECANGYLELLDHVLLTLQKPSPEL
KQQLTGHSKRVAGSVTELIQAAEAMKGTEWVDPEDPTVIAENELLGAAAAIEAAAKKLEQL
KPRAKPKEADESLNFEEQILEAAKSIAAATSALVKAASAAQRELVAQGKVGAIPANALDDGQ
WSQGLISAARMVAAATNNLCEAANAAVQGHASQEKLISSAKQVAASTAQLLVACKVKADQ
DSEAMKRLQAAGNAVKRASDNLVKAAQKAAAFEEQENETVVVKEKMVGGIAQIIAAQEEM
LRKERELEEARKKLAQIRQQQYKFLPSELRDEH
NP_003185.1 TATA-box-binding protein isoform 1
(SEQ ID NO: 96)
MDQNNSLPPYAQGLASPQGAMTPGIPIFSPMMPYGTGLTPQPIQNTNSLSILEEQQRQQQ
QQQQQQQQQQQQQQQQQQQQQQQQQQQQQQQQQQQAVAAAAVQQSTSQQATQGTSGQ
APQLFHSQTLTTAPLPGTTPLYPSPMTPMTPITPATPASESSGIVPQLQNIVSTVNLGCKLDLKT
IALRARNAEYNPKRFAAVIMRIREPRTTALIFSSGKMVCTGAKSEEQSRLAARKYARVVQKL
GFPAKFLDFKIQNMVGSCDVKFPIRLEGLVLTHQQFSSYEPELFPGLIYRMIKPRIVLLIFVSGK
VVLTGAKVRAEIYEAFENIYPILKGFRKTT
NP_001165556.1 TATA-box-binding protein isoform 2
(SEQ ID NO: 97)
MTPGIPIFSPMMPYGTGLTPQPIQNTNSLSILEEQQRQQQQQQQQQQQQQQQQQQQQQQQQ
QQQQQQQQQQQQQQAVAAAAVQQSTSQQATQGTSGQAPQLFHSQTLTTAPLPGTTPLYPSP
MTPMTPITPATPASESSGIVPQLQNIVSTVNLGCKLDLKTIALRARNAEYNPKRFAAVIMRIRE
PRTTALIFSSGKMVCTGAKSEEQSRLAARKYARVVQKLGFPAKFLDFKIQNMVGSCDVKFPI
RLEGLVLTHQQFSSYEPELFPGLIYRMIKPRIVLLIFVSGKVVLTGAKVRAE
IYEAFENIYPILKGFRKTT
NP_057254.1 T-cell leukemia homeobox protein 2
(SEQ ID NO: 98)
MEPGMLGPHNLPHHEPISFGIDQILSGPETPGGGLGLGRGGQGHGENGAFSGGYHGASGYGP
AGSLAPLPGSSGVGPGGVIRVPAHRPLPVPPPAGGAPAVPGPSGLGGAGGLAGLTFPWMDSG
RRFAKDRLTAALSPFSGTRRIGHPYQNRTPPKRKKPRTSFSRSQVLELERRFLRQKYLASAER
AALAKALRMTDAQVKTWFQNRRTKWRRQTAEEREAERHRAGRLLLHLQQDALPRPLRPPL
PPDPLCLHNSSLFALQNLQPWAEDNKVASVSGLASVV
NP_000607.1 T-cell surface glycoprotein CD4 isoform 1 precursor
(SEQ ID NO: 99)
MNRGVPFRHLLLVLQLALLPAATQGKKVVLGKKGDTVELTCTASQKKSIQFHWKNSNQIKI
LGNQGSFLTKGPSKLNDRADSRRSLWDQGNFPLIIKNLKIEDSDTYICEVEDQKEEVQLLVFG
LTANSDTHLLQGQSLTLTLESPPGSSPSVQCRSPRGKNIQGGKTLSVSQLELQDSG
TWTCTVLQNQKKVEFKIDIVVLAFQKASSIVYKKEGEQVEFSFPLAFTVEKLTGSGELWWQA
ERASSSKSWITFDLKNKEVSVKRVTQDPKLQMGKKLPLHLTLPQALPQYAGSGNLTLALEA
KTGKLHQEVNLVVMRATQLQKNLTCEVWGPTSPKLMLSLKLENKEAKVSKREKAVWVLN
PEAGMWQCLLSDSGQVLLESNIKVLPTWSTPVQPMALIVLGGVAGLLLFIGLGIFFCVRCRHR
RRQAERMSQIKRLLSEKKTCQCPHRFQKTCSPI
NP_059523.2 telomeric repeat-binding factor 1 isoform 1
(SEQ ID NO: 100)
MAEDVSSAAPSPRGCADGRDADPTEEQMAETERNDEEQFECQELLECQVQVGAPEEEEEEE
EDAGLVAEAEAVAAGWMLDFLCLSLCRAFRDGRSEDFRRTRNSAEAIIHGLSSLTACQLRTI
YICQFLTRIAAGKTLDAQFENDERITPLESALMIWGSIEKEHDKLHEEIQNLIKIQA
IAVCMENGNFKEAEEVFERIFGDPNSHMPFKSKLLMIISQKDTFHSFFQHFSYNHMMEKI
KSYVNYVLSEKSSTFLMKAAAKVVESKRTRTITSQDKPSGNDVEMETEANLDTRKSVSDKQ
SAVTESSEGTVSLLRSHKNLFLSKLQHGTQQQDLNKKERRVGTPQSTKKKKESRRATESRIPV
SKSQPVTPEKHRARKRQAWLWEEDKNLRSGVRKYGEGNWSKILLHYKFNNRTSVMLKDR
WRTMKKLKLISSDSED
NP_005643.1 telomeric repeat-binding factor 2
(SEQ ID NO: 101)
MAGGGGSSDGSGRAAGRRASRSSGRARRGRHEPGLGGPAERGAGEARLEEAVNRWVLKFY
FHEALRAFRGSRYGDFRQIRDIMQALLVRPLGKEHTVSRLLRVMQCLSRIEEGENLDCSFDM
EAELTPLESAINVLEMIKTEFTLTEAVVESSRKLVKEAAVIICIKNKEFEKASKILKKHMSKDP
TTQKLRNDLLNIIREKNLAHPVIQNFSYETFQQKMLRFLESHLDDAEPYLLTMAKKALKSESA
ASSTGKEDKQPAPGPVEKPPREPARQLRNPPTTIGMMTLKAAFKTLSGAQDSEAAFAKLDQK
DLVLPTQALPASPALKNKRPRKDENESSAPADGEGGSELQPKNKRMTISRLVLEEDSQSTEPS
AGLNSSQEAASAPPSKPTVLNQPLPGEKNPKVPKGKWNSSNGVEEKETWVEEDELFQVQAA
PDEDSTTNITKKQKWTVEESEWVKAGVQKYGEGNWAAISKNYPFVNRTAVMIKDRWRTM
KRLGMN
NP_060575.1 THAP domain-containing protein 1 isoform 1
(SEQ ID NO: 102)
MVQSCSAYGCKNRYDKDKPVSFHKFPLTRPSLCKEWEAAVRRKNFKPTKYSSICSEHFTPDC
FKRECNNKLLKENAVPTIFLCTEPHDKKEDLLEPQEQLPPPPLPPPVSQVDAAIGLLM
PPLQTPVNLSVFCDHNYTVEDTMHQRKRIHQLEQQVEKLRKKLKTAQQRCRRQERQLEKLK
EVVHFQKEKDDVSERGYVILPNDYFEIVEVPA
NP_002219.1 transcription factor AP-1
(SEQ ID NO: 103)
MTAKMETTFYDDALNASFLPSESGPYGYSNPKILKQSMTLNLADPVGSLKPHLRAKNSDLLT
SPDVGLLKLASPELERLIIQSSNGHITTTPTPTQFLCPKNVTDEQEGFAEGFVRALAE
LHSQNTLPSVTSAAQPVNGAGMVAPAVASVAGGSGSGGFSASLHSEPPVYANLSNFNPGALS
SGGGAPSYGAAGLAFPAQPQQQQQPPHHLPQQMPVQHPRLQALKEEPQTVPEMPGETPPLSP
IDMESQERIKAERKRMRNRIAASKCRKRKLERIARLEEKVKTLKAQNSELASTANMLREQVA
QLKQKVMNHVNSGCQLMLTQQLQTF
NP_003097.1 transcription factor SOX-2
(SEQ ID NO: 104)
MYNMMETELKPPGPQQTSGGGGGNSTAAAAGGNQKNSPDRVKRPMNAFMVWSRGQRRK
MAQENPKMHNSEISKRLGAEWKLLSETEKRPFIDEAKRLRALHMKEHPDYKYRPRRKTKTL
MKKDKYTLPGGLLAPGGNSMASGVGVGAGLGAGVNQRMDSYAHMNGWSNGSYSMMQD
QLGYPQHPGLNAHGAAQMQPMHRYDVSALQYNSMTSSQTYMNGSPTYSMSYSQQGTPGM
ALGSMGSVVKSEASSSPPVVTSSSHSRAPCQAGDLRDMISMYLPGAEVPEPAAPSRLHMSQH
YQSGPVPGTAINGTLPLSHM
NP_003100.1 transcription factor Sp1 isoform b
(SEQ ID NO: 105)
MDEMTAVVKIEKGVGGNNGGNGNGGGAFSQARSSSTGSSSSTGGGGQESQPSPLALLAATC
SRIESPNENSNNSQGPSQSGGTGELDLTATQLSQGANGWQIISSSSGATPTSKEQSGSSTNGSN
GSESSKNRTVSGGQYVVAAAPNLQNQQVLTGLPGVMPNIQYQVIPQFQTVDGQQLQFAATG
AQVQQDGSGQIQIIPGANQQIITNRGSGGNIIAAMPNLLQQAVPLQGLANNVLSGQTQYVTN
VPVALNGNITLLPVNSVSAATLTPSSQAVTISSSGSQESGSQPVTSGTTISSASLVSSQASSSSFF
TNANSYSTTTTTSNMGIMNFTTSGSSGTNSQGQTPQRVSGLQGSDALNIQQNQTSGGSLQAG
QQKEGEQNQQTQQQQILIQPQLVQGGQALQALQAAPLSGQTFTTQAISQETLQNLQLQAVPN
SGPIIIRTPTVGPNGQVSWQTLQLQNLQVQNPQAQTITLAPMQGVSLGQTSSSNTTLTPIASAA
SIPAGTVTVNAAQLSSMPGLQTINLSALGTSGIQVHPIQGLPLAIANAPGDHGAQLGLHGAGG
DGIHDDTAGGEEGENSPDAQPQAGRRTRREACTCPYCKDSEGRGSGDPGKKKQHICHIQGC
GKVYGKTSHLRAHLRWHTGERPFMCTWSYCGKRFTRSDELQRHKRTHTGEKKFACPECPK
RFMRSDHLSKHIKTHQNKKGGPGVALSVGTLPLDSGAGSEGSGTATPSALITTNMVAMEAIC
PEGIARLANSGINVMQVADLQSINISGNGF
NP_001123645.1 transcriptional activator Myb isoform 1
(SEQ ID NO: 106)
MARRPRHSIYSSDEDDEDFEMCDHDYDGLLPKSGKRHLGKTRWTREEDEKLKKLVEQNGT
DDWKVIANYLPNRTDVQCQHRWQKVLNPELIKGPWTKEEDQRVIELVQKYGPKRWSVIAK
HLKGRIGKQCRERWHNHLNPEVKKTSWTEEEDRIIYQAHKRLGNRWAEIAKLLPGRTDNAI
KNHWNSTMRRKVEQEGYLQESSKASQPAVATSFQKNSHLMGFAQAPPTAQLPATGQPTVN
NDYSYYHISEAQNVSSHVPYPVALHVNIVNVPQPAAAAIQRHYNDEDPEKEKRIKELELLLM
STENELKGQQVLPTQNHTCSYPGWHSTTIADHTRPHGDSAPVSCLGEHHSTPSLPADPGSLPE
ESASPARCMIVHQGTILDNVKNLLEFAETLQFIDSDSSSWCDLSSFEFFEEADFSPSQHHTGKA
LQLQQREGNGTKPAGEPSPRVNKRMLSESSLDPPKVLPPARHSTIPLVILRKKRGQASPLATG
DCSSFIFADVSSSTPKRSPVKSLPFSPSQFLNTSSNHENSDLEMPSLTSTPLIGHKLTVTTPFHRD
QTVKTQKENTVFRTPAIKRSILESSPRTPTPFKHALAAQEIKYGPLKMLPQTPSHLVEDLQDVI
KQESDESGIVAEFQENGPPLLKKIKQEVESPTDKSGNFFCSHHWEGDSLNTQLFTQTSPVADA
PNILTSSVLMAPASEDEDNVLKAFTVPKNRSLASPLQPCSSTWEPASCGKMEEQMTSSSQAR
KYVNAFSARTLVM
NP_001155128.1 transcriptional activator Myb isoform 4
(SEQ ID NO: 107)
MARRPRHSIYSSDEDDEDFEMCDHDYDGLLPKSGKRHLGKTRWTREEDEKLKKLVEQNGT
DDWKVIANYLPNRTDVQCQHRWQKVLNPELIKGPWTKEEDQRVIELVQKYGPKRWSVIAK
HLKGRIGKQCRERWHNHLNPEVKKTSWTEEEDRIIYQAHKRLGNRWAEIAKLLPGRTDNAI
KNHWNSTMRRKVEQEGYLQESSKASQPAVATSFQKNSHLMGFAQAPPTAQLPATGQPTVN
NDYSYYHISEAQNVSSHVPYPVALHVNIVNVPQPAAAAIQRHYNDEDPEKEKRIKELELLLM
STENELKGQQTQNHTCSYPGWHSTTIADHTRPHGDSAPVSCLGEHHSTPSLPADPGSLPEESA
SPARCMIVHQGTILDNVKNLLEFAETLQFIDSDSSSWCDLSSFEFFEEADFSPSQHHTGKALQL
QQREGNGTKPAGEPSPRVNKRMLSESSLDPPKVLPPARHSTIPLVILRKKRGQASPLATGDCS
SFIFADVSSSTPKRSPVKSLPFSPSQFLNTSSNHENSDLEMPSLTSTPLIGHKLTVTTPFHRDQT
VKTQKENTVFRTPAIKRSILESSPRTPTPFKHALAAQEIKYGPLKMLPQTPSHLVEDLQDVIKQ
ESDESGIVAEFQENGPPLLKKIKQEVESPTDKSGNFFCSHHWEGDSLNTQLFTQTSPVADAPNI
LTSSVLMAPASEDEDNVLKAFTVPKNRSLASPLQPCSSTWEPASCGKMEEQMTSSSQARKYV
NAFSARTLVM
NP_443177.1 tumor necrosis factor receptor superfamily member 13C
(SEQ ID NO: 108)
MRRGPRSLRGRDAPAPTPCVPAECFDLLVRHCVACGLLRTPRPKPAGASSPAPRTALQPQ
ESVGAGAGEAALPLPGLLFGAPALLGLALVLALVLVGLVSWRRRQRRLRGASSAEAPDGDK
DAPEPLDKVIILSPGISDATAPAWPPPGEDPGTTPPGHSVPVPATELGSTELVTTKTAG
PEQQ
NP_004587.1 U1 small nuclear ribonucleoprotein A
(SEQ ID NO: 109)
MAVPETRPNHTIYINNLNEKIKKDELKKSLYAIFSQFGQILDILVSRSLKMRGQAFVIFK
EVSSATNALRSMQGFPFYDKPMRIQYAKTDSDIIAKMKGTFVERDRKREKRKPKSQETPATK
KAVQGGGATPVVGAVQGPVPGMPPMTQAPRIMHHMPGQPPYMPPPGMIPPPGLAPGQIPPG
AMPPQQLMPGQMPPAQPLSENPPNHILFLTNLPEETNELMLSMLFNQFPGFKEVRLVPGRHDI
AFVEFDNEVQAGAARDALQGFKITQNNAMKISFAKK
NP_001161097.1 voltage-dependent L-type calcium channel subunit alpha-1C
isoform 23
(SEQ ID NO: 110)
MVNENTRMYIPEENHQGSNYGSPRPAHANMNANAAAGLAPEHIPTPGAALSWQAAIDAAR
QAKLMGSAGNATISTVSSTQRKRQQYGKPKKQGSTTATRPPRALLCLTLKNPIRRACISIVEW
KPFEIIILLTIFANCVALAIYIPFPEDDSNATNSNLERVEYLFLIIFTVEAFLKVIAYGLLFHPNAY
LRNGWNLLDFIIVVVGLFSAILEQATKADGANALGGKGAGFDVKALRAFRVLRPLRLVSGVP
SLQVVLNSIIKAMVPLLHIALLVLFVIIIYAIIGLELFMGKMHKTCYNQEGIADVPAEDDPSPCA
LETGHGRQCQNGTVCKPGWDGPKHGITNFDNFAFAMLTVFQCITMEGWTDVLYWMQDAM
GYELPWVYFVSLVIFGSFFVLNLVLGVLSGEFSKEREKAKARGDFQKLREKQQLEEDLKGYL
DWITQAEDIDPENEDEGMDEEKPRNMSMPTSETESVNTENVAGGDIEGENCGARLAHRISKS
KFSRYWRRWNRFCRRKCRAAVKSNVFYWLVIFLVFLNTLTIASEHYNQPNWLTEVQDTAN
KALLALFTAEMLLKMYSLGLQAYFVSLFNRFDCFVVCGGILETILVETKIMSPLGISVLRCVR
LLRIFKITRYWNSLSNLVASLLNSVRSIASLLLLLFLFIIIFSLLGMQLFGGKFNFDEMQTRRSTF
DNFPQSLLTVFQILTGEDWNSVMYDGIMAYGGPSFPGMLVCIYFIILFICGNYILLNVFLAIAV
DNLADAESLTSAQKEEEEEKERKKLARTASPEKKQELVEKPAVGESKEEKIELKSITADGESP
PATKINMDDLQPNENEDKSPYPNPETTGEEDEEEPEMPVGPRPRPLSELHLKEKAVPMPEASA
FFIFSSNNRFRLQCHRIVNDTIFTNLILFFILLSSISLAAEDPVQHTSFRNHILFYFDIVFTTIFTIEI
ALKMTAYGAFLHKGSFCRNYFNILDLLVVSVSLISFGIQSSAINVVKILRVLRVLRPLRAINRA
KGLKHVVQCVFVAIRTIGNIVIVTTLLQFMFACIGVQLFKGKLYTCSDSSKQTEAECKGNYIT
YKDGEVDHPIIQPRSWENSKFDFDNVLAAMMALFTVSTFEGWPELLYRSIDSHTEDKGPIYN
YRVEISIFFIIYIIIIAFFMMNIFVGFVIVTFQEQGEQEYKNCELDKNQRQCVEYALKARPLRRYI
PKNQHQYKVWYVVNSTYFEYLMFVLILLNTICLAMQHYGQSCLFKIAMNILNMLFTGLFTV
EMILKLIAFKPKHYFCDAWNTFDALIVVGSIVDIAITEVNNAEENSRISITFFRLFRVMRLVKLL
SRGEGIRTLLWTFIKSFQALPYVALLIVMLFFIYAVIGMQVFGKIALNDTTEINRNNNFQTFPQ
AVLLLFRCATGEAWQDIMLACMPGKKCAPESEPSNSTEGETPCGSSFAVFYFISFYMLCAFLII
NLFVAVIMDNFDYLTRDWSILGPHHLDEFKRIWAEYDPEAKGRIKHLDVVTLLRRIQPPLGF
GKLCPHRVACKRLVSMNMPLNSDGTVMFNATLFALVRTALRIKTEGNLEQANEELRAIIKKI
WKRTSMKLLDQVVPPAGDDEVTVGKFYATFLIQEYFRKFKKRKEQGLVGKPSQRNALSLQA
GLRTLHDIGPEIRRAISGDLTAEEELDKAMKEAVSAASEDDIFRRAGGLFGNHVSYYQSDGRS
AFPQTFTTQRPLHINKAGSSQGDTESPSHEKLVDSTFTPSSYSSTGSNANINNANNTALGRLPR
PAGYPSTVSTVEGHGPPLSPAIRVQEVAWKLSSNRMHCCDMLDGGTFPPALGPRRAPPCLHQ
QLQGSLAGLREDTPCIVPGHASLCCSSRVGEWLPAGCTAPQHARCHSRESQAAMAGQEETS
QDETYEVKMNHDTEACSEPSLLSTEMLSYQDDENRQLTLPEEDKRDIRQSPKRGFLRSASLG
RRASFHLECLKRQKDRGGDISQKTVLPLHLVHHQALAVAGLSPLLQRSHSPASFPRPFATPPA
TPGSRGWPPQPVPTLRLEGVESSEKLNSSFPSIHCGSWAETTPGGGGSSAARRVRPVSLMVPS
QAGAPGRQFHGSASSLVEAVLISEGLGQFAQDPKFIEVTTQELADACDMTIEEMESAADNILS
GGAPQSPNGALLPFVNC
RDAGQDRAGGEEDAGCVRARGRPSEEELQDSRVYVSSL
NP_005446.2 zinc finger Ran-binding domain-containing protein 2 isoform 2
(SEQ ID NO: 111)
MSTKNFRVSDGDWICPDKKCGNVNFARRTSCNRCGREKTTEAKMMKAGGTEIGKTLAEKS
RGLFSANDWQCKTCSNVNWARRSECNMCNTPKYAKLEERTGYGGGFNERENVEYIEREES
DGEYDEFGRKKKKYRGKAVGPASILKEVEDKESEGEEEDEDEDLSKYKLDEDEDEDDADLS
KYNLDASEEEDSNKKKSNRRSRSKSRSSHSRSSSRSSSPSSSRSRSRSRSRSSSSSQSRSRSSSRE
RSRSRGSKSRSSSRSHRGSSSPRKRSYSSSSSSPERNRKRSRSRSSSSGDRKK
RRTRSRSPESQVIGENTKQP
NP_005185.2 CCAAT/enhancer-binding protein beta
(SEQ ID NO: 112)
MQRLVAWDPACLPLPPPPPAFKSMEVANFYYEADCLAAAYGGKAAPAAPPAARPGPRPPAG
ELGSIGDHERAIDFSPYLEPLGAPQAPAPATATDTFEAAPPAPAPAPASSGQHHDFLSDLFSDD
YGGKNCKKPAEYGYVSLGRLGAAKGALHPGCFAPLHPPPPPPPPPAELKAEPGFEPADCKRK
EEAGAPGGGAGMAAGFPYALRAYLGYQAVPSGSSGSLSTSSSSSPPGTPSPADAKAPPTACY
AGAAPAPSQVKSKAKKTVDKHSDEYKIRRERNNIAVRKSRDKAKMRNLETQHKVLELTAEN
ERLQKKVEQLSRELSTLRNLFKQLPEPLLASSGHC
NP_061820.1 cytochrome c
(SEQ ID NO: 113)
MGDVEKGKKIFIMKCSQCHTVEKGGKHKTGPNLHGLFGRKTGQAPGYSYTAANKNKGIIW
GEDTLMEYLENPKKYIPGTKMIFVGIKKKEERADLIAYLKKATNE
NP_004505.2 forkhead box protein K2
(SEQ ID NO: 114)
MAAAAAALSGAGTPPAGGGAGGGGAGGGGSPPGGWAVARLEGREFEYLMKKRSVTIGRNS
SQGSVDVSMGHSSFISRRHLEIFTPPGGGGHGGAAPELPPAQPRPDAGGDFYLRCLGKNGVF
VDGVFQRRGAPPLQLPRVCTFRFPSTNIKITFTALSSEKREKQEASESPVKAVQPHISPLTINIP
DTMAHLISPLPSPTGTISAANSCPSSPRGAGSSGYKVGRVMPSDLNLMADNSQPENEKEASG
GDSPKDDSKPPYSYAQLIVQAITMAPDKQLTLNGIYTHITKNYPYYRTADKGWQNSIRHNLS
LNRYFIKVPRSQEEPGKGSFWRIDPASESKLIEQAFRKRRPRGVPCFRTPLGPLSSRSAPASPN
HAGVLSAHSSGAQTPESLSREGSPAPLEPEPGAAQPKLAVIQEARFAQSAPGSPLSSQPVLITV
QRQLPQAIKPVTYTVATPVTTSTSQPPVVQTVHVVHQIPAVSVTSVAGLAPANTYTVSGQAV
VTPAAVLAPPKAEAQENGDHREVKVKVEPIPAIGHATLGTASRIIQTAQTTPVQTVTIVQQAP
LGQHQLPIKTVTQNGTHVASVPTAVHGQVNNAAASPLHMLATHASASASLPTKRHNGDQPE
QPELKRIKTEDGEGIVIALSVDTPPAAVREKGVQN
NP_002986.1 lymphotactin precursor
(SEQ ID NO: 115)
MRLLILALLGICSLTAYIVEGVGSEVSDKRTCVSLTTQRLPVSRIKTYTITEGSLRAVIF
ITKRGLKVCADPQATWVRDVVRSMDRKSNTRNNMIQTKPTGTQQSTNTAVTLTG
NP_004166.1 small nuclear ribonucleoprotein Sm D3
(SEQ ID NO: 116)
MSIGVPIKVLHEAEGHIVTCETNTGEVYRGKLIEAEDNMNCQMSNITVTYRDGRVAQLEQVY
IRGSKIRFLILPDMLKNAPMLKSMKNKNQGSGAGRGKAAILKAQVAARGRGRGMGRGNIFQ
KRR
NP_001029058.1 stromal cell-derived factor 1 isoform gamma
(SEQ ID NO: 117)
MNAKVVVVLVLVLTALCLSDGKPVSLSYRCPCRFFESHVARANVKHLKILNTPNCALQIVAR
LKNNNRQVCIDPKLKWIQEYLEKALNKGRREEKVGKKEKIGKKKRQKKRKAAQKRKN
NP_001091046.1 angiogenin precursor
(SEQ ID NO: 118)
MVMGLGVLLLVFVLGLGLTPPTLAQDNSRYTHFLTQHYDAKPQGRDDRYCESIMRRRGLTS
PCKDINTFIHGNKRSIKAICENKNGNPHRENLRISKSSFQVTTCKLHGGSPWPPCQYRATAGF
RNVVVACENGLPVHLDQSIFRRP
NP_005209.1 beta-defensin 1 preproprotein
(SEQ ID NO: 119)
MRTSYLLLFTLCLLLSEMASGGNFLTGLGHRSDHYNCVSSGGQCLYSACPIFTKIQGTCY
RGKAKCCK
NP_001137288.1 brain-derived neurotrophic factor isoform a preproprotein
(SEQ ID NO: 120)
MTILFLTMVISYFGCMKAAPMKEANIRGQGGLAYPGVRTHGTLESVNGPKAGSRGLTSLAD
TFEHVIEELLDEDQKVRPNEENNKDADLYTSRVMLSSQVPLEPPLLFLLEEYKNYLDAANMS
MRVRRHSDPARRGELSVCDSISEWVTAADKKTAVDMSGGTVTVLEKVPVSKGQLKQYFYE
TKCNPMGYTKEGCRGIDKRHWNSQCRTTQSYVRALTMDSKKRIGWRFIRIDTSCVCTLTIKR
GR
NP_665905.1 C-C motif chemokine 23 isoform CKbeta8 precursor
(SEQ ID NO: 121)
MKVSVAALSCLMLVTALGSQARVTKDAETEFMMSKLPLENPVLLDRFHATSADCCISYTPR
SIPCSLLESYFETNSECSKPGVIFLTKKGRRFCANPSDKQVQVCVRMLKLDTRIKTRKN
NP_001720.1 probetacellulin precursor
(SEQ ID NO: 122)
MDRAARCSGASSLPLLLALALGLVILHCVVADGNSTRSPETNGLLCGDPEENCAATTTQS
KRKGHFSRCPKQYKHYCIKGRCRFVVAEQTPSCVCDEGYIGARCERVDLFYLRGDRGQILVI
CLIAVMVVFIILVIGVCTCCHPLRKRRKRKKKEEEMETLGKDITPINEDIEETNIA
NP_001029058.1 stromal cell-derived factor 1 isoform gamma
(SEQ ID NO: 123)
MNAKVVVVLVLVLTALCLSDGKPVSLSYRCPCRFFESHVARANVKHLKILNTPNCALQIVAR
LKNNNRQVCIDPKLKWIQEYLEKALNKGRREEKVGKKEKIGKKKRQKKRKAAQKRKN
NP_060575.1 THAP domain-containing protein 1 isoform 1
(SEQ ID NO: 124)
MVQSCSAYGCKNRYDKDKPVSFHKFPLTRPSLCKEWEAAVRRKNFKPTKYSSICSEHFTPDC
FKRECNNKLLKENAVPTIFLCTEPHDKKEDLLEPQEQLPPPPLPPPVSQVDAAIGLLM
PPLQTPVNLSVFCDHNYTVEDTMHQRKRIHQLEQQVEKLRKKLKTAQQRCRRQERQLEKLK
EVVHFQKEKDDVSERGYVILPNDYFEIVEVPA
NP_001129687.1 artemin isoform 3 precursor
(SEQ ID NO: 125)
MELGLGGLSTLSHCPWPRQQAPLGLSAQPALWPTLAALALLSSVAEASLGSAPRSPAPRE
GPPPVLASPAGHLPGGRTARWCSGRARRPPPQPSRPAPPPPAPPSALPRGGRAARAGGPG
SRARAAGARGCRLRSQLVPVRALGLGHRSDELVRFRFCSGSCRRARSPHDLSLASLLGAGAL
RPPPGSRPVSQPCCRPTRYEAVSFMDVNSTWRTVDRLSATACGCLG
NP_001121128.1 cysteine and glycine-rich protein 3
(SEQ ID NO: 126)
MPNWGGGAKCGACEKTVYHAEEIQCNGRSFHKTCFHCMACRKALDSTTVAAHESEIYCKV
CYGRRYGPKGIGYGQGAGCLSTDTGEHLGLQFQQSPKPARSVTTSNPSKFTAKFGESEKCPR
CGKSVYAAEKVMGGGKPWHKTCFRCAICGKSLESTNVTDKDGELYCKVCYAKNFGPTGIG
FGGLTQQVEKKE
NP_002383.2 E3 ubiquitin-protein ligase Mdm2 isoform MDM2
(SEQ ID NO: 127)
MVRSRQMCNTNMSVPTDGAVTTSQIPASEQETLVRPKPLLLKLLKSVGAQKDTYTMKEVLF
YLGQYIMTKRLYDEKQQHIVYCSNDLLGDLFGVPSFSVKEHRKIYTMIYRNLVVVNQQESSD
SGTSVSENRCHLEGGSDQKDLVQELQEEKPSSSHLVSRPSTSSRRRAISETEENSDELSGERQR
KRHKSDSISLSFDESLALCVIREICCERSSSSESTGTPSNPDLDAGVSEHSGD
WLDQDSVSDQFSVEFEVESLDSEDYSLSEEGQELSDEDDEVYQVTVYQAGESDTDSFEEDPEI
SLADYWKCTSCNEMNPPLPSHCNRCWALRENWLPEDKGKDKGEISEKAKLENSTQAEEGFD
VPDCKKTIVNDSRESCVEENDDKITQASQSQESEDYSQPSTSSSIIYSSQEDVKEF
EREETQDKEESVESSLPLNAIEPCVICQGRPKNGCIVHGKTGHLMACFTCAKKLKKRNKP
CPVCRQPIQMIVLTYFP
NP_002384.2 protein Mdm4 isoform 1
(SEQ ID NO: 128)
MTSFSTSAQCSTSDSACRISPGQINQVRPKLPLLKILHAAGAQGEMFTVKEVMHYLGQYI
MVKQLYDQQEQHMVYCGGDLLGELLGRQSFSVKDPSPLYDMLRKNLVTLATATTDAAQTL
ALAQDHSMDIPSQDQLKQSAEESSTSRKRTTEDDIPTLPTSEHKCIHSREDEDLIENLAQDETS
RLDLGFEEWDVAGLPWWFLGNLRSNYTPRSNGSTDLQTNQDVGTAIVSDTTDDLWFLNESV
SEQLGVGIKVEAADTEQTSEEVGKVSDKKVIEVGKNDDLEDSKSLSDDTDVEVTSEDEWQC
TECKKFNSPSKRYCFRCWALRKDWYSDCSKLTHSLSTSDITAIPEKENEGNDVPDCRRTISAP
VVRPKDAYIKKENSKLFDPCNSVEFLDLAHSSESQETISSMGEQLDNLSEQRTDTENMEDCQ
NLLKPCSLCEKRPRDGNIIHGRTGHLVTCFHCARRLKKAGASCPICKKEIQLVIKVFIA
NP003055.1 antileukoproteinase precursor
(SEQ ID NO: 129)
MKSSGLFPFLVLLALGTLAPWAVEGSGKSFKAGVCPPKKSAQCLRYKKPECQSDWQCPGKK
RCCPDTCGIKCLDPVDTPNPTRRKPGKCPVTYGQCLMLNPPNFCEMDGQCKRDLKCCMGM
CGKSCVSPVKA
NP_055408.2 cpG-binding protein isoform 2
(SEQ ID NO: 130)
MEGDGSDPEPPDAGEDSKSENGENAPIYCICRKPDINCFMIGCDNCNEWFHGDCIRITEK
MAKAIREWYCRECREKDPKLEIRYRHKKSRERDGNERDSSEPRDEGGGRKRPVPDPDLQRR
AGSGTGVGAMLARGSASPHKSSPQPLVATPSQHHQQQQQQIKRSARMCGECEACRRTEDCG
HCDFCRDMKKFGGPNKIRQKCRLRQCQLRARESYKYFPSSLSPVTPSESLPRPRRPLPTQQQP
QPSQKLGRIREDEGAVASSTVKEPPEATATPEPLSDEDLPLDPDLYQDFCAGAFDDHGLPWM
SDTEESPFLDPALRKRAVKVKHVKRREKKSEKKKEERYKRHRQKQKHKDKWKHPERADAK
DPASLPQCLGPGCVRPAQPSSKYCSDDCGMKLAANRIYEILPQRIQQWQQSPCIAEEHGKKLL
ERIRREQQSARTRLQEMERRFHELEAIILRAKQQAVREDEESNEGDSDDTDLQIFCVSCGHPIN
PRVALRHMERCYAKYESQTSFGSMYPTRIEGATRLFCDVYNPQSKTYCKRLQVLCPEHSRDP
KVPADEVCGCPLVRDVFELTGDFCRLPKRQCNRHYCWEKLRRAEVDLERVRVWYKLDELF
EQERNVRTAMTNRAGLLALMLHQTIQHDPLTTDLRSSADR
NP_002926.2 eosinophil cationic protein precursor
(SEQ ID NO: 131)
MVPKLFTSQICLLLLLGLMGVEGSLHARPPQFTRAQWFAIQHISLNPPRCTIAMRAINNY
RWRCKNQNTFLRTTFANVVNVCGNQSIRCPHNRTLNNCHRSRFRVPLLHCDLINPGAQNISN
CTYADRPGRRFYVVACDNRDPRDSPRYPVVPVHLDTTI
NP_001127757.1 estrogen-related receptor gamma isoform 2
(SEQ ID NO: 132)
MSNKDRHIDSSCSSFIKTEPSSPASLTDSVNHHSPGGSSDASGSYSSTMNGHQNGLDSPP
LYPSAPILGGSGPVRKLYDDCSSTIVEDPQTKCEYMLNSMPKRLCLVCGDIASGYHYGVA
SCEACKAFFKRTIQGNIEYSCPATNECEITKRRRKSCQACRFMKCLKVGMLKEGVRLDRVRG
GRQKYKRRIDAENSPYLNPQLVQPAKKPYNKIVSHLLVAEPEKIYAMPDPTVPDSDIKALTTL
CDLADRELVVIIGWAKHIPGFSTLSLADQMSLLQSAWMEILILGVVYRSLSFEDE
LVYADDYIMDEDQSKLAGLLDLNNAILQLVKKYKSMKLEKEEFVTLKAIALANSDSMHIED
VEAVQKLQDVLHEALQDYEAGQHMEDPRRAGKMLMTLPLLRQTSTKAVQHFYNIKLEGKV
PMHKLFLEMLEAKV
NP_002948.1 retinoic acid receptor RXR-alpha
(SEQ ID NO: 133)
MDTKHFLPLDFSTQVNSSLTSPTGRGSMAAPSLHPSLGPGIGSPGQLHSPISTLSSPING
MGPPFSVISSPMGPHSMSVPTTPTLGFSTGSPQLSSPMNPVSSSEDIKPPLGLNGVLKVP
AHPSGNMASFTKHICAICGDRSSGKHYGVYSCEGCKGFFKRTVRKDLTYTCRDNKDCLIDKR
QRNRCQYCRYQKCLAMGMKREAVQEERQRGKDRNENEVESTSSANEDMPVERILEAELAV
EPKTETYVEANMGLNPSSPNDPVTNICQAADKQLFTLVEWAKRIPHFSELPLDDQVILLRAG
WNELLIASFSHRSIAVKDGILLATGLHVHRNSAHSAGVGAIFDRVLTELVSKMRDMQMDKT
ELGCLRAIVLFNPDSKGLSNPAEVEALREKVYASLEAYCKHKYPEQPGRFAKLLLRLPALRSI
GLKCLEHLFFFKLIGDTPIDTFLMEMLEAPHQMT
NP_115961.2 ribonuclease 7 precursor
(SEQ ID NO: 134)
MAPARAGFCPLLLLLLLGLWVAEIPVSAKPKGMTSSQWFKIQHMQPSPQACNSAMKNINKH
TKRCKDLNTFLHEPFSSVAATCQTPKIACKNGDKNCHQSHGAVSLTMCKLTSGKYPNCRYK
EKRQNKSYVVACKPPQKKDSQQFHLVPVHLDRVL
NP_003394.1 transcriptional repressor protein YY1
(SEQ ID NO: 135)
MASGDTLYIATDGSEMPAEIVELHEIEVETIPVETIETTVVGEEEEEDDDDEDGGGGDHG
GGGGHGHAGHHHHHHHHHHHPPMIALQPLVTDDPTQVHHHQEVILVQTREEVVGGDDSDG
LRAEDGFEDQILIPVPAPAGGDDDYIEQTLVTVAAAGKSGGGGSSSSGGGRVKKGGGKKSG
KKSYLSGGAGAAGGGGADPGNKKWEQKQVQIKTLEGEFSVTMWSSDEKKDIDHETVVEEQ
IIGENSPPDYSEYMTGKKLPPGGIPGIDLSDPKQLAEFARMKPRKIKEDDAPRTIACPHKGCTK
MFRDNSAMRKHLHTHGPRVHVCAECGKAFVESSKLKRHQLVHTGEKPFQCTFEGCGKRFSL
DFNLRTHVRIHTGDRPYVCPFDGCNKKFAQSTNLKSHILTHAKAKNNQ
NP_001020539.2 vascular endothelial growth factor A isoform d
(SEQ ID NO: 136)
MTDRQTDTAPSPSYHLLPGRRRTVDAAASRGQGPEPAPGGGVEGVGARGVALKLFVQLLGC
SRFGGAVVRAGEAEPSGAARSASSGREEPQPEEGEEEEEKEEERGPQWRLGARKPGSWTGE
AAVCADSAPAARAPQALARASGRGGRVARRGAEESGPPHSPSRRGSASRAGPGRASETMNF
LLSWVHWSLALLLYLHHAKWSQAAPMAEGGGQNHHEVVKFMDVYQRSYCHPIETLVDIFQ
EYPDEIEYIFKPSCVPLMRCGGCCNDEGLECVPTEESNITMQIMRIKPHQGQHIGEMSFLQHN
KCECRPKKDRARQENPCGPCSERRKHLFVQDPQTCKCSCKNTDSRCKARQLELNERTCRCD
KPRR
NP_077742.2 Wilms tumor protein isoform B
(SEQ ID NO: 137)
MQDPASTCVPEPASQHTLRSGPGCLQQPEQQGVRDPGGIWAKLGAAEASAERLQGRRSRGA
SGSEPQQMGSDVRDLNALLPAVPSLGGGGGCALPVSGAAQWAPVLDFAPPGASAYGSLGGP
APPPAPPPPPPPPPHSFIKQEPSWGGAEPHEEQCLSAFTVHFSGQFTGTAGACRYGPFGPPPPSQ
ASSGQARMFPNAPYLPSCLESQPAIRNQGYSTVTFDGTPSYGHTPSHHAAQFPNHSFKHEDP
MGQQGSLGEQQYSVPPPVYGCHTPTDSCTGSQALLLRTPYSSDNLYQMTSQLECMTWNQM
NLGATLKGVAAGSSSSVKWTEGQSNHSTGYESDNHTTPILCGAQYRIHTHGVFRGIQDVRRV
PGVAPTLVRSASETSEKRPFMCAYPGCNKRYFKLSHLQMHSRKHTGEKPYQCDFKDCERRF
SRSDQLKRHQRRHTGVKPFQCKTCQRKFSRSDHLKTHTRTHTGEKPFSCRWPSCQKKFARS
DELVRHHNMHQRNMTKLQLAL
NP_004371.2 CREB-binding protein isoform a
(SEQ ID NO: 138)
MAENLLDGPPNPKRAKLSSPGFSANDSTDFGSLFDLENDLPDELIPNGGELGLLNSGNLV
PDAASKHKQLSELLRGGSGSSINPGIGNVSASSPVQQGLGGQAQGQPNSANMASLSAMGKSP
LSQGDSSAPSLPKQAASTSGPTPAASQALNPQAQKQVGLATSSPATSQTGPGICMNANFNQT
HPGLLNSNSGHSLINQASQGQAQVMNGSLGAAGRGRGAGMPYPTPAMQGASSSVLAETLT
QVSPQMTGHAGLNTAQAGGMAKMGITGNTSPFGQPFSQAGGQPMGATGVNPQLASKQSM
VNSLPTFPTDIKNTSVTNVPNMSQMQTSVGIVPTQAIATGPTADPEKRKLIQQQLVLLLHAHK
CQRREQANGEVRACSLPHCRTMKNVLNHMTHCQAGKACQVAHCASSRQIISHWKNCTRHD
CPVCLPLKNASDKRNQQTILGSPASGIQNTIGSVGTGQQNATSLSNPNPIDPSSMQRAYAALG
LPYMNQPQTQLQPQVPGQQPAQPQTHQQMRTLNPLGNNPMNIPAGGITTDQQPPNLISESAL
PTSLGATNPLMNDGSNSGNIGTLSTIPTAAPPSSTGVRKGWHEHVTQDLRSHLVHKLVQAIFP
TPDPAALKDRRMENLVAYAKKVEGDMYESANSRDEYYHLLAEKIYKIQKELEEKRRSRLHK
QGILGNQPALPAPGAQPPVIPQAQPVRPPNGPLSLPVNRMQVSQGMNSFNPMSLGNVQLPQA
PMGPRAASPMNHSVQMNSMGSVPGMAISPSRMPQPPNMMGAHTNNMMAQAPAQSQFLPQ
NQFPSSSGAMSVGMGQPPAQTGVSQGQVPGAALPNPLNMLGPQASQLPCPPVTQSPLHPTPP
PASTAAGMPSLQHTTPPGMTPPQPAAPTQPSTPVSSSGQTPTPTPGSVPSATQTQSTPTVQAA
AQAQVTPQPQTPVQPPSVATPQSSQQQPTPVHAQPPGTPLSQAAASIDNRVPTPSSVASAETN
SQQPGPDVPVLEMKTETQAEDTEPDPGESKGEPRSEMMEEDLQGASQVKEETDIAEQKSEPM
EVDEKKPEVKVEVKEEEESSSNGTASQSTSPSQPRKKIFKPEELRQALMPTLEALYRQDPESL
PFRQPVDPQLLGIPDYFDIVKNPMDLSTIKRKLDTGQYQEPWQYVDDVWLMFNNAWLYNR
KTSRVYKFCSKLAEVFEQEIDPVMQSLGYCCGRKYEFSPQTLCCYGKQLCTIPRDAAYYSYQ
NRYHFCEKCFTEIQGENVTLGDDPSQPQTTISKDQFEKKKNDTLDPEPFVDCKECGRKMHQI
CVLHYDIIWPSGFVCDNCLKKTGRPRKENKFSAKRLQTTRLGNHLEDRVNKFLRRQNHPEA
GEVFVRVVASSDKTVEVKPGMKSRFVDSGEMSESFPYRTKALFAFEEIDGVDVCFFGMHVQ
EYGSDCPPPNTRRVYISYLDSIHFFRPRCLRTAVYHEILIGYLEYVKKLGYVTGHIWACPPSEG
DDYIFHCHPPDQKIPKPKRLQEWYKKMLDKAFAERIIHDYKDIFKQATEDRLTSAKELPYFEG
DFWPNVLEESIKELEQEEEERKKEESTAASETTEGSQGDSKNAKKKNNKKTNKNKSSISRAN
KKKPSMPNVSNDLSQKLYATMEKHKEVFFVIHLHAGPVINTLPPIVDPDPLLSCDLMDGRDA
FLTLARDKHWEFSSLRRSKWSTLCMLVELHTQGQDRFVYTCNECKHHVETRWHCTVCEDY
DLCINCYNTKSHAHKMVKWGLGLDDEGSSQGEPQSKSPQESRRLSIQRCIQSLVHACQCRNA
NCSLPSCQKMKRVVQHTKGCKRKTNGGCPVCKQLIALCCYHAKHCQENKCPVPFCLNIKHK
LRQQQIQHRLQQAQLMRRRMATMNTRNVPQQSLPSPTSAPPGTPTQQPSTPQTPQPPAQPQP
SPVSMSPAGFPSVARTQPPTTVSTGKPTSQVPAPPPPAQPPPAAVEAARQIEREAQQQQHLYR
VNINNSMPPGRTGMGTPGSQMAPVSLNVPRPNQVSGPVMPSMPPGQWQQAPLPQQQPMPG
LPRPVISMQAQAAVAGPRMPSVQPPRSISPSALQDLLRTLKSPSSPQQQQQVLNILKSNPQLM
AAFIKQRTAKYVANQPGMQPQPGLQSQPGMQPQPGMHQQPSLQNLNAMQAGVPRPGVPPQ
QQAMGGLNPQGQALNIMNPGHNPNMASMNPQYREMLRRQLLQQQQQQQQQQQQQQQQQ
QGSAGMAGGMAGHGQFQQPQGPGGYPPAMQQQQRMQQHLPLQGSSMGQMAAQMGQLG
QMGQPGLGADSTPNIQQALQORILQQQQMKQQIGSPGQPNPMSPQQHMLSGQPQASHLPGQ
QIATSLSNQVRSPAPVQSPRPQSQPPHSSPSPRIQPQPSPHHVSPQTGSPHPGLAVTMASSIDQG
HLGNPEQSAMLPQLNTPSRSALSSELSLVGDTTGDTLEKFVEGL
NP_004371.2 CREB-binding protein isoform a
(SEQ ID NO: 139)
MAENLLDGPPNPKRAKLSSPGFSANDSTDFGSLFDLENDLPDELIPNGGELGLLNSGNLV
PDAASKHKQLSELLRGGSGSSINPGIGNVSASSPVQQGLGGQAQGQPNSANMASLSAMGKSP
LSQGDSSAPSLPKQAASTSGPTPAASQALNPQAQKQVGLATSSPATSQTGPGICMNANFNQT
HPGLLNSNSGHSLINQASQGQAQVMNGSLGAAGRGRGAGMPYPTPAMQGASSSVLAETLT
QVSPQMTGHAGLNTAQAGGMAKMGITGNTSPFGQPFSQAGGQPMGATGVNPQLASKQSM
VNSLPTFPTDIKNTSVTNVPNMSQMQTSVGIVPTQAIATGPTADPEKRKLIQQQLVLLLHAHK
CQRREQANGEVRACSLPHCRTMKNVLNHMTHCQAGKACQVAHCASSRQIISHWKNCTRHD
CPVCLPLKNASDKRNQQTILGSPASGIQNTIGSVGTGQQNATSLSNPNPIDPSSMQRAYAALG
LPYMNQPQTQLQPQVPGQQPAQPQTHQQMRTLNPLGNNPMNIPAGGITTDQQPPNLISESAL
PTSLGATNPLMNDGSNSGNIGTLSTIPTAAPPSSTGVRKGWHEHVTQDLRSHLVHKLVQAIFP
TPDPAALKDRRMENLVAYAKKVEGDMYESANSRDEYYHLLAEKIYKIQKELEEKRRSRLHK
QGILGNQPALPAPGAQPPVIPQAQPVRPPNGPLSLPVNRMQVSQGMNSFNPMSLGNVQLPQA
PMGPRAASPMNHSVQMNSMGSVPGMAISPSRMPQPPNMMGAHTNNMMAQAPAQSQFLPQ
NQFPSSSGAMSVGMGQPPAQTGVSQGQVPGAALPNPLNMLGPQASQLPCPPVTQSPLHPTPP
PASTAAGMPSLQHTTPPGMTPPQPAAPTQPSTPVSSSGQTPTPTPGSVPSATQTQSTPTVQAA
AQAQVTPQPQTPVQPPSVATPQSSQQQPTPVHAQPPGTPLSQAAASIDNRVPTPSSVASAETN
SQQPGPDVPVLEMKTETQAEDTEPDPGESKGEPRSEMMEEDLQGASQVKEETDIAEQKSEPM
EVDEKKPEVKVEVKEEEESSSNGTASQSTSPSQPRKKIFKPEELRQALMPTLEALYRQDPESL
PFRQPVDPQLLGIPDYFDIVKNPMDLSTIKRKLDTGQYQEPWQYVDDVWLMLMFNNAWLYNR
KTSRVYKFCSKLAEVFEQEIDPVMQSLGYCCGRKYEFSPQTLCCYGKQLCTIPRDAAYYSYQ
NRYHFCEKCFTEIQGENVTLGDDPSQPQTTISKDQFEKKKNDTLDPEPFVDCKECGRKMHQI
CVLHYDIIWPSGFVCDNCLKKTGRPRKENKFSAKRLQTTRLGNHLEDRVNKFLRRQNHPEA
GEVFVRVVASSDKTVEVKPGMKSRFVDSGEMSESFPYRTKALFAFEEIDGVDVCFFGMHVQ
EYGSDCPPPNTRRVYISYLDSIHFFRPRCLRTAVYHEILIGYVKKEGYVTGHIWACPPSEG
DDYIFHCHPPDQKIPKPKRLQEWYKKMLDKAFAERIIHDYKDIFKQATEDRLTSAKELPYFEG
DFWPNVLEESIKELEQEEEERKKEESTAASETTEGSQGDSKNAKKKNNKKTNKNKSSISRAN
KKKPSMPNVSNDLSQKLYATMEKHKEVFFVIHLHAGPVINTLPPIVDPDPLLSCDLMDGRDA
FLTLARDKHWEFSSLRRSKWSTLCMLVELHTQGQDRFVYTCNECKHHVETRWHCTVCEDY
DLCINCYNTKSHAHKMVKWGLGLDDEGSSQGEPQSKSPQESRRLSIQRCIQSLVHACQCRNA
NCSLPSCQKMKRVVQHTKGCKRKTNGGCPVCKQLIALCCYHAKHCQENKCPVPFCLNIKHK
LRQQQIQHRLQQAQLMRRRMATMNTRNVPQQSLPSPTSAPPGTPTQQPSTPQTPQPPAQPQP
SPVSMSPAGFPSVARTQPPTTVSTGKPTSQVPAPPPPAQPPPAAVEAARQIEREAQQQQHLYR
VNINNSMPPGRTGMGTPGSQMAPVSLNVPRPNQVSGPVMPSMPPGQWQQAPLPQQQPMPG
LPRPVISMQAQAAVAGPRMPSVQPPRSISPSALQDLLRTLKSPSSPQQQQQVLNILKSNPQLM
AAFIKQRTAKYVANQPGMQPQPGLQSQPGMQPQPGMHQQPSLQNLNAMQAGVPRPGVPPQ
QQAMGGLNPQGQALNIMNPGHNPNMASMNPQYREMLRRQLLQQQQQQQQQQQQQQQQQ
QGSAGMAGGMAGHGQFWPOGPGGYPPAMQQQQRMQQHLPLQGSSMGQMAAQMGQLG
QMGQPGLGADSTPNIQQALQQRILQQQQMKQQIGSPGQPNPMSPQQHMLSGQPQASHLPGQ
QIATSLSNQVRSPAPVQSPRPQSQPPHSSPSPRIQPQPSPHHVSPQTGSPHPGLAVTMASSIDQG
HLGNPEQSAMLPQLNTPSRSALSSELSLVGDTTGDTLEKFVEGL
NP_001116214.1 estrogen receptor isoform 4
(SEQ ID NO: 140)
MTMTLHTKASGMALLHQIQGNELEPLNRPQLKIPLERPLGEVYLDSSKPAVYNYPEGAAYEF
NAAAAANAQVYGQTGLPYGPGSEAAAFGSNGLGGFPPLNSVSPSPLMLLHPPPQLSPFLQPH
GQQVPYYLENEPSGYTVREAGPPAFYRPNSDNRRQGGRERLASTNDKGSMAMESAKETRYC
AVCNDYASGYHYGVWSCEGCKAFFKRSIQGHNDYMCPATNQCTIDKNRRKSCQACRLRKC
YEVGMMKGGIRKDRRGGRMLKHKRQRDDGEGRGEVGSAGDMRAANLWPSPLMIKRSKKN
SLALSLTADQMVSALLDAEPPILYSEYDPTRPFSEASMMGLLTNLADRELVHMINWAKRVPG
FVDLTLHDQVHLLECAWLEILMIGLVWRSMEHPGKLLFAPNLLLDRNQGKCVEGMVEIFDM
LLATSSRFRMMNLQGEEFVCLKSIILLNSGVYTFLSSTLKSLEEKDHIHRVLDKITDTLIHLMA
KAGLTLQQQHQRLAQLLLILSHIRHMSNKGMEHLYSMKCKNVVPLYDLLLEMLDAHRLHA
PTSRGGASVEETDQSHLATAGSTSSHSLQKYYITGEAEGFPATV
NP_849180.1 hepatocyte nuclear factor 4-alpha isoform a
(SEQ ID NO: 141)
MRLSKTLVDMDMADYSAALDPAYTTLEFENVQVLTMGNDTSPSEGTNLNAPNSLGVSALC
AICGDRATGKHYGASSCDGCKGFFRRSVRKNHMYSCRFSRQCVVDKDKRNQCRYCRLKKC
FRAGMKKFAVQNERDRISTRRSSYRDSSLPSINALLQAEVLSRQITSPVSGINGDIRAKKIASIA
DVCESMKEQLLVLVEWAKYIPAFCELPLDDQVALLRAHAGEHLLLGATKRSMVFKDVLLLG
NDYIVPRHCPELAEMSRVSIRILDELVLPFQELQIDDNEYAYLKAIIFFDPDAKGLSDPGKIKRL
RSQVQVSLEDYINDRQYDSRGRFGELLLLLPTLQSITWQMIEQIQFIKLFGMAKIDNLLQEML
LGGSPSDAPHAHHPLHPHLMQEHMGTNVIVANTMPTHLSNGQMSTPETPQPSPPGGSGSEPY
KLLPGAVATIVKPLSAIPQPTITKQEVI
NP_001420.2 histone acetyltransferase p300
(SEQ ID NO: 142)
MAENVVEPGPPSAKRPKLSSPALSASASDGTDFGSLFDLEHDLPDELINSTELGLTNGGD
INQLQTSLGMVQDAASKHKQLSELLRSGSSPNLNMGVGGPGQVMASQAQQSSPGLGLINSM
VKSPMTQAGLTSPNMGMGTSGPNQGPTQSTGMMNSPVNQPAMGMNTGMNAGMNPGMLA
AGNGQGIMPNQVMNGSIGAGRGRQNMQYPNPGMGSAGNLLTEPLQQGSPQMGGQTGLRG
PQPLKMGMMNNPNPYGSPYTQNPGQQIGASGLGLQIQTKTVLSNNLSPFAMDKKAVPGGG
MPNMGOQPAPQVQQPGLVTPVAQGMGSGAHTADPEKRKLIQQQLVLLLHAHKQQRREQA
NGEVRQCNLPHCRTMKNVLNHMTHCQSGKSCQVAHCASSRQIISHWKNCTRHDCPVCLPL
KNAGDKRNQPILTGAPVGLGNPSSLGVGQQSAPNLSTVSQIDPSSIERAYAALGLPYQVNQ
MPTQPQVQAKNQQNQQPGQSPQGMRPMSNMSASPMGVNGGVGVQTPSLLSDSMLHSAINS
QNPMMSENASVPSLGPMPTAAQPSTTGIRKQWHEDITQDLRNHLVHKLVQAIFPTPDPAALK
DRRMENLVAYARKVEGDMYESANNRAEYYHLLAEKIYKIQKELEEKRRTRLQKQNMLPNA
AGMVPVSMNPGPNMGQPQPGMTSNGPLPDPSMIRGSVPNQMMPRITPQSGLNQFGQMSMA
QPPIVPRQTPPLQHHGQLAQPGALNPPMGYGPRMQQPSNQGQFLPQTQFPSQGMNVTNIPLA
PSSGQAPVSQAQMSSSSCPVNSPIMPPGSQGSHIHCPQLPQPALHQNSPSPVPSRTPTPHHTPPS
IGAQQPPATTIPAPVPTPPAMPPGPQSQALHPPPRQTPTPPTTQLPQQVQPSLPAAPSADQPQQ
QPRSQQSTAASVPTPTAPLLPPQPATPLSQPAVSIEGQVSNPPSTSSTEVNSQAIAEKQPSQEVK
MEAKMEVDQPEPADTQPEDISESKVEDCKMESTETEERSTELKTEIKEEEDQPSTSATQSSPA
PGQSKKKIFKPEELRQALMPTLEALYRQDPESLPFRQPVDPQLLGIPDYFDIVKSPMDLSTIKR
KLDTGQYQEPWQYVDDIWLMFNNAWLYNRKTSRVYKYCSKLSEVFEQEIDPVMQSLGYCC
GRKLEFSPQTLCCYGKQLCTIPRDATYYSYQNRYHFCEKCFNEIQGESVSLGDDPSQPQTTIN
KEQFSKRKNDTLDPELFVECTECGRKMHQICVLHHEIIWPAGFVCDGCLKKSARTRKENKFS
AKRLPSTRLGTFLENRVNDFLRRQNHPESGEVTVRVVHASDKTVEVKPGMKARFVDSGEMA
ESFPYRTKALFAFEEIDGVDLCFFGMHVQEYGSDCPPPNORRVYISYLDSVHFFRPKCLRTAV
YHEILIGYLEYVKKLGYTTGHIWACPPSEGDDYIFHCHPPDQKIPKPKRLQEWYKKMLDKAV
SERIVHDYKDIFKQATEDRLTSAKELPYFEGDFWPNVLEESIKELEQEEEERKREENTSNESTD
VTKGDSKNAKKKNNKKTSKNKSSLSRGNKKKPGMPNVSNDLSQKLYATMEKHKEVFFVIR
LIAGPAANSLPPIVDPDPLIPCDLMDGRDAFLTLARDKHLEFSSLRRAQWSTMCMLVELHTQS
QDRFVYTCNECKHHVETRWHCTVCEDYDLCITCYNTKNHDHKMEKLGLGLDDESNNQQA
AATQSPGDSRRLSIQRCIQSLVHACQCRNANCSLPSCQKMKRVVQHTKGCKRKTNGGCPICK
QLIALCCYHAKHCQENKCPVPFCLNIKQKLRQQQLQHRLQQAQMLRRRMASMQRTGVVGQ
QQGLPSPTPATPTTPTGQQPTTPQTPQPTSQPQPTPPNSMPPYLPRTQAAGPVSQGKAAGQVT
PPTPPQTAQPPLPGPPPAAVEMAMQIQRAAETQRQMAHVQIFQRPIQHQMPPMTPMAPMGM
NPPPMTRGPSGHLEPGMGPTGMQQQPPWSQGGLPQPQQLQSGMPRPAMMSVAQHGQPLN
MAPQPGLGQVGISPLKPGTVSQQALQNLLRTLRSPSSPLQQQQVLSILHANPQLLAAFIKQRA
AKYANSNPQPIPGQPGMPQGQPGLQPPTMPGQQGVHSNPAMQNMNPMQAGVQRAGLPQQ
QPQQQLQPPMGGMSPQAQQMNMNHNTMPSQFRDILRRQQMMQQQQQQGAGPGIGPGMA
NHNQFQQPQGVGYPPQQQQRMQHHMQQMQQGNMGQIGQLPQALGAEAGASLQAYQQRL
LQQQMGSPVQPNPMSPQQHMLPNQAQSPHLQGQQIPNSLSNQVRSPQPVPSPRPQSQPPHSSP
SPRMQPQPSPHHVSPQTSSPHPGLVAAQANPMEQGHFASPDQNSMLSQLASNPGMANLHGA
SATDLGLSTDNSDLNSNLSQSTLDIH
NP_001420.2 histone acetyltransferase p300
(SEQ ID NO: 143)
MAENVVEPGPPSAKRPKLSSPALSASASDGTDFGSLFDLEHDLPDELINSTELGLTNGGD
INQLQTSLGMVQDAASKHKQLSELLRSGSSPNLNMGVGGPGQVMASQAQQSSPGLGLINSM
VKSPMTQAGLTSPNMGMGTSGPNQGPTQSTGMMNSPVNQPAMGMNTGMNAGMNPGMLA
AGNGQGIMPNQVMNGSIGAGRGRQNMQYPNPGMGSAGNLLTEPLQQGSPQMGGQTGLRG
PQPLKMGMMNNPNPYGSPYTQNPGQQIGASGLGLQIQTKTVLSNNLSPFAMDKKAVPGGG
MPNMGQQPAPQVQQPGLVTPVAQGMGSGAHTADPEKRKLIQQQLVLLLHAHKCQRREQA
NGEVRQCNLPMCRTMKNVLNHMTHCQSGKSCQVAHCASSRQIISHWKNCTRHDCTVCLPL
KNAGDKRNQQPILTGAPVGLGNPSSLGVGQQSAPNLSTVSQIDPSSIERAYAALGLPYQVNQ
MPTQPQVQAKNQQNQQPGQSPQGMRPMSNMSASPMGVNGGVGVQTPSLLSDSMLHSAINS
QNPMMSENASVPSLGPMPTAAQPSTTGIRKQWHEDITQDLRNHLVHKLVQAIFPTPDPAALK
DRRMENLVAYARKVEGDMYESANNRAEYYHLLAEKIYKIOKELEEKRRTRLQKQNMLPNA
AGMVPVSMNPGPNMGQPQPGMTSNGPLPDPSMIRGSVPNQMMPRITPQSGLNQFGQMSMA
QPPIVPRQTPPLQHHGQLAQPGALNPPMGYGPRMQQPSNQGQFLPQTQFPSQGMNVTNIPLA
PSSGQAPVSQAQMSSSSCPVNSPIMPPGSQGSHIHCPQLPQPALHQNSPSPVPSRTPTPHHTPPS
IGAQQPPATTIPAPVPTPPAMPPGPQSQALHPPPRQTPTPPTTQLPQQVQPSLPAAPSADQPQQ
QPRSQQSTAASVPTPTAPLLPPQPATPLSQPAVSIEGQVSNPPSTSSTEVNSQAIAEKQPSQEVK
MEAKMEVDQPEPADTQPEDISESKVEDCKMESTETEERSTELKTEIKEEEDQPSTSATQSSPA
PGQSKKKIFKPEELRQALMPTLEALYRQDPESLPFRQPVDPQLLGIPDYFDIVKSPMDLSTIKR
KLDTGQYQEPWQYVDDIWLMFNNAWLYNRKTSRVYKYCSKLSEVFEQEIDPVMQSLGYCC
GRKLEFSPQTLCCYGKQLCTIPRDATYYSYQNRYHFCEKCFNEIQGESVSLGDDPSQPQTTIN
KEQFSKRKNDTLDPELFVECTECGRKMHQICVLHHEIIWPAGFVCDGCLKKSARTRKENKFS
AKRLPSTRLGTFLENRVNDFLRRQNHPESGEVTVRVVHASDKTVEVKPGMKARFVDSGEMA
ESFPYRTKALFAFEEIDGVDLCFFGMHVQEYGSDCPPPNQRRVYISYLDSVHFFRPKCLRTAV
YHEILIGYLEYVKKLGYTTGHIWACPPSEGDDYIFHCHPPDQKIPKPKRLQEWYKKMLDKAV
SERIVHDYKDIFKQATEDRLTSAKELPYFEGDFWPNVLEESIKELEQEEEERKREENTSNESTD
VTKGDSKNAKKKNNKKTSKNKSSLSRGNKKKPGMPNVSNDLSQKLYATMEKHKEVFFVIR
LIAGPAANSLPPIVDPDPLIPCDLMDGRDAFLTLARDKHLEFSSLRRAQWSTMCMLVELHTQS
QDRFVYTCNECKHHVETRWHCTVCEDYDLCITCYNTKNHDHKMEKLGLGLDDESNNQQA
AATQSPGDSRRLSIQRCIQSLVHACQCRNANCSLPSCQKMKRVVQHTKGCKRKTNGGCPICK
QLIALCCYHAKHCQENKCPVPFCLNIKQKLRQQQLQHRLQQAQMLRRRMASMQRTGVVGQ
QQGLPSPTPATPTTPTGQQPTTPQTPQPTSQPQPTPPNSMPPYLPRTQAAGPVSQGKAAGQVT
PPTPPQTAQPPLPGPPPAAVEMAMQIQRAAETQRQMAHVQIFQRPIQHQMPPMTPMAPMGM
NPPPMTRGPSGHLEPGMGPTGMQQQPPWSQGGLPQPQQLQSGMPRPAMMSVAQIIGQPLN
MAPQPGLGQVGISPLKPGTVSQQALQNLLRTLRSPSSPLQQQQVLSILHANPQLLAAFIKQRA
AKYANSNPQPIPGQPGMPQGQPGLQPPTMPGQQGVHSNPAMQNMNPMQAGVQRAGLPQQ
QPQQQLQPPMGGMSPQAQQMNMNHNTMPSQFRDILRRQQMMQQQQQQGAGPGIGPGMA
NHNQFQQPQGVGYPPQQQQRMQHHMQQMQQGNMGQIGQLPQALGAEAGASLQAYQQRL
LQQQMGSPVQPNPMSPQQHMLPNQAQSPHLQGQQIPNSLSNQVRSPQPVPSPRPQSQPPHSSP
SPRMQPQPSPHHVSPQTSSPHPGLVAAQANPMEQGHFASPDQNSMLSQLASNPGMANLHGA
SATDLGLSTDNSDLNSNLSQSTLDIH
NP_005924.2 histone-lysine N-methyltransferase MLL isoform 2 precursor
(SEQ ID NO: 144)
MAHSCRWRFPARPGTTGGGGGGGRRGLGGAPRQRVPALLLPPGPPVGGGGPGAPPSPPAVA
AAAAAAGSSGAGVPGGAAAASAASSSSASSSSSSSSSASSGPALLRVGPGFDAALQVSAAIGT
NLRRFRAVFGESGGGGGSGEDEQFLGFGSDEEVRVRSPTRSPSVKTSPRKPRGRPRSGSDRNS
AILSDPSVFSPLNKSETKSGDKIKKKDSKSIEKKRGRPPTFPGVKIKITHGKDISELPKGNKEDS
LKKIKRTPSATFQQATKIKKLRAGKLSPLKSKFKTGKLQIGRKGVQIVRRRGRPPSTERIKTPS
GLLINSELEKPQKVRKDKEGTPPLTKEDKTVVRQSPRRIKPVRIIPSSKRTDATIAKQLLQRAK
KGAQKKIEKEAAQLQGRKVKTQVKNIRQFIMPVVSAISSRIIKTPRRFIEDEDYDPPIKIARLES
TPNSRFSAPSCGSSEKSSAASQHSSQMSSDSSRSSSPSVDTSTDSQASEEIQVLPEERSDTPEVH
PPLPISQSPENESNDRRSRRYSVSERSFGSRTTKKLSTLQSAPQQQTSSSPPPPLLTPPPPLQPAS
SISDHTPWLMPPTIPLASPFLPASTAPMQGKRKSILREPTFRWTSLKHSRSEPQYFSSAKYAKE
GLIRKPIFDNFRPPPLTPEDVGFASGFSASGTAASARLFSPLHSGTRFDMHKRSPLLRAPRFTPS
EAHSRIFESVTLPSNRTSAGTSSSGVSNRKRKRKVFSPIRSEPRSPSHSMRTRSGRLSSSELSPLT
PPSSVSSSLSISVSPLATSALNPTFTFPSHSLTQSGESAEKNQRPRKQTSAPAEPFSSSSPTPLFP
WFTPGSQTERGRNKDKAPEELSKDRDADKSVEKDKSRERDREREKENKRESRKEKRKKGSE
IQSSSALYPVGRVSKEKVVGEDVATSSSAKKATGRKKSSSHDSGTDITSVTLGDTTAVKTKIL
IKKGRGNLEKTNLDLGPTAPSLEKEKTLCLSTPSSSTVKHSTSSIGSMLAQADKLPMTDKRVA
SLLKKAKAQLCKIEKSKSLKQTDQPKAQGQESDSSETSVRGPRIKHVCRRAAVALGRKRAVF
PDDMPTLSALPWEEREKILSSMGNDDKSSIAGSEDAEPLAPPIKPIKPVTRNKAPQEPPVKKGR
RSRRCGQCPGCQVPEDCGVCTNCLDKPKFGGRNIKKQCCKMRKCQNLQWMPSKAYLQKQ
AKAVKKKEKKSKTSEKKDSKESSVVKNVVDSSQKPTPSAREDPAPKKSSSEPPPRKPVEEKS
EEGNVSAPGPESKQATTPASRKSSKQVSQPALVIPPQPPTTGPPRKEVPKTTPSEPKKKQPPPP
ESGPEQSKQKKVAPRPSIPVKQKPKEKEKPPPVNKQENAGTLNILSTLSNGNSSKQKIPADGV
HRIRVDFKEDCEAENVWEMGGLGILTSVPITPRVVCFLCASSGHVEFVYCQVCCEPFHKFCLE
ENERPLEDQLENWCCRRCKFCHVCGRQHQATKQLLECNKCRNSYHPECLGPNYPTKPTKKK
KVWICTKCVRCKSCGSTTPGKGWDAQWSHDFSLCHDCAKLFAKGNFCPLCDKCYDDDDYE
SKMMQCGKCDRWVHSKCENLSDEMYEILSNLPESVAYTCVNCTERHPAEWRLALEKELQIS
LKQVLTALLNSRTTSHLLRYRQAAKPPDLNPETEESIPSRSSPEGPDPPVLTEVSKQDDQQPLD
LEGVKRKMDQGNYTSVLEFSDDIVKIIQAAINSDGGQPEIKKANSMVKSFFIRQMERVFPWFS
VKKSRFWEPNKVSSNSGMLPNAVLPPSLDHNYAQWQEREENSHTEQPPLMKKIIPAPKPKGP
GEPDSPTPLHPPTPPILSTDRSREDSPELNPPPGIEDNRQCALCLTYGDDSANDAGRLLYIGQN
EWTHVNCALWSAEVFEDDDGSLKNVHMAVIRGKQLRCEFCQKPGATVGCCLTSCTSNYHF
MCSRAKNCVFLDDKKVYCQRHRDLIKGEVVPENGFEVFRRVFVDFEGISLRRKFLNGLEPEN
IHMMIGSMTIDCLGILNDLSDCEDKLFPIGYQCSRVYWSTTDARKRCVYTCKIVECRPPVVEP
DINSTVEHDENRTIAHSPTSFTESSSKESQNTAEIISPPSPDRPPHSQTSGSCYYHVISKVPRIRTP
SYSPTQRSPGCRPLPSAGSPTPTTHEIVTVGDPLLSSGLRSIGSRRHSTSSLSPQRSKLRIMSPMR
TGNTYSRNNVSSVSTTGTATDLESSAKVVDHVLGPLNSSTSLGQNTSTSSNLQRTVVTVGNK
NSHLDGSSSSEMKQSSASDLVSKSSSLKGEKTKVLSSKSSEGSAHNVAYPGIPKLAPQVHNTT
SRELNVSKIGSFAEPSSVSFSSKEALSFPHLHLRGQRNDRDQHTDSTQSANSSPDEDTEVK
TLKLSGMSNRSSIINEHMGSSSRDRRQKGKKSCKETFKEKHSSKSFLEPGQVTTGEEGNL
KPEFMDEVLTPEYMGQRPCNNVSSDKIGDKGLSMPGVPKAPPMQVEGSAKELQAPRKRTVK
VTLTPLKMENESQSKNALKESSPASPLQIESTSPTEPISASENPGDGPVAQPSPNNTSC
QDSQSNNYQNLPVQDRNLMLPDGPKPQEDGSFKRRYPRRSARARSNMFFGLTPLYGVRSYG
EEDIPFYSSSTGKKRGKRSAEGQVDGADDLSTSDEDDLYYYNFTRTVISSGGEERLASHNLFR
EEEQCDLPKISQLDGVDDGTESDTSVTATTRKSSQIPKRNGKENGTENLKIDRPED
AGEKEHVTKSSVGHKNEPKMDNCHSVSRVKTQGQDSLEAQLSSLESSRRVHTSTPSDKNLL
DTYNTELLKSDSDNNNSDDCGNILPSDIMDFVLKNTPSMQALGESPESSSSELLNLGEGLGLD
SNREKDMGLFEVFSQQLPTTEPVDSSVSSSISAEEQFELPLELPSDLSVLTTRSPTVPSQNPSRL
AVISDSGEKRVTITEKSVASSESDPALLSPGVDPTPEGHMTPDHFIQGHMDADHISSPPCGSVE
QGHGNNQDLTRNSSTPGLQVPVSPTVPIQNQKYVPNSTDSPGPSQISNAAVQTTPPHLKPATE
KLIVVNQNMQPLYVLQTLPNGVTQKIQLTSSVSSTPSVMETNTSVLGPMGGGLTLTTGLNPS
LPTSQSLFPSASKGLLPMSHHQHLHSFPAATQSSFPPNISNPPSGLLIGVQPPPDPQLLVSESSQ
RTDLSTTVATPSSGLKKRPISRLQTRKNKKLAPSSTPSNIAPSDVVSNMTLINFTPSQLPNHPSL
LDLGSLNTSSHRTVPNIIKRSKSSIMYFEPAPLLPQSVGGTAATAAGTSTISQDTSHLTSGSVSG
LASSSSVLNVVSMQTTTTPTSSASVPGHVTLTNPRLLGTPDIGSISNLLIKASQQSLGIQDQPVA
LPPSSGMFPQLGTSQTPSTAAITAASSICVLPSTQTTGITAASPSGEADEHYQLQHVNQLLASK
TGIHSSQRDLDSASGPQVSNFTQTVDAPNSMGLEQNKALSSAVQASPTSPGGSPSSPSSGQRS
ASPSVPGPTKPKPKTKRFQLPLDKGNGKKHKVSHLRTSSSEAHIPDQETTSLTSGTGTPGAEA
EQQDTASVEQSSQKECGQPAGQVAVLPEVQVTQNPANEQESAEPKTVEEEESNFSSPLMLW
LQQEQKRKESITEKKPKKGLVFEISSDDGFQICAESIEDAWKSLTDKVQEARSNARLKQLSFA
GVNGLRMLGILHDAVVFLIEQLSGAKHCRNYKFRFHKPEEANEPPLNPHGSARAEVHLRKSA
FDMFNFLASKHRQPPEYNPNDEEEEEVQLKSARRATSMDLPMPMRFRHLKKTSKEAVGVYR
SPIHGRGLFCKRNIDAGEMVIEYAGNVIRSIQTDKREKYYDSKGIGCYMFRIDDSEVVDATM
HGNAARFINHSCEPNCYSRVINIDGQKHIVIFAMRKIYRGEELTYDYKFPIEDASNKLPCNCG
AKKCRKFLN
NP_068370.1 nuclear receptor subfamily 1 group D member 1
(SEQ ID NO: 145)
MTTLDSNNNTGGVITYIGSSGSSPSRTSPESLYSDNSNGSFQSLTQGCPTYFPPSPTGSLTQDPA
RSFGSIPPSLSDDGSPSSSSSSSSSSSSFYNGSPPGSLQVAMEDSSRVSPSKSTSNITKLNGMVLL
CKVCGDVASGFHYGVHACEGCKGFFRRSIQQNIQYKRCLKNENCSIVRINRNRCQQCRFKKC
LSVGMSRDAVRFGRIPKREKQRMLAEMQSAMNLANNQLSSQCPLETSPTQHPTPGPMGPSPP
PAPVPSPLVGFSQFPQQLTPPRSPSPEPTVEDVISQVARAHREIFTYAHDKLGSSPGNFNANHA
SGSPPATTPHRWENQGCPPAPNDNNTLAAQRHNEALNGLRQAPSSYPPTWPPGPAHHSCHQ
SNSNGHRLCPTHVYAAPEGKAPANSPRQGNSKNVLLACPMNMYPHGRSGRTVQEIWEDFS
MSFTPAVREVVEFAKHIPGFRDLSQHDQVTLLKAGTFEVLMVRFASLFNVKDQTVMFLSRTT
YSLQELGAMGMGDLLSAMFDFSEKLNSLALTEEELGLFTAVVLVSADRSGMENSASVEQLQ
ETLLRALRALVLKNRPLETSRFTKLLLKLPDLRTLNNMHSEKLLSFRVDAQ
NP_003813.1 nuclear receptor subfamily 5 group A member 2 isoform 2
(SEQ ID NO: 146)
MSSNSDTGDLQESLKHGLTPIVSQFKMVNYSYDEDLEELCPVCGDKVSGYHYGLLTCESCK
GFFKRTVQNNKRYTCIENQNCQIDKTQRKRCPYCRFQKCLSVGMKLEAVRADRMRGGRNK
FGPMYKRDRALKQQKKALIRANGLKLEAMSQVIQAMPSDLTISSAIQNIHSASKGLPLNHAA
LPPTDYDRSPFVTSPISMTMPPHGSLQGYQTYGHFPSRAIKSEYPDPYTSSPESIMGYSYMDSY
QTSSPASIPHLILELLKCEPDEPQVQAKIMAYLQQEQANRSKHEKLSTFGLMCKMADQTLFSI
VEWARSSIFFRELKVDDQMKLLQNCWSELLILDHIYRQVVHGKEGSIFLVTGQQVDYSIIASQ
AGATLNNLMSHAQELVAKLRSLQFDQREFVCLKFLVLFSLDVKNLENFQLVEGVQEQVNAA
LLDYTMCNYPQQTEKFGQLLLRLPEIRAISMQAEEYLYYKHLNGDVPYNNLLIEMLHAKRA
NP_001138773.1 retinoic acid receptor alpha isoform 1
(SEQ ID NO: 147)
MASNSSSCPTPGGGHLNGYPVPPYAFFFPPMLGGLSPPGALTTLQHQLPVSGYSTPSPAT
IETQSSSSEEIVPSPPSPPPLPRIYKPCFVCQDKSSGYHYGVSACEGCKGFFRRSIQKNM
VYTCHRDKNCIINKVTRNRCQYCRLQKCFEVGMSKESVRNDRNKKKKEVPKPECSESYTLT
PEVGELIEKVRKAHQETFPALCQLGKYTTNNSSEQRVSLDIDLWDKFSELSTKCIIKTV
EFAKQLPGFTTLTIADQITLLKAACLDILILRICTRYTPEQDTMTFSDGLTLNRTQMHNA
GFGPLTDLVFAFANQLLPLEMDDAETGLLSAICLICGDRQDLEQPDRVDMLQEPLLEALK
VYVRKRRPSRPHMFPKMLMKITDLRSISAKGAERVITLKMEIPGSMPPLIQEMLENSEGL
DTLSGQPGGGGRDGGGLAPPPGSCSPSLSPSSNRSSPATHSP
NP_002948.1 retinoic acid receptor RXR-alpha
(SEQ ID NO: 148)
MDTKHFLPLDFSTQVNSSLTSPTGRGSMAAPSLHPSLGPGIGSPGQLHSPISTLSSPINGMGPPF
SVISSPMGPHSMSVPTTPTLGFSTGSPQLSSPMNPVSSSEDIKPPLGLNGVLKVPAHPSGNMAS
FTKHICAICGDRSSGKHYGVYSCEGCKGFFKRTVRKDLTYTCRDNKDCLIDKRQRNRCQYC
RYQKCLAMGMKREAVQEERQRGKDRNENEVESTSSANEDMPVERILEAELAVEPKTETYVE
ANMGLNPSSPNDPVTNICQAADKQLFTLVEWAKRIPHFSELPLDDQVILLRAGWNELLIASFS
HRSIAVKDGILLATGLHVHRNSAHSAGVGAIFDRVLTELVSKMRDMQMDKTELGCLRAIVL
FNPDSKGLSNPAEVEALREKVYASLEAYCKHKYPEQPGRFAKLLLRLPALRSIGLKCLEHLFF
FKLIGDTPIDTFLMEMLEAPHQMT
NP_001017536.1 vitamin D3 receptor isoform VDRB1
(SEQ ID NO: 149)
MEWRNKKRSDWLSMVLRTAGVEEAFGSEVSVRPHRRAPLGSTYLPPAPSGMEAMAASTSLP
DPGDFDRNVPRICGVCGDRATGFHFNAMTCEGCKGFFRRSMKRKALFTCPFNGDCRITKDN
RRHCQACRLKRCVDIGMMKEFILTDEEVQRKREMILKRKEEEALKDSLRPKLSEEQQRIIAIL
LDAHHKTYDPTYSDFCQFRPPVRVNDGGGSHPSRPNSRHTPSFSGDSSSSCSDHCITSSDMMD
SSSFSNLDLSEEDSDDPSVTLELSQLSMLPHLADLVSYSIQKVIGFAKMIPGFRDLTSEDQIVLL
KSSAIEVIMLRSNESFTMDDMSWTCGNQDYKYRVSDVTKAGHSLELIEPLIKFQVGLKKLNL
HEEEHVLLMAICIVSPDRPGVQDAALIEAIQDRLSNTLQTYIRCRHPPPGSHLLYAKMIQKLA
DLRSLNEEHSKQYRCLSFQPECSMKLTPLVLEVFGNEIS
NP_000917.3 progesterone receptor isoform
(SEQ ID NO: 150)
BMTELKAKGPRAPHVAGGPPSPEVGSPLLCRPAAGPFPGSQTSDTLPEVSAIPISLDGLLFPRP
CQGQDPSDEKTQDQQSLSDVEGAYSRAEATRGAGGSSSSPPEKDSGLLDSVLDTLLAPSGPG
QSQPSPPACEVTSSWCLFGPELPEDPPAAPATQRVLSPLMSRSGCKVGDSSGTAAAHKVLPR
GLSPARQLLLPASESPHWSGAPVKPSPQAAAVEVEEEDGSESEESAGPLLKGKPRALGGAAA
GGGAAAVPPGAAAGGVALVPKEDSRFSAPRVALVEQDAPMAPGRSPLATTVMDFIHVPILPL
NHALLAARTRQLLEDESYDGGAGAASAFAPPRSSPCASSTPVAVGDFPDCAYPPDAEPKDDA
YPLYSDFQPPALKIKEEEEGAEASARSPRSYLVAGANPAAFPDFPLGPPPPLPPRATPSRPGEA
AVTAAPASASVSSASSSGSTLECILYKAEGAPPQQGPFAPPPCKAPGASGCLLPRDGLPSTSAS
AAAAGAAPALYPALGLNGLPQLGYQAAVLKEGLPQVYPPYLNYLRPDSEASQSPQYSFESLP
QKICLICGDEASGCHYGVLTCGSCKVFFKRAMEGQHNYLCAGRNDCIVDKIRRKNCPACRL
RKCCQAGMVLGGRKFKKFNKVRVVRALDAVALPQPVGVPNESQALSQRFTFSPGQDIQLIPP
LINLLMSIEPDVIYAGHDNTKPDTSSSLLTSLNQLGERQLLSVVKWSKSLPGFRNLHIDDQITLI
QYSWMSLMVFGLGWRSYKHVSGQMLYFAPDLILNEQRMKESSFYSLCLTMWQIPQEFVKL
QVSQEEFLCMKVLLLLNTIPLEGLRSQTQFEEMRSSYIRELIKAIGLRQKGVVSSSQRFYQLTK
LLDNLHDLVKQLHLYCLNTFIQSRALSVEFPEMMSEVIAAQLPKILAGMVKPLLFHKK
NP_001073315.1 CREB-binding protein isoform b
(SEQ ID NO: 151)
MAENLLDGPPNPKRAKLSSPGFSANDSTDFGSLFDLENDLPDELIPNGGELGLLNSGNLVPDA
ASKHKQLSELLRGGSGSSINPGIGNVSASSPVQQGLGGQAQGQPNSANMASLSAMGKSPLSQ
GDSSAPSLPKQAASTSGPTPAASQALNPQAQKQVGLATSSPATSQTGPGICMNANFNQTHPG
LLNSNSGHSLINQASQGQAQVMNGSLGAAGRGRGAGMPYPTPAMQGASSSVLAETLTQVSP
QMTGHAGLNTAQAGGMAKMGITGNTSPFGQPFSQAGGQPMGATGVNPQLASKQSMVNSLP
TFPTDIKNTSVTNVPNMSQMQTSVGIVPTQAIATGPTADPEKRKLIQQQLVLLLHAHKCQRRE
QANGEVRACSLPHCRTMKNVLNHMTHCQAGKACQAILGSPASGIQNTIGSVGTGQQNATSL
SNPNPIDPSSMQRAYAALGLPYMNQPQTQLQPQVPGQQPAQPQTHQQMRTLNPLGNNPMNI
PAGGITTDQQPPNLISESALPTSLGATNPLMNDGSNSGNIGTLSTIPTAAPPSSTGVRKGWHEH
VTQDLRSHLVHKLVQAIFPTPDPAALKDRRMENLVAYAKKVEGDMYESANSRDEYYHLLA
EKIYKIQKELEEKRRSRLHKQGILGNQPALPAPGAQPPVIPQAQPVRPPNGPLSLPVNRMQVS
QGMNSFNPMSLGNVQLPQAPMGPRAASPMNHSVQMNSMGSVPGMAISPSRMPQPPNMMG
AHTNNMMAQAPAQSQFLPQNQFPSSSGAMSVGMGQPPAQTGVSQGQVPGAALPNPLNMLG
PQASQLPCPPVTQSPLHPTPPPASTAAGMPSLQHTTPPGMTPPQPAAPTQPSTPVSSSGQTPTP
TPGSVPSATQTQSTPTVQAAAQAQVTPQPQTPVQPPSVATPQSSQQQPTPVHAQPPGTPLSQA
AASIDNRVPTPSSVASAETNSQQPGPDVPVLEMKTETQAEDTEPDPGESKGEPRSEMMEEDL
QGASQVKEETDIAEQKSEPMEVDEKKPEVKVEVKEEEESSSNGTASQSTSPSQPRKKIFKPEE
LRQALMPTLEALYRQDPESLPFRQPVDPQLLGIPDYFDIVKNPMDLSTIKRKLDTGQYQEPW
QYVDDVWLMFNNAWLYNRKTSRVYKFCSKLAEVFEQEIDPVMQSLGYCCGRKYEFSPQTL
CCYGKQLCTIPRDAAYYSYQNRYHFCEKCFTEIQGENVTLGDDPSQPQTTISKDQFEKKKND
TLDPEPFVDCKFCGRKMHQICVLHYDIIWPSGFVCDNCLKKTGRPRKFNKFSAKRLQTTRLG
NHLEDRVNKFLRRQNHPEAGEVFVRVVASSDKTVEVKPGMKSRFVDSGEMSESFPYRTKAL
FAFEEIDGVDVCFFGMHVQEYGSDCPPPNTRRVYISYLDSIHFFRPRCLRTAVYHEILIGYLEY
VKKLGYVTGHIWACPPSEGDDYIFHCHPPDQKIPKPKRLQEWYKKMLDKAFAERIIHDYKDI
FKQATEDRLTSAKELPYFEGDFWPNVLEESIKELEQEEEERKKEESTAASETTEGSQGDSKNA
KKKNNKKTNKNKSSISRANKKKPSMPNVSNDLSQKLYATMEKHKEVFFVIHLHAGPVINTLP
PIVDPDPLLSCDLMDGRDAFLTLARDKHWEFSSLRRSKWSTLCMLVELHTQGQDRFVYTCN
ECKHHVETRWHCTVCEDYDLCINCYNTKSHAHKMVKWGLGLDDEGSSOGEPOSKSPQESR
RLSIQRCIQSLVHACQCRNANCSLPSCQKMKRVVQHTKGCKRKTNGGCPVCKQLIALCCYH
AKHCQFNKCPVPFCLNIKHKLRQQQIQHRLQQAQLMRRRMATMNTRNVPQQSLPSPTSAPP
GTPTQQPSTPQTPQPPAQPQPSPVSMSPAGFPSVARTQPPTTVSTGKPTSQVPAPPPPAQPPPA
AVEAARQIEREAQQQQHLYRVNINNSMPPGRTGMGTPGSQMAPVSLNVPRPNQVSGPVMPS
MPPGQWQQAPLPQQQPMPGLPRPVISMQAQAAVAGPRMPSVQPPRSISPSALQDLLRTLKSP
SSPQQQQQVLNILKSNPQLMAAFIKQRTAKYVANQPGMQPQPGLQSQPGMQPQPGMHQQPS
LQNLNAMQAGVPRPGVPPQQQAMGGLNPQGQALNIMNPGHNPNMASMNPQYREMLRRQL
LQQQQQQQQQQQQQQQQQQGSAGMAGGMAGHGQFQQPQGPGGYPPAMQQQQRMQQHL
PLQGSSMGQMAAQMGQLGQMGQPGLGADSTPNIQQALQQRILQQQQMKQQIGSPGQPNPM
SPQQHMLSGQPQASHLPGQQIATSLSNQVRSPAPVQSPRPQSQPPHSSPSPRIQPQPSPHHVSP
QTGSPHPGLAVTMASSIDQGHLGNPEQSAMLPQLNTPSRSALSSELSLVGDTTGDTLEKFVEG
L
NP_001420.2 histone acetyltransferase p300
(SEQ ID NO: 152)
MAENVVEPGPPSAKRPKLSSPALSASASDGTDFGSLFDLEHDLPDELINSTELGLTNGGD
INQLQTSLGMVQDAASKHKQLSELLRSGSSPNLNMGVGGPGQVMASQAQQSSPGLGLINSM
VKSPMTQAGLTSPNMGMGTSGPNQGPTQSTGMMNSPVNQPAMGMNTGMNAGMNPGMLA
AGNGQGIMPNQVMNGSIGAGRGRQNMQYPNPGMGSAGNLLTEPLQQGSPQMGGQTGLRG
PQPLKMGMMNNPNPYGSPYTQNPGQQIGASGLGLQIQTKTVLSNNLSPFAMDKKAVPGGG
MPNMGQQPAPQVQQPGLVTPVAQGMGSGAHTADPEKRKLIQQQLVLLLHAHKCQRREQA
NGEVRQCNLPHCRTMKNVLNHMTHCQSGKSCQVAHCASSRQIISHWKNCTRHDCPVCLPL
KNAGDKRNQQPILTGAPVGLGNPSSLGVGQQSAPNLSTVSQIDPSSIERAYAALGLPYQVNQ
MPTQPQVQAKNQQNQQPCQSPQGMRPMSNMSASPMGVNCGVGVQTPSLLSDSMLHSAINS
QNPMMSENASVPSLGPMPTAAQPSTTGIRKQWHEDITQDLRNHLVHKLVQAIFPTPDPAALK
DRRMENLVAYARKVEGDMYESANNRAEYYHLLAEKIYKIQKELEEKRRTRLQKQNMLPNA
AGMVPVSMNPGPNMGQPQPGMTSNGPLPDPSMIRGSVPNQMMPRITPQSGLNQFGQMSMA
QPPIVPRQTPPLQHHGQLAQPGALNPPMGYGPRMQQPSNQGQFLPQTQFPSQGMNVTNIPLA
PSSGQAPVSQAQMSSSSCPVNSPIMPPGSQGSHIHCPQLPQPALHQNSPSPVPSRTPTPHHTPPS
IGAQQPPATTIPAPVPTPPAMPPGPQSQALHPPPRQTPTPPTTQLPQQVQPSLPAAPSADQPQQ
QPRSQQSTAASVPTPTAPLLPPQPATPLSQPAVSIEGQVSNPPSTSSTEVNSQAIAEKQPSQEVK
MEAKMEVDQPEPADTQPEDISESKVEDCKMESTETEERSTELKTEIKEEEDQPSTSATQSSPA
PGQSKKKIFKPEELRQALMPTLEALYRQDPESLPFRQPVDPQLLGIPDYFDIVKSPMDLSTIKR
KLDTGQYQEPWQYVDDIWLMFNNAWLYNRKTSRVYKYCSKLSEVFEQEIDPVMQSLGYCC
GRKLEFSPQTLCCYGKQLCTIPRDATYYSYQNRYHFCEKCFNEIQGESVSLGDDPSQPQTTIN
KEQFSKRKNDTLDPELFVECTECGRKMHQICVLHHEIIWPAGFVCDGCLKKSARTRKENKFS
AKRLPSTRLGTFLENRVNDFLRRQNHPESGEVTVRVVHASDKTVEVKPGMKARFVDSGEMA
ESFPYRTKALFAFEEIDGVDLCFFGMHVQEYGSDCPPPNQRRVYISYLDSVHFFRPKCLRTAV
YHEILIGYLEYVKKLGYTTGHIWACPPSEGDDYIFHCHPPDQKIPKPKRLQEWKKMLDKAV
SERIVHDYKDIFKQATRDRLTSAKELPYFEGDFWPNVLEESIKELEQEEEERKREENTSNESTD
VTKGDSKNAKKKNNKKTSKNKSSLSRGNKKKPGMPNVSNDLSOKLYATMEKHKEVFFVIR
LIAGPAANSLPPIVDPDPLIPCDLMDGRDAFLTLARDKHLEFSSLRRAQWSTMCMLVELHTQS
QDRFVYTCNECKHHVETRWHCTVCEDYDLCITCYNTKNHDHKMEKLGLGLDDESNNQQA
AATQSPGDSRRLSIQRCIQSLVHACQCRNANCSLPSCQKMKRVVQHTKGCKRKTNGGCPICK
QLIALCCYHAKHCQENKCPVPFCLNIKQKLRQQQLQHRLQQAQMLRRRMASMQRTGVVGQ
QQGLPSPTPATPTTPTGQQPTTPQTPQPTSQPQPTPPNSMPPYLPRTQAAGPVSQGKAAGQVT
PPTPPQTAQPPLPGPPPAAVEMAMQIQRAAETQRQMAHVQIFQRPIQHQMPPMTPMAPMGM
NPPPMTRGPSGHLEPGMGPTGMQQQPPWSQGGLPQPQQLQSGMPRPAMMSVAQHGQPLN
MAPQPGLGQVGISPLKPGTVSQQALQNLLRTLRSPSSPLQQQQVLSILHANPQLLAAFIKQRA
AKYANSNPQPIPGQPGMPQGQPGLQPPTMPGQQGVHSNPAMQNMNPMQAGVQRAGLPQQ
QPQQQLQPPMGGMSPQAQQMNMNHNTMPSQFRDILRRQQMMQQQQQQGAGPGIGPGMA
NHNQFQQPQGVGYPPQQQQRMQHHMQQMQQGNMGQIGQLPQALGAEAGASLQAYQQRL
LQQQMGSPVQPNPMSPQQHMLPNQAQSPHLQGQQIPNSLSNQVRSPQPVPSPRPQSQPPHSSP
SPRMQPQPSPHHVSPQTSSPHPGLVAAQANPMEQGHFASPDQNSMLSQLASNPGMANLHGA
SATDLGLSTDNSDLNSNLSQSTLDIH
NP_001155201.1 phospholipase A2, membrane associated precursor
(SEQ ID NO: 153)
MKTLLLLAVIMIFGLLQAHGNLVNFHRMIKLTTGKEAALSYGFYGCHCGVGGRGSPKDATD
RCCVTHDCCYKRLEKRGCGTKFLSYKFSNSGSRITCAKQDSCRSQLCECDKAAATCFARNKT
TYNKKYQYYSNKHCRGSTPRC
NP_613075.1 core histone macro-H2A.1 isoform 1
(SEQ ID NO: 153)
MSSRGGKKKSTKTSRSAKAGVIFPVGRMLRYIKKGHPKYRIGVGAPVYMAAVLEYLTAEILE
LAGNAARDNKKGRVTPRHILLAVANDEELNQLLKGVTIASGGVLPNIHPELLAKKRGSKGKL
EAIITPPPAKKAKSPSQKKPVSKKAGGKKGARKSKKKQGEVSKAASADSTTEGTPADGFTVL
STKSLFLGQKLQVVQADIASIDSDAVVHPTNTDFYIGGEVGNTLEKKGGKEFVEAVLELRKK
NGPLEVAGAAVSAGHGLPAKFVIHCNSPVWGADKCEELLEKTVKNCLALADDKKLKSIAFP
SIGSGRNGFPKQTAAQLILKAISSYFVSTMSSSIKTVYFVLFDSESIGIYVQEMAKLDAN
NP_003521.2 histone cluster 1, H3d
(SEQ ID NO: 154)
MARTKQTARKSTGGKAPRKQLATKAARKSAPATGGVKKPHRYRPGTVALREIRRYQKSTEL
LIRKLPFQRLVREIAQDFKTDLRFQSSAVMALQEACEAYLVGLFEDTNLCAIHAKRVTIMPKD
IQLARRIRGERA
NP_003504.2 histone H2A type 1-B/E
(SEQ ID NO: 155)
MSGRGKQGGKARAKAKTRSSRAGLQFPVGRVHRLLRKGNYSERVGAGAPVYLAAVLEYLT
AEILELAGNAARDNKKTRIIPRHLQLAIRNDEELNKLLGRVTIAQGGVLPNIQAVLLPKKTES
HHKAKGK
NP_808760.1 histone H2A.J
(SEQ ID NO: 156)
MSGRGKQGGKVRAKAKSRSSRACLQFPVGRVHRLLRKGNYAERVGAGAPVYLAAVLEYLT
AEILELAGNAARDNKKTRIIPRHLQLAIRNDEELNKLLGKVTIAQGGVLPNIQAVLLPKKTES
QKTKSK
NP_002097.1 histone H2A.Z
(SEQ ID NO: 157)
MAGGKAGKDSGKAKTKAVSRSQRAGLQFPVGRIHRHLKSRTTSHGRVGATAAVYSAAILEY
LTAEVLELAGNASKDLKVKRITPRHLQLAIRGDEELDSLIKATIAGGGVIPHIHKSLIG
KKGQQKTV
NP_066406.1 histone H2B type 1-B
(SEQ ID NO: 158)
MPEPSKSAPAPKKGSKKAITKAQKKDGKKRKRSRKESYSIYVYKVLKQVHPDTGISSKAMGI
MNSFVNDIFERIAGEASRLAHYNKRSTITSREIQTAVRLLLPGELAKHAVSEGTKAVT
KYTSSK
NP_066402.2 histone H2B type 1-J
(SEQ ID NO: 159)
MPEPAKSAPAPKKGSKKAVTKAQKKDGKKRKRSRKESYSIYVYKVLKQVHPDTGISSKAMG
IMNSFVNDIFERIAGEASRLAHYNKRSTITSREIQTAVRLLLPGELAKHAVSEGTKAVTKYTSA
K
NP_542160.1 histone H2B type 1-K
(SEQ ID NO: 160)
MPEPAKSAPAPKKGSKKAVTKAQKKDGKKRKRSRKESYSVYVYKVLKQVHPDTGISSKAM
GIMNSFVNDIFERIAGEASRLAHYNKRSTITSREIQTAVRLLLPGELAKHAVSEGTKAVTKYTS
AK
NP_003518.2 histone H2B type 1-0
(SEQ ID NO: 161)
MPDPAKSAPAPKKGSKKAVTKAQKKDGKKRKRSRKESYSIYVYKVLKQVHPDTGISSKAM
GIMNSFVNDIFERIAGEASRLAHYNKRSTITSREIQTAVRLLLPGELAKHAVSEGTKAVTKYTS
SK
NP_003484.1 histone H3.1t
(SEQ ID NO: 162)
MARTKQTARKSTGGKAPRKQLATKVARKSAPATGGVKKPHRYRPGTVALREIRRYQKSTEL
LIRKLPFQRLMREIAQDFKTDLRFQSSAVMALQEACESYLVGLFEDTNLCVIHAKRVTIMPKD
IQLARRIRGERA
NP_001116847.1 histone H3.2
(SEQ ID NO: 163)
MARTKQTARKSTGGKAPRKQLATKAARKSAPATGGVKKPHRYRPGTVALREIRRYQKSTEL
LIRKLPFQRLVREIAQDFKTDLRFQSSAVMALQEASEAYLVGLFEDTNLCAIHAKRVTIMPKD
IQLARRIRGERA
NP_005315.1 histone H3.3
(SEQ ID NO: 164)
MARTKQTARKSTGGKAPRKQLATKAARKSAPSTGGVKKPHRYRPGTVALREIRRYQKSTEL
LIRKLPFQRLVREIAQDFKTDLRFQSAAIGALQEASEAYLVGLFEDTNLCAIHAKRVTIMPKDI
QLARRIRGERA
NP_001029249.1 histone H4
(SEQ ID NO: 165)
MSGRGKGGKGLGKGGAKRHRKVLRDNIQGITKPAIRRLARRGGVKRISGLIYEETRGVLKVF
LENVIRDAVTYTEHAKRKTVTAMDVVYALKRQGRTLYGFGG
NP_001902.1 cathepsin G preproprotein
(SEQ ID NO: 166)
MQPLLLLLAFLLPTGAEAGEIIGGRESRPHSRPYMAYLQIQSPAGQSRCGGFLVREDFVL
TAAHCWGSNINVTLGAHNIQRRENTQQHITARRAIRHPQYNQRTIQNDIMLLQLSRRVRR
NRNVNPVALPRAQEGLRPGTLCTVAGWGRVSMRRGTDTLREVQLRVQRDRQCLRIFGSYDP
RRQICVGDRRERKAAFKGDSGGPLLCNNVAHGIVSYGKSSGVPPEVFTRVSSFLPWIRTTMR
SFKLLDQMETPL
NP_002119.1 high mobility group protein B1
(SEQ ID NO: 167)
MGKGDPKKPRGKMSSYAFFVQTCREEHKKKHPDASVNFSEFSKKCSERWKTMSAKEKGKF
EDMAKADKARYEREMKTYIPPKGETKKKFKDPNAPKRPPSAFFLFCSEYRPKIKGEHPGLSIG
DVAKKLGEMWNNTAADDKQPYEKKAAKLKEKYEKDIAAYRAKGKPDAAKKGVVKAEKS
KKKKEEEEDEEDEEDEEEEEDEEDEDEEEDDDDE
NP_001997.5 heparin-binding growth factor 2
(SEQ ID NO: 168)
MVGVGGGDVEDVTPRPGGCQISGRGARGCNGIPGAAAWEAALPRRRPRRHPSVNPRSRAAG
SPRTRGRRTEERPSGSRLGDRGRGRALPGGRLGGRGRGRAPERVGGRGRGRGTAAPRAAPA
ARGSRPGPAGTMAAGSITTLPALPEDGGSGAFPPGHFKDPKRLYCKNGGFFLRIHPDGRVDG
VREKSDPHIKLQLQAEERGVVSIKGVCANRYLAMKEDGRLLASKCVTDECFFFERLESNNYN
TYRSRKYTSWYVALKRTGQYKLGSKTGPGQKAILFLPMSAKS
NP_001155201.1 phospholipase A2, membrane associated precursor
(SEQ ID NO: 169)
MKTLLLLAVIMIFGLLQAHGNLVNFHRMIKLTTGKEAALSYGFYGCHCGVGGRGSPKDATD
RCCVTHDCCYKRLEKRGCGTKFLSYKFSNSGSRITCAKQDSCRSQLCECDKAAATCFARNKT
TYNKKYQYYSNKHCRGSTPRC
NP_002719.3 bone marrow proteoglycan preproprotein
(SEQ ID NO: 170)
MKLPLLLALLFGAVSALHLRSETSTFETPLGAKTLPEDEETPEQEMEETPCRELEEEEEW
GSGSEDASKKDGAVESISVPDMVDKNLTCPEEEDTVKVVGIPGCQTCRYLLVRSLQTFSQ
AWFTCRRCYRGNLVSIHNFNINYRIQCSVSALNQGQVWIGGRITGSGRCRRFQWVDGSRWN
FAYWAAHQPWSRGGHCVALCTRGGHWRRAHCLRRLPFICSY
NP_008869.1 small nuclear ribonucleoprotein Sm D1
(SEQ ID NO: 171)
MKLVRFLMKLSHETVTIELKNGTQVHGTITGVDVSMNTHLKAVKMTLKNREPVQLETLSIR
GNNIRYFILPDSLPLDTLLVDVEPKVKSKKREAVAGRGRGRGRGRGRGRGRGRGGPRR
NP_060771.3 RNA-binding protein 41 isoform 1
(SEQ ID NO: 172)
MKRVNSCVKSDEHVLEELETEGERQLKSLLQHQLDTSVSIEECMSKKESFAPGTMYKPFGKE
AAGTMTLSQFQTLHEKDQETASLRELGLNETEILIWKSHVSGEKKTKLRATPEAIQNRLQDIE
ERISERQRILCLPQRFAKSKQLTRREMEIEKSLFQGADRHSFLKALYYQDEPQKKNKGDPMN
NLESFYQEMIMKKRLEEFQLMRGEPFASHSLVSATSVGDSGTAESPSLLQDKGKQAAQGKGP
SLHVANVIDFSPEQCWTGPKKLTQPIEFVPEDEIQRNRLSEEEIRKIPMFSSYNPGEPNKVLYL
KNLSPRVTERDLVSLFARFQEKKGPPIQFRMMTGRMRGQAFITFPNKEIAWQALHLVNGYKL
HGKILVIEFGKNKKQRSNLQATSLISCATGSTTEISGS
NP_619520.1 putative ATP-dependent RNA helicase DHX30 isoform 1
(SEQ ID NO: 173)
MFSLDSFRKDRAQHRQRQCKLPPPRLPPMCVNPTPGGTISRASRDLLKEFPQPKNLLNSVIGR
ALGISHAKDKLVYVHTNGPKKKKVTLHIKWPKSVEVEGYGSKKIDAERQAAAAACQLFKG
WGLLGPRNELFDAAKYRVLADRFGSPADSWWRPEPTMPPTSWRQLNPESIRPGGPGGLSRSL
GREEEEDEEEELEEGTIDVTDFLSMTQQDSHAPLRDSRGSSFEMTDDDSAIRALTQFPLPKNL
LAKVIQIATSSSTAKNLMQFHTVGTKTKLSTLTLLWPCPMTFVAKGRRKAEAENKAAALAC
KKLKSLGLVDRNNEPLTHAMYNLASLRELGETQRRPCTIQVPEPILRKIETFLNHYPVESSWI
APELRLQSDDILPLGKDSGPLSDPITGKPYVPLLEAEEVRLSQSLLELWRRRGPVWQEAPQLP
VDPHRDTILNAIEQHPVVVISGDTGCGKTTRIPQLLLERYVTEGRGARCNVIITQPRRISAVSV
AQRVSHELGPSLRRNVGFQVRLESKPPSRGGALLFCTVGILLRKLQSNPSLEGVSHVIVDEVH
ERDVNTDFLLILLKGLQRLNPALRLVLMSATGDNERFSRYFGGCPVIKVPGFMYPVKEHYLE
DILAKLGKHQYLHRHRHHESEDECALDLDLVTDLVLHIDARGEPGGILCFLPGWQEIKGVQQ
RLQEALGMHESKYLILPVHSNIPMMDQKAIFQQPPVGVRKIVLATNIAETSITINDIVHVVDSG
LHKEERYDLKTKVSCLETVWVSRANVIQRRGRAGRCQSGFAYHLFPRSRLEKMVPFQVPEIL
RTPLENLVLQAKIHMPEKTAVEFLSKAVDSPNIKAVDEAVILLQEIGVLDQREYLTTLGQRLA
HISTDPRLAKAIVLAAIFRCLHPLLVVVSCLTRDPFSSSLQNRAEVDKVKALLSHDSGSDHLA
FVRAVAGWEEVLRWQDRSSRENYLEENLLYAPSLRFIHGLIKQFSENIYEAFLVGKPSDCTLA
SAQCNEYSEEEELVKGVLMAGLYPNLIQVRQGKVTRQGKFKPNSVTYRTKSGNILLHKSTIN
REATRLRSRWLTYFMAVKSNGSVFVRDSSQVHPLAVLLLTDGDVHIRDDGRRATISLSDSDL
LRLEGDSRTVRLLKELRRALGRMVERSLRSELAALPPSVQEEHGQLLALLAELLRGPCGSFD
VRKTADD
NP_001129248.1 zinc finger and BTB domain-containing protein 43
(SEQ ID NO: 174)
MEPGTNSFRVEFPDFSSTILQKLNQQRQQGQLCDVSIVVQGHIFRAHKAVLAASSPYFCDQVL
LKNSRRIVLPDVMNPRVFENILLSSYTGRLVMPAPEIVSYLTAASFLQMWHVVDKCTEVLEG
NPTVLCQKLNHGSDHQSPSSSSYNGLVESFELGSGGHTDFPKAQELRDGENEEESTKDELSSQ
LTEHEYLPSNSSTEHDRLSTEMASQDGEEGASDSAEFHYTRPMYSKPSIMAHKRWIHVKPER
LEQACEGMDVHATYDEHQVTESINTVQTEHTVQPSGVEEDFHIGEKKVEAEFDEQADESNY
DEQVDFYGSSMEEFSGERSDGNLIGHRQEAALAAGYSENIEMVTGIKEEASHLGFSATDKLY
PCQCGKSFTHKSQRDRHMSMHLGLRPYGCGVCGKKFKMKHHLVGHMKIHTGIKPYECNIC
AKRFMWRDSFHRHVTSCTKSYEAAKAEQNTTEAN
NP_001721.2 Krueppel-like factor 5
(SEQ ID NO: 175)
MATRVLSMSARLGPVPQPPAPQDEPVFAQLKPVLGAANPARDAALFPGEELKHAHHRPQAQ
PAPAQAPQPAQPPATGPRLPPEDLVQTRCEMEKYLTPQLPPVPIIPEHKKYRRDSASVVDQFF
TDTEGLPYSINMNVFLPDITHLRTGLYKSQRPCVTHIKTEPVAIFSHQSETTAPPPAPTQALPEF
TSIFSSHQTAAPEVNNIFIKQELPTPDLHLSVPTQQGHLYQLLNTPDLDMPSSTNQTAAMDTL
NVSMSAAMAGLNTHTSAVPQTAVKQFQGMPPCTYTMPSQFLPQQATYFPPSPPSSEPGSPDR
QAEMLQNLTPPPSYAATIASKLAIHNPNLPTTLPVNSQNIQPVRYNRRSNPDLEKRRIHYCDY
PGCTKVYTKSSHLKAHLRTHTGEKPYKCTWEGCDWRFARSDELTRHYRKHTGAKPFQCGV
CNRSFSRSDHLALHMKRHQN
NP_001129687.1 artemin isoform 3 precursor
(SEQ ID NO: 176)
MELGLGGLSTLSHCPWPRQQAPLGLSAQPALWPTLAALALLSSVAEASLGSAPRSPAPREGP
PPVLASPAGHLPGGRTARWCSGRARRPPPQPSRPAPPPPAPPSALPRGGRAARAGGPGSRAR
AAGARGCRLRSQLVPVRALGLGHRSDELVRFRFCSGSCRRARSPHDLSLASLLGAGALRPPP
GSRPVSQPCCRPTRYEAVSFMDVNSTWRTVDRLSATACGCLG
NP_113623.1 THAPdomain-containing protein 2
(SEQ ID NO: 177)
MPTNCAAAGCATTYNKHINISFHRFPLDPKRRKEWVRLVRRKNFVPGKHTFLCSKHFEASCF
DLTGQTRRLKMDAVPTIFDFCTHIKSMKLKSRNLLKKNNSCSPAGPSNLKSNISSQQVLLEHS
YAFRNPMEAKKRIIKLEKEIASLRRKMKTCLQKERRATRRWIKATCLVKNLEANSVLPKGTS
EHMLPTALSSLPLEDFKILEQDQQDKTLLSLNLKQTKSTFI
NP_001020092.1 60S ribosomal protein L9
(SEQ ID NO: 178)
MKTILSNQTVDIPENVDITLKGRTVIVKGPRGTLRRDFNHINVELSLLGKKKKRLRVDKWWG
NRKELATVRTICSHVQNMIKGVTLGFRYKMRSVYAHFPINVVIQENGSLVEIRNFLGEKYIRR
VRMRPGVACSVSQAQKDELILEGNDIELVSNSAALIQQATTVKNKDIRKFLDGIYVSEKGTV
QQADE
NP_775956.1 E3 SUMO-protein ligase NSE2
(SEQ ID NO: 179)
MPGRSSSNSGSTGFISFSGVESALSSLKNFQACINSGMDTASSVALDLVESQTEVSSEYSMDK
AMVEFATLDRQLNHYVKAVQSTINHVKEERPEKIPDLKLLVEKKFLALQSKNSDADFQNNE
KFVQFKQQLKELKKQCGLQADREADGTEGVDEDIIVTQSQTNFTCPITKEEMKKPVKNKVC
GHTYEEDAIVRMIESRQKRKKKAYCPQIGCSHTDIRKSDLIQDEALRRAIENHNKKRHRHSE
NP_060667.2 zinc finger protein 64 isoform a
(SEQ ID NO: 180)
MNASSEGESFAGSVQIPGGTTVLVELTPDIHICGICKQQFNNLDAFVAHKQSGCQLTGTS
AAAPSTVQFVSEETVPATQTQTTTRTITSETQTITVSAPEFVFEHGYQTYLPTESNENQT
ATVISLPAKSRTKKPTTPPAQKRLNCCYPGCQFKTAYGMKDMERHLKIHTGDKPHKCEVCG
KCFSRKDKLKTHMRCHTGVKPYKCKTCDYAAADSSSLNKHLRIHSDERPFKCQICPYASRNS
SQLTVHLRSHTGDAPFQCWLCSAKFKISSDLKRHMRVHSGEKPFKCEFCNVRCTMKGNLKS
HIRIKHSGNNFKCPHCDFLGDSKATLRKHSRVHQSEHPEKCSECSYSCSSKAALRIHERIHCTD
RPFKCNYCSFDTKQPSNLSKHMKKFHGDMVKTEALERKDTGRQSSRQVAKLDAKKSFHCDI
CDASFMREDSLRSHKRQHSEYSESKNSDVTVLQFQIDPSKQPATPLTVGHLQVPLQPSQVPQF
SEGRVKIIVGHQVPQANTIVQAAAAAVNIVPPALVAQNPEELPGNSRLQILRQVSLIAPPQSSR
CPSEAGAMTQPAVLLTTHEQTDGATLHQTLIPTASGGPQEGSGNQTFITSSGITCTDFEGLNA
LIQEGTAEVTVVSDGGQNIAVATTAPPVFSSSSQQELPKQTYSIIQGAAHPALLCPADSIPD
NP_114440.1 POZ-, AT hook-, and zinc finger-containing protein 1 short
isoform
(SEQ ID NO: 181)
MERVNDASCGPSGCYTYQVSRHSTEMLHNLNQQRKNGGRFCDVLLRVGDESFPAHRAVLA
ACSEYFESVFSAQLGDGGAADGGPADVGGATAAPGGGAGGSRELEMHTISSKVFGDILDFA
YTSRIVVRLESFPELMTAAKFLLMRSVIEICQEVIKQSNVQILVPPARADIMLFRPPGTSDLGFP
LDMTNGAALAANSNGIAGSMQPEEEAARAAGAAIAGQASLPVLPGVDRLPMVAGPLSPQLL
TSPFPSVASSAPPLTGKRGRGRPRKANLLDSMFGSPGGLREAGILPCGLCGKVFTDANRLRQH
EAQHGVTSLQLGYIDLPPPRLGENGLPISEDPDGPRKRSRTRKQVACEICGKIFRDVYHLNRH
KLSHSGEKPYSCPVCGLRFKRKDRMSYHVRSHDGSVGKPYICQSCGKGFSRPDHLNGHIKQV
HTSERPHKCQVWVGSSSGLPPLEPLPSDLPSWDFAQPALWRSSHSVPDTAFSLSLKKSFPALE
NLGPAHSSNTLFCPAPPGYLRQGWTTPEGSRAFTQWPVG
NP_149105.3 zinc finger CCHC-type and RNA-binding motif-containing protein 1
(SEQ ID NO: 182)
MSGGLAPSKSTVYVSNLPFSLTNNDLYRIFSKYGKVVKVTIMKDKDTRKSKGVAFILFLD
KDSAQNCTRAINNKQLFGRVIKASIAIDNGRAAEFIRRRNYFDKSKCYECGESGHLSYAC
PKNMLGEREPPKKKEKKKKKKAPEPEEEIEEVEESEDEGEDPALDSLSQAIAFQQAKIEE
EQKKWKPSSGVPSTSDDSRRPRIKKSTYFSDEEELSD
NP_036389.2 HMG box-containing protein 1
(SEQ ID NO: 183)
MVWEVKTNQMPNAVQKLLLVMDKRASGMNDSLELLQCNENLPSSPGYNSCDEHMELDDL
PELQAVQSDPTQSGMYQLSSDVSHQEYPRSSWNQNTSDIPETTYRENEVDWLTELANIATSP
QSPLMQCSFYNRSSPVHIIATSKSLHSYARPPPVSSSSKSEPAFPHHHWKEETPVRHERANSES
ESGIFCMSSLSDDDDLGWCNSWPSTVWHCFLKGTRLCFHKGSNKEWQDVEDFARAEGCDN
EEDLQMGIHKGYGSDGLKLLSHEESVSFGESVLKLTFDPGTVEDGLLTVECKLDHPFYVKNK
GWSSFYPSLTVVQHGIPCCEVHIGDVCLPPGHPDAINFDDSGVFDTFKSYDFTPMDSSAVYVL
SSMARQRRASLSCGGPGGQDFARSGFSKNCGSPGSSQLSSNSLYAKAVKNHSSGTVSATSPN
KCKRPMNAFMLFAKKYRVEYTQMYPGKDNRAISVILGDRWKKMKNEERRMYTLEAKALA
EEQKRLNPDCWKRKRTNSGSQQH
NP_115672.2 FLYWCH-type zinc finger-containing protein 1 isoform a
(SEQ ID NO: 184)
MPLPEPSEQEGESVKAGQEPSPKPGTDVIPAAPRKPREFSKLVLLTASDQDEDGVGSKPQ
EVHCVLSLEMAGPATLASTLQILPVEEQGGVVQPALEMPEQKCSKLDAAPQSLEFLRTPF
GGRLLVLESFLYKQEKAVGDKVYWKCRQHAELGCRGRAITRGLRATVMRGHCHAPDEQGL
EARRQREKLPSLALPEGLGEPQGPEGPGGRVEEPLEGVGPWQCPEEPEPTPGLVLSKPALEEE
EAPRALSLLSLPPKKRSILGLGQARPLEFLRTCYGGSFLVHESFLYKREKAVGDKVYWTCRD
HALHGCRSRAITQGQRVTVMRGHCHQPDMEGLEARRQQEKAVETLQAGQDGPGSQVDTLL
RGVDSLLYRRGPGPLTLTRPRPRKRAKVEDQELPTQPEAPDEHQDMDADPGGPEFLKTPLGG
SFLVYESFLYRREKAAGEKVYWTCRDQARMGCRSRAITQGRRVTVMRGHCHPPDLGGLEA
LRQREKRPNTAQRGSPGGPEFLKTPLGGSFLVYESFLYRREKAAGEKVYWTCRDQARMGCR
SRAITQGRRVMVMRRHCHPPDLGGLEALRQREHFPNLAQWDSPDPLRPLEFLRTSLGGRFLV
HESFLYRKEKAAGEKVYWMCRDQARLGCRSRAITQGHRIMVMRSHCHQPDLAGLEALRQR
ERLPTTAQQEDPEKIQVQLCFKTCSPESQQIYGDIKDVRLDGESQ
NP_005645.1 COUP transcription factor 1
(SEQ ID NO: 185)
MAMVVSSWRDPQDDVAGGNPGGPNPAAQAARGGGGGAGEQQQQAGSGAPHTPQTPGQPG
APATPGTAGDKGQGPPGSGQSQQHIECVVCGDKSSGKHYGQFTCEGCKSFFKRSVRRNLTY
TCRANRNCPIDQHHRNQCQYCRLKKCLKVGMRREAVQRGRMPPTQPNPGQYALTNGDPLN
GHCYLSGYISLLLRAEPYPTSRYGSQCMQPNNIMGIENICELAARLLFSAVEWARNIPFFPDLQ
ITDQVSLLRLTWSELFVLNAAQCSMPLHVAPLLAAAGLHASPMSADRVVAFMDHIRIFQEQV
EKLKALHVDSAEYSCLKAIVLFTSDACGLSDAAHIESLQEKSQCALEEYVRSQYPNQPSRFGK
LLLRLPSLRTVSSSVIEQLFFVRLVGKTPIETLIRDMLLSGSSFNWPYMSIQCS
NP_006457.2 DNA-directed RNA polymerase III subunit RPC6
(SEQ ID NO: 186)
MAEVKVKVQPPDADPVEIENRIIELCHQFPHGITDQVIQNEMPHIEAQQRAVAINRLLSM
GQLDLLRSNTGLLYRIKDSQNAGKMKGSDNQEKLVYQIIEDAGNKGIWSRDIRYKSNLPLTEI
NKILKNLESKKLIKAVKSVAASKKKVYMLYNLQPDRSVTGGAWYSDQDFESEFVEVLNQQC
FKFLQSKAETARESKQNPMIQRNSSFASSHEVWKYICELGISKVELSMEDIETILNTLIYDGKV
EMTIIAAKEGTVGSVDGHMKLYRAVNPIIPPTGLVRAPCGLCPVFDDCHEGGEISPSNCIYMT
EWLEF
NP_005333.2 high mobility group protein B3
(SEQ ID NO: 187)
MAKGDPKKPKGKMSAYAFFVQTCREEHKKKNPEVPVNFAEFSKKCSERWKTMSGKEKSKF
DEMAKADKVRYDREMKDYGPAKGGKKKKDPNAPKRPPSGFFLFCSEFRPKIKSTNPGISIGD
VAKKLGEMWNNLNDSEKQPYITKAAKLKEKYEKDVADYKSKGKFDGAKGPAKVARKKVE
EEDEEEEEEEEEEEEEEDE
NP_001165290.1 peroxisome proliferator-activated receptor delta isoform 3
(SEQ ID NO: 188)
MHQRDLSRSSSPPSLLDQLQMGCDGASCGSLNMECRVCGDKASGFHYGVHACEGCKGFFR
RTIRMKLEYEKCERSCKIQKKNRNKCQYCRFQKCLALGMSHNAIRFGRMPEAEKRKLVAGL
TANEGSQYNPQVADLKAFSKHIYNAYLKNFNMTKKKARSILTGKASHTAPFVIHDIETLWQA
EKGLVWKQLVNGLPPYKEISVHVFYRCQCTTVETVRELTEFAKSIPSFSSLFLNDQVTLLKYG
VHEAIFAMLASIVNKDGLLVANGSGFVTREFLRSLRKPFSDIIEPKFEFAVKFNALELDDSDLA
LFIAAIILCGDRPGLMNVPRVEAIQDTILRALEFHLQANHPDAQYLFPKLLQKMADLRQLVTE
HAQMMQRIKKTETETSLHPLLQEIYKDMY
NP_003783.1 endothelial differentiation-related factor 1 isoform alpha
(SEQ ID NO: 189)
MAESDWDTVTVLRKKGPTAAQAKSKQAILAAQRRGEDVETSKKWAAGQNKQHSITKNTA
KLDRETEELHHDRVTLEVGKVIQQGRQSKGLTQKDLATKINEKPQVIADYESGRAIPNNQVL
GKIERAIGLKLRGKDIGKPIEKGPRAK
NP_620410.3 homeobox protein TGIF2LX
(SEQ ID NO: 190)
MEAAADGPAETQSPVEKDSPAKTQSPAQDTSIMSRNNADTGRVLALPEHKKKRKGNLPAES
VKILRDWMYKHRFKAYPSEEEKQMLSEKTNLSLLQISNWFINARRRILPDMLQQRRNDPIIGH
KTGKDAHATHLQSTEASVPAKSGPSGPDNVQSLPLWPLPKGQMSREKQPDPESAPSQKLTGI
AQPKKKVKVSVTSPSSPELVSPEEHADFSSFLLLVDAAVQRAAELELEKKQEPNP
NP_003131.1 sex-determining region Y protein
(SEQ ID NO: 191)
MQSYASAMLSVFNSDDYSPAVQENIPALRRSSSFLCTESCNSKYQCETGENSKGNVQDRVKR
PMNAFIVWSRDQRRKMALENPRMRNSEISKQLGYQWKMLTEAEKWPFFQEAQKLQAMHR
EKYPNYKYRPRRKAKMLPKNCSLLPADPASVLCSEVQLDNRLYRDDCTKATHSRMEHQLG
HLPPINAASSPQQRDRYSHWTKL
NP_000956.2 retinoic acid receptor beta isoform 1
(SEQ ID NO: 191)
MFDCMDVLSVSPGQILDFYTASPSSCMLQEKALKACFSGLTQTEWQHRHTAQSIETQSTS
SEELVPSPPSPLPPPRVYKPCFVCQDKSSGYHYGVSACEGCKGFFRRSIQKNMIYTCHRD
KNCVINKVTRNRCQYCRLQKCFEVGMSKESVRNDRNKKKKETSKQECTESYEMTAELDDL
TEKIRKAHQETFPSLCQLGKYTTNSSADHRVRLDLGLWDKFSELATKCIIKIVEFAKRLPGFT
GLTIADQITLLKAACLDILILRICTRYTPEQDTMTFSDGLTLNRTQMHNAGFGPLTD
LVFTFANQLLPLEMDDTETGLLSAICLICGDRQDLEEPTKVDKLQEPLLEALKIYIRKRR
PSKPHMFPKILMKITDLRSISAKGAERVITLKMEIPGSMPPLIQEMLENSEGHEPLTPSS
SGNTAEHSPSISPSSVENSGVSQSPLVQ
NP_064589.2 LIM/homeobox protein Lhx9 isoform 1
(SEQ ID NO: 192)
MEIVGCRAEDNSCPFRPPAMLFHGISGGHIQGIMEEMERRSKTEARLAKGAQLNGRDAGMPP
LSPEKPALCAGCGGKISDRYYLLAVDKQWHLRCLKCCECKLALESELTCFAKDGSIYCKEDY
YRRFSVQRCARCHLGISASEMVMRARDSVYHLSCFTCSTCNKTLTTGDHFGMKDSLVYCRA
HFETLLQGEYPPQLSYTELAAKSGGLALPYFNGTGTVQKGRPRKRKSPALGVDIVNYNSGCN
ENEADHLDRDQQPYPPSQKTKRMRTSFKHHQLRTMKSYFAINHNPDAKDLKQLAQKTGLT
KRVLQVWFQNARAKFRRNLLRQENGGVDKADGTSLPAPPSADSGALTPPGTATTLTDLTNP
TITVVTSVTSNMDSHESGSPSQTTLTNLF
NP_067545.3 homeobox protein BarH-like 1
(SEQ ID NO: 193)
MQRPGEPGAARFGPPEGCADHRPHRYRSFMIEEILTEPPGPKGAAPAAAAAAAGELLKFGVQ
ALLAARPFHSHLAVLKAEQAAVFKFPLAPLGCSGLSSALLAAGPGLPGAAGAPHLPLELQLR
GKLEAAGPGEPGTKAKKGRRSRTVFTELQLMGLEKRFEKQKYLSTPDRIDLAESLGLSQLQV
KTWYQNRRMKWKKIVLQGGGLESPTKPKGRPKKNSIPTSEQLTEQERAKDAEKPAEVPGEP
SDRSRED
NP997704.1 protein kinase C delta type
(SEQ ID NO: 194)
MAPFLRIAFNSYELGSLQAEDEANQPFCAVKMKEALSTERGKTLVQKKPTMYPEWKSTFDA
HIYEGRVIQIVLMRAAEEPVSEVTVGVSVLAERCKKNNGKAEFWLDLQPQAKVLMSVQYFL
EDVDCKQSMRSEDEAKFPTMNRRGAIKQAKIHYIKNHEFIATFFGQPTFCSVCKDFVWGLNK
QGYKCRQCNAAIHKKCIDKIIGRCTGTAANSRDTIFQKERFNIDMPHRFKVHNYMSPTFCDH
CGSLLWGLVKQGLKCEDCGMNVHHKCREKVANLCGINQKLLAEALNQVTQRASRRSDSAS
SEPVGIYQGFEKKTGVAGEDMQDNSGTYGKIWEGSSKCNINNFIFHKVLGKGSFGKVLLGEL
KGRGEYFAIKALKKDVVLIDDDVECTMVEKRVLTLAAENPFLTHLICTFQTKDHLFFVMEFL
NGGDLMYHIQDKGRFELYRATFYAAEIMCGLQFLHSKGIIYRDLKLDNVLLDRDGHIKIADF
GMCKENIFGESRASTFCGTPDYIAPEILQGLKYTFSVDWWSFGVLLYEMLIGQSPFHGDDEDE
LFESIRVDTPHYPRWITKESKDILEKLFEREPTKRLGVTGNIKIHPFFKTINWTLLEKRRLEPPF
RPKVKSPRDYSNFDQEFLNEKARLSYSDKNLIDSMDQSAFAGFSFVNPKFEHLLED
NP_079507.1 zinc finger and SCAN domain-containing protein 16
(SEQ ID NO: 195)
MTTALEPEDQKGLLIIKAEDHYWGQDSSSQKCSPHRRELYRQHFRKLCYQDAPGPREALTQL
WELCRQWLRPECHTKEQILDLLVLEQFLSILPKDLQAWVRAHHPETGEEAVTVLEDLERELD
EPGKQVPGNSERRDILMDKLAPLGRPYESLTVQLHPKKTQLEQEAGKPQRNGDKTRTKNEE
LFQKEDMPKDKEFLGEINDRLNKDTPQHPKSKDIIENEGRSEWQQRERRRYKCDECGKSFSH
SSDLSKHRRTHTGEKPYKCDECGKAFIQRSHLIGHHRVHTGVKPYKCKECGKDFSGRTGLIQ
HQRIHTGEKPYECDECGRPFRVSSALIRHQRIHTANKLY
NP_001002261.1 zinc finger FYVE domain-containing protein 27 isoform a
(SEQ ID NO: 196)
MQTSEREGSGPELSPSVMPEAPLESPPFPTKSPAFDLFNLVLSYKRLEIYLEPLKDAGDG
VRYLLRWQMPLCSLLTCLGLNVLFLTLNEGAWYSVGALMISVPALLGYLQEVCRARLPDSE
LMRRKYHSVRQEDLQRGRLSRPEAVAEVKSFLIQLEAFLSRLCCTCEAAYRVLHWENPVVSS
QFYGALLGTVCMLYLLPLCWVLTLLNSTLFLGNVEFFRVVSEYRASLQQRMNPKQEEHAFE
SPPPPDVGGKDGLMDSTPALTPTESLSSQDLTPGSVEEAEEAEPDEEFKDAIEETHLVVLEDD
EGAPCPAEDELALQDNGFLSKNEVLRSKVSRLTERLRKRYPTNNFGNCTGCSATFSVLKKRR
SCSNCGNSFCSRCCSFKVPKSSMGATAPEAQRETVFVCASCNQTLSK
NP_001556.2 C-X-C motif chemokine 10 precursor
(SEQ ID NO: 197)
MNQTAILICCLIFLTLSGIQGVPLSRTVRCTCISISNQPVNPRSLEKLEIIPASQFCPRV
EIIATMKKKGEKRCLNPESKAIKNLLKAVSKERSKRSP
NP_006248.1 protein kinase C theta type
(SEQ ID NO: 198)
MSPFLRIGLSNFDCGSCQSCQGEAVNPYCAVLVKEYVESENGQMYIQKKPTMYPPWDSTFD
AHINKGRVMQIIVKGKNVDLISETTVELYSLAERCRKNNGKTEIWLELKPQGRMLMNARYFL
EMSDTKDMNEFETEGFFALHQRRGAIKQAKVHHVKCHEFTATFFPQPTFCSVCHEFVWGLN
KQGYQCRQCNAAIHKKCIDKVIAKCTGSAINSRETMFHKERFKIDMPHRFKVYNYKSPTFCE
HCGTLLWGLARQGLKCDACGMNVHHRCQTKVANLCGINQKLMAEALAMIESTQQARCLR
DTEQIFREGPVEIGLPCSIKNEARPPCLPTPGKREPQGISWESPLDEVDKMCHLPEPELNKERPS
LQIKLKIEDFILHKMLGKGSFGKVFLAEFKKTNQFFAIKALKKDVVLMDDDVECTMVEKRVL
SLAWEHPFLTHMFCTFQTKENLFFVMEYLNGGDLMYHIQSCHKFDLSRATFYAAEIILGLQF
LHSKGIVYRDLKLDNILLDKDGHIKIADFGMCKENMLGDAKTNTFCGTPDYIAPEILLGQKY
NHSVDWWSFGVLLYEMLIGQSPFHGQDEEELFHSIRMDNPFYPRWLEKEAKDLLVKLFVRE
PEKRLGVRGDIRQHPLFREINWEELERKEIDPPFRPKVKSPFDCSNFDKEFLNEKPRLSFADRA
LINSMDQNMFRNFSFMNPGMERLIS
NP_055850.1 zinc fingers and homeoboxes protein 3
(SEQ ID NO: 199)
MASKRKSTTPCMIPVKTVVLQDASMEAQPAETLPEGPQQDLPPEASAASSEAAQNPSSTD
GSTLANGHRSTLDGYLYSCKYCDFRSHDMTQFVGHMNSEHTDFNKDPTFVCSGCSFLAKTP
EGLSLHNATCHSGEASFVWNVAKPDNHVVVEQSIPESTSTPDLAGEPSAEGADGQAEIIITKT
PIMKIMKGKAEAKKIHTLKENVPSQPVGEALPKLSTGEMEVREGDHSFINGAVPVSQASASS
AKNPHAANGPLIGTVPVLPAGIAQFLSLQQQPPVHAQHHVHQPLPTAKALPKVMIPLSSIPTY
NAAMDSNSFLKNSFHKFPYPTKAELCYLTVVTKYPEEQLKIWFTAQRLKQGISWSPEEIEDA
RKKMFNTVIQSVPQPTITVLNTPLVASAGNVQHLIQAALPGHVVGQPEGTGGGLLVTQPLMA
NGLQATSSPLPLTVTSVPKQPGVAPINTVCSNTTSAVKVVNAAQSLLTACPSITSQAFLDASIY
KNKKSHEQLSALKGSFCRNQFPGQSEVEHLTKVTGLSTREVRKWFSDRRYHCRNLKGSRAM
IPGDHSSIIIDSVPEVSFSPSSKVPEVTCIPTTATLATHPSAKRQSWHQTPDFTPTKYKERAPEQL
RALESSFAQNPLPLDEELDRLRSETKMTRREIDSWFSERRKKVNAEETKKAEENASQEEEEA
AEDEGGEEDLASELRVSGENGSLEMPSSHILAERKVSPIKINLKNLRVTEANGRNEIPGLGAC
DPEDDESNKLAEQLPGKVSCKKTAQQRHLLRQLFVQTQWPSNQDYDSIMAQTGLPRPEVVR
WFGDSRYALKNGQLKWYEDYKRGNFPPGLLVIAPGNRELLQDYYMTHKMLYEEDLQNLC
DKTQMSSQQVKQWFAEKMGEETRAVADTGSEDQGPGTGELTAVHKGMGDTYSEVSENSES
WEPRVPEASSEPFDTSSPQAGRQLETD
NP_659501.1 SH3 and cysteine-rich domain-containing protein 3
(SEQ ID NO: 200)
MTEKEVLESPKPSFPAETRQSGLQRLKQLLRKGSTGTKEMELPPEPQANGEAVGAGGGPI
YYIYEEEEEEEEEEEEPPPEPPKLVNDKPHKFKDHFFKKPKFCDVCARMIVLNNKFGLRC
KNCKTNIHEHCQSYVEMQRCFGKIPPGFHRAYSSPLYSNQQYACVKDLSAANRNDPVFETLR
TGVIMANKERKKGQADKKNPVAAMMEEEPESARPEEGKPQDGNPEGDKKAEKKTPDDKH
KQPGFQQSHYFVALYRFKALEKDDLDFPPGEKITVIDDSNEEWWRGKIGEKVGFFPPNFIIRV
RAGERVHRVTRSFVGNREIGQITLKKDQIVVQKGDEAGGYVKVYTGRKVGLFPTDFLEEI
NP_001167539.1 synaptotagmin-like protein 4
(SEQ ID NO: 201)
MSELLDLSFLSEEEKDLILSVLQRDEEVRKADEKRIRRLKNELLEIKRKGAKRGSQHYSD
RTCARCQESLGRLSPKTNTCRGCNHLVCRDCRIQESNGTWRCKVCAKEIELKKATGDWFYD
QKVNRFAYRTGSEIIRMSLRHKPAVSKRETVGQSLLHQTQMGDIWPGRKIIQERQKEPSVLFE
VPKLKSGKSALEAESESLDSFTADSDSTSRRDSLDKSGLFPEWKKMSAPKSQVEKETQPGGQ
NVVFVDEGEMIFKKNTRKILRPSEYTKSVIDLRPEDVVHESGSLGDRSKSVPGLNVDMEEEEE
EEDIDHLVKLHRQKLARSSMQSGSSMSTIGSMMSIYSEAGDFGNIFVTGRIAFSLKYEQQTQS
LVVHVKECHQLAYADEAKKRSNPYVKTYLLPDKSRQGKRKTSIKRDTINPLYDETLRYEIPE
SLLAQRTLQFSVWHHGRFGRNTFLGEAEIQMDSWKLDKKLDHCLPLHGKISAESPTGLPSHK
GELVVSLKYIPASKTPVGGDRKKSKGGEGGELQVWIKEAKNLTAAKAGGTSDSFVKGYLLP
MRNKASKRKTPVMKKTLNPHYNHTFVYNGVRLEDLQHMCLELTVWDREPLASNDFLGGV
RLGVGTGISNGEVVDWMDSTGEEVSLWQKMRQYPGSWAEGTLQLRSSMAKQKLGL
NP_001078956.1 histone H2A deubiquitinase MYSM1
(SEQ ID NO: 202)
MAAEEADVDIEGDVVAAAGAQPGSGENTASVLQKDHYLDSSWRTENGLIPWTLDNTISEEN
RAVIEKMLLEEEYYLSKKSQPEKVWLDQKEDDKKYMKSLQKTAKIMVHSPTKPASYSVKW
TIEEKELFEQGLAKFGRRWTKISKLIGSRTVLQVKSYARQYFKNKVKCGLDKETPNQKTGHN
LQVKNEDKGTKAWTPSCLRGRADPNLNAVKIEKLSDDEEVDITDEVDELSSQTPQKNSSSDL
LLDFPNSKMHETNQGEFITSDSQEALFSKSSRGCLQNEKQDETLSSSEITLWTEKQSNGDKKSI
ELNDQKFNELIKNCNKHDGRGIIVDARQLPSPEPCEIQKNLNDNEMLFHSCQMVEESHEEEEL
KPPEQEIEIDRNIIQEEEKQAIPEFFEGRQAKTPERYLKIRNYILDQWEICKPKYLNKTSVRPGL
KNCGDVNCIGRIHTYLELIGAINFGCEQAVYNRPQTVDKVRIRDRKDAVEAYQLAQRLQSM
RTRRRRVRDPWGNWCDAKDLEGQTFEHLSAEELAKRREEEKGRPVKSLKVPRPTKSSFDPF
QLIPCNFFSEEKQEPFQVKVASEALLIMDLHAHVSMAEVIGLLGGRYSEVDKVVEVCAAEPC
NSLSTGLQCEMDPVSQTQASETLAVRGFSVIGWYHSHPAFDPNPSLRDIDTQAKYQSYFSRG
GAKFIGMIVSPYNRNNPLPYSQITCLVISEEISPDGSYRLPYKFEVQQMLEEPQWGLVFEKTR
WIIEKYRLSHSSVPMDKIFRRDSDLTCLQKLLECMRKTLSKVTNCFMAEEFLTEIENLFLSNY
KSNQENGVTEENCTKELLM
NP_004562.2 homeobox protein PKNOX1
(SEQ ID NO: 203)
MMATQTLSIDSYQDGQQMQVVTELKTEQDPNCSEPDAEGVSPPPVESQTPMDVDKQAIYRH
PLFPLLALLFEKCEQSTQGSEGTTSASFDVDIENFVRKQEKEGKPFFCEDPETDNLMVKAIQV
LRIHLLELEKVNELCKDFCSRYIACLKTKMNSETLLSGEPGSPYSPVQSQQIQSAITGTISPQGI
VVPASALQQGNVAMATVAGGTVYQPVTVVTPQGQVVTQTLSPGTIRIQNSQLQLQLNQDLS
ILHQDDGSSKNKRGVLPKHATNVMRSWLFQHIGHPYPTEDEKKQIAAQTNLTLLQVNNWFI
NARRRILQPMLDSSCSETPKTKKKTAQNRPVQRFWPDSIASGVAQPPPSELTMSEGAVVTITT
PVNMNVDSLQSLSSDGATLAVQQVMMAGQSEDESVDSTEEDAGALAPAHISGLVLENSDSL
Q
NP_001027453.1 Krueppel-like factor 10 isoform b
(SEQ ID NO: 204)
MEERMEMISERPKESMYSWNKTAEKSDFEAVEALMSMSCSWKSDFKKYVENRPVTPVSDL
SEEENLLPGTPDFHTIPAFCLTPPYSPSDFEPSQVSNLMAPAPSTVHFKSLSDTAKPHIAAPFKE
EEKSPVSAPKLPKAQATSVIRHTADAQLCNHQTCPMKAASILNYQNNSFRRRTHLNVEAARK
NIPCAAVSPNRSKCERNTVADVDEKASAALYDFSVPSSETVICRSQPAPVSPQQKSVLVSPPA
VSAGGVPPMPVICQMVPLPANNPVVTTVVPSTPPSQPPAVCPPVVFMGTQVPKGAVMFVVP
QPVVQSSKPPVVSPNGTRLSPIAPAPGFSPSAAKVTPQIDSSRIRSHICSHPGCGKTYFKSSHLK
AHTRTHTGEKPFSCSWKGCERRFARSDELSRHRRTHTGEKKFACPMCDRRFMRSDHLTKHA
RRHLSAKKLPNWQMEVSKLNDIALPPTPAPTQ
NP002720.1 hematopoietically-expressed homeobox protein HHEX
(SEQ ID NO: 205)
MQYPHPGPAAGAVGVPLYAPTPLLQPAHPTPFYIEDILGRGPAAPTPAPTLPSPNSSFTSLVSP
YRTPVYEPTPIHPAFSHHSAAALAAAYGPGGFGGPLYPFPRTVNDYTHALLRHDPLGKPLLW
SPFLQRPLHKRKGGQVRFSNDQTIELEKKFETQKYLSPPERKRLAKMLQLSERQVKTWFQNR
RAKWRRLKQENPQSNKKEELESLDSSCDQRQDLPSEQNKGASLDSSQCSPSPASQEDLESEIS
EDSDQEVDIEGDKSYFNAG
NP_006352.2 homeobox protein Hox-B13
(SEQ ID NO: 206)
MEPGNYATLDGAKDIEGLLGAGGGRNLVAHSPLTSHPAAPTLMPAVNYAPLDLPGSAEPPK
QCHPCPGVPQGTSPAPVPYGYFGGGYYSCRVSRSSLKPCAQAATLAAYPAETPTAGEEYPSR
PTEFAFYPGYPGTYQPMASYLDVSVVQTLGAPGEPRHDSLLPVDSYQSWALAGGWNSQMC
CQGEQNPPGPFWKAAFADSSGQHPPDACAFRRGRKKRIPYSKGQLRELEREYAANKFITKDK
RRKISAATSLSERQITIWFQNRRVKEKKVLAKVKNSATP
NP_597721.2 zinc finger protein 483 isoform a
(SEQ ID NO: 207)
MQAVVPLNKMTAISPEPQTLASTEQNEVPRVVTSGEQEAILRGNAADAESFRQRFRWFCYSE
VAGPRKALSQLWELCNQWLRPDIHTKEQILELLVFEQFLTILPGEIRIWVKSQHPESS
EEVVTLIEDLTQMLEEKDPVSQDSTVSQEENSKEDKMVTVCPNTESCESITLKDVAVNFS
RGEWKKLEPFQKELYKEVLLENLRNLEFLDFPVSKLELISQLKWVELPWLLEEVSKSSRL
DESALDKIIERCLRDDDHGLMEESQQYCGSSEEDHGNQGNSKGRVAQNKTLGSGSRGKKFD
PDKSPFGHNFKETSDLIKHLRVYLRKKSRRYNESKKPFSFHSDLVLNRKEKTAGEKSRKSND
GGKVLSHSSALTEHQKRQKIHLGDRSQKCSKCGIIFIRRSTLSRRKTPMCEKCRKDSCQEAAL
NKDEGNESGEKTHKCSKCGKAFGYSASLTKHRRIHTGEKPYMCNECGKAFSDSSSLTPHHRT
HSGEKPFKCDDCGKGFTLSAHLIKHQRIHTGEKPYKCKDCGRPFSDSSSLIQHQRIHTGEKPY
TCSNCGKSFSHSSSLSKHQRIHTGEKPYKCGECGKAFRQNSCLTRHQRIHTGEKPYLCNDCG
MTFSHFTSVIYHQRLHSGEKPYKCNQCEKAFPTHSLLSRHQRIHTGVKPYKCKECGKSFSQSS
SLNEHHRIHTGEKPYECNYCGATFSRSSILVEHLKIHTGRREYECNECEKTFKSNSGLIRHRGF
HSAE
NP_001502.1 growth-regulated alpha protein precursor
(SEQ ID NO: 208)
MARAALSAAPSNPRLLRVALLLLLLVAAGRRAAGASVATELRCQCLQTLQGIHPKNIQSVNV
KSPGPHCAQTEVIATLKNGRKACLNPASPIVKKIIEKMLNSDKSN
NP_001244.1 cell division cycle 5-like protein
(SEQ ID NO: 209)
MPRIMIKGGVWRNTEDEILKAAVMKYGKNQWSRIASLLHRKSAKQCKARWYEWLDPSIKK
TEWSREEEEKLLHLAKLMPTQWRTIAPIIGRTAAQCLEHYEFLLDKAAQRDNEEETTDDPRK
LKPGEIDPNPETKPARPDPIDMDEDELEMLSEARARLANTQGKKAKRKAREKQLEEARRLAA
LQKRRELRAAGIEIQKKRKRKRGVDYNAEIPFEKKPALGFYDTSEENYQALDADFRKLRQQD
LDGELRSEKEGRDRKKDKQHLKRKKESDLPSAILQTSGVSEFTKKRSKLVLPAPQISDAELQE
VVKVGQASEIARQTAEESGITNSASSTLLSEYNVTNNSVALRTPRTPASQDRILQEAQNLMAL
TNVDTPLKGGLNTPLHESDFSGVTPQRQVVQTPNTVLSTPFRTPSNGAEGLTPRSGTTPKPVI
NSTPGRTPLRDKLNINPEDGMADYSDPSYVKQMERESREHLRLGLLGLPAPKNDFEIVLPEN
AEKELEEREIDDTYIEDAADVDARKQAIRDAERVKEMKRMHKAVQKDLPRPSEVNETILRPL
NVEPPLTDLQKSEELIKKEMITMLHYDLLHHPYEPSGNKKGKTVGFGTNNSEHITYLEHNPY
EKFSKEELKKAQDVLVQEMEVVKQGMSHGELSSEAYNQVWEECYSQVLYLPGQSRYTRAN
LASKKDRIESLEKRLEINRGHMTTEAKRAAKMEKKMKILLGGYQSRAMGLMKQLNDLWDQ
IEQAHLELRTFEELKKHEDSAIPRRLECLKEDVQRQQEREKELQHRYADLLLEKETLKSKF
NP_115816.2 ligand-dependent corepressor isoform 1
(SEQ ID NO: 210)
MQRMIQQFAAEYTSKNSSTQDPSQPNSTKNQSLPKASPVTTSPTAATTQNPVLSKLLMADQD
SPLDLTVRKSQSEPSEQDGVLDLSTKKSPCAGSTSLSHSPGCSSTQGNGRPGRPSQYRPDGLR
SGDGVPPRSLQDGTREGFGHSTSLKVPLARSLQISEELLSRNQLSTAASLGPSGL
QNHGQHLILSREASWAKPHYEFNLSRMKFRGNGALSNISDLPFLAENSAFPKMALQAKQDG
KKDVSHSSPVDLKIPQVRGMDLSWESRTGDQYSYSSLVMGSQTESALSKKLRAILPKQSRKS
MLDAGPDSWGSDAEQSTSGQPYPTSDQEGDPGSKQPRKKRGRYRQYNSEILEEAISVVMSG
KMSVSKAQSIYGIPHSTLEYKVKERLGTLKNPPKKKMKLMRSEGPDVSVKIELDPQGEAAQS
ANESKNE
NP_001502.1 growth-regulated alpha protein precursor
(SEQ ID NO: 211)
MARAALSAAPSNPRLLRVALLLLLLVAAGRRAAGASVATELRCQCLQTLQGIHPKNIQSVNV
KSPGPHCAQTEVIATLKNGRKACLNPASPIVKKIIEKMLNSDKSN
NP_005212.1 homeobox protein DLX-5
(SEQ ID NO: 212)
MTGVFDRRVPSIRSGDFQAPFQTSAAMHHPSQESPTLPESSATDSDYYSPTGGAPHGYCS
PTSASYGKALNPYQYQYHGVNGSAGSYPAKAYADYSYASSYHQYGGAYNRVPSATNQPEK
EVTEPEVRMVNGKPKKVRKPRTIYSSFQLAALQRRFQKTQYLALPERAELAASLGLTQTQVK
IWFQNKRSKIKKIMKNGEMPPEHSPSSSDPMACNSPQSPAVWEPQGSSRSLSHHPHAHPPTSN
QSPASSYLENSASWYTSAASSINSHLPPPGSLQHPLALASGTLY
NP_001124296.1 transcription elongation factor SPT5 isoform a
(SEQ ID NO: 213)
MSDSEDSNFSEEEDSERSSDGEEAEVDEERRSAAGSEKEEEPEDEEEEEEEEEYDEEEEE
EDDDRPPKKPRHGGFILDEADVDDEYEDEDQWEDGAEDILEKEEIEASNIDNVVLDEDRS
GARRLQNLWRDQREEELGEYYMKKYAKSSVGETVYGGSDELSDDITQQQLLPGVKDPNLW
TVKCKIGEERATAISLMRKFIAYQFTDTPLQIKSVVAPEHVKGYIYVEAYKQTHVKQAIEGVG
NLRLGYWNQQMVPIKEMTDVLKVVKEVANLKPKSWVRLKRGIYKDDIAQVDYVEPSQNTI
SLKMIPRIDYDRIKARMSLKDWFAKRKKFKRPPQRLFDAEKIRSLGGDVASDGDFLIFEGNRY
SRKGFLFKSFAMSAVITEGVKPTLSELEKFEDQPEGIDLEVVTESTGKEREHNFQPGDNVEVC
EGELINLQGKILSVDGNKITIMPKHEDLKDMLEFPAQELRKYFKMGDHVKVIAGRFEGDTGLI
VRVEENFVILFSDLTMHELKVLPRDLQLCSETASGVDVGGQHEWGELVQLDPQTVGVIVRLE
RETFQVLNMYGKVVTVRHQAVTRKKDNRFAVALDSEQNNIHVKDIVKVIDGPHSGREGEIR
HLFRSFAFLHCKKLVENGGMFVCKTRHLVLAGGSKPRDVTNFTVGGFAPMSPRISSPMHPSA
GGQRGGFGSPGGGSGGMSRGRGRRDNELIGQTVRISQGPYKGYIGVVKDATESTARVELHST
CQTISVDRQRLTTVGSRRPGGMTSTYGRTPMYGSQTPMYGSGSRTPMYGSQTPLQDGSRTP
HYGSQTPLHDGSRTPAQSGAWDPNNPNTPSRAEEEYEYAFDDEPTPSPQAYGGTPNPQTPGY
PDPSSPQVNPQYNPQTPGTPAMYNTDQFSPYAAPSPQGSYQPSPSPQSYHQVAPSPAGYQNT
HSPASYHPTPSPMAYQASPSPSPVGYSPMTPGAPSPGGYNPHTPGSGIEQNSSDWVTTDIQVK
VRDTYLDTQVVGQTGVIRSVTGGMCSVYLKDSEKVVSISSEHLEPITPTKNNKVKVILGEDRE
ATGVLLSIDGEDGIVRMDLDEQLKILNLRFLGKLLEA
NP_001193954.1 POU domain, class 2, transcription factor 2 isoform 1
(SEQ ID NO: 214)
MVHSSMGAPEIRMSKPLEAEKQGLDSPSEHTDTERNGPDTNHQNPQNKTSPFSVSPTGPS
TKIKAEDPSGDSAPAAPLPPQPAQPHLPQAQLMLTGSQLAGDIQQLLQLQQLVLVPGHHL
QPPAQFLLPQAQQSQPGLLPTPNLFQLPQQTQGALLTSQPRAGLPTQAVTRPTLPDPHLS
HPQPPKCLEPPSHPEEPSDLEELEQFARTFKQRRIKLGFTQGDVGLAMGKLYGNDFSQTT
ISRFEALNLSFKNMCKLKPLLEKWLNDAETMSVDSSLPSPNQLSSPSLGFDGLPGRRRKK
RTSIETNVRFALEKSFLANQKPTSEEILLIAEQLHMEKEVIRVWFCNRRQKEKRINPCSA
APMLPSPGKPASYSPHMVTPQGGAGTLPLSQASSSLSTTVTTLSSAVGTLHPSRTAGGGG
GGGGAAPPLNSIPSVTPPPPATTNSTNPSPQGSHSAIGLSGLNPSTGPGLWWNPAPYQP
NP_057588.1 39S ribosomal protein L27, mitochondrial
(SEQ ID NO: 215)
MASVVLALRTRTAVTSLLSPTPATALAVRYASKKSGGSSKNLGGKSSGRRQGIKKMEGHYV
HAGNIIATQRHFRWHPGAHVGVGKNKCLYALEEGIVRYTKEVYVPHPRNTEAVDLITRLPK
GAVLYKTFVHVVPAKPEGTFKLVAML
NP_000177.2 complement factor H isoform a precursor
(SEQ ID NO: 216)
MRLLAKIICLMLWAICVAEDCNELPPRRNTEILTGSWSDQTYPEGTQAIYKCRPGYRSLG
NVIMVCRKGEWVALNPLRKCQKRPCGHPGDTPFGTFTLTGGNVFEYGVKAVYTCNEGYQL
LGEINYRECDTDGWTNDIPICEVVKCLPVTAPENGKIVSSAMEPDREYHFGQAVRFVCNSGY
KIEGDEEMHCSDDGFWSKEKPKCVEISCKSPDVINGSPISQKIIYKENERFQYKCNMGYEYSE
RGDAVCTESGWRPLPSCEEKSCDNPYIPNGDYSPLRIKHRTGDEITYQCRNGFYPATRGNTA
KCTSTGWIPAPRCTLKPCDYPDIKHGGLYHENMRRPYFPVAVGKYYSYYCDEHFETPSGSY
WDHIHCTQDGWSPAVPCLRKCYFPYLENGYNQNHGRKFVQGKSIDVACHPGYALPKAQTT
VTCMENGWSPTPRCIRVKTCSKSSIDIENGFISESQYTYALKEKAKYQCKLGYVTADGETSGS
ITCGKDGW SAQPTCIKSCDIPVFMNARTKNDFTWFKLNDTLDYECHDGYESNTGSTTGSIVC
GYNGWSDLPICYERECELPKIDVHLVPDRKKDQYKVGEVLKFSCKPGFTIVGPNSVQCYHFG
LSPDLPICKEQVQSCGPPPELLNGNVKEKTKEEYGHSEVVEYYCNPRFLMKGPNKIQCVDGE
WTTLPVCIVEESTCGDIPELEHGWAQLSSPPYYYGDSVEFNCSESFTMIGHRSITCIHGVWTQL
PQCVAIDKLKKCKSSNLIILEEHLKNKKEFDHNSNIRYRCRGKEGWIHTVCINGRWDPEVNCS
MAQIQLCPPPPQIPNSHNMTTTLNYRDGEKVSVLCQENYLIQEGEEITCKDGRWQSIPLCVEK
IPCSQPPQIEHGTINSSRSSQESYAHGTKLSYTCEGGFRISEENETTCYMGKWSSPPQCEGLPC
KSPPEISHGVVAHMSDSYQYGEEVTYKCFEGFGIDGPAIAKCLGEKWSHPPSCIKTDCLSLPSF
ENAIPMGEKKDVYKAGEQVTYTCATYYKMDGASNVTCINSRWTGRPTCRDTSCVNPPTVQ
NAYIVSRQMSKYPSGERVRYQCRSPYEMFGDEEVMCLNGNWTEPPQCKDSTGKCGPPPPID
NGDITSFPLSVYAPASSVEYQCQNLYQLEGNKRITCRNGQWSEPPKCLHPCVISREIMENYNI
ALRWTAKQKLYSRTGESVEFVCKRGYRLSSRSHTLRTTCWDGKLEYPTCAKR
NP_000177.2 complement factor H isoform a precursor
(SEQ ID NO: 217)
MRLLAKIICLMLWAICVAEDCNELPPRRNTEILTGSWSDQTYPEGTQAIYKCRPGYRSLGNVI
MVCRKGEWVALNPLRKCQKRPCGHPGDTPFGTFTLTGGNVFEYGVKAVYTCNEGYQLLGE
INYRECDTDGWTNDIPICEVVKCLPVTAPENGKIVSSAMEPDREYHFGQAVRFVCNSGYKIE
GDEEMHCSDDGFWSKEKPKCVEISCKSPDVINGSPISQKIIYKENERFQYKCNMGYEYSERGD
AVCTESGWRPLPSCEEKSCDNPYIPNGDYSPLRIKHRTGDEITYQCRNGFYPATRGNTAKCTS
TGWIPAPRCTLKPCDYPDIKHGGLYHENMRRPYFPVAVGKYYSYYCDEHFETPSGSYWDHI
HCTQDGWSPAVPCLRKCYFPYLENGYNQNHGRKFVQGKSIDVACHPGYALPKAQTTVTCM
ENGWSPTPRCIRVKTCSKSSIDIENGFISESQYTYALKEKAKYQCKLGYVTADGETSGSITCGK
DGWSAQPTCIKSCDIPVFMNARTKNDFTWFKLNDTLDYECHDGYESNTGSTTGSIVCGYNG
WSDLPICYERECELPKIDVHLVPDRKKDQYKVGEVLKFSCKPGFTIVGPNSVQCYHFGLSPDL
PICKEQVQSCGPPPELLNGNVKEKTKEEYGHSEVVEYYCNPRFLMKGPNKIQCVDGEWTTLP
VCIVEESTCGDIPELEHGWAQLSSPPYYYGDSVEFNCSESFTMIGHRSITCIHGVWTQLPQCV
AIDKLKKCKSSNLIILEEHLKNKKEFDHNSNIRYRCRGKEGWIHTVCINGRWDPEVNCSMAQI
QLCPPPPQIPNSHNMTTTLNYRDGEKVSVLCQENYLIQEGEEITCKDGRWQSIPLCVEKIPCSQ
PPQIEHGTINSSRSSQESYAHGTKLSYTCEGGFRISEENETTCYMGKWSSPPQCEGLPCKSPPEI
SHGVVAHMSDSYQYGEEVTYKCFEGFGIDGPAIAKCLGEKWSHPPSCIKTDCLSLPSFENAIP
MGEKKDVYKAGEQVTYTCATYYKMDGASNVTCINSRWTGRPTCRDTSCVNPPTVQNAYIV
SRQMSKYPSGERVRYQCRSPYEMFGDEEVMCLNGNWTEPPQCKDSTGKCGPPPPIDNGDITS
FPLSVYAPASSVEYQCQNLYQLEGNKRITCRNGQWSEPPKCLHPCVISREIMENYNIALRWT
AKQKLYSRTGESVEFVCKRGYRLSSRSHTLRTTCWDGKLEYPTCAKR
NP_002105.2 zinc finger protein 40
(SEQ ID NO: 218)
MPRTKQIHPRNLRDKIEEAQKELNGAEVSKKEILQAGVKGTSESLKGVKRKKIVAENHLKKI
PKSPLRNPLQAKHKQNTEESSFAVLHSASESHKKQNYIPVKNGKQFTKQNGETPGIIA
EASKSEESVSPKKPLFLQQPSELRRWRSEGADPAKFSDLDEQCDSSSLSSKTRTDNSECI
SSHCGTTSPSYTNTAFDVLLKAMEPELSTLSQKGSPCAIKTEKLRPNKTARSPPKLKNSS
MDAPNQTSQELVAESQSSCTSYTVHMSAAQKNEQGAMQSASHLYHQHEHFVPKSNQHNQQ
LPGCSGFTGSLTNLQNQENAKLEQVYNIAVTSSVGLTSPSSRSQVTPQNQQMDSASPLSISPA
NSTQSPPMPIYNSTHVASVVNQSVEQMCNLLLKDQKPKKQGKYICEYCNRACAKPSVLLKHI
RSHTGERPYPCVTCGFSFKTKSNLYKHKKSHAHTIKLGLVLQPDAGGLFLSHESPKALSIHSD
VEDSGESEEEGATDERQHDLGAMELQPVHIIKRMSNAETLLKSSFTPSSPENVIGDFLLQDRS
AESQAVTELPKVVVHHVTVSPLRTDSPKAMDPKPELSSAQKQKDLQVTNVQPLSANMSQGG
VSRLETNENSHQKGDMNPLEGKQDSHVGTVHAQLQRQQATDYSQEQQGKLLSPRSLGSTDS
GYFSRSESADQTVSPPTPFARRLPSTEQDSGRSNGPSAALVTTSTPSALPTGEKALLLPGQMRP
PLATKTLEERISKLISDNEALVDDKQLDSVKPRRTSLSRRGSIDSPKSYIFKDSFQFDLKPVGRR
TSSSSDIPKSPFTPTEKSKQVFLLSVPSLDCLPITRSNSMPTTGYSAVPANIIPPPHPLRGSQSFD
DKIGTFYDDVFVSGPNAPVPQSGHPRTLVRQAAIEDSSANESHVLGTGQSLDESHQGCHAAG
EAMSVRSKALAQGPHIEKKKSHQGRGTMFECETCRNRYRKLENFENHKKFYCSELHGPKTK
VAMREPEHSPVPGGLQPQILHYRVAGSSGIWEQTPQIRKRRKMKSVGDDEELQQNESGTSPK
SSEGLQFQNALGCNPSLPKHNVTIRSDQQHKNIQLQNSHIHLVARGPEQTMDPKLSTIMEQQI
SSAAQDKIELQRHGTGISVIQHTNSLSRPNSFDKPEPFERASPVSFQELNRTGKSGSLKVIGISQ
EESHPSRDGSHPHQLALSDALRGELQESSRKSPSERHVLGQPSRLVRQHNIQVPEILVTEEPDR
DLEAQCHDQEKSEKFSWPQRSETLSKLPTEKLPPKKKRLRLAEIEHSSTESSFDSTLSRSLSRE
SSLSHTSSFSASLDIEDVSKTEASPKIDFLNKAEFLMIPAGLNTLNVPGCHREMRRTASEQINC
TQTSMEVSDLRSKSFDCGSITPPQTTPLTELQPPSSPSRVGVTGHVPLLERRRGPLVRQISLNIA
PDSHLSPVHPTSFQNTALPSVNAVPYQGPQLTSTSLAEFSANTLHSQTQVKDLQAETSNSSST
NVFPVQQLCDINLLNQIHAPPSHQSTQLSLQVSTQGSKPDKNSVLSGSSKSEDCFAPKYQLHC
QVFTSGPSCSSNPVHSLPNQVISDPVGTDHCVTSATLPTKLIDSMSNSHPLLPPELRPLGSQVQ
KVPSSFMLPIRLQSSVPAYCFATLTSLPQILVTQDLPNQPICQTNHSVVPISEEQNSVPTLQKGH
QNALPNPEKEFLCENVFSEMSQNSSLSESLPITQKISVGRLSPQQESSASSKRMLSPANSLDIA
MEKHQKRAKDENGAVCATDVRPLEALSSRVNEASKQKKPILVRQVCTTEPLDGVMLEKDV
FSQPEISNEAVNLTNVLPADNSSTGCSKFVVIEPISELQEFENIKSSTSLTLTVRSSPAPSENTHIS
PLKCTDNNQERKSPGVKNQGDKVNIQEQSQQPVTSLSLFNIKDTQQLAFPSLKTTTNFTWCY
LLRQKSLHLPQKDQKTSAYTDWTVSASNPNPLGLPTKVALALLNSKQNTGKSLYCQAITTHS
KSDLLVYSSKWKSSLSKRALGNQKSTVVEFSNKDASEINSEQDKENSLIKSEPRRIKIFDGGY
KSNEEYVYVRGRGRGKYICEECGIRCKKPSMLKKHIRTHTDVRPYHCTYCNFSFKTKGNLTK
HMKSKAHSKKCVDLGVSVGLIDEQDTEESDEKQRFSYERSGYDLEESDGPDEDDNENEDDD
EDSQAESVLSATPSVTASPQHLPSRSSLQDPVSTDEDVRITDCFSGVHTDPMDVLPRALLTRM
TVLSTAQSDYNRKTLSPGKARQRAARDENDTIPSVDTSRSPCHQMSVDYPESEEILRSSMAG
KAVAITQSPSSVRLPPAAAEHSPQTAAGMPSVASPHPDPQEQKQQITLQPTPGLPSPHTHLFSH
LPLHSQQQSRTPYNMVPVGGIHVVPAGLTYSTFVPLQAGPVQLTIPAVSVVHRTLGTHRNTV
TEVSGTTNPAGVAELSSVVPCIPIGQIRVPGLQNLSTPGLQSLPSLSMETVNIVGLANTNMAPQ
VHPPGLALNAVGLQVLTANPSSQSSPAPQAHIPGLQILNIALPTLIPSVSQVAVDAQGAPEMP
ASQSKACETQPKQTSVASANQVSRTESPQGLPTVQRENAKKVLNPPAPAGDHARLDGLSKM
DTEKAASANHVKPKPELTSIQGQPASTSQPLLKAHSEVFTKPSGQQTLSPDRQVPRPTALPRR
QPTVHFSDVSSDDDEDRLVIAT
NP_443177.1 tumor necrosis factor receptor superfamily member 13C
(SEQ ID NO: 219)
MRRGPRSLRGRDAPAPTPCVPAECFDLLVRHCVACGLLRTPRPKPAGASSPAPRTALQPQ
ESVGAGAGEAALPLPGLLFGAPALLGLALVLALVLVGLVSWRRRQRRLRGASSAEAPDGDK
DAPEPLDKVIILSPGISDATAPAWPPPGEDPGTTPPGHSVPVPATELGSTELVTTKTAG
PEQQ
NP_059523.2 telomeric repeat-binding factor 1 isoform 1
(SEQ ID NO: 220)
MAEDVSSAAPSPRGCADGRDADPTEEQMAETERNDEEQFECQELLECQVQVGAPEEEEEEE
EDAGLVAEAEAVAAGWMLDFLCLSLCRAFRDGRSEDFRRTRNSAEAIIHGLSSLTACQLRTI
YICQFLTRIAAGKTLDAQFENDERITPLESALMIWGSIEKEHDKLHEEIQNLIKIQAIAVCMEN
GNFKEAEEVFERIFGDPNSHMPFKSKLLMIISQKDTFHSFFQHFSYNHMMEKIKSYVNYVLSE
KSSTFLMKAAAKVVESKRTRTITSQDKPSGNDVEMETEANLDTRKSVSDKQSAVTESSEGTV
SLLRSHKNLFLSKLQHGTQQQDLNKKERRVGTPQSTKKKKESRRATESRIPVSKSQPVTPEK
HRARKRQAWLWEEDKNLRSGVRKYGEGNWSKILLHYKFNNRTSVMLKDRWRTMKKLKLI
SSDSED
NP_116262.2 ubiquitin-associated and SH3 domain-containing protein B
(SEQ ID NO: 221)
MAQYGHPSPLGMAAREELYSKVTPRRNRQQRPGTIKHGSALDVLLSMGFPRARAQKALAST
GGRSVQAACDWLFSHVGDPFLDDPLPREYVLYLRPTGPLAQKLSDFWQQSKQICGKNKAHN
IFPHITLCQFFMCEDSKVDALGEALQTTVSRWKCKFSAPLPLELYTSSNFIGLFVKEDSAEVLK
KFAADFAAEAASKTEVHVEPHKKQLHVTLAYHFQASHLPTLEKLAQNIDVKLGCDWVATIF
SRDIRFANHETLQVIYPYTPQNDDELELVPGDFIFMSPMEQTSTSEGWIYGTSLTTGCSGLLPE
NYITKADECSTWIFHGSYSILNTSSSNSLTFGDGVLERRPYEDQGLGETTPLTIICQPMQPLRV
NSQPGPQKRCLFVCRHGERMDVVFGKYWLSQCFDAKGRYIRTNLNMPHSLPQRSGGFRDYE
KDAPITVFGCMQARLVGEALLESNTIIDHVYCSPSLRCVQTAHNILKGLQQENHLKIRVEPGL
FEWTKWVAGSTLPAWIPPSELAAANLSVDTTYRPHIPISKLVVSESYDTYISRSFQVTKEIISEC
KSKGNNILIVAHASSLEACTCQLQGLSPQNSKDFVQMVRKIPYLGFCSCEELGETGIWQLTDP
PILPLTHGPTGGFNWRETLLQE
NP_001136156.1 zinc finger MYM-type protein 5 isoform 3
(SEQ ID NO: 222)
MEKCSVGGLELTEQTPALLGNMAMATSLMDIGDSFGHPACPLVSRSRNSPVEDDDDDDDVV
FIESIQPPSISAPAIADQRNFIFASSKNEKPQGNYSVIPPSSRDLASQKGNISETIVIDDEEDIETN
GGAEKKSSCFIEWGLPGTKNKTNDLDFSTSSLSRSKTKTGVRPFNPGRMNVAGDLFQNGEFA
THHSPDSWISQSASFPSNQKQPGVDSLSPVALLRKQNFQPTAQQQLTKPAKITCANCKKPLQ
KGQTAYQRKGSAHLFCSTTCLSSFSHKRTQNTRSIICKKDASTKKANVILPVESSKSFQEFYST
SCLSPCENNWNLKKGVFNKSRCTICSKLAEIRHEVSVNNVTHKLCSNHCFNKYRLANGLIMN
CCEHCGEYMPSKSTGNNILVIGGQQKRFCCQSCINEYKQMMETKSKKLTASENRKRNAFREE
NEKQLYGSSNTLLKKIEGIPEKKEKTSQLQLSVECGTDTLLIQENVNLPPSSTSTIADTFQEQLE
EKNFEDSIVPVVLSADPGTWPRILNIKQRDTLVENVPPQVRNFNFPKDNTGRKFSETYYTRILP
NGEKTTRSWLLYSTSKDSVFCLYCKLFGEGKNQLKNENGCKDWQHLSHILSKHEESEMHVN
NSVKYSKLKSDLKKNKAIDAAEHRLYENEKNDGVLLLYT
NP_004882.1 39S ribosomal protein L33, mitochondrial isoform a
(SEQ ID NO: 223)
MFLSAVFFAKSKSKNILVRMVSEAGTGFCFNTKRNRLREKLTLLHYDPVVKQRVLFVEKKKI
RSL
NP_008841.2 E3 ubiquitin-protein ligase RBBP6 isoform 1
(SEQ ID NO: 224)
MSCVHYKFSSKLNYDTVTFDGLHISLCDLKKQIMGREKLKAADCDLQITNAQTKEEYTDDN
ALIPKNSSVIVRRIPIGGVKSTSKTYVISRTEPAMATTKAIDDSSASISLAQLTKTANLAEANAS
EEDKIKAMMSQSGHEYDPINYMKKPLGPPPPSYTCFRCGKPGHYIKNCPTNGDKNFESGPRIK
KSTGIPRSFMMEVKDPNMKGAMLTNTGKYAIPTIDAEAYAIGKKEKPPFLPEEPSSSSEEDDPI
PDELLCLICKDIMTDAVVIPCCGNSYCDECIRTALLESDEHTCPTCHQNDVSPDALIANKFLRQ
AVNNFKNETGYTKRLRKQLPPPPPPIPPPRPLIQRNLQPLMRSPISRQQDPLMIPVTSSSTHPAP
SISSLTSNQSSLAPPVSGNPSSAPAPVPDITATVSISVHSEKSDGPFRDSDNKILPAAALASEHSK
GTSSIAITALMEEKGYQVPVLGTPSLLGQSLLHGQLIPTTGPVRINTARPGGGRPGWEHSNKL
GYLVSPPQQIRRGERSCYRSINRGRHHSERSQRTQGPSLPATPVFVPVPPPPLYPPPPHTLPLPP
GVPPPQFSPQFPPGQPPPAGYSVPPPGFPPAPANLSTPWVSSGVQTAHSNTIPTTQAPPLSREEF
YREQRRLKEEEKKKSKLDEFTNDFAKELMEYKKIQKERRRSFSRSKSPYSGSSYSRSSYTYSK
SRSGSTRSRSYSRSFS
RSHSRSYSRSPPYPRRGRGKSRNYRSRSRSHGYHRSRSRSPPYRRYHSRSRSPQAFRGQS
PNKRNVPQGETEREYFNRYREVPPPYDMKAYYGRSVDFRDPFEKERYREWERKYREWYEK
YYKGYAAGAQPRPSANRENFSPERFLPLNIRNSPFTRGRREDYVGGQSHRSRNIGSNYPE
KLSARDGHNQKDNTKSKEKESENAPGDGKGNKHKKHRKRRKGEESEGFLNPELLETSRKSR
EPTGVEENKTDSLFVLPSRDDATPVRDEPMDAESITFKSVSEKDKRERDKPKAKGDKTKRKN
DGSAVSKKENIVKPAKGPQEKVDGERERSPRSEPPIKKAKEETPKTDNTKSSSSSQKDEKITG
TPRKAHSKSAKEHQETKPVKEEKVKKDYSKDVKSEKLTTKEEKAKKPNEKNKPLDNKGEK
RKRKTEEKGVDKDFESSSMKISKLEVTEIVKPSPKRKMEPDTEKMDRTPEKDKISLSAPAKKI
KLNRETGKKIGSTENISNTKEPSEKLESTSSKVKQEKVKGKVRRKVTGTEGSSSTLVDYTSTS
STGGSPVRKSEEKTDTKRTVIKTMEEYNNDNTAPAEDVIIMIQVPQSKWDKDDFESEEEDVK
STQPISSVGKPASVIKNVSTKPSNIVKYPEKESEPSEKIQKFTKDVSHEIIQHEVKSSKNSASSEK
GKTKDRDYSVLEKENPEKRKNSTQPEKESNLDRLNEQGNFKSLSQSSKEARTSDKHDSTRAS
SNKDFTPNRDKKTDYDTREYSSSKRRDEKNELTRRKDSPSRNKDSASGQKNKPREERDLPKK
GTGDSKKSNSSPSRDRKPHDHKATYDTKRPNEETKSVDKNPCKDREKHVLEARNNKESSGN
KLLYILNPPETQVEKEQITGQIDKSTVKPKPQLSHSSRLSSDLTRETDEAAFEPDYNESDSESN
VSVKEEESSGNISKDLKDKIVEKAKESLDTAAVVQVGISRNQSHSSPSVSPSRSHSPSGSQTRS
HSSSASSAESQDSKKKKKKKEKKKHKKHKKHKKHKKHAGTEVELEKSQKHKHKKKKSKK
NKDKEKEKEKDDQKVKSVTV
NP_004764.1 zinc finger HIT domain-containing protein 3
(SEQ ID NO: 225)
MASLKCSTVVCVICLEKPKYRCPACRVPYCSVVCFRKHKEQCNPETRPVEKKIRSALPTK
TVKPVENKDDDDSIADFLNSDEEEDRVSLQNLKNLGESATLRSLLLNPHLRQLMVNLDQGE
DKAKLMRAYMQEPLFVEFADCCLGIVEPSQNEES
NP_001663.2 agouti-signaling protein precursor
(SEQ ID NO: 226)
MDVTRLLLATLLVFLCFFTANSHLPPEEKLRDDRSLRSNSSVNLLDVPSVSIVALNKKSK
QIGRKAAEKKRSSKKEASMKKVVRPRTPLSAPCVATRNSCKPPAPACCDPCASCQCRFFRSA
CSCRVLSLNC
NP_002334.2 lactotransferrin isoform 1 precursor
(SEQ ID NO: 227)
MKLVFLVLLFLGALGLCLAGRRRSVQWCAVSQPEATKCFQWQRNMRKVRGPPVSCIKRDSP
IQCIQAIAENRADAVTLDGGFIYEAGLAPYKLRPVAAEVYGTERQPRTHYYAVAVVKKGGSF
QLNELQGLKSCHTGLRRTAGWNVPIGTLRPFLNWTGPPEPIEAAVARFFSASCVPGADKGQF
PNLCRLCAGTGENKCAFSSQEPYFSYSGAFKCLRDGAGDVAFIRESTVFEDLSDEAERDEYEL
LCPDNTRKPVDKFKDCHLARVPSHAVVARSVNGKEDAIWNLLRQAQEKFGKDKSPKFQLFG
SPSGQKDLLFKDSAIGFSRVPPRIDSGLYLGSGYFTAIQNLRKSEEEVAARRARVVWCAVGEQ
ELRKCNQWSGLSEGSVTCSSASTTEDCIALVLKGEADAMSLDGGYVYTAGKCGLVPVLAEN
YKSQQSSDPDPNCVDRPVEGYLAVAVVRRSDTSLTWNSVKGKKSCHTAVDRTAGWNIPMG
LLFNQTGSCKFDEYFSQSCAPGSDPRSNLCALCIGDEQGENKCVPNSNERYYGYTGAFRCLA
ENAGDVAFVKDVTVLQNTDGNNNEAWAKDLKLADFALLCLDGKRKPVTEARSCHLAMAP
NHAVVSRMDKVERLKQVLLHQQAKFGRNGSDCPDKFCLFQSETKNLLFNDNTECLARLHG
KTTYEKYLGPQYVAGITNLKKCSTSPLLEACEFLRK
NP_002257.1 importin subunit alpha-2
(SEQ ID NO: 228)
MSTNENANTPAARLHRFKNKGKDSTEMRRRRIEVNVELRKAKKDDQMLKRRNVSSFPDDA
TSPLQENRNNQGTVNWSVDDIVKGINSSNVENQLQATQAARKLLSREKQPPIDNIIRAGLIPK
FVSFLGRTDCSPIQFESAWALTNIASGTSEQTKAVVDGGAIPAFISLLASPHAHISE
QAVWALGNIAGDGSVFRDLVIKYGAVDPLLALLAVPDMSSLACGYLRNLTWTLSNLCRNK
NPAPPIDAVEQILPTLVRLLHHDDPEVLADTCWAISYLTDGPNERIGMVVKTGVVPQLVKLL
GASELPIVTPALRAIGNIVTGTDEQTQVVIDAGALAVFPSLLTNPKTNIQKEATWTMSNITAG
RQDQIQQVVNHGLVPFLVSVLSKADFKTQKEAVWAVTNYTSGGTVEQIVYLVHCGIIEPLM
NLLTAKDTKIILVILDAISNIFQAAEKLGETEKLSIMIEECGGLDKIEALQNHENESVYKASLSL
IEKYFSVEEEEDQNVVPETTSEGYTFQVQDGAPGTFNF
NP_114440.1 POZ-, AT hook-, and zinc finger-containing protein 1 short
isoform
(SEQ ID NO: 229)
MERVNDASCGPSGCYTYQVSRHSTEMLHNLNQQRKNGGRFCDVLLRVGDESFPAHRAVLA
ACSEYFESVFSAQLGDGGAADGGPADVGGATAAPGGGAGGSRELEMHTISSKVFGDILDFA
YTSRIVVRLESFPELMTAAKFLLMRSVIEICQEVIKQSNVQILVPPARADIMLFRPPGTSDLGFP
LDMTNGAALAANSNGIAGSMQPEEEAARAAGAAIAGQASLPVLPGVDRLPMVAGPLSPQLL
TSPFPSVASSAPPLTGKRGRGRPRKANLLDSMFGSPGGLREAGILPCGLCGKVFTDANRLRQH
EAQHGVTSLQLGYIDLPPPRLGENGLPISEDPDGPRKRSRTRKQVACEICGKIFRDVYHLNRH
KLSHSGEKPYSCPVCGLRFKRKDRMSYHVRSHDGSVGKPYICQSCGKGFSRPDHLNGHIKQV
HTSERPHKCQVWVGSSSGLPPLEPLPSDLPSWDFAQPALWRSSHSVPDTAFSLSLKKSFPALE
NLGPAHSSNTLFCPAPPGYLRQGWTTPEGSRAFTQWPVG
NP_001159896.1 gastricsin isoform 2 preproprotein
(SEQ ID NO: 230)
MKWMVVVLVCLQLLEAAVVKVPLKKFKSIRETMKEKGLLGEFLRTHKYDPAWKYRFGDLS
VTYEPMAYMDAAYFGEISIGTPPQNFLVLFDTGSSNLWVPSVYCQSQACTSHSRFNPSESSTY
STNGQTFSLQYGSGSLTGFFGYDTLTVQSIQVPNQEFGLSENEPGTNFVYAQFDGIMGLAYPA
LSVDEATTAMQGMVQEGALTSPVFSVYLSNLVLESSGLGPLLTPSRAAPPSSTLQLPEKPLEQ
TWNILTPFTKTLPVSNLSRKVTSWAGVGIPVTCLPEAGSGGERRAECGLGVPTTRGPPRSQHH
SGA
NP_001036053.1 snurportin-1
(SEQ ID NO: 231)
MEELSQALASSFSVSQDLNSTAAPHPRLSQYKSKYSSLEQSERRRRLLELQKSKRLDYVN
HARRLAEDDWTGMESEEENKKDDEEMDIDTVKKLPKHYANQLMLSEWLIDVPSDLGQEWI
VVVCPVGKRALIVASRGSTSAYTKSGYCVNRFSSLLPGGNRRNSTAKDYTILDCIYNEVNQT
YYVLDVMCWRGHPFYDCQTDFRFYWMHSKLPEEEGLGEKTKLNPFKFVGLKNFPCTPESLC
DVLSMDFPFEVDGLLFYHKQTHYSPGSTPLVGWLRPYMVSDVLGVAVPAGPLTTKPDYAGH
QLQQIMEHKKSQKEGMKEKLTHKASENGHYELEHLSTPKLKGSSHSPDHPGCLMEN
NP_054798.1 Krueppel-like factor 15
(SEQ ID NO: 232)
MVDHLLPVDENFSSPKCPVGYLGDRLVGRRAYHMLPSPVSEDDSDASSPCSCSSPDSQALCS
CYGGGLGTESQDSILDFLLSQATLGSGGGSGSSIGASSGPVAWGPWRRAAAPVKGEHFCLPE
FPLGDPDDVPRPFQPTLEEIEEFLEENMEPGVKEVPEGNSKDLDACSQLSAGPHKSHLHPGSS
GRERCSPPPGGASAGGAQGPGGGPTPDGPIPVLLQIQPVPVKQESGTGPASPG
QAPENVKVAQLLVNIQGQTFALVPQVVPSSNLNLPSKFVRIAPVPIAAKPVGSGPLGPGP
AGLLMGQKFPKNPAAELIKMHKCTFPGCSKMYTKSSHLKAHLRRHTGEKPFACTWPGCGW
RFSRSDELSRHRRSHSGVKPYQCPVCEKKFARSDHLSKHIKVHRFPRSSRSVRSVN
NP_001006657.1 zinc finger protein 473
(SEQ ID NO: 233)
MAEEFVTLKDVGMDFTLGDWEQLGLEQGDTFWDTALDNCQDLFLLDPPRPNLTSHPDGSE
DLEPLAGGSPEATSPDVTETKNSPLMEDFFEEGFSQEIIEMLSKDGFWNSNFGEACIEDTWLD
SLLGDPESLLRSDIATNGESPTECKSHELKRGLSPVSTVSTGEDSMVHNVSEKTLTP
AKSKEYRGEFFSYSDHSQQDSVQEGEKPYQCSECGKSFSGSYRLTQHWITHTREKPTVHQEC
EQGFDRNASLSVYPKTHTGYKFYVCNEYGTTFSQSTYLWHQKTHTGEKPCKSQDSDHPPSH
DTQPGEHQKTHTDSKSYNCNECGKAFTRIFHLTRHQKIHTRKRYECSKCQATFNLRKHLIQH
QKTHAAKTTSECQECGKIFRHSSLLIEHQALHAGEEPYKCNERGKSFRHNSTLKIHQRVHSGE
KPYKCSECGKAFHRHTHLNEHRRIHTGYRPHKCQECVRSFSRPSHLMRHQAIHTAEKPYSCA
ECKETFSDNNRLVQHQKMHTVKTPYECQECGERFICGSTLKCHESVHAREKQGFFVSGKILD
QNPEQKEKCFKCNKCEKTFSCSKYLTQHERIHTRGVKPFECDQCGKAFGQSTRLIHHQRIHSR
VRLYKWGEQGKAISSASLIKLQSFHTKEHPFKCNECGKTFSHSAHLSKHQLIHAGENPFKCSK
CDRVFTQRNYLVQHERTHARKKPLVCNECGKTFRQSSCLSKHQRIHSGEKPYVCDYCGKAF
GLSAELVRHQRIHTGEKPYVCQECGKAFTQSSCLSIHRRVHTGEKPYRCGECGKAFAQKANL
TQHQRIHTGEKPYSCNVCGKAFVLSAHLNQHLRVHTQETLYQCQRCQKAFRCHSSLSRHQR
VHNKQQYCL
NP_002219.1 transcription factor AP-1
(SEQ ID NO: 234)
MTAKMETTFYDDALNASFLPSESGPYGYSNPKILKQSMTLNLADPVGSLKPHLRAKNSDLLT
SPDVGLLKLASPELERLIIQSSNGHITTTPTPTQFLCPKNVTDEQEGFAEGFVRALAELHSQNT
LPSVTSAAQPVNGAGMVAPAVASVAGGSGSGGFSASLHSEPPVYANLSNFNPGALSSGGGA
PSYGAAGLAFPAQPQQQQQPPHHLPQQMPVQHPRLQALKEEPQTVPEMPGETPPLSPIDMES
QERIKAERKRMRNRIAASKCRKRKLERIARLEEKVKTLKAQNSELASTANMLREQVAQLKQ
KVMNHVNSGCQLMLTQQLQTF
NP_113674.1 zinc finger protein 484 isoform a
(SEQ ID NO: 235)
MTKSLESVSFKDVTVDFSRDEWQQLDLAQKSLYREVMLENYFNLISVGCQVPKPEVIFSL
EQEEPCMLDGEIPSQSRPDGDIGFGPLQQRMSEEVSFQSEININLFTRDDPYSILEELWKDDEH
TRKCGENQNKPLSRVVFINKKTLANDSIFEYKDIGEIVHVNTHLVSSRKRPHNCNSCGKNLEP
IITLYNRNNATENSDKTIGDGDIFTHLNSHTEVTACECNQCGKPLHHKQALIQQQKIHTRESL
YLFSDYVNVFSPKSHAFAHESICAEEKQHECHECEAVFTQKSQLDGSQRVYAGICTEYEKDF
SLKSNRQKTPYEGNYYKCSDYGRAFIQKSDLFRCQRIHSGEKPYEYSECEKNLPQNSNLNIHK
KIHTGGKHFECTECGKAFTRKSTLSMHQKIHTGEKPYVCTECGKAFIRKSHFITHERIHTGEKP
YECSDCGKSFIKKSQLHVHQRIHTGENPFICSECGKVFTHKTNLIIHQKIHTGERPYICTVCGK
AFTDRSNLIKHQKIHTGEKPYKCSDCGKSFTWKSRLRIHQKCHTGERHYECSECGKAFIQKST
LSMHQRIHRGEKPYVCTECGKAFFHKSHFITHERIHTGEKPYECSICGKSFTKKSQLHVHQQI
HTGEKPYRCAECGKAFTDRSNLFTHQKIHTGEKPYKCSDCGKAFTRKSGLHIHQQSHTGERH
YECSECGKAFARKSTLIMHQRIHTGEKPYICNECGKSFIQKSHLNRHRRIHTGEKPYECSDCG
KSFIKKSQLHEHHRIHTGEKPYICAECGKAFTIRSNLIKHQKIHTKQKPYKCSDLGKALNWKP
QLSMPQKSDNGEVECSMPQLWCGDSEGDQGQLSSI
NP_001166146.1 zinc finger protein 347 isoform a
(SEQ ID NO: 236)
MALTQGQVTFRDVAIEFSQEEWTCLDPAQRTLYRDVMLENYRNLASLAGISCFDLSIISM
LEQGKEPFTLESQVQIAGNPDGWEWIKAVITALSSEFVMKDLLHKGKSNTGEVFQTVMLER
QESQDIEGCSFREVQKNTHGLEYQCRDAEGNYKGVLLTQEGNLTHGRDEHDKRDARNKLIK
NQLGLSLQSHLPELQLFQYEGKIYECNQVEKSFNNNSSVSPPQQMPYNVKTHISKKYLKDFIS
SLLLTQGQKANNWGSPYKSNGCGMVFPQNSHLASHQRSHTKEKPYKCYECGKAFRTRSNLT
THQVIHTGEKRYKCNECGKVFSRNSQLSQHQKIHTGEKPYKCNECGKVFTQNSHLVRHRGIH
TGEKPYKCNECGKAFRARSSLAIHQATHSGEKPYKCNECGKVFTQNSHLTNHWRIHTGEKP
YKCNECGKAFGVRSSLAIHLVIHTGEKPYKCHECGKVFRRNSHLARHQLIHTGEKPYKCNEC
GKAFRAHSNLTTHQVIHTGEKPYKCNECGKVFTQNSHLANHQRIHTGVKPYMCNECGKAFS
VYSSLTTHQVIHTGEKPYKCNECGKVFTQNSHLARHRGIHTGEKPYKCNECGKVFRHNSYLS
RHQRIHTGEKPYKYNEYGKAFSEHSNLTTHQVIHTGEKPYKCNECGKVFTQNSHLARHRRV
HTGGKPYQCNECGKAFSQTSKLARHQRVHTGEKPYECNQCGKAFSVRSSLTTHQAIHTGKK
PYKCNECGKVFTQNSHLARHRGIHTGEKPYKCNECGKAFSQTSKLARHQRIHTGEKPYECG
KPFSICSSLTTHQTIHTGGKPYKCNVWKVLKSEFKPCKPSQNS
NP_065879.1 zinc finger protein 28 homolog
(SEQ ID NO: 237)
MRGAASASVREPTPLPGRGAPRTKPRAGRGPTVGTPATLALPARGRPRSRNGLASKGQRGA
APTGPGHRALPSRDTALPQERNKKLEAVGTGIEPKAMSQGLVTFGDVAVDFSQEEWEWLNP
IQRNLYRKVMLENYRNLASLGLCVSKPDVISSLEQGKEPWTVKRKMTRAWCPDLKAVWKI
KELPLKKDFCEGKLSQAVITERLTSYNLEYSLLGEHWDYDALFETQPGLVTIKNLAVDFRQQ
LHPAQKNFCKNGIWENNSDLGSAGHCVAKPDLVSLLEQEKEPWMVKRELTGSLFSGQRSVH
ETQELFPKQDSYAEGVTDRTSNTKLDCSSFRENWDSDYVFGRKLAVGQETQFRQEPITHNKT
LSKERERTYNKSGRWFYLDDSEEKVHNRDSIKNFQKSSVVIKQTGIYAGKKLFKCNECKKTF
TQSSSLTVHQRIHTGEKPYKCNECGKAFSDGSSFARHQRCHTGKKPYECIECGKAFIQNTSLI
RHWRYYHTGEKPFDCIDCGKAFSDHIGLNQHRRIHTGEKPYKCDVCHKSFRYGSSLTVHQRI
HTGEKPYECDVCRKAFSHHASLTQHQRVHSGEKPFKCKECGKAFRQNIHLASHLRIHTGEKP
FECAECGKSFSISSQLATHQRIHTGEKPYECKVCSKAFTQKAHLAQHQKTHTGEKPYECKEC
GKAFSQTTHLIQHQRVHTGEKPYKCMECGKAFGDNSSCTQHQRLHTGQRPYECIECGKAFK
TKSSLICHRRSHTGEKPYECSVCGKAFSHRQSLSVHQRIHSGKKPYECKECRKTFIQIGHLNQ
HKRVHTGERSYNYKKSRKVFRQTAHLAHHQRIHTGESSTCPSLPSTSNPVDLFPKFLWNPSSL
PSP
NP_113674.1 zinc finger protein 484 isoform a
(SEQ ID NO: 238)
MTKSLESVSFKDVTVDFSRDEWQQLDLAQKSLYREVMLENYFNLISVGCQVPKPEVIFSLEQ
EEPCMLDGEIPSQSRPDGDIGFGPLQQRMSEEVSFQSEININLFTRDDPYSILEELWKDDEHTR
KCGENQNKPLSRVVFINKKTLANDSIFEYKDIGEIVHVNTHLVSSRKRPHNCNSCGKNLEPIIT
LYNRNNATENSDKTIGDGDIFTHLNSHTEVTACECNQCGKPLHHKQALIQQQKIHTRESLYLF
SDYVNVFSPKSHAFAHESICAEEKQHECHECEAVFTQKSQLDGSQRVYAGICTEYEKDFSLK
SNRQKTPYEGNYYKCSDYGRAFIQKSDLFRCQRIHSGEKPYEYSECEKNLPQNSNLNIHKKIH
TGGKHFECTECGKAFTRKSTLSMHQKIHTGEKPYVCTECGKAFIRKSHFITHERIHTGEKPYE
CSDCGKSFIKKSQLHVHQRIHTGENPFICSECGKVFTHKTNLIIHQKIHTGERPYICTVCGKAFT
DRSNLIKHQKIHTGEKPYKCSDCGKSFTWKSRLRIHQKCHTGERHYECSECGKAFIQKSTLS
MHQRIHRGEKPYVCTECGKAFFHKSHFITHERIHTGEKPYECSICGKSFTKKSQLHVHQQIHT
GEKPYRCAECGKAFTDRSNLFTHQKIHTGEKPYKCSDCGKAFTRKSGLHIHQQSHTGERHYE
CSECGKAFARKSTLIMHQRIHTGEKPYICNECGKSFIQKSHLNRHRRIHTGEKPYECSDCGKSF
IKKSQLHEHHRIHTGEKPYICAECGKAFTIRSNLIKHQKIHTKQKPYKCSDLGKALNWKPQLS
MPQKSDNGEVECSMPQLWCGDSEGDQGQLSSI
NP_001159354.1 zinc finger protein 268 isoform c
(SEQ ID NO: 239)
MDVFVDFTWEEWQLLDPAQKCLYRSVMLENYSNLVSLGYQHTKPDIIFKLEQGEELCMVQ
AQVPNQTCPNTVWKIDDLMDWHQENKDKLGSTAKSFECTTFGKLCLLSTKYLSRQKPHKC
GTHGKSLKYIDFTSDYARNNPNGFQVHGKSFFHSKHEQTVIGIKYCESIESGKTVNKKSQLM
CQQMYMGEKPFGCSCCEKAFSSKSYLLVHQQTHAEEKPYGCNECGKDFSSKSYLIVHQRIHT
GEKLHECSECRKTFSFHSQLVIHQRIHTGENPYECCECGKVFSRKDQLVSHQKTHSGQKPYV
CNECGKAFGLKSQLIIHERIHTGEKPYECNECQKAFNTKSNLMVHQRTHTGEKPYVCSDCGK
AFTFKSQLIVHQGIHTGVKPYGCIQCGKGFSLKSQLIVHQRSHTGMKPYVCNECGKAFRSKS
YLIIHTRTHTGEKLHECNNCGKAFSFKSQLIIHQRIHTGENPYECHECGKAFSRKYQLISHQRT
HAGEKPYECTDCGKAFGLKSQLIIHQRTHTGEKPFECSECQKAFNTKSNLIVHQRTHTGEKPY
SCNECGKAFTFKSQLIVHKGVHTGVKPYGCSQCAKTFSLKSQLIVHQRSHTGVKPYGCSECG
KAFRSKSYLIIHMRTHTGEKPHECRECGKSFSFNSQLIVHQRIHTGENPYECSECGKAFNRKD
QLISHQRTHAGEKPYGCSECGKAFSSKSYLIIHMRTHSGEKPYECNECGKAFIWKSLLIVHER
THAGVNPYKCSQCEKSFSGKLRLLVHQRMHTREKPYECSECGKAFIRNSQLIVHQRTHSGEK
PYGCNECGKTFSQKSILSAHQRTHTGEKPCKCTECGKAFCWKSQLIMHQRTHVDDKH
NP_001166146.1 zinc finger protein 347 isoform a
(SEQ ID NO: 240)
MALTQGQVTFRDVAIEFSQEEWTCLDPAQRTLYRDVMLENYRNLASLAGISCFDLSIISM
LEQGKEPFTLESQVQIAGNPDGWEWIKAVITALSSEFVMKDLLHKGKSNTGEVFQTVMLER
QESQDIEGCSFREVQKNTHGLEYQCRDAEGNYKGVLLTQEGNLTHGRDEHDKRDARNKLIK
NQLGLSLQSHLPELQLFQYEGKIYECNQVEKSFNNNSSVSPPQQMPYNVKTHISKKYLKDFIS
SLLLTQGQKANNWGSPYKSNGCGMVFPQNSHLASHQRSHTKEKPYKCYECGKAFRTRSNLT
THQVIHTGEKRYKCNECGKVFSRNSQLSQHQKIHTGEKPYKCNECGKVFTQNSHLVRHRGIH
TGEKPYKCNECGKAFRARSSLAIHQATHSGEKPYKCNECGKVFTQNSHLTNHWRIHTGEKP
YKCNECGKAFGVRSSLAIHLVIHTGEKPYKCHECGKVFRRNSHLARHQLIHTGEKPYKCNEC
GKAFRAHSNLTTHQVIHTGEKPYKCNECGKVFTQNSHLANHQRIHTGVKPYMCNECGKAFS
VYSSLTTHQVIHTGEKPYKCNECGKVFTQNSHLARHRGIHTGEKPYKCNECGKVFRHNSYLS
RHQRIHTGEKPYKYNEYGKAFSEHSNLTTHQVIHTGEKPYKCNECGKVFTQNSHLARHRRV
HTGGKPYQCNECGKAFSQTSKLARHQRVHTGEKPYECNQCGKAFSVRSSLTTHQAIHTGKK
PYKCNECGKVFTQNSHLARHRGIHTGEKPYKCNECGKAFSQTSKLARHQRIHTGEKPYECG
KPFSICSSLTTHQTIHTGGKPYKCNVWKVLKSEFKPCKPSQNS
NP_037530.2 zinc finger protein 224
(SEQ ID NO: 241)
MTTFKEAMTFKDVAVVFTEEELGLLDLAQRKLYRDVMLENFRNLLSVGHQAFHRDTFHFLR
EEKIWMMKTAIQREGNSGDKIQTEMETVSEAGTHQEWSFQQIWEKIASDLTRSQDLMINSSQ
FSKEGDFPCQTEAGLSVIHTRQKSSQGNGYKPSFSDVSHFDFHQQLHSGEKSHTCDECGKNF
CYISALRIHQRVHMGEKCYKCDVCGKEFSQSSHLQTHQRVHTGEKPFKCVECGKGFSRRSAL
NVHHKLHTGEKPYNCEECGKAFIHDSQLQEHQRIHTGEKPFKCDICGKSFCGRSRLNRHSMV
HTAEKPFRCDTCDKSFRQRSALNSHRMIHTGEKPYKCEECGKGFICRRDLYTHHMVHTGEKP
YNCKECGKSFRWASCLLKHQRVHSGEKPFKCEECGKGFYTNSQCYSHQRSHSGEKPYKCVE
CGKGYKRRLDLDFHQRVHTGEKLYNCKECGKSFSRAPCLLKHERLHSGEKPFQCEECGKRF
TQNSHLHSHQRVHTGEKPYKCEKCGKGYNSKFNLDMHQKVHTGERPYNCKECGKSFGWAS
CLLKHQRLHSGEKPFKCEECGKRFTQNSQLHSHQRVHTGEKPYKCDECGKGFSWSSTRLTH
QRRHSRETPLKCEQHGKNIVQNSFSKVQEKVHSVEKPYKCEDCGKGYNRRLNLDMHQRVH
MGEKTWKCRECDMCFSQASSLRLHQNVHVGEKP
NP_113674.1 zinc finger protein 484 isoform a
(SEQ ID NO: 242)
MTKSLESVSFKDVTVDFSRDEWQQLDLAQKSLYREVMLENYFNLISVGCQVPKPEVIFSLEQ
EEPCMLDGEIPSQSRPDGDIGFGPLQQRMSEEVSFQSEININLFTRDDPYSILEELWKDDEHTR
KCGENQNKPLSRVVFINKKTLANDSIFEYKDIGEIVHVNTHLVSSRKRPHNCNSCGKNLEPIIT
LYNRNNATENSDKTIGDGDIFTHLNSHTEVTACECNQCGKPLHHKQALIQQQKIHTRESLYLF
SDYVNVFSPKSHAFAHESICAEEKQHECHECEAVFTQKSQLDGSQRVYAGICTEYEKDFSLK
SNRQKTPYEGNYYKCSDYGRAFIQKSDLFRCQRIHSGEKPYEYSECEKNLPQNSNLNIHKKIH
TGGKHFECTECGKAFTRKSTLSMHQKIHTGEKPYVCTECGKAFIRKSHFITHERIHTGEKPYE
CSDCGKSFIKKSQLHVHQRIHTGENPFICSECGKVFTHKTNLIIHQKIHTGERPYICTVCGKAFT
DRSNLIKHQKIHTGEKPYKCSDCGKSFTWKSRLRIHQKCHTGERHYECSECGKAFIQKSTLS
MHQRIHRGEKPYVCTECGKAFFHKSHFITHERIHTGEKPYECSICGKSFTKKSQLHVHQQIHT
GEKPYRCAECGKAFTDRSNLFTHQKIHTGEKPYKCSDCGKAFTRKSGLHIHQQSHTGERHYE
CSECGKAFARKSTLIMHQRIHTGEKPYICNECGKSFIQKSHLNRHRRIHTGEKPYECSDCGKSF
IKKSQLHEHHRIHTGEKPYICAECGKAFTIRSNLIKHQKIHTKQKPYKCSDLGKALNWKPQLS
MPQKSDNGEVECSMPQLWCGDSEGDQGQLSSI
NP_001006657.1 zinc finger protein 473
(SEQ ID NO: 243)
MAEEFVTLKDVGMDFTLGDWEQLGLEQGDTFWDTALDNCQDLFLLDPPRPNLTSHPDGSE
DLEPLAGGSPEATSPDVTETKNSPLMEDFFEEGFSQEIIEMLSKDGFWNSNFGEACIEDTWLD
SLLGDPESLLRSDIATNGESPTECKSHELKRGLSPVSTVSTGEDSMVHNVSEKTLTPAKSKEY
RGEFFSYSDHSQQDSVQEGEKPYQCSECGKSFSGSYRLTQHWITHTREKPTVHQECEQGFDR
NASLSVYPKTHTGYKFYVCNEYGTTFSQSTYLWHQKTHTGEKPCKSQDSDHPPSHDTQPGE
HQKTHTDSKSYNCNECGKAFTRIFHLTRHQKIHTRKRYECSKCQATFNLRKHLIQHQKTHAA
KTTSECQECGKIFRHSSLLIEHQALHAGEEPYKCNERGKSFRHNSTLKIHQRVHSGEKPYKCS
ECGKAFHRHTHLNEHRRIHTGYRPHKCQECVRSFSRPSHLMRHQAIHTAEKPYSCAECKETF
SDNNRLVQHQKMHTVKTPYECQECGERFICGSTLKCHESVHAREKQGFFVSGKILDQNPEQ
KEKCFKCNKCEKTFSCSKYLTQHERIHTRGVKPFECDQCGKAFGQSTRLIHHQRIHSRVRLY
KWGEQGKAISSASLIKLQSFHTKEHPFKCNECGKTFSHSAHLSKHQLIHAGENPFKCSKCDRV
FTQRNYLVQHERTHARKKPLVCNECGKTFRQSSCLSKHQRIHSGEKPYVCDYCGKAFGLSA
ELVRHQRIHTGEKPYVCQECGKAFTQSSCLSIHRRVHTGEKPYRCGECGKAFAQKANLTQH
QRIHTGEKPYSCNVCGKAFVLSAHLNQHLRVHTQETLYQCQRCQKAFRCHSSLSRHQRVHN
KQQYCL
NP_065879.1 zinc finger protein 28 homolog
(SEQ ID NO: 244)
MRGAASASVREPTPLPGRGAPRTKPRAGRGPTVGTPATLALPARGRPRSRNGLASKGQRGA
APTGPGHRALPSRDTALPQERNKKLEAVGTGIEPKAMSQGLVTFGDVAVDFSQEEWEWLNP
IQRNLYRKVMLENYRNLASLGLCVSKPDVISSLEQGKEPWTVKRKMTRAWCPDLKAVWKI
KELPLKKDFCEGKLSQAVITERLTSYNLEYSLLGEHWDYDALFETQPGLVTIKNLAVDFRQQ
LHPAQKNFCKNGIWENNSDLGSAGHCVAKPDLVSLLEQEKEPWMVKRELTGSLFSGQRSVH
ETQELFPKQDSYAEGVTDRTSNTKLDCSSFRENWDSDYVFGRKLAVGQETQFRQEPITHNKT
LSKERERTYNKSGRWFYLDDSEEKVHNRDSIKNFQKSSVVIKQTGIYAGKKLFKCNECKKTF
TQSSSLTVHQRIHTGEKPYKCNECGKAFSDGSSFARHQRCHTGKKPYECIECGKAFIQNTSLI
RHWRYYHTGEKPFDCIDCGKAFSDHIGLNQHRRIHTGEKPYKCDVCHKSFRYGSSLTVHQRI
HTGEKPYECDVCRKAFSHHASLTQHQRVHSGEKPFKCKECGKAFRQNIHLASHLRIHTGEKP
FECAECGKSFSISSQLATHQRIHTGEKPYECKVCSKAFTQKAHLAQHQKTHTGEKPYECKEC
GKAFSQTTHLIQHQRVHTGEKPYKCMECGKAFGDNSSCTQHQRLHTGQRPYECIECGKAFK
TKSSLICHRRSHTGEKPYECSVCGKAFSHRQSLSVHQRIHSGKKPYECKECRKTFIQIGHLNQ
HKRVHTGERSYNYKKSRKVFRQTAHLAHHQRIHTGESSTCPSLPSTSNPVDLFPKFLWNPSSL
PSP
NP_001166146.1 zinc finger protein 347 isoform a
(SEQ ID NO: 245)
MALTQGQVTFRDVAIEFSQEEWTCLDPAQRTLYRDVMLENYRNLASLAGISCFDLSIISMLE
QGKEPFTLESQVQIAGNPDGWEWIKAVITALSSEFVMKDLLHKGKSNTGEVFQTVMLERQES
QDIEGCSFREVQKNTHGLEYQCRDAEGNYKGVLLTQEGNLTHGRDEHDKRDARNKLIKNQL
GLSLQSHLPELQLFQYEGKIYECNQVEKSFNNNSSVSPPQQMPYNVKTHISKKYLKDFISSLL
LTQGQKANNWGSPYKSNGCGMVFPQNSHLASHQRSHTKEKPYKCYECGKAFRTRSNLTTH
QVIHTGEKRYKCNECGKVFSRNSQLSQHQKIHTGEKPYKCNECGKVFTQNSHLVRHRGIHTG
EKPYKCNECGKAFRARSSLAIHQATHSGEKPYKCNECGKVFTQNSHLTNHWRIHTGEKPYK
CNECGKAFGVRSSLAIHLVIHTGEKPYKCHECGKVFRRNSHLARHQLIHTGEKPYKCNECGK
AFRAHSNLTTHQVIHTGEKPYKCNECGKVFTQNSHLANHQRIHTGVKPYMCNECGKAFSVY
SSLTTHQVIHTGEKPYKCNECGKVFTQNSHLARHRGIHTGEKPYKCNECGKVFRHNSYLSRH
QRIHTGEKPYKYNEYGKAFSEHSNLTTHQVIHTGEKPYKCNECGKVFTQNSHLARHRRVHT
GGKPYQCNECGKAFSQTSKLARHQRVHTGEKPYECNQCGKAFSVRSSLTTHQAIHTGKKPY
KCNECGKVFTQNSHLARHRGIHTGEKPYKCNECGKAFSQTSKLARHQRIHTGEKPYECGKPF
SICSSLTTHQTIHTGGKPYKCNVWKVLKSEFKPCKPSQNS
NP_659570.1 zinc finger protein with KRAB and SCAN domains 5
(SEQ ID NO: 246)
MIMTESREVIDLDPPAETSQEQEDLFIVKVEEEDCTWMQEYNPPTFETFYQRFRHFQYHEASG
PREALSQLRVLCCEWLRPELHTKEQILELLVLEQFLTILPEEFQPWVREHHPESGEEAVAVIEN
IQRELEERRQQIVACPDVLPRKMATPGAVQESCSPHPLTVDTQPEQAPQKPRLLEENALPVLQ
VPSLPLKDSQELTASLLSTGSQKLVKIEEVADVAVSFILEEWGHLDQSQKSLYRDDRKENYG
SITSMGYESRDNMELIVKQISDDSESHWVAPEHTERSVPQDPDFAEVSDLKGMVQRWQVNP
TVGKSRQNPSQKRDLDAITDISPKQSTHGERGHRCSDCGKFFLQASNFIQHRRIHTGEKPFKC
GECGKSYNQRVHLTQHQRVHTGEKPYKCQVCGKAFRVSSHLVQHHSVHSGERPYGCNECG
KNFGRHSHLIEHLKRHFREKSQRCSDKRSKNTKLSVKKKISEYSEADMELSGKTQRNVSQVQ
DFGEGCEFQGKLDRKQGIPMKEILGQPSSKRMNYSEVPYVHKKSSTGERPHKCNECGKSFIQ
SAHLIQHQRIHTGEKPFRCEECGKSYNQRVHLTQHQRVHTGEKPYTCPLCGKAFRVRSHLVQ
HQSVHSGERPFKCNECGKGFGRRSHLAGHLRLHSREKSHQCRECGEIFFQYVSLIEHQVLHM
GQKNEKNGICEEAYSWNLTVIEDKKIELQEQPYQCDICGKAFGYSSDLIQHYRTHTAEKPYQ
CDICRENVGQCSHTKQHQKIYSSTKSHQCHECGRGFTLKSHLNQHQRIHTGEKPFQCKECGM
NFSWSCSLFKHLRSHERTDPINTLSVEGSLL
NP_114440.1 POZ-, AT hook-, and zinc finger-containing protein 1 short
isoform
(SEQ ID NO: 247)
MERVNDASCGPSGCYTYQVSRHSTEMLHNLNQQRKNGGRFCDVLLRVGDESFPAHRAVLA
ACSEYFESVFSAQLGDGGAADGGPADVGGATAAPGGGAGGSRELEMHTISSKVFGDILDFA
YTSRIVVRLESFPELMTAAKFLLMRSVIEICQEVIKQSNVQILVPPARADIMLFRPPGTSDLGFP
LDMTNGAALAANSNGIAGSMQPEEEAARAAGAAIAGQASLPVLPGVDRLPMVAGPLSPQLL
TSPFPSVASSAPPLTGKRGRGRPRKANLLDSMFGSPGGLREAGILPCGLCGKVFTDANRLRQH
EAQHGVTSLQLGYIDLPPPRLGENGLPISEDPDGPRKRSRTRKQVACEICGKIFRDVYHLNRH
KLSHSGEKPYSCPVCGLRFKRKDRMSYHVRSHDGSVGKPYICQSCGKGFSRPDHLNGHIKQV
HTSERPHKCQVWVGSSSGLPPLEPLPSDLPSWDFAQPALWRSSHSVPDTAFSLSLKKSFPALE
NLGPAHSSNTLFCPAPPGYLRQGWTTPEGSRAFTQWPVG
NP_001006657.1 zinc finger protein 473
(SEQ ID NO: 248)
MAEEFVTLKDVGMDFTLGDWEQLGLEQGDTFWDTALDNCQDLFLLDPPRPNLTSHPDGSE
DLEPLAGGSPEATSPDVTETKNSPLMEDFFEEGFSQEIIEMLSKDGFWNSNFGEACIEDTWLD
SLLGDPESLLRSDIATNGESPTECKSHELKRGLSPVSTVSTGEDSMVHNVSEKTLTP
AKSKEYRGEFFSYSDHSQQDSVQEGEKPYQCSECGKSFSGSYRLTQHWITHTREKPTVHQEC
EQGFDRNASLSVYPKTHTGYKFYVCNEYGTTFSQSTYLWHQKTHTGEKPCKSQDSDHPPSH
DTQPGEHQKTHTDSKSYNCNECGKAFTRIFHLTRHQKIHTRKRYECSKCQATFNLRKHLIQH
QKTHAAKTTSECQECGKIFRHSSLLIEHQALHAGEEPYKCNERGKSFRHNSTLKIHQRVHSGE
KPYKCSECGKAFHRHTHLNEHRRIHTGYRPHKCQECVRSFSRPSHLMRHQAIHTAEKPYSCA
ECKETFSDNNRLVQHQKMHTVKTPYECQECGERFICGSTLKCHESVHAREKQGFFVSGKILD
QNPEQKEKCFKCNKCEKTFSCSKYLTQHERIHTRGVKPFECDQCGKAFGQSTRLIHHQRIHSR
VRLYKWGEQGKAISSASLIKLQSFHTKEHPFKCNECGKTFSHSAHLSKHQLIHAGENPFKCSK
CDRVFTQRNYLVQHERTHARKKPLVCNECGKTFRQSSCLSKHQRIHSGEKPYVCDYCGKAF
GLSAELVRHQRIHTGEKPYVCQECGKAFTQSSCLSIHRRVHTGEKPYRCGECGKAFAQKANL
TQHQRIHTGEKPYSCNVCGKAFVLSAHLNQHLRVHTQETLYQCQRCQKAFRCHSSLSRHQR
VHNKQQYCL
NP_001973.2 receptor tyrosine-protein kinase erbB-3 isoform 1 precursor
(SEQ ID NO: 249)
MRANDALQVLGLLFSLARGSEVGNSQAVCPGTLNGLSVTGDAENQYQTLYKLYERCEVVM
GNLEIVLTGHNADLSFLQWIREVTGYVLVAMNEFSTLPLPNLRVVRGTQVYDGKFAIFVMLN
YNTNSSHALRQLRLTQLTEILSGGVYIEKNDKLCHMDTIDWRDIVRDRDAEIVVKDNGRSCP
PCHEVCKGRCWGPGSEDCQTLTKTICAPQCNGHCFGPNPNQCCHDECAGGCSGPQDTDCFA
CRHFNDSGACVPRCPQPLVYNKLTFQLEPNPHTKYQYGGVCVASCPHNFVVDQTSCVRACP
PDKMEVDKNGLKMCEPCGGLCPKACEGTGSGSRFQTVDSSNIDGFVNCTKILGNLDFLITGL
NGDPWHKIPALDPEKLNVFRTVREITGYLNIQSWPPHMHNFSVFSNLTTIGGRSLYNRGFSLLI
MKNLNVTSLGFRSLKEISAGRIYISANRQLCYHHSLNWTKVLRGPTEERLDIKHNRPRRDCV
AEGKVCDPLCSSGGCWGPGPGQCLSCRNYSRGGVCVTHCNFLNGEPREFAHEAECFSCHPE
CQPMEGTATCNGSGSDTCAQCAHFRDGPHCVSSCPHGVLGAKGPIYKYPDVQNECRPCHEN
CTQGCKGPELQDCLGQTLVLIGKTHLTMALTVIAGLVVIFMMLGGTFLYWRGRRIQNKRAM
RRYLERGESIEPLDPSEKANKVLARIFKETELRKLKVLGSGVFGTVHKGVWIPEGESIKIPVCI
KVIEDKSGRQSFQAVTDHMLAIGSLDHAHIVRLLGLCPG
SSLQLVTQYLPLGSLLDHVRQHRGALGPQLLLNWGVQIAKGMYYLEEHGMVHRNLAARNV
LLKSPSQVQVADFGVADLLPPDDKQLLYSEAKTPIKWMALESIHFGKYTHQSDVWSYGVTV
WELMTFGAEPYAGLRLAEVPDLLEKGERLAQPQICTIDVYMVMVKCWMIDENIRPTFKELA
NEFTRMARDPPRYLVIKRESGPGIAPGPEPHGLTNKKLEEVELEPELDLDLDLEAEEDNLATT
TLGSALSLPVGTLNRPRGSQSLLSPSSGYMPMNQGNLGESCQESAVSGSSERCPRPVSLHPMP
RGCLASESSEGHVTGSEAELQEKVSMCRSRSRSRSPRPRGDSAYHSQRHSLLTPVTPLSPPGL
EEEDVNGYVMPDTHLKGTPSSREGTLSSVGLSSVLGTEEEDEDEEYEYMNRRRRHSPPHPPR
PSSLEELGYEYMDVGSDLSASLGSTQSCPLHPVPIMPTAGTTPDEDYEYMNRQRDGGGPGGD
YAAMGACPASEQGYEEMRAFQGPGHQAPHVHYARLKTLRSLEATDSAFDNPDYWHSRLFP
KANAQRT
NP_037530.2 zinc finger protein 224
(SEQ ID NO: 250)
MTTFKEAMTFKDVAVVFTEEELGLLDLAQRKLYRDVMLENFRNLLSVGHQAFHRDTFHFLR
EEKIWMMKTAIQREGNSGDKIQTEMETVSEAGTHQEWSFQQIWEKIASDLTRSQDLMINSSQ
FSKEGDFPCQTEAGLSVIHTRQKSSQGNGYKPSFSDVSHFDFHQQLHSGEKSHTCDECGKNF
CYISALRIHQRVHMGEKCYKCDVCGKEFSQSSHLQTHQRVHTGEKPFKCVECGKGFSRRSAL
NVHHKLHTGEKPYNCEECGKAFIHDSQLQEHQRIHTGEKPFKCDICGKSFCGRSRLNRHSMV
HTAEKPFRCDTCDKSFRQRSALNSHRMIHTGEKPYKCEECGKGFICRRDLYTHHMVHTGEKP
YNCKECGKSFRWASCLLKHQRVHSGEKPFKCEECGKGFYTNSQCYSHQRSHSGEKPYKCVE
CGKGYKRRLDLDFHQRVHTGEKLYNCKECGKSFSRAPCLLKHERLHSGEKPFQCEECGKRF
TQNSHLHSHQRVHTGEKPYKCEKCGKGYNSKFNLDMHQKVHTGERPYNCKECGKSFGWAS
CLLKHQRLHSGEKPFKCEECGKRFTQNSQLHSHQRVHTGEKPYKCDECGKGFSWSSTRLTH
QRRHSRETPLKCEQHGKNIVQNSFSKVQEKVHSVEKPYKCEDCGKGYNRRLNLDMHQRVH
MGEKTWKCRECDMCFSQASSLRLHQNVHVGEKP
NP_065879.1 zinc finger protein 28 homolog
(SEQ ID NO: 251)
MRGAASASVREPTPLPGRGAPRTKPRAGRGPTVGTPATLALPARGRPRSRNGLASKGQRGA
APTGPGHRALPSRDTALPQERNKKLEAVGTGIEPKAMSQGLVTFGDVAVDFSQEEWEWLNP
IQRNLYRKVMLENYRNLASLGLCVSKPDVISSLEQGKEPWTVKRKMTRAWCPDLKAVWKI
KELPLKKDFCEGKLSQAVITERLTSYNLEYSLLGEHWDYDALFETQPGLVTIKNLAVDFRQQ
LHPAQKNFCKNGIWENNSDLGSAGHCVAKPDLVSLLEQEKEPWMVKRELTGSLFSGQRSVH
ETQELFPKQDSYAEGVTDRTSNTKLDCSSFRENWDSDYVFGRKLAVGQETQFRQEPITHNKT
LSKERERTYNKSGRWFYLDDSEEKVHNRDSIKNFQKSSVVIKQTGIYAGKKLFKCNECKKTF
TQSSSLTVHQRIHTGEKPYKCNECGKAFSDGSSFARHQRCHTGKKPYECIECGKAFIQNTSLI
RHWRYYHTGEKPFDCIDCGKAFSDHIGLNQHRRIHTGEKPYKCDVCHKSFRYGSSLTVHQRI
HTGEKPYECDVCRKAFSHHASLTQHQRVHSGEKPFKCKECGKAFRQNIHLASHLRIHTGEKP
FECAECGKSFSISSQLATHQRIHTGEKPYECKVCSKAFTQKAHLAQHQKTHTGEKPYECKEC
GKAFSQTTHLIQHQRVHTGEKPYKCMECGKAFGDNSSCTQHQRLHTGQRPYECIECGKAFK
TKSSLICHRRSHTGEKPYECSVCGKAFSHRQSLSVHQRIHSGKKPYECKECRKTFIQIGHLNQ
HKRVHTGERSYNYKKSRKVFRQTAHLAHHQRIHTGESSTCPSLPSTSNPVDLFPKFLWNPSSL
PSP
NP_115973.2 zinc finger protein 347 isoform b
(SEQ ID NO: 252)
MALTQGQVTFRDVAIEFSQEEWTCLDPAQRTLYRDVMLENYRNLASLGISCFDLSIISML
EQGKEPFTLESQVQIAGNPDGWEWIKAVITALSSEFVMKDLLHKGKSNTGEVFQTVMLERQ
ESQDIEGCSFREVQKNTHGLEYQCRDAEGNYKGVLLTQEGNLTHGRDEHDKRDARNKLIKN
QLGLSLQSHLPELQLFQYEGKIYECNQVEKSFNNNSSVSPPQQMPYNVKTHISKKYLKDFISS
LLLTQGQKANNWGSPYKSNGCGMVFPQNSHLASHQRSHTKEKPYKCYECGKAFRTRSNLT
THQVIHTGEKRYKCNECGKVFSRNSQLSQHQKIHTGEKPYKCNECGKVFTQNSHLVRHRGIH
TGEKPYKCNECGKAFRARSSLAIHQATHSGEKPYKCNECGKVFTQNSHLTNHWRIHTGEKP
YKCNECGKAFGVRSSLAIHLVIHTGEKPYKCHECGKVFRRNSHLARHQLIHTGEKPYKCNEC
GKAFRAHSNLTTHQVIHTGEKPYKCNECGKVFTQNSHLANHQRIHTGVKPYMCNECGKAFS
VYSSLTTHQVIHTGEKPYKCNECGKVFTQNSHLARHRGIHTGEKPYKCNECGKVFRHNSYLS
RHQRIHTGEKPYKYNEYGKAFSEHSNLTTHQVIHTGEKPYKCNECGKVFTQNSHLARHRRV
HTGGKPYQCNECGKAFSQTSKLARHQRVHTGEKPYECNQCGKAFSVRSSLTTHQAIHTGKK
PYKCNECGKVFTQNSHLARHRGIHTGEKPYKCNECGKAFSQTSKLARHQRIHTGEKPYECG
KPFSICSSLTTHQTIHTGGKPYKCNVWKVLKSEFKPCKPSQNS
NP_001005368.1 zinc finger protein 32
(SEQ ID NO: 253)
MFGFPTATLLDCHGRYAQNVAFFNVMTEAHHKYDHSEATGSSSWDIQNSFRREKLEQKSPD
SKTLQEDSPGVRQRVYECQECGKSFRQKGSLTLHERIHTGQKPFECTHCGKSFRAKGNLVTH
QRIHTGEKPYQCKECGKSFSQRGSLAVHERLHTGQKPYECAICQRSFRNQSNLAVHRRVHSG
EKPYRCDQCGKAFSQKGSLIVHIRVHTGLKPYACTQCRKSFHTRGNCILHGKIHTGETPYLCG
QCGKSFTQRGSLAVHQRSCSQRLTL
NP_004243.1 Na(+)/H(+) exchange regulatory cofactor NHE-RF1
(SEQ ID NO: 254)
MSADAAAGAPLPRLCCLEKGPNGYGFHLHGEKGKLGQYIRLVEPGSPAEKAGLLAGDRLVE
VNGENVEKETHQQVVSRIRAALNAVRLLVVDPETDEQLQKLGVQVREELLRAQEAPGQAEP
PAAAEVQGAGNENEPREADKSHPEQRELRPRLCTMKKGPSGYGFNLHSDKSKPGQFIRSVDP
DSPAEASGLRAQDRIVEVNGVCMEGKQHGDVVSAIRAGGDETKLLVVDRETDEFFKKCRVI
PSQEHLNGPLPVPFTNGEIQKENSREALAEAALESPRPALVRSASSDTSEELNSQDSPPKQDST
APSSTSSSDPILDFNISLAMAKERAHQKRSSKRAPQMDWSKKNELFSNL
NP_001159354.1 zinc finger protein 268 isoform c
(SEQ ID NO: 255)
MDVFVDFTWEEWQLLDPAQKCLYRSVMLENYSNLVSLGYQHTKPDIIFKLEQGEELCMVQ
AQVPNQTCPNTVWKIDDLMDWHQENKDKLGSTAKSFECTTFGKLCLLSTKYLSRQKPHKC
GTHGKSLKYIDFTSDYARNNPNGFQVHGKSFFHSKHEQTVIGIKYCESIESGKTVNKKSQLM
CQQMYMGEKPFGCSCCEKAFSSKSYLLVHQQTHAEEKPYGCNECGKDFSSKSYLIVHQRIHT
GEKLHECSECRKTFSFHSQLVIHQRIHTGENPYECCECGKVFSRKDQLVSHQKTHSGQKPYV
CNECGKAFGLKSQLIIHERIHTGEKPYECNECQKAFNTKSNLMVHQRTHTGEKPYVCSDCGK
AFTFKSQLIVHQGIHTGVKPYGCIQCGKGFSLKSQLIVHQRSHTGMKPYVCNECGKAFRSKS
YLIIHTRTHTGEKLHECNNCGKAFSFKSQLIIHQRIHTGENPYECHECGKAFSRKYQLISHQRT
HAGEKPYECTDCGKAFGLKSQLIIHQRTHTGEKPFECSECQKAFNTKSNLIVHQRTHTGEKPY
SCNECGKAFTFKSQLIVHKGVHTGVKPYGCSQCAKTFSLKSQLIVHQRSHTGVKPYGCSECG
KAFRSKSYLIIHMRTHTGEKPHECRECGKSFSFNSQLIVHQRIHTGENPYECSECGKAFNRKD
QLISHQRTHAGEKPYGCSECGKAFSSKSYLIIHMRTHSGEKPYECNECGKAFIWKSLLIVHER
THAGVNPYKCSQCEKSFSGKLRLLVHQRMHTREKPYECSECGKAFIRNSQLIVHQRTHSGEK
PYGCNECGKTFSQKSILSAHQRTHTGEKPCKCTECGKAFCWKSQLIMHQRTHVDDKH
NP_001090.2 prostatic acid phosphatase isoform PAP precursor
(SEQ ID NO: 256)
MRAAPLLLARAASLSLGFLFLLFFWLDRSVLAKELKFVTLVFRHGDRSPIDTFPTDPIKESSWP
QGFGQLTQLGMEQHYELGEYIRKRYRKFLNESYKHEQVYIRSTDVDRTLMSAMTNLAALFP
PEGVSIWNPILLWQPIPVHTVPLSEDQLLYLPFRNCPRFQELESETLKSEEFQKRLHPYKDFIAT
LGKLSGLHGQDLFGIWSKVYDPLYCESVHNFTLPSWATEDTMTKLRELSELSLLSLYGIHKQ
KEKSRLQGGVLVNEILNHMKRATQIPSYKKLIMYSAHDTTVSGLQMALDVYNGLLPPYASC
HLTELYFEKGEYFVEMYYRNETQHEPYPLMLPGCSPSCPLERFAELVGPVIPQDWSTECMTT
NSHQGTEDSTD
NP_001006657.1 zinc finger protein 473
(SEQ ID NO: 257)
MAEEFVTLKDVGMDFTLGDWEQLGLEQGDTFWDTALDNCQDLFLLDPPRPNLTSHPDGSE
DLEPLAGGSPEATSPDVTETKNSPLMEDFFEEGFSQEIIEMLSKDGFWNSNFGEACIEDTWLD
SLLGDPESLLRSDIATNGESPTECKSHELKRGLSPVSTVSTGEDSMVHNVSEKTLTPAKSKEY
RGEFFSYSDHSQQDSVQEGEKPYQCSECGKSFSGSYRLTQHWITHTREKPTVHQECEQGFDR
NASLSVYPKTHTGYKFYVCNEYGTTFSQSTYLWHQKTHTGEKPCKSQDSDHPPSHDTQPGE
HQKTHTDSKSYNCNECGKAFTRIFHLTRHQKIHTRKRYECSKCQATFNLRKHLIQHQKTHAA
KTTSECQECGKIFRHSSLLIEHQALHAGEEPYKCNERGKSFRHNSTLKIHQRVHSGEKPYKCS
ECGKAFHRHTHLNEHRRIHTGYRPHKCQECVRSFSRPSHLMRHQAIHTAEKPYSCAECKETF
SDNNRLVQHQKMHTVKTPYECQECGERFICGSTLKCHESVHAREKQGFFVSGKILDQNPEQ
KEKCFKCNKCEKTFSCSKYLTQHERIHTRGVKPFECDQCGKAFGQSTRLIHHQRIHSRVRLY
KWGEQGKAISSASLIKLQSFHTKEHPFKCNECGKTFSHSAHLSKHQLIHAGENPFKCSKCDRV
FTQRNYLVQHERTHARKKPLVCNECGKTFRQSSCLSKHQRIHSGEKPYVCDYCGKAFGLSA
ELVRHQRIHTGEKPYVCQECGKAFTQSSCLSIHRRVHTGEKPYRCGECGKAFAQKANLTQH
QRIHTGEKPYSCNVCGKAFVLSAHLNQHLRVHTQETLYQCQRCQKAFRCHSSLSRHQRVHN
KQQYCL
NP_001159354.1 zinc finger protein 268 isoform c
(SEQ ID NO: 258)
MDVFVDFTWEEWQLLDPAQKCLYRSVMLENYSNLVSLGYQHTKPDIIFKLEQGEELCMVQ
AQVPNQTCPNTVWKIDDLMDWHQENKDKLGSTAKSFECTTFGKLCLLSTKYLSRQKPHKC
GTHGKSLKYIDFTSDYARNNPNGFQVHGKSFFHSKHEQTVIGIKYCESIESGKTVNKKSQLM
CQQMYMGEKPFGCSCCEKAFSSKSYLLVHQQTHAEEKPYGCNECGKDFSSKSYLIVHQRIHT
GEKLHECSECRKTFSFHSQLVIHQRIHTGENPYECCECGKVFSRKDQLVSHQKTHSGQKPYV
CNECGKAFGLKSQLIIHERIHTGEKPYECNECQKAFNTKSNLMVHQRTHTGEKPYVCSDCGK
AFTFKSQLIVHQGIHTGVKPYGCIQCGKGFSLKSQLIVHQRSHTGMKPYVCNECGKAFRSKS
YLIIHTRTHTGEKLHECNNCGKAFSFKSQLIIHQRIHTGENPYECHECGKAFSRKYQLISHQRT
HAGEKPYECTDCGKAFGLKSQLIIHQRTHTGEKPFECSECQKAFNTKSNLIVHQRTHTGEKPY
SCNECGKAFTFKSQLIVHKGVHTGVKPYGCSQCAKTFSLKSQLIVHQRSHTGVKPYGCSECG
KAFRSKSYLIIHMRTHTGEKPHECRECGKSFSFNSQLIVHQRIHTGENPYECSECGKAFNRKD
QLISHQRTHAGEKPYGCSECGKAFSSKSYLIIHMRTHSGEKPYECNECGKAFIWKSLLIVHER
THAGVNPYKCSQCEKSFSGKLRLLVHQRMHTREKPYECSECGKAFIRNSQLIVHQRTHSGEK
PYGCNECGKTFSQKSILSAHQRTHTGEKPCKCTECGKAFCWKSQLIMHQRTHVDDKH
NP_001192195.1 beta-defensin 4B
(SEQ ID NO: 259)
MRVLYLLFSFLFIFLMPLPGVFGGIGDPVTCLKSGAICHPVFCPRRYKQIGTCGLPGTKC
CKKP
NP_001167629.1 zinc finger protein ZFAT isoform 4
(SEQ ID NO: 260)
MCKCCNLFSPNQSELLSHVSEKHMEEGVNVDEIIIPLRPLSTPEPPNSSKTGDEFLVMKRKRG
RPKGSTKKSSTEEELAENIVSPTEDSPLAPEEGNSLPPSSLECSKCCRKFSNTRQLRKHICIIVLN
LGEEEGEAGNESDLELEKKCKEDDREKASKRPRSQKTEKVQKISGKEARQLSGAKKPIISVVL
TAHEAIPGATKIVPVEAGPPETGATNSETTSADLVPRRGYQEYAIQQTPYEQPMKSSRLGPTQ
LKIFTCEYCNKVFKFKHSLQAHLRIHTNEKPYKCPQCSYASAIKANLNVHLRKHTGEKFACD
YCSFTCLSKGHLKVHIERVHKKIKQHCRFCKKKYSDVKNLIKHIRDAHDPQDKKVKEALDEL
CLMTREGKRQLLYDCHICERKFKNELDRDRHMLVHGDKWPFACELCGHGATKYQALELHV
RKHPFVYVCAVCRKKFVSSIRLRTHIKEVHGAAQEALVFTSSINQSFCLLEPGGDIQQEALGD
QLQLVEEEFALQGVNALKEEACPGDTQLEEGRKEPEAPGEMPAPAVHLASPQAESTALPPCE
LETTVVSSSDLHSQEVVSDDFLLKNDTSSAEAHAAPEKPPDMQHRSSVQTQGEVITLLLSKA
QSAGSDQESHGAQSPLGEGQNMAVLSAGDPDPSRCLRSNPAEASDLLPPVAGGGDTITHQPD
SCKAAPEHRSGITAFMKVLNSLQKKQMNTSLCERIRKVYGDLECEYCGKLFWYQVHFDMH
VRTHTREHLYYCSQCHYSSITKNCLKRHVIQKHSNILLKCPTDGCDYSTPDKYKLQAHLKVH
TALDKRSYSCPVCEKSFSEDRLIKSHIKTNHPEVSMSTISEVLGRRVQLKGLIGKRAMKCPYC
DFYFMKNGSDLQRHIWAHEGVKPFKCSLCEYATRSKSNLKAHMNRHSTEKTHLCDMCGKK
FKSKGTLKSHKLLHTADGKQFKCTVCDYTAAQKPQLLRHMEQHVSFKPFRCAHCHYSCNIS
GSLKRHYNRKHPNEEYANVGTGELAAEVLIQQGGLKCPVCSFVYGTKWEFNRHLKNKHGL
KVVEIDGDPKWEVTEEEPSSNHTVMIQETVQQASVELAEQHHLVVSSDDVEGIETVTVYTQG
GEASEFIVYVQEAMQPVEEQAVEQPAQEL
NP_000333.1 band 3 anion transport protein
(SEQ ID NO: 261)
MEELQDDYEDMMEENLEQEEYEDPDIPESQMEEPAAHDTEATATDYHTTSHPGTHKVYVEL
QELVMDEKNQELRWMEAARWVQLEENLGENGAWGRPHLSHLTFWSLLELRRVFTKGTVL
LDLQETSLAGVANQLLDRFIFEDQIRPQDREELLRALLLKHSHAGELEALGGVKPAVLTRSG
DPSQPLLPQHSSLETQLFCEQGDGGTEGHSPSGILEKIPPDSEATLVLVGRADFLEQPVLGFVR
LQEAAELEAVELPVPIRFLFVLLGPEAPHIDYTQLGRAAATLMSERVFRIDAYMAQSRGELLH
SLEGFLDCSLVLPPTDAPSEQALLSLVPVQRELLRRRYQSSPAKPDSSFYKGLDLNGGPDDPL
QQTGQLFGGLVRDIRRRYPYYLSDITDAFSPQVLAAVIFIYFAALSPAITFGGLLGEKTRNQM
GVSELLISTAVQGILFALLGAQPLLVVGFSGPLLVFEEAFFSFCE
TNGLEYIVGRVWIGFWLILLVVLVVAFEGSFLVRFISRYTQEIFSFLISLIFIYETFSKL
IKIFQDHPLQKTYNYNVLMVPKPQGPLPNTALLSLVLMAGTFFFAMMLRKFKNSSYFPGKLR
RVIGDFGVPISILIMVLVDFFIQDTYTQKLSVPDGFKVSNSSARGWVIHPLGLRSEFPIWMMFA
SALPALLVFILIFLESQITTLIVSKPERKMVKGSGFHLDLLLVVGMGGVAALFGMPWLSATTV
RSVTHANALTVMGKASTPGAAAQIQEVKEQRISGLLVAVLVGLSILMEPILSRIPLAVLFGIFL
YMGVTSLSGIQLFDRILLLFKPPKYHPDVPYVKRVKTWRMHLFTGIQIICLAVLWVVKSTPAS
LALPFVLILTVPLRRVLLPLIFRNVELQCLDADDAKATFDEEEG
RDEYDEVAMPV
NP_001167629.1 zinc finger protein ZFAT isoform 4
(SEQ ID NO: 262)
MCKCCNLFSPNQSELLSHVSEKHMEEGVNVDEIIIPLRPLSTPEPPNSSKTGDEFLVMKRKRG
RPKGSTKKSSTEEELAENIVSPTEDSPLAPEEGNSLPPSSLECSKCCRKFSNTRQLRKHICIIVLN
LGEEEGEAGNESDLELEKKCKEDDREKASKRPRSQKTEKVQKISGKEARQLSGAKKPIISVVL
TAHEAIPGATKIVPVEAGPPETGATNSETTSADLVPRRGYQEYAIQQTPYEQPMKSSRLGPTQ
LKIFTCEYCNKVFKFKHSLQAHLRIHTNEKPYKCPQCSYASAIKANLNVHLRKHTGEKFACD
YCSFTCLSKGHLKVHIERVHKKIKQHCRFCKKKYSDVKNLIKHIRDAHDPQDKKVKEALDEL
CLMTREGKRQLLYDCHICERKFKNELDRDRHMLVHGDKWPFACELCGHGATKYQALELHV
RKHPFVYVCAVCRKKFVSSIRLRTHIKEVHGAAQEALVFTSSINQSFCLLEPGGDIQQEALGD
QLQLVEEEFALQGVNALKEEACPGDTQLEEGRKEPEAPGEMPAPAVHLASPQAESTALPPCE
LETTVVSSSDLHSQEVVSDDFLLKNDTSSAEAHAAPEKPPDMQHRSSVQTQGEVITLLLSKA
QSAGSDQESHGAQSPLGEGQNMAVLSAGDPDPSRCLRSNPAEASDLLPPVAGGGDTITHQPD
SCKAAPEHRSGITAFMKVLNSLQKKQMNTSLCERIRKVYGDLECEYCGKLFWYQVHFDMH
VRTHTREHLYYCSQCHYSSITKNCLKRHVIQKHSNILLKCPTDGCDYSTPDKYKLQAHLKVH
TALDKRSYSCPVCEKSFSEDRLIKSHIKTNHPEVSMSTISEVLGRRVQLKGLIGKRAMKCPYC
DFYFMKNGSDLQRHIWAHEGVKPFKCSLCEYATRSKSNLKAHMNRHSTEKTHLCDMCGKK
FKSKGTLKSHKLLHTADGKQFKCTVCDYTAAQKPQLLRHMEQHVSFKPFRCAHCHYSCNIS
GSLKRHYNRKHPNEEYANVGTGELAAEVLIQQGGLKCPVCSFVYGTKWEFNRHLKNKHGL
KVVEIDGDPKWEVTEEEPSSNHTVMIQETVQQASVELAEQHHLVVSSDDVEGIETVTVYTQG
GEASEFIVYVQEAMQPVEEQAVEQPAQEL
NP_065914.2 zinc finger protein ZFAT isoform 1
(SEQ ID NO: 263)
METRAAENTAIFMCKCCNLFSPNQSELLSHVSEKHMEEGVNVDEIIIPLRPLSTPEPPNSSKTG
DEFLVMKRKRGRPKGSTKKSSTEEELAENIVSPTEDSPLAPEEGNSLPPSSLECSKCCRKFSNT
RQLRKHICIIVLNLGEEEGEAGNESDLELEKKCKEDDREKASKRPRSQKTEKVQKISGKEARQ
LSGAKKPIISVVLTAHEAIPGATKIVPVEAGPPETGATNSETTSADLVPRRGYQEYAIQQTPYE
QPMKSSRLGPTQLKIFTCEYCNKVFKFKHSLQAHLRIHTNEKPYKCPQCSYASAIKANLNVH
LRKHTGEKFACDYCSFTCLSKGHLKVHIERVHKKIKQHCRFCKKKYSDVKNLIKHIRDAHDP
QDKKVKEALDELCLMTREGKRQLLYDCHICERKFKNELDRDRHMLVHGDKWPFACELCGH
GATKYQALELHVRKHPFVYVCAVCRKKFVSSIRLRTHIKEVHGAAQEALVFTSSINQSFCLLE
PGGDIQQEALGDQLQLVEEEFALQGVNALKEEACPGDTQLEEGRKEPEAPGEMPAPAVHLA
SPQAESTALPPCELETTVVSSSDLHSQEVVSDDFLLKNDTSSAEAHAAPEKPPDMQHRSSVQT
QGEVITLLLSKAQSAGSDQESHGAQSPLGEGQNMAVLSAGDPDPSRCLRSNPAEASDLLPPV
AGGGDTITHQPDSCKAAPEHRSGITAFMKVLNSLQKKQMNTSLCERIRKVYGDLECEYCGK
LFWYQVHFDMHVRTHTREHLYYCSQCHYSSITKNCLKRHVIQKHSNILLKCPTDGCDYSTPD
KYKLQAHLKVHTALDKRSYSCPVCEKSFSEDRLIKSHIKTNHPEVSMSTISEVLGRRVQLKGL
IGKRAMKCPYCDFYFMKNGSDLQRHIWAHEGVKPFKCSLCEYATRSKSNLKAHMNRHSTE
KTHLCDMCGKKFKSKGTLKSHKLLHTADGKQFKCTVCDYTAAQKPQLLRHMEQHVSFKPF
RCAHCHYSCNISGSLKRHYNRKHPNEEYANVGTGELAAEVLIQQGGLKCPVCSFVYGTKWE
FNRHLKNKHGLKVVEIDGDPKWETATEAPEEPSTQYLHITEAEEDVQGTQAAVAALQDLRY
TSESGDRLDPTAVNILQQIIELGAETHDATALASVVAMAPGTVTVVKQVTEEEPSSNHTVMIQ
ETVQQASVELAEQHHLVVSSDDVEGIETVTVYTQGGEASEFIVYVQEAMQPVEEQAVEQPA
QEL

Section 2 of Sequence Listing: Amino acid sequence information for exemplary domains of naturally occurring polypeptides having size and charge characteristics of Surf+ Penetrating Polypeptides, and referenced by PDB number and chain in FIGS. 1 and 2.

### Amino Acid Sequence Information for Specific Domains Identified by PDB Identifier and Chain in Figures

2J2S: A histone-lysine N-methyltransferase MLL isoform 1 precursor
(SEQ ID NO: 264)
GGSVKKGRRSRRCGQCPGCQVPEDCGVCTNCLDKPKFGGRNIKKQCCKMRKCQNLQWMP
SKAYLQKQAKAVK
1FOS: F transcription factor AP-1
(SEQ ID NO: 265)
KAERKRMRNRIAASKSRKRKLERIARLEEKVKTLKAQNSELASTANMLREQVAQLKQKVM
NH
1G2S: A C-C motif chemokine 26 precursor
(SEQ ID NO: 266)
TRGSDISKTCCFQYSHKPLPWTWVRSYEFTSNSCSQRAVIFTTKRGKKVCTHPRKKWVQKY
ISLLKTPKQL
1XDT: R proheparin-binding EGF-like growth factor precursor
(SEQ ID NO: 267)
GSHMRVTLSSKPQALATPNKEEHGKRKKKGKGLGKKRDPCLRKYKDFCIHGECKYVKELR
APSCICHPGYHGERCHGLS
2JX3: A protein DEK isoform 1
(SEQ ID NO: 268)
FTIAQGKGQKLCEIERIHFFLSKKKTDELRNLHKLLYNRPGTVSSLKKNVGQFSGFPFEK
GSVQYKKKEEMLKKFRNAMLKSICEVLDLERSGVNSELVKRILNFLMHPKPSGKPLPKSKK
TCSKGSKKER
2HGF: A hepatocyte growth factor isoform 1 preproprotein
(SEQ ID NO: 269)
GQRKRRNTIHEFKKSAKTTLIKIDPALKIKTKKVNTADQCANRCTRNKGLPFTCKAFVFD
KARKQCLWFPFNSMSSGVKKEFGHEFDLYENKDYIRN
1KJ6: A beta-defensin 103 precursor
(SEQ ID NO: 270)
GIINTLQKYYCRVRGGRCAVLSCLPKEEQIGKCSTRGRKCCRRKK
1TDH: A Endonuclease VIII-like 1
(SEQ ID NO: 271)
MPEGPELHLASQFVNEACRALVFGGCVEKSSVSRNPEVPFESSAYRISASARGKELRLIL
SPLPGAQPQQEPLALVFRFGMSGSFQLVPREELPRHAHLRFYTAPPGPRLALCFVDIRRF
GRWDLGGKWQPGRGPCVLQEYQQFRESVLRNLADKAFDRPICEALLDQRFFNGIGNYLRA
EILYRLKIPPFEKARSVLEALQQHRPSPELTLSQKIRTKLQNPDLLELCHSVPKEVVQLGGRG
YGSESGEEDFAAFRAWLRCYGMPGMSSLQDRHGRTIWFQGDPGPLAPKGRKSRKKKSKAT
QLSPEDRVEDALPPSKAPSRTRRAKRDLPKRKGRQAASGHCRPRKVKADIPSLEPEGTSAS
1J3S: A cytochrome c
(SEQ ID NO: 272)
GDVEKGKKIFIMKCSQCHTVEKGGKHKTGPNLHGLFGRKTGQAPGYSYTAANKNKGIIWG
EDTLMEYLENPKKYIPGTKMIFVGIKKKEERADLIAYLKKATNE
1NUN: A fibroblast growth factor 10 precursor
(SEQ ID NO: 273)
GRHVRSYNHLQGDVRWRKLFSFTKYFLKIEKNGKVSGTKKENCPYSILEITSVEIGVVAV
KAINSNYYLAMNKKGKLYGSKEFNNDCKLKERIEENGYNTYASFNWQHNGRQMYVALNG
KGAPRRGQKTRRKNTSAHFLPMVVHS
1EIG: A C-C motif chemokine 24 precursor
(SEQ ID NO: 274)
VVIPSPCCMFFVSKRIPENRVVSYQLSSRSTCLKAGVIFTTKKGQQSCGDPKQEWVQRYM
KNLDAKQKKASPR
1E8O: B signal recognition particle 14 kDa protein
(SEQ ID NO: 275)
VLLESEQFLTELTRLFQKCRTSGSVYITLKKYDGRTKPIPKKGTVEGFEPADNKCLLRAT
DGKKKISTVVSSKEVNKFQMAYSNLLRANMDGLKKRDKKNKTKKTK
1Z9I: A epidermal growth factor receptor isoform a precursor
(SEQ ID NO: 276)
RRRHIVRKRTLRRLLQERELVEPLTPSGEAPNQALLRILKETEFKKIKVLGSG
2HDL: A C-X-C motif chemokine 14 precursor
(SEQ ID NO: 277)
GSKCKCSRKGPKIRYSDVKKLEMKPKYPHCEEKMVIITTKSVSRYRGQEHCLHPKLQSTKR
FIKWYNAWNEKRRVYEE
1JXS: A forkhead box protein K2
(SEQ ID NO: 278)
DSKPPYSYAQLIVQAITMAPDKQLTLNGIYTHITKNYPYYRTADKGWQNSIRHNLSLNRY
FIKVPRSQEEPGKGSFWRIDPASESKLIEQAFRKRRPR
1UZC: A pre-mRNA-processing factor 40 homolog A
(SEQ ID NO: 279)
GSQPAKKTYTWNTKEEAKQAFKELLKEKRVPSNASWEQAMKMIINDPRYSALAKLSEKKQ
AFNAYKVQTEK
2Y9A: D small nuclear ribonucleoprotein Sm D3
(SEQ ID NO: 280)
MSIGVPIKVLHEAEGHIVTCETNTGEVYRGKLIEAEDNMNCQMSNITVTYRDGRVAQLEQV
YIRGSKIRFLILPDMLKNAPMLKSMKNKNQGSGAGRGKAAILKAQVAARGRGRGMGRGNI
FQKRR
2KKR: A ataxin-7 isoform a
(SEQ ID NO: 281)
GSKFLNKRLSEREFDPDIHCGVIDLDTKKPCTRSLTCKTHSLTQRRAVQGRRKRFDVLLA
EHKNKTREKELIRH
1V66: A E3 SUMO-protein ligase PIAS1
(SEQ ID NO: 282)
MADSAELKQMVMSLRVSELQVLLGYAGRNKHGRKHELLTKALHLLKAGCSPAVQMKIKE
LYRRRF
1PFM: A platelet factor 4 precursor
(SEQ ID NO: 283)
MSAKELRCQCVKTTSQVRPRHITSLEVIKAGPHCPTAQLIATLKNGRKICLDLQAPLYKK
IIKKLLES
2E5E: A advanced glycosylation end product-specific receptor isoform 2 precursor
(SEQ ID NO: 284)
AMAQNITARIGEPLVLKCKGAPKKPPQRLEWKLNTGRTEAWKVLSPQGGGPWDSVARVLP
NGSLFLPAVGIQDEGIFRCQAMNRNGKETKSNYRVRVYQIP
2FDB: M fibroblast growth factor 8 isoform B precursor
(SEQ ID NO: 285)
QVTVQSSPNFTQHVREQSLVTDQLSRRLIRTYQLYSRTSGKHVQVLANKRINAMAEDGDPF
AKLIVETDTFGSRVRVRGAETGLYICMNKKGKLIAKSNGKGKDCVFTEIVLENNYTALQNA
KYEGWYMAFTRKGRPRKGSKTRQHQREVHFMKRLPRGHHTTE
1UKL: C sterol regulatory element-binding protein 2
(SEQ ID NO: 286)
RSSINDKIIELKDLVXGTDAKXHKSGVLRKAIDYIKYLQQVNHKLRQENXVLKLANQKNKL
3HTU: B charged multivesicular body protein 6
(SEQ ID NO: 287)
GSRVTEQDKAILQLKQQRDKLRQYQKRIAQQLERERALA
2KOL: A stromal cell-derived factor 1 isoform gamma
(SEQ ID NO: 288)
KPVSLSYRCPCRFFESHVARANVKRLKILNTPNCALQIVARLKNNNRQVCIDPKLKWIQE
YLEKALNK
2K8F: A histone acetyltransferase p300
(SEQ ID NO: 289)
ATQSPGDSRRLSIQRAIQSLVHAAQCRNANCSLPSCQKMKRVVQHTKGCKRKTNGGCPICK
QLIALAAYHAKHCQENKCPVPFCLNIKQK
3IWN: C U1 small nuclear ribonucleoprotein A
(SEQ ID NO: 290)
TRPNHTIYINNLNEKIKKDELKKSLHAIFSRFGQILDILVSRSLKMRGQAFVIFKEVSSA
TNALRSMQGFPFYDKPMRIQYAKTDSDIIAK
1PUF: B pre-B-cell leukemia transcription factor 1 isoform 2
(SEQ ID NO: 291)
ARRKRRNFNKQATEILNEYFYSHLSNPYPSEEAKEELAKKCGITVSQVSNWFGNKRIRYK
KNIGKFQEEANIY
2L9R: A homeobox protein Nkx-3.1
(SEQ ID NO: 292)
MGHHHHHHSHMSHTQVIELERKFSHQKYLSAPERAHLAKNLKLTETQVKIWFQNRRYKTK
RKQLSSELG
1PUF: A homeobox protein Hox-A9
(SEQ ID NO: 293)
NNPAANWLHARSTRKKRCPYTKHQTLELEKEFLFNMYLTRDRRYEVARLLNLTERQVKIW
FQNRRMKMKKINKDRAK
2LCE: A B-cell lymphoma 6 protein isoform 1
(SEQ ID NO: 294)
MGHHHHHHSHMTHSDKPYKCDRCQASFRYKGNLASHKTVHTGEKPYRCNICGAQFNRPA
NLKTHTRIHSGEKPX
1BC7: C ETS domain-containing protein Elk-4 isoform a
(SEQ ID NO: 295)
MDSAITLWQFLLQLLQKPQNKHMICWTSNDGQFKLLQAEEVARLWGIRKNKPNMNYDKL
SRALRYYYVKNIIKKVNGQKFVYKFVSYPEILNM
1YZ8: P pituitary homeobox 3
(SEQ ID NO: 296)
GSQRRQRTHFTSQQLQQLEATFQRNRYPDMSTREEIAVWTNLTEARVRVWFKNRRAKWR
KREEFIVTD
1L9L: A granulysin isoform NKG5
(SEQ ID NO: 297)
XRDYRTCLTIVQKLKKMVDKPTQRSVSNAATRVCRTGRSRWRDVCRNFMRRYQSRVIQGL
VAGETAQQICEDLX
1127: A general transcription factor IIF subunit 1
(SEQ ID NO: 298)
GPLGSGDVQVTEDAVRRYLTRKPMTTKDLLKKFQTKKTGLSSEQTVNVLAQILKRLNPERK
MINDKMHFSLKE
2KDP: A histone deacetylase complex subunit SAP30
(SEQ ID NO: 299)
SNAGQLCCLREDGERCGRAAGNASFSKRIQKSISQKKVKIELDKSARHLYICDYHKNLIQ
SVRNRRKRKGS
2RQP: A heterochromatin protein 1-binding protein 3
(SEQ ID NO: 300)
GPGMASSPRPKMDAILTEAIKACFQKSGASVVAIRKYIIHKYPSLELERRGYLLKQALKR
ELNRGVIKQVKGKGASGSFVVVQKSRKT
1EOT: A eotaxin precursor
(SEQ ID NO: 301)
GPASVPTTCCFNLANRKIPLQRLESYRRITSGKCPQKAVIFKTKLAKDICADPKKKWVQD
SMKYLDQKSPTPKP
2L1Q: A liver-expressed antimicrobial peptide 2 precursor
(SEQ ID NO: 302)
MTPFWRGVSLRPIGASCRDDSECITRLCRKRRCSLSVAQE
2W0T: A lethal(3)malignant brain tumor-like protein 2
(SEQ ID NO: 303)
GSGSEPAVCEMCGIVGTREAFFSKTKRFCSVSCSRSYSSNSKK
1J8I: A lymphotactin precursor
(SEQ ID NO: 304)
XGSEVSDKRTCVSLTTQRLPVSRIKTYTITEGSLRAVIFITKRGLKVCADPQATWVRDVV
RSMDRKSNTRNNMIQTKPTGTQQSTNTAVTLTG
1H89: A CCAAT/enhancer-binding protein beta
(SEQ ID NO: 305)
EYKIRRERNNIAVRKSRDKAKMRNLETQHKVLELTAENERLQKKVEQLSRELSTLRNLFKQ
LPE
1J1D: B troponin T, cardiac muscle isoform 2
(SEQ ID NO: 306)
HFGGYIQKQAQTERKSGKRQTEREKKKKILAERRKVLAIDHLNEDQLREKAKELWQTIYNL
EAEKFDLQEKFKQQKYEINVLRNRINDNQKVSKTRGKAKVTGRWK
1KBH: B CREB-binding protein isoform b
(SEQ ID NO: 307)
XNRSISPSALQDLLRTLKSPSSPQQQQQVLNILKSNPQLMAAFIKQRTAKYVANQPGMQ
1T2K: D cyclic AMP-dependent transcription factor ATF-2
(SEQ ID NO: 308)
KRRKFLERNRAAASRSRQKRKVWVQSLEKKAEDLSSLNGQLQSEVTLLRNEVAQLKQLLL
A
1TZS: P cathepsin E isoform a preproprotein
(SEQ ID NO: 309)
GSLHRVPLRRHPSLKKKLRARSQLSEFWKSHNLDM
1VRY: A glycine receptor subunit alpha-1 isoform 1 precursor
(SEQ ID NO: 310)
LPARVGLGITTVLTLTTQSSGSRASLPKVSYVKAIDIWLAVCLLFVFSALLEYAAVNFVS
RKKKKHRLLEHHHHHH
1ZOQ: C CREB-binding protein isoform b
(SEQ ID NO: 310)
SALQDLLRTLKSPSSPQQQQQVLNILKSNPQLMAAFIKQRTAKYVAN
2D2P: A pituitary adenylate cyclase-activating polypeptide precursor
(SEQ ID NO: 311)
HSDGIFTDSYSRYRKQMAVKKYLAAVLGKRYKQRVKNKX
2F8X: M mastermind-like protein 1
(SEQ ID NO: 312)
GLPRHSAVMERLRRRIELCRRHHSTCEARYEAVSPERLELERQHTFALHQRCIQAKAKRA
GKH
2J5D: A BCL2/adenovirus E1B 19 kDa protein-interacting protein 3
(SEQ ID NO: 313)
RNTSVMKKGGIFSAEFLKVFLPSLLLSHLLAIGLGIYIGRRLTTS
2K6O: A cathelicidin antimicrobial peptide
(SEQ ID NO: 314)
LLGDFFRKSKEKIGKEFKRIVQRIKDFLRNLVPRTES
2KLU: A T-cell surface glycoprotein CD4 isoform 3
(SEQ ID NO: 315)
GPLVPRGSMALIVLGGVAGLLLFIGLGIFFSVRSRHRRRQAERMSQIKRLLSEKKTSQSP
HRFQKTHSPI
2KS1: B epidermal growth factor receptor isoform a precursor
(SEQ ID NO: 316)
EGCPTNGPKIPSIATGMVGALLLLLVVALGIGLFMRRRHIVRKR
2KZ5: A transcription factor NF-E2 45 kDa subunit isoform 2
(SEQ ID NO: 317)
MGHHHHHHSHMAKPTARGEAGSRDERRALAMKIPFPTDKIVNLPVDDFNELLARYPLTES
QLALVRDIRRRGKNKVAAQNYRKRKLETIVQ
3BEG: B serine/arginine-rich splicing factor 1 isoform 1
(SEQ ID NO: 318)
GGAPRGRYGPPSRRSENRVVVSGLPPSGSWQDLKDHMREAGDVCYADVYRDGTGVVEFV
RKEDMTYAVRKLDNTKFRSHEGETAYIRVKVDGPRSPSYGRSRSRSRSRSRSRSRS
3FFD: P parathyroid hormone-related protein isoform 2 preproprotein
(SEQ ID NO: 319)
AVSEHQLLHDKGKSIQDLRRRFFLHHLIAEIHTAEIRATSEVSPNSKPSPNTKNHPVRFG
SDDEGRYLTQETNKVETYKEQPLKTPGKKKKGKPGKRKEQEKKKRRTR
3G9W: C integrin beta-1 isoform 1D precursor
(SEQ ID NO: 320)
GPKLLMIIHDRREFAKFEKEKMNAKWDTQENPIYKSPINNFKNPNYGRKAGL
2PA2: A 60S ribosomal protein L10
(SEQ ID NO: 321)
GSFDLGRKKAKVDEFPLCGHMVSDEYEQLSSEALEAARICANKYMVKSCGKDGFHIRVRL
HPFHVIRINKMLSCAGADRLQTGMRGAFGKPQGTVARVHIGQVIMSIRTKLQNKEHVIEAL
RRAKFKFPGRQKIHISKKWGFTKFNADEFE
2L7U: A advanced glycosylation end product-specific receptor isoform 2 precursor
(SEQ ID NO: 322)
GSAQNITARIGEPLVLKCKGAPKKPPQRLEWKLNTGRTEAWKVLSPQGGGPWDSVARVLP
NGSLFLPAVGIQDEGIFRCQAMNRNGKETKSNYRVRVYQIPGKPE
2RA4: A C-C motif chemokine 13 precursor
(SEQ ID NO: 323)
MQPDALNVPSTCCFTFSSKKISLQRLKSYVITTSRCPQKAVIFRTKLGKEICADPKEKWV
QNYMKHLGRKAHTLKT
2VXW: A C-C motif chemokine 5 precursor
(SEQ ID NO: 324)
FSPLSSQSSACCFAYIARPLPRAHIKEYFYTSGKCSNPAVVFVTRKNRQVCANPEKKWVR
EYINSLEMS
1BO0: A C-C motif chemokine 7 precursor
(SEQ ID NO: 325)
XPVGINTSTTCCYRFINKKIPKQRLESYRRTTSSHCPREAVIFKTKLDKEICADPTQKWV
QDFMKHLDKKTQTPKL
1QNK: A C-X-C motif chemokine 2
(SEQ ID NO: 326)
XELRCQCLQTLQGIHLKNIQSVKVKSPGPHCAQTEVIATLKNGQKACLNPASPMVKKIIE
KMLKNGKSN
3CO7: C forkhead box protein O1
(SEQ ID NO: 327)
SKSSSSRRNAWGNLSYADLITKAIESSAEKRLTLSQIYEWMVKSVPYFKDKGDSNSSAGWK
NSIRHNLSLHSKFIRVQNEGTGKSSWWMLNPEGGKSGKSPRRRAASMDNNSKFAKS
2K86: A forkhead box protein O3
(SEQ ID NO: 328)
GSSSRRNAWGNLSYADLITRAIESSPDKRLTLSQIYEWMVRCVPYFKDKGDSNSSAGWKNS
IRHNLSLHSRFMRVQNEGTGKSSWWIINPDGGKSGKAPRRRA
1E17: A forkhead box protein O4 isoform 1
(SEQ ID NO: 329)
GSSHHHHHHSSGLVPRGSHMLEDPGAVTGPRKGGSRRNAWGNQSYAELISQAIESAPEKRL
TLAQIYEWMVRTVPYFKDKGDSNSSAGWKNSIRHNLSLHSKFIKVHNEATGKSSWWMLNP
EGGKSGKAPRRRAASMDSSSKLLRGRSKA
1NHA: A general transcription factor IIF subunit 1
(SEQ ID NO: 330)
STPQPPSGKTTPNSGDVQVTEDAVRRYLTRKPMTTKDLLKKFQTKKTGLSSEQTVNVLAQI
LKRLNPERKMINDKMHFSLKE
2K7L: A general transcription factor IIF subunit 1
(SEQ ID NO: 331)
DVQVTEDAVRRYLTRKPMTTKDLLKKFQTKKTGLSSEQTVNVLAQILKRLNPERKMINDK
MHFSLKE
1BFF: A heparin-binding growth factor 2
(SEQ ID NO: 332)
KDPKRLYCKNGGFFLRIHPDGRVDGVREKSDPHIKLQLQAEERGVVSIKGVCANRYLAMKE
DGRLLASKCVTDECFFFERLESNNYNTYRSRKYTSWYVALKRTGQYKLGSKTGPGQKAILF
LPMSAKS
1CVS: A heparin-binding growth factor 2
(SEQ ID NO: 333)
GHFKDPKRLYCKNGGFFLRIHPDGRVDGVREKSDPHIKLQLQAEERGVVSIKGVSANRYLA
MKEDGRLLASKSVTDECFFFERLESNNYNTYRSRKYTSWYVALKRTGQYKLGSKTGPGQK
AILFLPMSAKS
3HMS: A hepatocyte growth factor isoform 1 preproprotein
(SEQ ID NO: 334)
GSYAEGQRKRRNTIHEFKKSAKTTLIKIDPALKIKTKKVNTADQCANRCTRNKGLPFTCK
AFVFDKARKQCLWFPFNSMSSGVKKEFGHEFDLYENKDYIR
1M36: A histone acetyltransferase MYST3
(SEQ ID NO: 335)
GSRLPKLYLCEFCLKYMKSRTILQQHMKKCGWF
3R45: A histone H3-like centromeric protein A isoform a
(SEQ ID NO: 336)
MGSSHHHHHHSQDPNSMGPRRRSRKPEAPRRRSPSPTPTPGPSRRGPSLGASSHQHSRRR
QGWLKEIRKLQKSTHLLIRKLPFSRLAREICVKFTRGVDFNWQAQALLALQEAAEAFLVHLF
EDAYLLTLHAGRVTLFPKDVQLARRIRGLEEGLG
3AN2: A histone H3-like centromeric protein A isoform a
(SEQ ID NO: 337)
GSHMGPRRRSRKPEAPRRRSPSPTPTPGP SRRGPSLGASSHQHSRRRQGWLKEIRKLQKS
THLLIRKLPFSRLAREICVKFTRGVDFNWQAQALLALQEAAEAFLVHLFEDAYLLTLHAG
RVTLFPKDVQLARRIRGLEEGLG
3NQU: A histone H3-like centromeric protein A isoform a
(SEQ ID NO: 338)
MGPRRRSRKPEAPRRRSPSPTPTPGPSRRGPSLGASSHQHSRRRQGWLKEIRKLQKSTHL
LIRKLPFSRLAREICVKFTRGVDFNWQAQALLALQEAAEAFLVHLFEDAYLLTLHAGRVTLF
PKDVQLARRIRGLEEGLG
1B72: A homeobox protein Hox-B1
(SEQ ID NO: 339)
MEPNTPTARTFDWMKVKRNPPKTAKVSEPGLGSPSGLRTNFTTRQLTELEKEFHFNKYLSR
ARRVEIAATLELNETQVKIWFQNRRMKQKKREREGG
2KT0: A homeobox protein NANOG
(SEQ ID NO: 340)
SKQPTSAENSVAKKEDKVPVKKQKTRTVFSSTQLCVLNDRFQRQKYLSLQQMQELSNILNL
SYKQVKTWFQNQRMKSKRWQKNN
1HLV: A major centromere autoantigen B
(SEQ ID NO: 341)
MGPKRRQLTFREKSRIIQEVEENPDLRKGEIARRFNIPPSTLSTILKNKRAILASERKYG
VASTCRKTNKLSPYDKLEGLLIAWFQQIRAAGLPVKGIILKEKALRIAEELGMDDFTASN
GWLDRFRRRRS
1BW6: A major centromere autoantigen B
(SEQ ID NO: 342)
MGPKRRQLTFREKSRIIQEVEENPDLRKGEIARRFNIPPSTLSTILKNKRAILASE
3OA6: A male-specific lethal 3 homolog isoform a
(SEQ ID NO: 343)
MKKHHHHHHMSASEGMKFKFHSGEKVLCFEPDPTKARVLYDAKIVDVIVGKDEKGRKIPE
YLIHFNGWNRSWDRWAAEDHVLRDTDENRRLQRKLARKAVARLRSTGRKK
1NLW: A max dimerization protein 1 isoform 2
(SEQ ID NO: 344)
SRSTHNEMEKNRRAHLRLSLEKLKGLVPLGPDS SRHTTLSLLTKAKLHIKKLEDSDRKAV
HQIDQLQREQRHLKRQLEKL
1K99: A nucleolar transcription factor 1 isoform a
(SEQ ID NO: 345)
MKKLKKHPDFPKKPLTPYFRFFMEKRAKYAKLHPEMSNLDLTKILSKKYKELPEKKKMKYI
QDFQREKQEFERNLARFREDHPDLIQNAKKLEHHHHHH
2LB3: A peptidyl-prolyl cis-trans isomerase NIMA-interacting 1
(SEQ ID NO: 346)
KLPPGWEKRMSRSSGRVYYFNHITNASQWERPSGNS
2KCF: A peptidyl-prolyl cis-trans isomerase NIMA-interacting 1
(SEQ ID NO: 347)
GSKLPPGWEKRMSRSSGRVYYFNHITNASQWERPSG
2JOD: B pituitary adenylate cyclase-activating polypeptide precursor
(SEQ ID NO: 348)
FTDSYSRYRKQMAVKKYLAAVLGKRYKQRVKNK
1CQT: I POU domain class 2-associating factor 1
(SEQ ID NO: 349)
MLWQKPTAPEQAPAPARPYQGVRVKEPVKELLRRKRGHASSGAA
1POG: A POU domain, class 2, transcription factor 1 isoform 3
(SEQ ID NO: 350)
RGSHMRRRKKRTSIETNIRVALEKSFLENQKPTSEEITMIADQLNMEKEVIRVWFCNRRQ
KEKRIDI
1B72: B pre-B-cell leukemia transcription factor 1 isoform 2
(SEQ ID NO: 351)
ARRKRRNFNKQATEILNEYFYSHLSNPYPSEEAKEELAKKCGITVSQVSNWFGNKRIRYK
KNIGKFQEEANIYAAKTAVTATNVSAH
3KUC: B RAF proto-oncogene serine/threonine-protein kinase
(SEQ ID NO: 352)
PSKTSNTIRVFLPNKQRTVVRVRNGMSLHDCLMKKLKVRGLQPECCAVFRLLHEHKGKKA
RLDWNTDAASLIGEELQVDFL
2JWA: A receptor tyrosine-protein kinase erbB-2 isoform b
(SEQ ID NO: 353)
GCPAEQRASPLTSIISAVVGILLVVVLGVVFGILIKRRQQKIRK
2L2T: A receptor tyrosine-protein kinase erbB-4 isoform JM-a/
CVT-2 precursor
(SEQ ID NO: 354)
STLPQHARTPLIAAGVIGGLFILVIVGLTFAVYVRRKSIKKKRA
2AZE: C retinoblastoma-associated protein
(SEQ ID NO: 355)
SRILVSIGESFGTSEKFQKINQMVCNSDRVLKRSAEGSNPPKPLKK
3BSU: A ribonuclease H1
(SEQ ID NO: 356)
GSHMFYAVRRGRKTGVFLTWNECRAQVDRFPAARFKKFATEDEAWAFVRKSAS
3IXS: B RING1 and YY1-binding protein
(SEQ ID NO: 357)
GTRPRLKNVDRSTAQQLAVTVGNVTVIITDFKEKTRS
2FY1: A RNA-binding motif protein, Y chromosome, family 1 member B
(SEQ ID NO: 358)
MVEADHPGKLFIGGLNRETNEKMLKAVFGKHGPISEVLLIKDRTSKSRGFAFITFENPAD
AKNAAKDMNGKSLHGKAIKVEQAKKPSFQSGGRRRPPASSRNRSPSGSLEHHHHHH
1YO5: C SAM pointed domain-containing Ets transcription factor
(SEQ ID NO: 359)
GSLDALGSQPIHLWQFLKELLLKPHSYGRFIRWLNKEKGIFKIEDSAQVARLWGIRKNRP
AMNYDKLSRSIRQYYKKGIIRKPDISQRLVYQFVHPI
1K6O: B serum response factor
(SEQ ID NO: 360)
GAKPGKKTRGRVKIKMEFIDNKLRRYTTFSKRKTGIMKKAYELSTLTGTQVLLLVASETGH
VYTFATRKLQPMITSETGKALIQTCLNSPDSPPRSDPTTDQR
1HBX: A serum response factor
(SEQ ID NO: 361)
SGAKPGKKTRGRVKIKMEFIDNKLRRYTTFSKRKTGIMKKAYELSTLTGTQVLLLVASET
GHVYTFATRKLQPMITSETGKALIQTCLNSPD
1J46: A sex-determining region Y protein
(SEQ ID NO: 362)
MQDRVKRPMNAFIVWSRDQRRKMALENPRMRNSEISKQLGYQWKMLTEAEKWPFFQEAQ
KLQAMHREKYPNYKYRPRRKAKMLPK
1J47: A sex-determining region Y protein
(SEQ ID NO: 363)
MQDRVKRPINAFIVWSRDQRRKMALENPRMRNSEISKQLGYQWKMLTEAEKWPFFQEAQ
KLQAMHREKYPNYKYRPRRKAKMLPK
1B34: B small nuclear ribonucleoprotein Sm D2 isoform 1
(SEQ ID NO: 364)
MSLLNKPKSEMTPEELQKREEEEFNTGPLSVLTQSVKNNTQVLINCRNNKKLLGRVKAFDR
HCNMVLENVKEMWTEVPKSGKGKKKSKPVNKDRYISKMFLRGDSVIVVLRNPLIAGK
1AM9: A sterol regulatory element-binding protein 1 isoform a
(SEQ ID NO: 365)
QSRGEKRTAHNAIEKRYRSSINDKIIELKDLVVGTEAKLNKSAVLRKAIDYIRFLQHSNQ
KLKQENLSLRTAVHKSKSLKDL
3S90: C talin- 1
(SEQ ID NO: 366)
GPLGSASARTANPTAKRQFVQSAKEVANSTANLVKTIKAL
1NVP: A TATA-box-binding protein isoform 1
(SEQ ID NO: 367)
SGIVPQLQNIVSTVNLGCKLDLKTIALRARNAEYNPKRFAAVIMRIREPRTTALIFSSGK
MVCTGAKSEEQSRLAARKYARVVQKLGFPAKFLDFKIQNMVGSCDVKFPIRLEGLVLTHQ
QFSSYEPELFPGLIYRMIKPRIVLLIFVSGKVVLTGAKVRAEIYEAFENIYPILKGFRKT
T
1CDW: A TATA-box-binding protein isoform 1
(SEQ ID NO: 368)
SGIVPQLQNIVSTVNLGCKLDLKTIALRARNAEYNPKRFAAVIMRIREPRTTALIFSSGK
MVCTGAKSEENSRLAARKYARVVQKLGFPAKFLDFKIQNMVGSCDVKFPIRLEGLVLTHQ
QFSSYEPELFPGLIYRMIKPRIVLLIFVSGKVVLTGAKVRAEIYEAFENIYPILKGFRK
1TGH: A TATA-box-binding protein isoform 1
(SEQ ID NO: 369)
GSRGSGIVPQLQNIVSTVNLGCKLDLKTIALRARNAEYNPKRFAAVIMRIREPRTTALIF
SSGKMVCTGAKSEEQSRLAARKYARVVQKLGFPAKFLDFKIQNMVGSCDVKFPIRLEGLVL
THQQFSSYEPELFPGLIYRMIKPRIVLLIFVSGKVVLTGAKVRAEIYEAFENIYPILKG
FRKTT
1JFI: C TATA-box-binding protein isoform 2
(SEQ ID NO: 370)
GSHMSGIVPQLQNIVSTVNLGCKLDLKTIALRARNAEYNPKRFAAVIMRIREPRTTALIF
SSGKMVCTGAKSEEQSRLAARKYARVVQKLGFPAKFLDFKIQNMVGSCDVKFPIRLEGLVL
THQQFSSYEPELFPGLIYRMIKPRIVLLIFVSGKVVLTGAKVRAEIYEAFENIYPILKG
FRKTT
1C9B: B TATA-box-binding protein isoform 2
(SEQ ID NO: 371)
GSGIVPQLQNIVSTVNLGCKLDLKTIALRARNAEYNPKRFAAVIMRIREPRTTALIFSSG
KMVCTGAKSEEQSRLAARKYARVVQKLGFPAKFLDFKIQNMVGSCDVKFPIRLEGLVLTH
QQFSSYEPELFPGLIYRMIKPRIVLLIFVSGKVVLTGAKVRAEIYEAFENIYPILKGFRK
3A03: A T-cell leukemia homeobox protein 2
(SEQ ID NO: 372)
MTSFSRSQVLELERRFLRQKYLASAERAALAKALRMTDAQVKTWFQNRRTKWRRQT
1Q68: A T-cell surface glycoprotein CD4 isoform 1 precursor
(SEQ ID NO: 373)
RCRHRRRQAERLSQIKRLLSEKKTCQCPHRFQKTCSPI
1IV6: A telomeric repeat-binding factor 1 isoform 1
(SEQ ID NO: 374)
MTPEKHRARKRQAWLWEEDKNLRSGVRKYGEGNWSKILLHYKFNNRTSVMLKDRWRTM
KKLKLISSDSED
1ITY: A telomeric repeat-binding factor 1 isoform 1
(SEQ ID NO: 375)
TPEKHRARKRQAWLWEEDKNLRSGVRKYGEGNWSKILLHYKFNNRTSVMLKDRWRTMK
KLKLISSDSED
1W0T: A telomeric repeat-binding factor 1 isoform 1
(SEQ ID NO: 376)
KRQAWLWEEDKNLRSGVRKYGEGNWSKILLHYKFNNRTSVMLKDRWRTMKKLK
1W0U: A telomeric repeat-binding factor 2
(SEQ ID NO: 377)
KKQKWTVEESEWVKAGVQKYGEGNWAAISKNYPFVNRTAVMIKDRWRTMKRLGMN
2KO0: A THAP domain-containing protein 1 isoform 1
(SEQ ID NO: 378)
MVQSCSAYGCKNRYDKDKPVSFHKFPLTRP SLCKEWEAAVRRKNFKPTKYSSICSEHFTPD
SFKRESNNKLLKENAVPTIFLELVPR
1S9K: E transcription factor AP-1
(SEQ ID NO: 379)
RKRMRNRIAASKCRKRKLERIARLEEKVKTLKAQNSELASTANMLREQVAQL
1A02: J transcription factor AP-1
(SEQ ID NO: 380)
MKAERKRMRNRIAASKSRKRKLERIARLEEKVKTLKAQNSELASTANMLREQVAQL
1T2K: C transcription factor AP-1
(SEQ ID NO: 381)
MKAERKRMRNRIAASKSRKRKLERIARLEEKVKTLKAQNSELASTANMLREQVAQLKQKV
MN
1O4X: B transcription factor SOX-2
(SEQ ID NO: 382)
GSHMPDRVKRPMNAFMVWSRGQRRKMAQENPKMHNSEISKRLGAEWKLLSETEKRPFID
EAKRLRALHMKEHPDYKYRPRRKTKTLMK
2LE4: A transcription factor SOX-2
(SEQ ID NO: 383)
SDRVKRPMNAFMVWSRGQRRKMAQENPKMHNSEISKRLGAEWKLLSETEKRPFIDEAKRL
RALHMKEHPDYKYRPRRKTKT
1GT0: D transcription factor SOX-2
(SEQ ID NO: 384)
DRVKRPMNAFMVWSRGQRRKMAQENPKMHNSEISKRLGAEWKLLSETEKRPFIDEAKRLR
ALHMKEHPDYKYRPRRKTKT
1VA1: A transcription factor Sp1 isoform b
(SEQ ID NO: 385)
MDPGKKKQHICHIQGCGKVYGKTSHLRAHLRWHTGEX
1H88: C transcriptional activator Myb isoform 1
(SEQ ID NO: 386)
MGHLGKTRWTREEDEKLKKLVEQNGTDDWKVIANYLPNRTDVQCQHRWQKVLNPELIKG
PWTKEEDQRVIKLVQKYGPKRWSVIAKHLKGRIGKQCRERWHNHLNPEVKKTSWTEEEDR
IIYQAHKRLGNRWAEIAKLLPGRTDNAIKNHWNSTMRRKV
1H8A: C transcriptional activator Myb isoform 4
(SEQ ID NO: 387)
MEAVIKNRTDVQCQHRWQKVLNPELNKGPWTKEEDQRVIEHVQKYGPKRWSDIAKHLKG
RIGKQCRERWHNHLNPEVKKTSWTEEEDRIIYQAHKRLGNRWAEIAKLLPGRTDNAVKNH
WNSTMRRKV
2HFG: R tumor necrosis factor receptor superfamily member 13C
(SEQ ID NO: 388)
GSYSLRGRDAPAPTPCNPAECFDPLVRHCVACGLLRTPRPKPAGASSPAPR
1OSX: A tumor necrosis factor receptor superfamily member 13C
(SEQ ID NO: 389)
MRRGPRSLRGRDAPAPTPCVPAECFDLLVRHCVACGLLRTPRPKPAGASSPAPRTALQPQ
E
3L3C: A U1 small nuclear ribonucleoprotein A
(SEQ ID NO: 390)
RPNHTIYINNLNEKIKKDELKKSLHAIFSRFGQILDILVSRSLKMRGQAFVIFKEVSSAT
NALRSMQGFPFYDKPMRIQYAKTDSDIIAK
1FHT: A U1 small nuclear ribonucleoprotein A
(SEQ ID NO: 391)
AVPETRPNHTIYINNLNEKIKKDELKKSLYAIFSQFGQILDILVSRSLKMRGQAFVIFKE
VSSATNALRSMQGFPFYDKPMRIQYAKTDSDIIAKMKGTFVERDRKREKRKPKSQE
2BE6: D voltage-dependent L-type calcium channel subunit
alpha-1C isoform 23
(SEQ ID NO: 392)
GHMDEVTVGKFYATFLIQEYFRKFKKRKEQGLVGKPS
1N0Z: A zinc finger Ran-binding domain-containing protein 2
isoform 2
(SEQ ID NO: 393)
GSMSTKNFRVSDGDWICPDKKCGNVNFARRTSCDRCGREKTTGPI
1HJB: A CCAAT/enhancer-binding protein beta
(SEQ ID NO: 394)
VKSKAKKTVDKHSDEYKIRRERNNIAVRKSRDKAKMRNLETQHKVLELTAENERLQKKVE
QLSRELSTLRNLFKQLPEPLLASSGHC
2E43: A CCAAT/enhancer-binding protein beta
(SEQ ID NO: 395)
VKSKAKKTVDAHSDEYKIRRERNNIAVRKSRDKAKMRNLETQHKVLELTAENERLQKKVE
QLSRELSTLRNLFKQLPE
1CI6: B CCAAT/enhancer-binding protein beta
(SEQ ID NO: 396)
MEYKMRRERNNIAVRKSRDKAKMRNLETQHKVLELTAENERLQKKVEQLSRELSTLRNLF
KQL
1GTW: A CCAAT/enhancer-binding protein beta
(SEQ ID NO: 397)
VKSKAKKTVDKHSDEYKIRRERNNIAVRKSRDKAKMRNLETQHKVLELTAENERLQKKVE
QLSRELSTLRNLFKQLPE
2E42: A CCAAT/enhancer-binding protein beta
(SEQ ID NO: 398)
VKSKAKKTVDKHSDEYKIRRERNNIAARKSRDKAKMRNLETQHKVLELTAENERLQKKVE
QLSRELSTLRNLFKQLPE
3NWV: A cytochrome c
(SEQ ID NO: 399)
GDVEKGKKIFIMKCSQCHTVEKGGKHKTGPNLHGLFGRKTSQAPGYSYTAANKNKGIIWG
EDTLMEYLENPKKYIPGTKMIFVGIKKKEERADLIAYLKKATNE
2C6Y: A forkhead box protein K2
(SEQ ID NO: 400)
ASMTGGQQMGRGSDSKPPYSYAQLIVQAITMAPDKQLTLNGIYTHITKNYPYYRTADKGW
QNSIRHNLSLNRYFIKVPRSQEEPGKGSFWRIDPASESKLIEQAFRKRRPR
2HDM: A lymphotactin precursor
(SEQ ID NO: 401)
GSEVSDKRTCVSLTTQRLPCSRIKTYTITEGSLRAVIFITKRGLKVCADPQATWVRDCVR
SMDRKSNTRNNMIQTKPTGTQQSTNTAVTLTG
3CW1: D small nuclear ribonucleoprotein Sm D3
(SEQ ID NO: 402)
MSIGVPIKVLHEAEGHIVTCETNTGEVYRGKLIEAEDNMNCQMSNITVTYRDGRVAQLEQV
YIRGCKIRFLILPDMLKNAPMLKSMKNKNQGSGAGRGKAAILKAQVAARGRGRGMGRGNI
FQKRR
2J7Z: A stromal cell-derived factor 1 isoform gamma
(SEQ ID NO: 403)
KPVSLSYRCPCRFFESHVARANVKHLKILNTPNCALQIVARLKNNNRQVCIDPKLKWIQE
YLEKALNK
2KEE: A stromal cell-derived factor 1 isoform gamma
(SEQ ID NO: 404)
GSKPVSLSYRCPCRFFESHVARANVKHLKILNTPNCALQIVARLKNNNRQVCIDPKLKWI
QEYLEKALNK
2KEC: A stromal cell-derived factor 1 isoform gamma
(SEQ ID NO: 405)
GMKPVSLSYRCPCRFFESHVARANVKHLKILNTPNCALQIVARLKNNNRQVCIDPKLKWIQ
EYLEKALNK
1QG7: A stromal cell-derived factor 1 isoform gamma
(SEQ ID NO: 406)
KPVSLSYRCPCRFFESHVARANVKHLKILNTPNCALQIVARLKNNNRQVCIDPKLKWIQE
YLEKALN
2NWG: A stromal cell-derived factor 1 isoform gamma
(SEQ ID NO: 407)
MKPVSLSYRCPCRFFESHIARANVKHLKILNTPNCALQIVARLKNNNRQVCIDPKLKWIQ
EYLEKALN
3HP3: A stromal cell-derived factor 1 isoform gamma
(SEQ ID NO: 408)
MKPVSLSYRCPCRFFESHVARANVKHLKILNTPNCALQIVARLKNNNRQVCIDPKLKWIQE
YLEKALN
1VMC: A stromal cell-derived factor 1 isoform gamma
(SEQ ID NO: 409)
SDYKPVSLSYRCPCRFFESHVARANVKHLKILNTPNCALQIVARLKNNNRQVCIDPKLKWIQ
EYLEKALNK
3GV3: A stromal cell-derived factor 1 isoform gamma
(SEQ ID NO: 410)
LSYRCPCRFFESHIARANVKHLKILNTPNCALQIVARLKNNNRQVCIDPKLKWIQEYLEK
ALN
1UN5: A angiogenin precursor
(SEQ ID NO: 411)
MQDNSRYTHFLTQHYDAKPQGRDDRYCESIMRRRGLTSDRCKPINTFIHGNKRSIKAICE
NKNGNPHRENLRISKSSFQVTTCKLHGGSPWPPCQYRATAGFRNVVVACENGLPVHLDQSI
FRRP
2PLZ: A beta-defensin 1 preproprotein
(SEQ ID NO: 412)
DHYNCVSSGGQCLYSACPIFTRIQGTCYRGRARCCR
1BND: A brain-derived neurotrophic factor isoform a preproprotein
(SEQ ID NO: 413)
HSDPARRGQLSVCDSISEWVTAADKKTAVDMSGGTVTVLEKVPVSKGQLKQYFYETKCNP
MGYTKEGCRGIDKRHWNSQCRTTQSYVRALTMDSKKRIGWRFIRIDTSCVCTLTIKRGR
1G91: A C-C motif chemokine 23 isoform CKbeta8 precursor
(SEQ ID NO: 414)
MDRFHATSADCCISYTPRSIPCSLLESYFETNSECSKPGVIFLTKKGRRFCANPSDKQVQ
VCMRMLKLDTRIKTRKN
1IOX: A probetacellulin precursor
(SEQ ID NO: 415)
RKGHFSRCPKQYKHYCIKGRCRFVVAEQTPSCVCDEGYIGARCERVDLFX
2K01: A stromal cell-derived factor 1 isoform gamma
(SEQ ID NO: 416)
GMKPVSLSYRCPCRFFESHVARANVKHLKILNTPNCACQIVARLKNNNRQVCIDPKLKWIQ
EYLEKCLNK
2JTG: A THAP domain-containing protein 1 isoform 1
(SEQ ID NO: 417)
MVQSCSAYGCKNRYDKDKPVSFHKFPLTRPSLCKEWEAAVRRKNFKPTKYSSICSEHFTPD
CFKRECNNKLLKENAVPTIFLELVPR
2ASK: A artemin isoform 3 precursor
(SEQ ID NO: 418)
AGGPGSRARAAGARGCRLRSQLVPVRALGLGHRSDELVRFRFCSGSCRRARSPHDLSLASL
LGAGALRPPPGSRPVSQPCCRPTRYEAVSFMDVNSTWRTVDRLSATACGCLG
2O13: A cysteine and glycine-rich protein 3
(SEQ ID NO: 419)
KCPRCGKSVYAAEKVMGGGKPWHKTCFRCAICGKSLESTNVTDKDGELYCKVCYAKNF
2VJE: A E3 ubiquitin-protein ligase Mdm2 isoform MDM2
(SEQ ID NO: 420)
SSLPLNAIEPCVICQGRPKNGCIVHGKTGHLMACFTCAKKLKKRNKPCPVCRQPIQMIVL
TYFP
2HDP: A E3 ubiquitin-protein ligase Mdm2 isoform MDM2
(SEQ ID NO: 421)
SLPLNAIEPCVICQGRPKNGCIVHGKTGHLMACFTCAKKLKKRNKPCPVCRQPIQMIVLT
YFP
2VJE: B protein Mdm4 isoform 1
(SEQ ID NO: 422)
EDCQNLLKPCSLCEKRPRDGNIIHGRTGHLVTCFHCARRLKKAGASCPICKKEIQLVIKV
FIA
2Z7F: I antileukoproteinase precursor
(SEQ ID NO: 423)
RRKPGKCPVTYGQCLMLNPPNFCEMDGQCKRDLKCCMGMCGKSCVSPVKA
3QMB: A cpG-binding protein isoform 2
(SEQ ID NO: 424)
MHHHHHHSSRENLYFQGQIKRSARMCGECEACRRTEDCGHCDFCRDMKKFGGPNKIRQKC
RLRQCQLRARESYKYFPSS
1H1H: A eosinophil cationic protein precursor
(SEQ ID NO: 425)
MRPPQFTRAQWFAIQHISLNPPRCTIAMRAINNYRWRCKNQNTFLRTTFANVVNVCGNQSI
RCPHNRTLNNCHRSRFRVPLLHCDLINPGAQNISNCRYADRPGRRFYVVACDNRDPRDSPR
YPVVPVHLDTTI
1DYT: A eosinophil cationic protein precursor
(SEQ ID NO: 426)
RPPQFTRAQWFAIQHISLNPPRCTIAMRAINNYRWRCKNQNTFLRTTFANVVNVCGNQSI
RCPHNRTLNNCHRSRFRVPLLHCDLINPGAQNISNCRYADRPGRRFYVVACDNRDPRDSPR
YPVVPVHLDTTI
1LO1: A estrogen-related receptor gamma isoform 2
(SEQ ID NO: 427)
XIPKRLCLVCGDIASGYHYGVASCEACKAFFKRTIQGNIEYSCPATNECEITKRRRKSCQ
ACRFMKALKVGMLKEGVRLDRVRGGRQKYKRRLDSENS
1RXR: A retinoic acid receptor RXR-alpha
(SEQ ID NO: 428)
XTKHICAICGDRSSGKHYGVYSCEGCKGFFKRTVRKDLTYTCRDNKDCLIDKRQRNRCQYC
RYQKALAMGMKREAVQEERQRG
2HKY: A ribonuclease 7 precursor
(SEQ ID NO: 429)
MKPKGMTSSQWFKIQHMQPSPQACNSAMKNINKHTKRCKDLNTFLHEPFSSVAATCQTPKI
ACKNGDKNCHQSHGPVSLTMCKLTSGKYPNCRYKEKRQNKSYVVACKPPQKKDSQQFHL
VPVHLDRVL
1UBD: C transcriptional repressor protein YY1
(SEQ ID NO: 430)
MEPRTIACPHKGCTKMFRDNSAMRKHLHTHGPRVHVCAECGKAFVESSKLKRHQLVHTGE
KPFQCTFEGCGKRFSLDFNLRTHVRIHTGDRPYVCPFDGCNKKFAQSTNLKSHILTHAKAKN
NQ
1KMX: A vascular endothelial growth factor A isoform d
(SEQ ID NO: 431)
ARQENPCGPCSERRKHLFVQDPQTCKC SCKNTDSRCKARQLELNERTCRCDKPRR
2JP9: A Wilms tumor protein isoform B
(SEQ ID NO: 432)
ASEKRPFMCAYPGCNKRYFKLSHLQMHSRKHTGEKPYQCDFKDCERRFSRSDQLKRHQRR
HTGVKPFQCKTCQRKF SRSDHLKTHTRTHTGEKPFSCRWPSCQKKFARSDELVRHHNMH
1R8U: B CREB-binding protein isoform a
(SEQ ID NO: 433)
ATGPTADPEKRKLIQQQLVLLLHAHKCQRREQANGEVRACSLPHCRTMKNVLNHMTHCQ
AGKACQVAHCASSRQIISHWKNCTRHDCPVCLPLKNASDKX
1L8C: A CREB-binding protein isoform a
(SEQ ID NO: 434)
ADPEKRKLIQQQLVLLLHAHKCQRREQANGEVRACSLPHCRTMKNVLNHMTHCQAGKAC
QVAHCASSRQIISHWKNCTRHDCPVCLPLKNASDKX
1HCQ: A estrogen receptor isoform 4
(SEQ ID NO: 435)
MKETRYCAVCNDYASGYHYGVWSCEGCKAFFKRSIQGHNDYMCPATNQCTIDKNRRKSC
QACRLRKCYEVGMMKGGIRKDRRGG
1HCP: A estrogen receptor isoform 4
(SEQ ID NO: 436)
MKETRYCAVCNDYASGYHYGVWSCEGCKAFFKRSIQGHNDYMCPATNQCTIDKNRRKSC
QACRLRKCYEVGMMKGG
3CBB: A hepatocyte nuclear factor 4-alpha isoform a
(SEQ ID NO: 437)
ALCAICGDRATGKHYGASSCDGCKGFFRRSVRKNHMYSCRFSRQCVVDKDKRNQCRYCR
LKKCFRAGMKKEAVQNERD
3IO2: A histone acetyltransferase p300
(SEQ ID NO: 438)
ATQSPGDSRRLSIQRAIQSLVHAAQCRNANCSLPSCQKMKRVVQHTKGCKRKTNGGCPICK
QLIALAAYHAKHCQENKCPVPFCLNIKQKLRQQQLQHRLQQAQMLRRRMASMQ
1L3E: B histone acetyltransferase p300
(SEQ ID NO: 439)
MGSGAHTADPEKRKLIQQQLVLLLHAHKCQRREQANGEVRQCNLPHCRTMKNVLNHMTH
CQSGKSCQVAHCASSRQIISHWKNCTRHDCPVCLPLKNAGDK
2KKF: A histone-lysine N-methyltransferase MLL isoform 1 precursor
(SEQ ID NO: 440)
GSKKGRRSRRCGQCPGCQVPEDCGVCTNCLDKPKFGGRNIKKQCCKMRKCQNLQWMPSK
2JYI: A histone-lysine N-methyltransferase MLL isoform 2 precursor
(SEQ ID NO: 441)
KKGRRSRRCGQCPGCQVPEDCGVCTNCLDKPKFGGRNIKKQCCKMRKCQNLQWMPSK
1A6Y: A nuclear receptor subfamily 1 group D member 1
(SEQ ID NO: 442)
TKLNGMVLLCKVCGDVASGFHYGVLACEGCKGFFRRSIQQNIQYKRCLKNENCSIVRINRN
RCQQCRFKKCLSVGMSRDAVRFGRIPKREKQRM
2A66: A nuclear receptor subfamily 5 group A member 2 isoform 2
(SEQ ID NO: 443)
GEFGDEDLEELCPVCGDKVSGYHYGLLTCESCKGFFKRTVQNNKRYTCIENQNCQIDKTQR
KRCPYCRFQKCLSVGMKLEAVRADRMRGGRNKFGPMYKRDRALKQQKKALIR
1DSZ: A retinoic acid receptor alpha isoform 1
(SEQ ID NO: 444)
PRIYKPCFVCQDKSSGYHYGVSACEGCKGFFRRSIQKNMVYTCHRDKNCIINKVTRNRCQY
CRLQKCFEVGMSKESVRNDRNKKKK
1DSZ: B retinoic acid receptor RXR-alpha
(SEQ ID NO: 445)
GSFTKHICAICGDRSSGKHYGVYSCEGCKGFFKRTVRKDLTYTCRDNKDCLIDKRQRNRCQ
YCRYQKCLAMGMKREAVQEERQRG
1BY4: A retinoic acid receptor RXR-alpha
(SEQ ID NO: 446)
GSFTKHICAICGDRSSGKHYGVYSCEGCKGFFKRTVRKDLTYTCRDNKDCLIDKRQRNRCQ
YCRYQKCLAMGMKREAVQEER
1R0N: A retinoic acid receptor RXR-alpha
(SEQ ID NO: 447)
FTKHICAICGDRSSGKHYGVYSCEGCKGFFKRTVRKDLTYTCRDNKDCLIDKRQRNRCQYC
RYQKCLAMGMKREAVQAAAA
2NLL: A retinoic acid receptor RXR-alpha
(SEQ ID NO: 448)
CAICGDRSSGKHYGVYSCEGCKGFFKRTVRKDLTYTCRDNKDCLIDKRQRNRCQYCRYQK
CLAMGM
1KB2: A vitamin D3 receptor isoform VDRB1
(SEQ ID NO: 449)
FDRNVPRICGVCGDRATGFHFNAMTCEGCKGFFRRSMKRKALFTCPFNGDCRITKDNRRHC
QACRLKRCVDIGMMKEFILTDEEVQRKREMILKRKEEEALKDSLRPKLS
1YNW: A vitamin D3 receptor isoform VDRB1
(SEQ ID NO: 450)
FDRNVPRICGVCGDRATGFHFNAMTCEGCKGFFRRSMKRKALFTCAANGDCRITKDNRRA
CQACRLKRCVDIGMMKEFILTDEEVQRKREMILKRKEEEALKDSLRPKLS
2C7A: A progesterone receptor isoform B
(SEQ ID NO: 451)
PQKICLICGDEASGCHYGVLTCGSCKVFFKRAMEGQHNYLCAGRNDCIVDKIRRKNCPACR
LRKCCQAGMVLGGRKFK
2KA6: A CREB-binding protein isoform b
(SEQ ID NO: 452)
SPQESRRLSIQRCIQSLVHACQCRNANCSLPSCQKMKRVVQHTKGCKRKTNGGCPVCKQLI
ALCCYHAKHCQENKCPVPFCLNIKHKLRQQQ
3P57: P histone acetyltransferase p300
(SEQ ID NO: 453)
HMSPGDSRRLSIQRCIQSLVHACQCRNANCSLPSCQKMKRVVQHTKGCKRKTNGGCPICKQ
LIALCCYHAKHCQENKCPVPFCLNIKQKLRQQQLQHRLQQAQMLRRRMASM
1N29: A phospholipase A2, membrane associated precursor
(SEQ ID NO: 454)
ALVNFHRMIKLTTGKEAALSYGFYGCHCGVGGRGSPKDATDRCCVTHDCCYKRLEKRGC
GTKFLSYKFSNSGSRITCAKQDSCRSQLCECDKAAATCFARNKTTYNKKYQYYSNKHCRGS
TPRC
1N28: A phospholipase A2, membrane associated precursor
(SEQ ID NO: 455)
ALVNFHRMIKLTTGKEAALSYGFYGCHCGVGGRGSPKDATDRCCVTQDCCYKRLEKRGC
GTKFLSYKFSNSGSRITCAKQDSCRSQLCECDKAAATCFARNKTTYNKKYQYYSNKHCRGS
TPRC
1U35: C core histone macro-H2A.1 isoform 1
(SEQ ID NO: 456)
MSSRGGKKKSTKTSRSAKAGVIFPVGRMLRYIKKGHPKYRIGVGAPVYMAAVLEYLTAEIL
ELAVNAARDNKKGRVTPRHILLAVANDEELNQLLKGVTIASGGVLPNIHPELLAKKRGS
3AFA: A histone cluster 1, H3d
(SEQ ID NO: 457)
GSHMARTKQTARKSTGGKAPRKQLATKAARKSAPATGGVKKPHRYRPGTVALREIRRYQK
STELLIRKLPFQRLVREIAQDFKTDLRFQSSAVMALQEACEAYLVGLFEDTNLCAIHAKRVTI
MPKDIQLARRIRGERA
1U35: A histone cluster 1, H3d
(SEQ ID NO: 458)
MARTKQTARKSTGGKAPRKQLATKAARKSAPATGGVKKPHRYRPGTVALREIRRYQKSTE
LLIRKLPFQRLVREIAQDFKTDLRFQSSAVMALQEACEAYLVGLFEDTNLCAIHAKRVTIMP
KDIQLARRIRGERA
2F8N: K histone H2A type 1-B/E
(SEQ ID NO: 459)
MGSSHHHHHHSSGLVPRGSMSGRGKQGGKARAKAKTRSSRAGLQFPVGRVHRLLRKGNY
SERVGAGAPVYLAAVLEYLTAEILELAGNAARDNKKTRIIPRHLQLAIRNDEELNKLLGRVT
IAQGGVLPNIQAVLLPKKTESHHKAKGK
3A6N: C histone H2A type 1-B/E
(SEQ ID NO: 460)
GSHMSGRGKQGGKARAKAKTRSSRAGLQFPVGRVHRLLRKGNYSERVGAGAPVYLAAVL
EYLTAEILELAGNAARDNKKTRIIPRHLQLAIRNDEELNKLLGRVTIAQGGVLPNIQAVLLPK
KTESHHKAKGK
2CV5: C histone H2A type 1-B/E
(SEQ ID NO: 461)
MSGRGKQGGKARAKAKTRSSRAGLQFPVGRVHRLLRKGNYSERVGAGAPVYLAAVLEYL
TAEILELAGNAARDNKKTRIIPRHLQLAIRNDEELNKLLGRVTIAQGGVLPNIQAVLLPKKTE
SHHKAKGK
1P34: C histone H2A type 1-B/E
(SEQ ID NO: 462)
SGRGKQGGKTRAKAKTRSSRAGLQFPVGRVHRLLRKGNYAERVGAGAPVYLAAVLEYLT
AEILELAGNAARDNKKTRIIPRHLQLAVRNDEELNKLLGRVTIAQGGVLPNIQSVLLPKKTES
AKSAKSK
1ZLA: C histone H2A.J
(SEQ ID NO: 463)
SGRGKQGGKTRAKAKTRSSRAGLQFPVGRVHRLLRKGNYAERVGAGAPVYLAAVLEYLT
AEILELAGNWERDNKKTRIIPRHLQLAVRNDEELNKLLGRVTIAQGGVLPNIQSVLLPKKTE
SSKSTKSK
1KX3: C histone H2A.J
(SEQ ID NO: 464)
SGRGKQGGKTRAKAKTRSSRAGLQFPVGRVHRLLRKGNYAERVGAGAPVYLAAVLEYLT
AEILELAGNAARDNKKTRIIPRHLQLAVRNDEELNKLLGRVTIAQGGVLPNIQSVLLPKKTES
SKSKSK
2NQB: C histone H2A.J
(SEQ ID NO: 465)
SGRGKGGKVKGKAKSRSNRAGLQFPVGRIHRLLRKGNYAERVGAGAPVYLAAVMEYLAA
EVLELAGNAARDNKKTRIIPRHLQLAIRNDEELNKLLSGVTIAQGGVLPNIQAVLLPKKTEK
KA
2PYO: C histone H2A.J
(SEQ ID NO: 466)
SGRGKGGKVKGKAKSRSNRAGLQFPVGRIHRLLRKGNYAERVGAGAPVYLAAVMEYLAA
EVLELAGNAARDNKKTRIIPRHLQLAIRNDEELNKLLSGVTIAQGGVLPNIQAVLLPKKTE
1F66: C histone H2A.Z
(SEQ ID NO: 467)
MAGGKAGKDSGKAKTKAVSRSQRAGLQFPVGRIHRHLKSRTTSHGRVGATAAVYSAAILE
YLTAEVLELAGNASKDLKVKRITPRHLQLAIRGDEELDSLIKATIAGGGVIPHIHKSLIG
KKGQQKTV
1U35: D histone H2B type 1-B
(SEQ ID NO: 468)
MPEPSRSTPAPKKGSKKAITKAQKKDGKKRKRGRKESYSIYVYKVLKQVHPDTGISSKAMG
IMNSFVNDIFERIASEASRLAHYNKRSTITSREVQTAVRLLLPGELAKHAVSEGTKAVTKYTS
SK
3A6N: D histone H2B type 1-J
(SEQ ID NO: 469)
GSHMPEPAKSAPAPKKGSKKAVTKAQKKDGKKRKRSRKESYSIYVYKVLKQVHPDTGISS
KAMGIMNSFVNDIFERIAGEASRLAHYNKRSTITSREIQTAVRLLLPGELAKHAVSEGTKAV
TKYTSAK
1F66: D histone H2B type 1-J
(SEQ ID NO: 470)
MPEPAKSAPAPKKGSKKAVTKTQKKDGKKRRKTRKESYAIYVYKVLKQVHPDTGISSKAM
SIMNSFVNDVFERIAGEASRLAHYNKRSTITSREIQTAVRLLLPGELAKHAVSEGTKAVTKY
TSAK
1KX3: D histone H2B type 1-J
(SEQ ID NO: 471)
PEPAKSAPAPKKGSKKAVTKTQKKDGKKRRKTRKESYAIYVYKVLKQVHPDTGISSKAMSI
MNSFVNDVFERIAGEASRLAHYNKRSTITSREIQTAVRLLLPGELAKHAVSEGTKAVTKYTS
AK
1P34: D histone H2B type 1-J
(SEQ ID NO: 472)
PEPAKSAPAPKKGSKKAVTKTQKKDGKKRRKSRKESYAIYVYKVLKQVHPDTGISSKAMSI
MNSFVNDVFERIAGEASRLAHYNKRSTITSREIQTAVRLLLPGELAKHAVSEGTKAVTKYTS
AK
2F8N: H histone H2B type 1-J
(SEQ ID NO: 473)
MAKSAPAPKKGSKKAVTKTQKKDGKKRRKTRKESYAIYVYKVLKQVHPDTGISSKAMSIM
NSFVNDVFERIAGEASRLAHYNKRSTITSREIQTAVRLLLPGELAKHAVSEGTKAVTKYTSA
K
2CV5: D histone H2B type 1-K
(SEQ ID NO: 474)
MPEPAKSAPAPKKGSKKAVTKAQKKDGKKRKRSRKESYSVYVYKVLKQVHPDTGISSKA
MGIMNSFVNDIFERIAGEASRLAHYNKRSTITSREIQTAVRLLLPGELAKHAVSEGTKAVTK
YTSAK
1ZLA: D histone H2B type 1-O
(SEQ ID NO: 475)
PDPAKSAPAAKKGSKKAVTKTQKKDGKKRRKSRKESYAIYVYKVLKQVHPDTGISSKAMS
IMNSFVNDVFERIAGEASRLAHYNKRSTITSREIQTAVRLLLPGELAKHAVSEGTKAVTKYT
SAK
3A6N: A histone H3.1t
(SEQ ID NO: 476)
GSHMARTKQTARKSTGGKAPRKQLATKVARKSAPATGGVKKPHRYRPGTVALREIRRYQK
STELLIRKLPFQRLMREIAQDFKTDLRFQSSAVMALQEACESYLVGLFEDTNLCVIHAKRVTI
MPKDIQLARRIRGERA
3AV1: A histone H3.2
(SEQ ID NO: 477)
GSHMARTKQTARKSTGGKAPRKQLATKAARKSAPATGGVKKPHRYRPGTVALREIRRYQK
STELLIRKLPFQRLVREIAQDFKTDLRFQSSAVMALQEASEAYLVGLFEDTNLCAIHAKRVTI
MPKDIQLARRIRGERA
1F66: A histone H3.2
(SEQ ID NO: 478)
MARTKQTARKSTGGKAPRKQLATKAARKSAPATGEVKKPHRYRPGTVALREIRRYQKSTE
LLIRKLPFQRLVREIAQDFKTDLRFQSSAVMALQEASEAYLVALFEDTNLCAIHAKRVTIMP
KDIQLARRIRGERA
2F8N: A histone H3.2
(SEQ ID NO: 479)
MARTKQTARKSTGGKAPRKQLATKAARKSAPATGGVKKPHRYRPGTVALREIRRYQKSTE
LLIRKLPFQRLVREIAQDFKTDLRFQSSAVMALQEASEAYLVGLFEDTNLCAIHAKRVTIMP
KDIQLARRIRGERA
1P3L: A histone H3.2
(SEQ ID NO: 480)
ARTKQTARKSTGGKAPRKQLATKAARKSAPATGESKKPHRYRPGTVALREIRRYQKSTELL
IRKLPFQRLVREIAQDFKTDLRFQSSAVMALQEASEAYLVALFEDTNLCAIHAKRVHIMPKD
IQLARRIRGERA
IP3M: A histone H3.2
(SEQ ID NO: 481)
ARTKQTARKSTGGKAPRKQLATKAARKSAPATGESKKPHRYRPGTVALREIRRYQKSTELL
IRKLPFQRLVREIAQDFKTDLRFQSSAVMALQEASEAYLVALFEDTNLCAIHAKRVIIM
PKDIQLARRIRGERA
IP3B: A histone H3.2
(SEQ ID NO: 482)
ARTKQTARKSTGGKAPRKQLATKAARKSAPATGESKKPHRYRPGTVALREIRRYQKSTELL
IRKLPFQRLVREIAQDFKTDLRFQSSAVMALQEASEAYLVAEFEDTNLCAIHAKRVTIMPKD
IQLARRIRGERA
1ZLA: A histone H3.2
(SEQ ID NO: 483)
ARTKQTARKSTGGKAPRKQLATKAARKSAPATGGVKKPHRYRPGTVALREIRRYQKSTEL
LIRKLPFQRLVREIAQDFKTDLRFQSSAVMALQEASEAYLVALFEDTNLCAIHAKRVHIMPK
DIQLARRIRGERA
1P3A: A histone H3.2
(SEQ ID NO: 484)
ARTKQTARKSTGGKAPRKQLATKAARKSAPATGESKKPHRYRPGTVALREIRRYQKSTELL
IRKLPFQRLVREIAQDFKTDLRFQSSAVMALQEASEAYLVALFEDTNLCAIHAKHVTIMPKD
IQLARRIRGERA
1P3K: A histone H3.2
(SEQ ID NO: 485)
ARTKQTARKSTQGKAPRKQLATKAARKSAPATGESKKPHRYRPOTVALREIRRYQKSTELL
IRKLPFQRLVREIAQDFKTDLRFQSSAVMALQEASEAYLVALFEDTNLCAIHAKRVAIMPKD
IQLARRIRGERA
1KX3: A histone H3.2
(SEQ ID NO: 486)
ARTKQTARKSTGGKAPRKQLATKAARKSAPATGGVKKPHRYRPGTVALREIRRYQKSTEL
LIRKLPFQRLVREIAQDFKTDLRFQSSAVMALQEASEAYLVALFEDTNLCAIHAKRVTIMPK
DIQLARRIRGERA
2IO5: B histone H3.2
(SEQ ID NO: 487)
ARTKQTARKSTGGKAPRKQLATKAARKSAPATGGVKKPHRYRPGTVALREIRRYQKSTEL
LIRKLPFQRLVREIAQDFKTDLRFQSSAVMALQEASEAYLVGLFEDTNLCAIHAKRVTIMPK
DIQLARRIRGERA
1P34: A histone H3.2
(SEQ ID NO: 488)
ARTKQTARKSTGGKAPRKQLATKAARKSAPATGESKKPHRYRPGTVALREIRRYQKSTELL
IRKLPFQRLVREIAQDFKTDLRFQSSAVMALQEASEAYLVALFEDTNLCAIHAKAVTIMPKD
IQLARRIRGERA
3AV2: A histone H3.3
(SEQ ID NO: 489)
GSHMARTKQTARKSTGGKAPRKQLATKAARKSAPSTGGVKKPHRYRPGTVALREIRRYQK
STELLIRKLPFQRLVREIAQDFKTDLRFQSAAIGALQEASEAYLVGLFEDTNLCAIHAKRVTI
MPKDIQLARRIRGERA
1ID3: A histone H3.3
(SEQ ID NO: 490)
ARTKQTARKSTGGKAPRKQLASKAARKSAPSTGGVKKPIIRYKPGTVALREIRRFQKSTELL
IRKLPFQRLVREIAQDFKTDLRFQSSAIGALQESVEAYLVSLFEDTNLAAIHAKRVTIQ
KKEIKLARRLRGERS
3A6N: B histone H4
(SEQ ID NO: 491)
GSHMSGRGKGGKGLGKGGAKRHRKVLRDNIQGITKPAIRRLARRGGVKRISGLIYEETRGV
LKVFLENVIRDAVTYTEHAKRKTVTAMDVVYALKRQGRTLYGFGG
1F66: B histone H4
(SEQ ID NO: 492)
MSGRGKGGKGLGKGGAKRHRKVLRDNIQGITKPAIRRLARRGGVKRISGLIYEETRGVLKV
FLENVIRDAVTYTEHAKRKTVTAMDVVYALKRQGRTLYGFGG
2NQB: B histone H4
(SEQ ID NO: 493)
ITGRGKGGKGLGKGGAKRHRKVLRDNIQGITKPAIRRLARRGGVKRISGLIYEETRGVLK
VFLENVIRDAVTYTEHAKRKTVTAMDVVYALKRQGRTLYGFGG
2PYO: B histone H4
(SEQ ID NO: 494)
TGRGKGGKGLGKGGAKRHRKVLRDNIQGITKPAIRRLARRGGVKRISGLIYEETRGVLKVF
LENVIRDAVTYTEHAKRKTVTAMDVVYALKRQGRTLYGFGG
1P3P: B histone H4
(SEQ ID NO: 495)
SGRGKGGKGLGKGGAKRHRKVLRDNIQGITKPAIRRLARRGGIKRISGLIYEETRGVLKV
FLENVIRDAVTYTEHAKRKTVTAMDVVYALKRQGRTLYGFGG
1KX3: B histone H4
(SEQ ID NO: 496)
SGRGKGGKGLGKGGAKRHRKVLRDNIQGITKPAIRRLARRGGVKRISGLIYEETRGVLKVFL
ENVIRDAVTYTEHAKRKTVTAMDVVYALKRQGRTLYGFGG
1ID3: B histone H4
(SEQ ID NO: 497)
SGRGKGGKGLGKGGAKRHRKILRDNIQGITKPAIRRLARRGGVKRISGLIYEEVRAVLKS
FLESVIRDSVTYTEHAKRKTVTSLDVVYALKRQGRTLYGFGG
1P3I: B histone H4
(SEQ ID NO: 498)
SGRGKGGKGLGKGGAKRHRKVLRDNIQGITKPAIRRLARRGGVKHISGLIYEETRGVLKVF
LENVIRDAVTYTEHAKRKTVTAMDVVYALKRQGRTLYGFGG
1P3O: B histone H4
(SEQ ID NO: 499)
SGRGKGGKGLGKGGAKRHRKVLRDNIQGITKPAIRRLARRGGAKRISGLIYEETRGVLKVFL
ENVIRDAVTYTEHAKRKTVTAMDVVYALKRQGRTLYGFGG
1P3G: B histone H4
(SEQ ID NO: 500)
SGRGKGGKGLGKGGAKRHRKVLRDNIQGITKPAIRRLARRGGVKEISGLIYEETRGVLKVFL
ENVIRDAVTYTEHAKRKTVTAMDVVYALKRQGRTLYGFGG
1P3F: B histone H4
(SEQ ID NO: 501)
SGRGKGGKGLGKGGAKRHRKVLRDNIQGITKPAIRRLARRGGVKCISGLIYEETRGVLKVFL
ENVIRDAVTYTEHAKRKTVTAMDVVYALKRQGRTLYGFGG
1P3B: B histone H4
(SEQ ID NO: 502)
SGRGKGGKGLGKGGAKRHRKVLRDNIQGITKPAIRRLARRGGVKAISGLIYEETRGVLKVF
LENVIRDAVTYTEHAKRKTVTAMDVVYALKRQGRTLYGFGG
3NQJ: B histone H4
(SEQ ID NO: 503)
MKVLRDNIQGITKPAIRRLARRGGVKRISGLIYEETRGVLKVFLENVIRDAVTYTEHAKR
KTVTAMDVVYALKRQGRTLYGFGG
1KYN: A cathepsin G preproprotein
(SEQ ID NO: 504)
IIGGRESRPHSRPYMAYLQIQSPAGQSRCGGFLVREDFVLTAAHCWGSNINVTLGAHNIQ
RRENTQQHITARRAIRHPQYNQRTIQNDIMLLQLSRRVRRNRNVNPVALPRAQEGLRPGT
LCTVAGWGRVSMRRGTDTLREVQLRVQRDRQCLRIFGSYDPRRQICVGDRRERKAAFKGD
SGGPLLCNNVAHGIVSYGKSSGVPPEVFTRVSSFLPWIRTTMRSFKLLDQMETPL
1AU8: A cathepsin G preproprotein
(SEQ ID NO: 505)
IIGGRESRPHSRPYMAYLQIQSPAGQSRCGGFLVREDFVLTAAHCWGSNINVTLGAHNIQ
RRENTQQHITARRAIRHPQYNQRTIQNDIMLLQLSRRVRRNRNVNPVALPRAQEGLRPGT
LCTVAGWGRVSMRRGTDTLREVQLRVQRDRQCLRIFGSYDPRRQICVGDRRERKAAFKGD
SGGPLLCNNVAHGIVSYGKSSGVPPEVFTRVSSFLPWIRTTMRS
2YRQ: A high mobility group protein B1
(SEQ ID NO: 506)
GSSGSSGMGKGDPKKPRGKMSSYAFFVQTCREEHKKKHPDASVNFSEFSKKCSERWKTMS
AKEKGKFEDMAKADKARYEREMKTYIPPKGETKKKFKDPNAPKRPPSAFFLFCSEYRPKIK
GEHPGLSIGDVAKKLGEMWNNTAADDKQPYEKKAAKLKEKYEKDIAAYRAKG
2BFH: A heparin-binding growth factor 2
(SEQ ID NO: 507)
DPKRLYCKNGGFFLRIHPDGRVDGVREKSDPHIKLQLQAEERGVVSIKGVCANRYLAMKED
GRLLASKCVTDECFFFERLESNNYNTYRSRKYTSWYVALKRTGQYKLGSKTGPGQKAILFL
PMSAKS
1AYP: A phospholipase A2, membrane associated precursor
(SEQ ID NO: 508)
NLVNFHRMIKLTTGKEAALSYGFYGCHCGVGGRGSPKDATDRCCVTHDCCYKRLEKRGC
GTKFLSYKFSNSGSRITCAKQDSCRSQLCECDKAAATCFARNKTTYNKKYQYYSNKHCRGS
TPRC
1H8U: A bone marrow proteoglycan preproprotein
(SEQ ID NO: 509)
TCRYLLVRSLQTFSQAWFTCRRCYRGNLVSIHNFNINYRIQCSVSALNQGQVWIGGRITG
SGRCRRFQWVDGSRWNFAYWAAHQPWSRGGHCVALCTRGGYWRRAHCLRRLPFICSY
1B34: A small nuclear ribonucleoprotein Sm D1
(SEQ ID NO: 510)
MKLVRFLMKLSHETVTIELKNGTQVHGTITGVDVSMNTHLKAVKMTLKNREPVQLETLSIR
GNNIRYFILPDSLPLDTLLVDVEPKVKSKKREAVAGRGRGRGRGRGRGRGRGRGGPRR
2CPX: A RNA-binding protein 41 isoform 1
(SEQ ID NO: 511)
GSSGSSGEEIRKIPMFSSYNPGEPNKVLYLKNLSPRVTERDLVSLFARFQEKKGPPIQFR
MMTGRMRGQAFITFPNKEIAWQALHLVNGYKLYGKILVIEFGKNKKQRSSGPSSG
2DB2: A putative ATP-dependent RNA helicase DHX30 isoform 1
(SEQ ID NO: 512)
GSSGSSGASRDLLKEFPQPKNLLNSVIGRALGISHAKDKLVYVHTNGPKKKKVTLHIKWP
KSVEVEGYGSKKIDAERQAAAAACQLFKGWGLLGPRNELFDAAKYRVLADRFGSGPSSG
2CSH: A zinc finger and BTB domain-containing protein 43
(SEQ ID NO: 513)
GSSGSSGDKLYPCQCGKSFTHKSQRDRHMSMHLGLRPYGCGVCGKKFKMKHHLVGHMKI
HTGIKPYECNICAKRFMWRDSFHRHVTSCTKSYEAAKAEQNTTEASGPSSG
2EBT: A Krueppel-like factor 5
(SEQ ID NO: 514)
GSSGSSGPDLEKRRIHYCDYPGCTKVYTKSSHLKAHLRTHTGEKPYKCTWEGCDWRFARS
DELTRHYRKHTGAKPFQCGVCNRSFSRSDHLALHMKRHQN
2GYR: A artemin isoform 3 precursor
(SEQ ID NO: 515)
GCRLRSQLVPVRALGLGHRSDELVRFRFCSGSCRRARSPHDLSLASLLGAGALRPPPGSR
PVSQPCCRPTRYEAVSFMDVNSTWRTVDRLSATACGCLGHHHHHH
2D8R: A THAP domain-containing protein 2
(SEQ ID NO: 516)
GSSGSSGMPTNCAAAGCATTYNKHINISFHRFPLDPKRRKEWVRLVRRKNFVPGKHTFLCS
KHFEASCFDLTGQTRRLKMDAVPTIFDFCTHISGPSSG
2CQL: A 60S ribosomal protein L9
(SEQ ID NO: 517)
GSSGSSGMKTILSNQTVDIPENVDITLKGRTVIVKGPRGTLRRDFNHINVELSLLGKKKK
RLRVDKWWGNRKELATVRTICSHVQNMIKGVTLGSGPSSG
2YU4: A E3 SUMO-protein ligase NSE2
(SEQ ID NO: 518)
GSSGSSGFTCPITKEEMKKPVKNKVCGHTYEEDAIVRMIESRQKRKKKAYCPQIGCSHTD
IRKSDLIQDEALRRAIENHNKKRHRHSESGPSSG
2DMD: A zinc finger protein 64 isoform a
(SEQ ID NO: 519)
GSSGSSGPHKCEVCGKCFSRKDKLKTHMRCHTGVKPYKCKTCDYAAADSSSLNKHLRIHS
DERPFKCQICPYASRNSSQLTVHLRSHTGDSGPSSG
2YT9: A POZ-, AT hook-, and zinc finger-containing protein 1 short
isoform
(SEQ ID NO: 520)
GSSGSSGVACEICGKIFRDVYHLNRHKLSHSGEKPYSCPVCGLRFKRKDRMSYHVRSHDGS
VGKPYICQSCGKGFSRPDHLNGHIKQVHSGPSSG
2E5H: A zinc finger CCHC-type and RNA-binding motif-containing
protein 1
(SEQ ID NO: 521)
GSSGSSGMSGGLAPSKSTVYVSNLPFSLTNNDLYRIFSKYGKVVKVTIMKDKDTRKSKGVA
FILFLDKDSAQNCTRAINNKQLFGRVIKASIAI
2E6O: A HMG box-containing protein 1
(SEQ ID NO: 522)
GSSGSSGGTVSATSPNKCKRPMNAFMLFAKKYRVEYTQMYPGKDNRAISVILGDRWKKM
KNEERRMYTLEAKALAEEQKRLNPDCWK
2RPR: A FLYWCH-type zinc finger-containing protein 1 isoform a
(SEQ ID NO: 523)
GSSGSSGLRPLEFLRTSLGGRFLVHESFLYRKEKAAGEKVYWMCRDQARLGCRSRAITQGH
RIMVMRSHCHQPDLAGLEALRQRERL
2EBL: A COUP transcription factor 1
(SEQ ID NO: 524)
GSSGSSGIECVVCGDKSSGKHYGQFTCEGCKSFFKRSVRRNLTYTCRANRNCPIDQHHRN
QCQYCRLKKCLKVGMRREAVQRGSGPSSG
2DK5: A DNA-directed RNA polymerase III subunit RPC6
(SEQ ID NO: 525)
GSSGSSGDSQNAGKMKGSDNQEKLVYQIIEDAGNKGIWSRDVRYKSNLPLTEINKILKNL
ESKKLIKAVKSVAASKKKVYMLYNLSGPSSG
2EQZ: A high mobility group protein B3
(SEQ ID NO: 526)
GSSGSSGMAKGDPKKPKGKMSAYAFFVQTCREEHKKKNPEVPVNFAEFSKKCSERWKTMS
GKEKSKFDEMAKADKVRYDREMKDYG
2ENV: A peroxisome proliferator-activated receptor delta isoform 3
(SEQ ID NO: 527)
GSSGSSGMECRVCGDKASGFHYGVHACEGCKGFFRRTIRMKLEYEKCERSCKIQKKNRNK
CQYCRFQKCLALGMSHNAIRFGSGPSSG
1X57: A endothelial differentiation-related factor 1 isoform alpha
(SEQ ID NO: 528)
GSSGSSGDRVTLEVGKVIQQGRQSKGLTQKDLATKINEKPQVIADYESGRAIPNNQVLGK
IERAIGLKLRGKDIGKPIEKGPRAKSGPSSG
2DMN: A homeobox protein TGIF2LX
(SEQ ID NO: 529)
GSSGSSGKKRKGNLPAESVKILRDWMYKHRFKAYPSEEEKQMLSEKTNLSLLQISNWFINA
RRRILPDMLQQRRNDPSGPSSG
1HRY: A sex-determining region Y protein
(SEQ ID NO: 530)
VQDRVKRPMNAFIVWSRDQRRKMALENPRMRNSEISKQLGYQWKMLTEAEKWPFFQEAQ
KLQAMHREKYPNYKYRP
1HRA: A retinoic acid receptor beta isoform 1
(SEQ ID NO: 531)
MPRVYKPCFVCQDKSSGYHYGVSACEGCKGFFRRSIQKNMIYTCHRDKNCVINKVTRNRC
QYCRLQKCFEVGMSKESVRN
2DMQ: A LIM/homeobox protein Lhx9 isoform 1
(SEQ ID NO: 532)
GSSGSSGKRMRTSFKHHQLRTMKSYFAINHNPDAKDLKQLAQKTGLTKRVLQVWFQNAR
AKFRRNLLRQENGGVSGPSSG
2DMT: A homeobox protein BarH-like 1
(SEQ ID NO: 533)
GSSGSSGGEPGTKAKKGRRSRTVFTELQLMGLEKRFEKQKYLSTPDRIDLAESLGLSQLQ
VKTWYQNRRMKWKKSGPSSG
2YUU: A protein kinase C delta type
(SEQ ID NO: 534)
GSSGSSGKQAKIHYIKNHEFIATFFGQPTFCSVCKDFVWGLNKQGYKCRQCNAAIHKKCI
DKIIGRCTGTAANSRDTSGPSSG
2COT: A zinc finger and SCAN domain-containing protein 16
(SEQ ID NO: 535)
GSSGSSGRSEWQQRERRRYKCDECGKSFSHSSDLSKHRRTHTGEKPYKCDECGKAFIQRSH
LIGHHRVHTGSGPSSG
1X4U: A zinc finger FYVE domain-containing protein 27 isoform a
(SEQ ID NO: 536)
GSSGSSGRYPTNNFGNCTGCSATFSVLKKRRSCSNCGNSFCSRCCSFKVPKSSMGATAPE
AQRETVFVCASCNQTLSKSGPSSG
1O7Y: A C-X-C motif chemokine 10 precursor
(SEQ ID NO: 537)
VPLSRTVRCTCISISNQPVNPRSLEKLEIIPASQFCPRVEIIATMKKKGEKRCLNPESKA
IKNLLKAVSKEMSKRSP
2ENN: A protein kinase C theta type
(SEQ ID NO: 538)
GSSGSSGQRRGAIKQAKVHHVKCHEFTATFFPQPTFCSVCHEFVWGLNKQGYQCRQCNAAI
HKKCIDKVIAKCTGSA
2DN0: A zinc fingers and homeoboxes protein 3
(SEQ ID NO: 539)
GSSGSSGASIYKNKKSHEQLSALKGSFCRNQFPGQSEVEHLTKVTGLSTREVRKWFSDRR
YHCRNLKGSRSGPSSG
2DB6: A SH3 and cysteine-rich domain-containing protein 3
(SEQ ID NO: 540)
GSSGSSGEPPKLVNDKPHKFKDHFFKKPKFCDVCARMIVLNNKFGLRCKNCKTNIHEHCQS
YVEMQRCSGPSSG
2CSZ: A synaptotagmin-like protein 4
(SEQ ID NO: 541)
GSSGSSGLLEIKRKGAKRGSQHYSDRTCARCQESLGRLSPKTNTCRGCNHLVCRDCRIQE
SNGTWRCKVCSGPSSG
2CU7: A histone H2A deubiquitinase MYSM1
(SEQ ID NO: 542)
GSSGSSGYSVKWTIEEKELFEQGLAKFGRRWTKISKLIGSRTVLQVKSYARQYFKNKVKCG
LDKETPNQKTG
1X2N: A homeobox protein PKNOX1
(SEQ ID NO: 543)
GSSGSSGKNKRGVLPKHATNVMRSWLFQHIGHPYPTEDEKKQIAAQTNLTLLQVNNWFINA
RRRILQSGPSSG
2EPA: A Krueppel-like factor 10 isoform b
(SEQ ID NO: 544)
GSSGSSGPQIDSSRIRSHICSHPGCGKTYFKSSHLKAHTRTHTGEKPFSCSWKGCERRFA
RSDELSRHRRTH
2E1O: A hematopoietically-expressed homeobox protein HHEX
(SEQ ID NO: 545)
GSSGSSGKGGQVRFSNDQTIELEKKFETQKYLSPPERKRLAKMLQLSERQVKTWFQNRRAK
WRRSGPSSG
2CRA: A homeobox protein Hox-B13
(SEQ ID NO: 546)
GSSGSSGRKKRIPYSKGQLRELEREYAANKFITKDKRRKISAATSLSERQITIWFQNRRV
KEKKSGPSSG
2CTU: A zinc finger protein 483 isoform a
(SEQ ID NO: 547)
GSSGSSGKRQKIHLGDRSQKCSKCGIIFIRRSTLSRRKTPMCEKCRKDSCQEAALNKDEG
NESGKKTSGPSSG
1MGS: A growth-regulated alpha protein precursor
(SEQ ID NO: 548)
ASVATELRCQCLQTLQGIHPKNIQSVNVKSPGPHCAQTEVIATLKNGRKACLNPASPIVK
KIIEKMLNSDKSN
2DIM: A cell division cycle 5-like protein
(SEQ ID NO: 549)
GSSGSSGKGGVWRNTEDEILKAAVMKYGKNQWSRIASLLHRKSAKQCKARWYEWLDPSI
KKTEWSGPSSG
2COB: A ligand-dependent corepressor isoform 1
(SEQ ID NO: 550)
GSSGSSGRGRYRQYNSEILEEAISVVMSGKMSVSKAQSIYGIPHSTLEYKVKERLGTLKN
PPKKKMKLMR
1MSG: A growth-regulated alpha protein precursor
(SEQ ID NO: 551)
ASVATELRCQCLQTLQGIHPKNIQSVNVKSPGPHCAQTEVIATLKNGRKACLNPASPIVK
KIIEKMLNSDKS
2DJN: A homeobox protein DLX-5
(SEQ ID NO: 552)
GSSGSSGRKPRTIYSSFQLAALQRRFQKTQYLALPERAELAASLGLTQTQVKIWFQNKRS
KIKKSGPSSG
2E70: A transcription elongation factor SPT5 isoform a
(SEQ ID NO: 553)
GSSGSSGMSRGRGRRDNELIGQTVRISQGPYKGYIGVVKDATESTARVELHSTCQTISVD
RQRLTTVGSRR
1HDP: A POU domain, class 2, transcription factor 2 isoform 1
(SEQ ID NO: 554)
RRKKRTSIETNVRFALEKSFLANQKPTSEEILLIAEQLHMEKEVIRVWFCNRRQKEKRIN
PCX
3IY9: O 39S ribosomal protein L27, mitochondrial
(SEQ ID NO: 555)
ASKKSGGS SKNLGGKSSGRRQGIKKMEGHYVHAGNIIATQRHFRWHPGAHVGVGKNKCL
YALEEGIVRY
2JGX: A complement factor H isoform a precursor
(SEQ ID NO: 556)
XRKCYFPYLENGYNQNYGRKFVQGKSIDVACHPGYALPKAQTTVTCMENGWSPTPRCIRV
K
2JGW: A complement factor H isoform a precursor
(SEQ ID NO: 557)
XRKCYFPYLENGYNQNHGRKFVQGKSIDVACHPGYALPKAQTTVTCMENGWSPTPRCIRV
K
1BBO: A zinc finger protein 40
(SEQ ID NO: 558)
KYICEECGIRXKKPSMLKKHIRTHTDVRPYHCTYCNFSFKTKGNLTKHMKSKAHSKK
1P0T: 0 tumor necrosis factor receptor superfamily member 13C
(SEQ ID NO: 559)
MRRGPRSLRGRDAPAPTPCVPAECFDLLVRHCVACGLLRTPRPKPAGASSPAPRTALQPQ
ESV
1BA5: A telomeric repeat-binding factor 1 isoform 1
(SEQ ID NO: 560)
RKRQAWLWEEDKNLRSGVRKYGEGNWSKILLHYKFNNRTSVMLKDRWRTMKKL
2CPW: A ubiquitin-associated and SH3 domain-containing protein B
(SEQ ID NO: 561)
GSSGSSGRNRQQRPGTIKHGSALDVLLSMGFPRARAQKALASTGGRSVQTACDWLFSHSGP
SSG
2DAS: A zinc finger MYM-type protein 5 isoform 3
(SEQ ID NO: 562)
GSSGSSGQPTAQQQLTKPAKITCANCKKPLQKGQTAYQRKGSAHLFCSTTCLSSFSSGPS
SG
3IY9: P 39S ribosomal protein L33, mitochondrial isoform a
(SEQ ID NO: 563)
AKSKSKNILVRMVSEAGTGFCFNTKRNRLREKLTLLHYDPVVKQRVLFVEKK
2YSA: A E3 ubiquitin-protein ligase RBBP6 isoform 1
(SEQ ID NO: 564)
GSSGSSGYTCFRCGKPGHYIKNCPTNGDKNFESGPRIKKSTGIPRSFMMEVKDPN
2YQQ: A zinc finger HIT domain-containing protein 3
(SEQ ID NO: 565)
GSSGSSGLKCSTVVCVICLEKPKYRCPACRVPYCSVVCFRKHKEQCNPETSGPSSG
2KZA: A agouti-signaling protein precursor
(SEQ ID NO: 566)
KKVVRPRTPLSAPCVATRNSCKPAAAACCDPCASCYCRFFRSACYCRVLSLNC
1Z6V: A lactotransferrin isoform 1 precursor
(SEQ ID NO: 567)
GRRRRSVQWCAVSQPEATKCFQWQRNMRKVRGPPVSCIKRDSPIQCIQA
1QGK: B importin subunit alpha-2
(SEQ ID NO: 568)
AARLHRFKNKGKDSTEMRRRRIEVNVELRKAKKDDQMLKRRNVS
2EPR: A POZ-, AT hook-, and zinc finger-containing protein 1 short
isoform
(SEQ ID NO: 569)
GSSGSSGRTRKQVACEICGKIFRDVYHLNRHKLSHSGEKPYSSGPSSG
1HTR: P gastricsin isoform 2 preproprotein
(SEQ ID NO: 570)
AVVKVPLKKFKSIRETMKEKGLLGEFLRTHKYDPAWKYRFGDL
2P8Q: B snurportin-1
(SEQ ID NO: 571)
HPRLSQYKSKYSSLEQSERRRRLLELQKSKRLDYVNHARR
2ENT: A Krueppel-like factor 15
(SEQ ID NO: 572)
GSSGSSGTGEKPFACTWPGCGWRFSRSDELSRHRRSHSGVKPSGPSSG
2EOY: A zinc finger protein 473
(SEQ ID NO: 573)
GSSGSSGQKEKCFKCNKCEKTFSCSKYLTQHERIHTRGVKSGPSSG
2YTD: A zinc finger protein 473
(SEQ ID NO: 574)
GSSGSSGSGEKPYKCSECGKAFHRHTHLNEHRRIHTGYRPSGPSSG
1JUN: A transcription factor AP-1
(SEQ ID NO: 575)
XCGGRIARLEEKVKTLKAQNSELASTANMLREQVAQLKQKVMNX
2EOV: A zinc finger protein 484 isoform a
(SEQ ID NO: 576)
GSSGSSGTGEKPYKCSDCGKSFTWKSRLRIHQKCHTGERHSGPSSG
2EN4: A zinc finger protein 347 isoform a
(SEQ ID NO: 577)
GSSGSSGTKEKPYKCYECGKAFRTRSNLTTHQVIHTGEKRSGPSSG
2EOH: A zinc finger protein 28 homolog
(SEQ ID NO: 578)
GSSGSSGSGKKPYECKECRKTFIQIGHLNQHKRVHTGERSSGPSSG
2ENE: A zinc finger protein 347 isoform a
(SEQ ID NO: 579)
GSSGSSGTGEKPYKCNECGKVFRHNSYLSRHQRIHTGEKPSGPSSG
2YTS: A zinc finger protein 484 isoform a
(SEQ ID NO: 580)
GSSGSSGTGEKPYICNECGKSFIQKSHLNRHRRIHTGEKPSGPSSG
2EQ0: A zinc finger protein 347 isoform a
(SEQ ID NO: 581)
GSSGSSGTGEKPYKCHECGKVFRRNSHLARHQLIHTGEKPSGPSSG
2EL6: A zinc finger protein 268 isoform c
(SEQ ID NO: 582)
GSSGSSGAGVNPYKCSQCEKSFSGKLRLLVHQRMHTREKPSGPSSG
2EMA: A zinc finger protein 347 isoform a
(SEQ ID NO: 583)
GSSGSSGTGEKRYKCNECGKVFSRNSQLSQHQKIHTGEKPSGPSSG
2EM9: A zinc finger protein 224
(SEQ ID NO: 584)
GSSGSSGTGEKPYNCKECGKSFRWASCLLKHQRVHSGEKPSGPSSG
2YTJ: A zinc finger protein 484 isoform a
(SEQ ID NO: 585)
GSSGSSGTGEKPYICAECGKAFTIRSNLIKHQKIHTKQKPSGPSSG
2EMB: A zinc finger protein 473
(SEQ ID NO: 586)
GSSGSSGHTRKRYECSKCQATFNLRKHLIQHQKTHAAKSGPSSG
2EM2: A zinc finger protein 28 homolog
(SEQ ID NO: 587)
GSSGSSGSGEKPFKCKECGKAFRQNIHLASHLRIHTGEKPSGPSSG
2EM4: A zinc finger protein 28 homolog
(SEQ ID NO: 588)
GSSGSSGTGQRPYECIECGKAFKTKSSLICHRRSHTGEKPSGPSSG
2EN9: A zinc finger protein 28 homolog
(SEQ ID NO: 589)
GSSGSSGAGKKLFKCNECKKTFTQSSSLTVHQRIHTGEKPSGPSSG
2YU8: A zinc finger protein 347 isoform a
(SEQ ID NO: 590)
GSSGSSGTGEKPYKCNECGKVFTQNSHLARHRRVHTGGKPSGPSSG
2EMZ: A zinc finger protein with KRAB and SCAN domains 5
(SEQ ID NO: 591)
GSSGSSGSGERPFKCNECGKGFGRRSHLAGHLRLHSREKSSGPSSG
2YTR: A zinc finger protein 347 isoform a
(SEQ ID NO: 592)
GSSGSSGTGEKPYKCNECGKAFSQTSKLARHQRIHTGEKPSGPSSG
2EPQ: A POZ-, AT hook-, and zinc finger-containing protein 1 short
isoform
(SEQ ID NO: 593)
GSSGSSGEKPYSCPVCGLRFKRKDRMSYHVRSHDGSVGKSGPSSG
2YU5: A zinc finger protein 473
(SEQ ID NO: 594)
GSSGSSGAGENPFKCSKCDRVFTQRNYLVQHERTHARKSGPSSG
2YTN: A zinc finger protein 347 isoform a
(SEQ ID NO: 595)
GSSGSSGTGKKPYKCNECGKVFTQNSHLARHRGIHTGEKPSGPSSG
2EOQ: A zinc finger protein 224
(SEQ ID NO: 596)
GSSGSSGTGEKPFKCDICGKSFCGRSRLNRHSMVHTAEKPSGPSSG
2L9U: A receptor tyrosine-protein kinase erbB-3 isoform 1 precursor
(SEQ ID NO: 597)
MGRTHLTMALTVIAGLVVIFMMLGGTFLYWRGRRHHHHHH
2ELY: A zinc finger protein 224
(SEQ ID NO: 598)
GSSGSSGTGEKPFKCVECGKGFSRRSALNVHHKLHTGEKPSGPSSG
2EML: A zinc finger protein 28 homolog
(SEQ ID NO: 599)
GSSGSSGTGEKPYECSVCGKAFSHRQSLSVHQRIHSGKKPSGPSSG
2EQ2: A zinc finger protein 347 isoform b
(SEQ ID NO: 600)
GSSGSSGTGGKPYQCNECGKAFSQTSKLARHQRVHTGEKPSGPSSG
2EPU: A zinc finger protein 32
(SEQ ID NO: 601)
GSSGSSGTGQKPFECTHCGKSFRAKGNLVTHQRIHTGEKSGPSSG
1SGH: B Na(+)/H(+) exchange regulatory cofactor NHE-RF1
(SEQ ID NO: 602)
CLDFNISLAMAKERAHQKRSSKRAPQMDWSKKNELFSNL
2EL4: A zinc finger protein 268 isoform c
(SEQ ID NO: 603)
GSSGSSGTGVKPYGCSQCAKTFSLKSQLIVHQRSHTGVKPSGPSSG
2L3H: A prostatic acid phosphatase isoform PAP precursor
(SEQ ID NO: 604)
GIHKQKEKSRLQGGVLVNEILNHMKRATQIPSYKKLIMX
2YRH: A zinc finger protein 473
(SEQ ID NO: 605)
GSSGSSGKKPLVCNECGKTFRQSSCLSKHQRIHSGEKPSGPSSG
2EOG: A zinc finger protein 268 isoform c
(SEQ ID NO: 606)
GSSGSSGVKPYGCSECGKAFRSKSYLIIHMRTHTGEKPSGPSSG
1FQQ: A beta-defensin 4B
(SEQ ID NO: 607)
XIGDPVTCLKSGAICHPVFCPRRYKQIGTCGLPGTKCCKKP
2ELU: A zinc finger protein ZFAT isoform 4
(SEQ ID NO: 608)
GSSGSSGIKQHCRFCKKKYSDVKNLIKHIRDAHDPQD
1BH7: A band 3 anion transport protein
(SEQ ID NO: 609)
XQLFDRILLLFKPPKYHPDVPYVKRVKTWRMHL
2ELM: A zinc finger protein ZFAT isoform 4
(SEQ ID NO: 610)
GSSGSSGHLYYCSQCHYSSITKNCLKRHVIQKHSNIL
2ELS: A zinc finger protein ZFAT isoform 1
(SEQ ID NO: 611)
GSSGSSGKIFTCEYCNKVFKFKHSLQAHLRIHTNEK
(+36)GFP
(SEQ ID NO: 663)
ASKGERLFRGKVPILVELKGDVNGHKFSVRGKGKGDATRGKLTLKFICTTGKLPVPWPTLV
TTLTYGVQCFSRYPKHMKRHDFFKSAMPKGYVQERTISFKKDGKYKTRAEVKFEGRTLVN
RIKLKGRDFKEKGNILGHKLRYNFNSHKVYITADKRKNGIKAKFKIRHNVKDGSVQLADHY
QQNTPIGRGPVLLPRNHYLSTRSKLSKDPKEKRDHMVLLEFVTAAGIKHGRDERYK
Myc-(+36)GFP-GS10-C4 scFv-His6
(SEQ ID NO: 664)
MEQKLISEEDLGSASKGERLFRGKVPILVELKGDVNGHKFSVRGKGKGDATRGKLTLKFICT
TGKLPVPWPTLVTTLTYGVQCFSRYPKHMKRHDFFKSAMPKGYVQERTISFKKDGKYKTR
AEVKFEGRTLVNRIKLKGRDFKEKGNILGHKLRYNFNSHKVYITADKRKNGIKAKFKIRHN
VKDGSVQLADHYQQNTPIGRGPVLLPRNHYLSTRSKLSKDPKEKRDHMVLLEFVTAAGIKH
GRDERYKGHGGGGSGGGGSQVQLQESGGGLVQPGGSLRLSCAASGFTFSSYSMSWVRQAP
GKGLEWVAVISYDGSNKYYADSVKGRFTISRDNSKNTLYLQMNSLRAEDTAVYYCARDRY
FDLWGRGTLVTVSSGGGGSGGGGSGGGGSQSALTQPASVSGSPGQSITISCTGTSSDIGAYN
YVSWYQQYPGKAPKLLIYDVSNRPSGISNRFSGSKSGDTASLTISGLQAEDEADYYCSSFAN
SGPLFGGGTKVTVLGGHGHHHHHH
Myc-(+36)GFP-His6
(SEQ ID NO: 665)
MEQKLISEEDLGSASKGERLFRGKVPILVELKGDVNGHKFSVRGKGKGDATRGKLTLKFICT
TGKLPVPWPTLVTTLTYGVQCFSRYPKHMKRHDFFKSAMPKGYVQERTISFKKDGKYKTR
AEVKFEGRTLVNRIKLKGRDFKEKGNILGHKLRYNFNSHKVYITADKRKNGIKAKFKIRHN
VKDGSVQLADHYQQNTPIGRGPVLLPRNHYLSTRSKLSKDPKEKRDHMVLLEFVTAAGIKH
GRDERYKGHGHHHHHH
Domain of FGF-10 (residues 64-208 of full length, unprocessed,
naturally occurring human FGF-10)
(SEQ ID NO: 666)
GRHVRSYNHLQGDVRWRKLFSFTKYFLKIEKNGKVSGTKKENCPYSILEITSVEIGVVAV
KAINSNYYLAMNKKGKLYGSKEFNNDCKLKERIEENGYNTYASFNWQHNGRQMYVALNG
KGAPRRGQKTRRKNTSAHFLPMVVHS
FGF10(Mut4)(variant of 64-208 fragment of full length, unprocessed,
naturally occurring human FGF-10)
(SEQ ID NO: 667)
GRHVRSYNHLQGDVAWRKLFSFTKYFLKIEKNGKVSGTKKENCPYSILEIRSVEIGVVAVK
AINSNYYLAMNKKGKLYGSKEFNNDCKLKERIEANGYNTYASFNWQHNGRQMYVALNGK
GAPRRGQKTRRANTSAHFLPMVVHS
Myc-FGF 10(mut4)-GS10-C4 scFv-His6
(SEQ ID NO: 668)
MEQKLISEEDLGSGRHVRSYNHLQGDVAWRKLFSFTKYFLKIEKNGKVSGTKKENCPYSIL
EIRSVEIGVVAVKAINSNYYLAMNKKGKLYGSKEFNNDCKLKERIEANGYNTYASFNWQH
NGRQMYVALNGKGAPRRGQKTRRANTSAHFLPMVVHSGHGGGGSGGGGSQVQLQESGG
GLVQPGGSLRLSCAASGFTFSSYSMSWVRQAPGKGLEWVAVISYDGSNKYYADSVKGRFTI
SRDNSKNTLYLQMNSLRAEDTAVYYCARDRYFDLWGRGTLVTVSSGGGGSGGGGSGGGG
SQSALTQPASVSGSPGQSITISCTGTSSDIGAYNYVSWYQQYPGKAPKLLIYDVSNRPSGISN
RFSGSKSGDTASLTISGLQAEDEADYYCSSFANSGPLFGGGTKVTVLGGHGHHHHHH
Myc-FGF10(mut4)-His6
(SEQ ID NO: 669)
MEQKLISEEDLGSGRHVRSYNHLQGDVAWRKLFSFTKYFLKIEKNGKVSGTKKENCPYSIL
EIRSVEIGVVAVKAINSNYYLAMNKKGKLYGSKEFNNDCKLKERIEANGYNTYASFNWQH
NGRQMYVALNGKGAPRRGQKTRRANTSAHFLPMVVHSGHGHHHHHH

### INCORPORATION BY REFERENCE

All publications and patents mentioned herein are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A fusion protein comprising
a Surf+ Penetrating Polypeptide having surface positive charge, net positive charge, a molecular weight of at least 4 kDa, and a charge per molecular weight ratio of greater than 0.75; and
an antibody or antibody-mimic moiety (AAM moiety) that binds to an intracellular target
and inhibits binding between the target and another protein, wherein the AAM moiety that binds the intracellular target is a single chain Fv (scFv) comprising a variable heavy chain (V_{H}) domain and a variable light chain (V_{L}) domain;
wherein the fusion protein penetrates cells and binds to the intracellular target to inhibit binding between the target and another protein inside the cells.

2. The fusion protein of claim 1, wherein the Surf+ Penetrating Polypeptide is a polypeptide engineered to comprise an overall charge from about +10 to about +40.

3. A fusion protein comprising
a Surf+ Penetrating Polypeptide, wherein the Surf+ Penetrating Polypeptide is a domain of a full length, naturally occurring human polypeptide, wherein the domain of the full length, naturally occurring human polypeptide has a molecular weight of at least 4 kDa and a charge per molecular weight ratio of greater than 0.75, and wherein the domain of a full length, naturally occurring human polypeptide has a charge/molecular weight ratio greater than that of the full length, naturally occurring human polypeptide; and
an antibody or antibody-mimic moiety (AAM moiety) that binds to an intracellular target and inhibits binding between the target and another protein, wherein the AAM moiety is a single chain Fv (scFv) comprising a variable heavy chain (V_{H}) domain and a variable light chain (V_{L}) domain;
wherein the fusion protein penetrates cells and binds to the intracellular target to inhibit binding between the target and another protein inside the cells; and
wherein the fusion protein does not include the full length, naturally occurring human polypeptide.

4. The fusion protein of claim 3, wherein the domain of a full length, naturally occurring human polypeptide has:
(a) a theoretical net charge of about +5 to +17 or about +10 to +20;
(b) a charge per molecular weight ratio greater than that of the full length, naturally occurring human polypeptide;
(c) a theoretical net charge of about +12; or
(d) a theoretical net charge of about +14; or
(e) a theoretical net charge of about +15;
(f) a theoretical net charge of about +16;
(g) a molecular weight of at least about 14 kDa;
(h) a molecular weight of at least about 15 kDa;
(i) less than 150 amino acid residues;
(j) a charge/molecular weight ratio of at least 1.0; or
(k) a charge/molecular weight ratio of at least 0.9.

5. (canceled)

6. The fusion protein of claim 3, wherein the full length, naturally occurring human polypeptide has a charge per molecular weight ratio of less than 0.75.

7-15. (canceled)

16. The fusion protein of claim 3, wherein the domain of a full length, naturally occurring human polypeptide is that of a full length, naturally occurring fibroblast growth factor receptor 10 (FGF-10).

17. The fusion protein of claim 3, wherein the domain is a variant comprising one, two, three, four, or five amino acid substitutions, deletions, and/or additions relative to the corresponding domain of the naturally occurring polypeptide.

18. The fusion protein of claim 1, wherein the Surf+ Penetrating Polypeptide is a domain of a full length, naturally occurring, human fibroblast growth factor receptor 10 (FGF-10).

19. The fusion protein of claim 3, wherein the domain has the amino acid sequence set forth in SEQ ID NO: 666.

20-21. (canceled)

22. The fusion protein of claim 1, wherein the Surf+ Penetrating Polypeptide and the AAM moiety are interconnected by a linker.

23-29. (canceled)

30. A nucleic acid comprising a nucleotide sequence encoding the fusion protein of claim 1.

31. A vector comprising the nucleic acid of claim 30.

32. A host cell comprising the vector of claim 31.

33. A method of making a fusion protein, comprising
(i) providing the host cell of claim 32 in culture media and culturing the host cell under suitable condition for expression of protein therefrom; and
(ii) expressing the fusion protein.

34. (canceled)

35. A method of inhibiting activity of an intracellular target in a cell, comprising
providing the fusion protein of claim 1, and
contacting cells that express the target with the complex.

36. A composition comprising the fusion protein of claim 1 and a pharmaceutically acceptable carrier.

37-38. (canceled)

39. A method of modulating activity of an intracellular target in a cell, comprising
providing the fusion protein of claim 1, which fusion protein penetrates cells, binds to the intracellular target and inhibits binding between the target and another protein; and
contacting cells that express the intracellular target with the complex.

40. A complex comprising
a Surf+ Penetrating Polypeptide having a molecular weight of at least 4 kDa and a charge per molecular weight ratio of greater than 0.75 and
an AAM moiety;
wherein the Surf+ Penetrating Polypeptide is associated with the AAM moiety,
wherein the AAM moiety binds to an intracellular target, and wherein the intracellular target is distinct from the Surf+ Penetrating Polypeptide.

41-42. (canceled)

43. The complex of claim 40, wherein the complex further comprises a linker.

44. (canceled)

45. The complex of claim 40, wherein the Surf+ Penetrating Polypeptide is:
(a) a human polypeptide;
(b) a full-length, naturally occurring human polypeptide;
(c) a domain of a full length, naturally occurring human polypeptide;
(d) a domain of a full length, naturally occurring human protein, and wherein the complex does not include the full length, naturally occurring human protein; or
(e) a domain of a full length, naturally occurring human protein, and wherein the complex does not include sufficient additional amino acid sequence from said full length, naturally occurring human protein contiguous with said domain such that the charge/molecular weight of the first portion would be less than 0.75.

46-47. (canceled)

48. The complex of claim 45, wherein the domain of a full length, naturally occurring human polypeptide has:
(a) a charge/molecular weight ratio greater than that of the full length, naturally occurring human polypeptide; and/or
(b) a charge/molecular weight ratio of at least 0.75 but the full length, naturally occurring human polypeptide has a charge/molecular weight ratio of less than 0.75.

49-58. (canceled)

59. The complex of claim 40, wherein the AAM moiety comprises:
(a) a full length antibody molecule;
(b) an antibody fragment;
(c) a camelid antibody, an IgNAR, or an antibody like molecule comprising a target binding domain engineered into an Fc domain of the antibody like molecule;
(d) a bispecific antibody;
(e) an antibody-mimic comprising a protein scaffold; or
(f) a DARPin polypeptide, an Adnectin® polypeptide or an Anticalin® polypeptide.

60-75. (canceled)

76. The complex of claim 40, wherein the Surf+ Penetrating Polypeptide has an overall net charge of +5 to +17.

77-82. (canceled)

83. The complex of claim 40, wherein the Surf+ Penetrating Polypeptide is:
(a) a naturally occurring human polypeptide that is modified to increase its overall net charge; and/or
(b) a polypeptide engineered to comprise an overall charge from about +10 to about +40.

84-102. (canceled)

103. A method of delivering an AAM moiety into a cell, comprising
providing the complex of claim 40 and
contacting cells with the complex.

104. A method of inhibiting activity of an intracellular target in a cell, comprising
providing a complex comprising
a first portion comprising a Surf+ Penetrating Polypeptide and
a second portion comprising an AAM moiety, which AAM moiety binds to and inhibits the intracellular target;
contacting cells that express the target with the complex.

105-132. (canceled)

133. A composition comprising the complex of claim 40 and a pharmaceutically acceptable carrier.

134-137. (canceled)

138. A fusion protein comprising
a Surf+ Penetrating Polypeptide and an AAM moiety that binds an intracellular target.

139-167. (canceled)
